(19) United States
(12) Patent Application Publication
(10) Pub. No.: US 2006/0252056 A1
(43) Pub. Date: Nov. 9, 2006

(54) Markers for responsiveness to an erbB receptor tyrosine kinase inhibitor

(75) Inventors: Takashi Tsuruo, Tokyo (JP); Yusuke Nakamura, Kanagawa (JP); Saburo Sone, Tokushima (JP); Masahiro Fukuoka, Nara (JP)

Correspondence Address: PALMER & DODGE, LLP;KATHLEEN M. WILLIAMS, 111 HUNTINGTON AVENUE, BOSTON, MA, 02199, US

(73) Assignees: AstraZeneca UK Limited; The University of Tokyo

(21) Appl. No.: 11/290,173

(22) Filed: Nov. 30, 2005

Related U.S. Application Data
(63) Continuation of application No. PCT/GB04/02316, filed on Jun. 1, 2004.

(30) Foreign Application Priority Data
May 30, 2003 (GB) 0312451.8
Sep. 26, 2003 (GB) 0322636.2
Nov. 21, 2003 (GB) 0327132.7

(51) Int. Cl. C12Q 1/68; C07H 21/04

(52) U.S. Cl. 435/6; 536/24.3

(57) ABSTRACT

The invention relates to a set of isolated marker genes comprising at least one gene identified as having differential expression as between patients who are responders and non responders to an erbB receptor tyrosine kinase inhibitor; said gene set comprising one or more genes selected from at least the group consisting of the 51 genes listed herein including gene-specific oligonucleotides derived from said genes; and uses of such sets in diagnostic applications.

### RELATED APPLICATIONS

This application is a continuation of International Application No. PCT/GB2004/002316, which was filed on Jun. 1, 2004, which designated the United States and was published in English, and which claims the benefit of United Kingdom applications GB0312451.8, filed 30 May 2003, GB0322636.2, filed Sep. 26, 2003, and GB0327132.7, filed Nov. 21, 2003. The above mentioned references are incorporated herein by reference.

### FIELD OF THE INVENTION

The present invention relates to a method of personalized cancer therapy which employs a set of marker genes to predict whether a patient will respond to a chemotherapeutic agent and a kit for use in said method.

In particular, the method predicts patient response to erbB tyrosine kinase inhibitors. More particularly the method relates to those patients with cancers mediated alone or in part by erbB tyrosine kinase, especially patients with advanced Non-small Cell Lung Cancer (NSCLC), for example adenocarcinoma, using the levels of a set of marker genes having differential expression between responders and non responders to the erbB tyrosine kinase inhibitor.

### BACKGROUND TO THE INVENTION

Lung cancer is the leading cause of cancer death and is therefore a major health problem worldwide. In the treatment of this disease, chemotherapy is the mainstay, because the majority has locally advanced stage 3 (44%) or metastatic stage 4 (32%) disease at diagnosis [1]. Nevertheless, the findings of large meta-analysis revealed that platinum-based chemotherapy contributed to prolong the median survival time of patients with advanced non-small cell lung cancer (NSCLC) by only about 6 weeks compared with best supportive care [2].

In the last decade, many new cytotoxic agents have been developed including paclitaxel, docetaxel, gemcitabine, and vinorelbine, and have offered multiple choices for patients with advanced lung cancer. However, each regimen served only modest survival benefit compared with the cisplatin-based therapies [3], [4]. More recently, new therapeutic strategies including a number of molecular-targeted agents have been developed in an effort to overcome the limitations of conventional cytotoxic agents [5] [6].

In recent years it has been discovered that certain growth factor tyrosine kinase enzymes are important in the transmission of biochemical signals which initiate cell replication. They are large proteins which span the cell membrane and possess an extracellular binding domain for growth factors such as epidermal growth factor (EGF) and an intracellular portion which functions as a kinase to phosphorylate tyrosine amino acids in proteins and hence to influence cell proliferation.

Various classes of receptor tyrosine kinases are known (Wilks, Advances in Cancer Research, 1993, 60, 43-73) based on families of growth factors which bind to different receptor tyrosine kinases. The classification includes Class I receptor tyrosine kinases comprising the EGF family of receptor tyrosine kinases. This includes receptors for the ligands EGF, TGFα (also referred to as TGFA), amphiregulin (also referred to as AREG), betacellulin, heparin binding EGF, epiregulin and the neuregulins (including NRG-1, NRG-2, NRG-3 and NRG-4). More specifically, these receptors include those with a functional kinase domain called erbB I (EGFR), erbB2 (Neu, Her2) and erbB4 (Her 4), and erbB3 (her3), which does not), Class II receptor tyrosine kinases comprising the insulin family of receptor tyrosine kinases such as the insulin and IGFI receptors and insulin-related receptor (IRR) and Class III receptor tyrosine kinases comprising the platelet-derived growth factor (PDGF) family of receptor tyrosine kinases such as the PDGFα, PDGFβ and colony-stimulating factor 1 (CSF1) receptors.

It is known that the erbB family of receptor tyrosine kinases, which include EGFR, erbB2, erbB3 and erbB4, are frequently involved in driving the proliferation and survival of tumour cells (reviewed in Olayioye et al., EMBO J., 2000, 19, 3159). One mechanism by which this can occur is over expression of the receptor at the protein level, generally as a result of gene amplification. This has been observed in many common human cancers (reviewed in Klapper et at., Adv. Cancer Res., 2000, 77, 25) such as, non-small cell lung cancers (NSCLCs) including adenocarcinomas (Cerny et al., Brit. J. Cancer, 1986, 54, 265; Reubi et al, Int. J. Cancer 1990, 45, 269; Rusch et al, Cancer Research, 1993, 53, 2379; Brabender et al, Clin. Cancer Res., 2001, 7, 1850) as well as other cancers of the lung (Hendler et al., Cancer Cells, 1989, 7, 347.

As a consequence of the mis-regulation of one or more of these receptors, it is widely believed that many tumours become clinically more aggressive and so correlate with a poorer prognosis for the patient (Brabender et al, Clin. Cancer Res., 2001, 7, 1850; Ross et al, Cancer Investigation, 2001, 19, 554, Yu et al., Bioessays, 2000, 22.7, 673). In addition to these clinical findings, a wealth of pre-clinical information suggests that the erbB family of receptor tyrosine kinases are involved in cellular transformation. In addition to this, a number of pre-clinical studies have demonstrated that anti-proliferative effects can be induced by knocking out one or more erbB activities by small molecule inhibitors, dominant negatives or inhibitory antibodies (reviewed in Mendelsohn et al., Oncogene, 2000, 19, 6550).

Thus it has been recognised that inhibitors of these receptor tyrosine kinases should be of value as a selective inhibitor of the proliferation of mammalian cancer cells (Yaish et al. Science, 1988, 242, 933, Kolibaba et al, Biochimica et Biophysica Acta, 1997, 133, F217-F248; Al-Obeidi et al, 2000, Oncogene, 19, 5690-5701; Mendelsohn et al, 2000, Oncogene, 19, 6550-6565). In addition to this pre-clinical data, findings using inhibitory antibodies against EGFR and erbB2 (c-225 and trastuzumab respectively) have proven to be beneficial in the clinic for the treatment of selected solid tumours (reviewed in Mendelsohn et al, 2000, Oncogene, 19, 6550-6565).

A number of small molecule inhibitors of erbB family of receptor tyrosine kinases are known, particularly inhibitors of EGF and erbB2 receptor tyrosine kinases. For example European Patent Application No. 0566226 and International Patent Applications WO 96/33980 and WO 97/30034 disclose that certain quinazoline derivatives which possess an anilino substituent at the 4-position possess EGFR tyrosine kinase inhibitory activity and are inhibitors of the proliferation of cancer tissue including prostate cancer. It has been disclosed by J R Woodburn et al. in Proc. Amer. Assoc. Cancer Research, 1997, 38, 633 and Pharmacol. Ther. 1999, 82, 241-250 that the compound N-(3-chloro-4-fluorophenyl)-7-methoxy-6-(3-morpholinopropoxy)quinazolin-4-amine is a potent EGFR tyrosine kinase inhibitor. This compound is also known as Iressa (registered trade mark), gefitinib (United States Adopted Name), by way of the code number ZD1839 and Chemical Abstracts Registry Number 184475-35-2. The compound is identified hereinafter as gefitinib. Gefitinib has recently been approved in Japan for the treatment of inoperable or recurrent non-small cell lung cancer (NSCLC) and in the USA as a monotherapy for the treatment of patients with locally advanced metastatic NSCLC after failure of both platinum and docetaxel chemotherapies.

It is further known from International Patent Application WO 96/30347 that certain structurally-related quinazoline derivatives possessing an anilino substituent at the 4-position also possess EGFR tyrosine kinase inhibitory activity. It has been disclosed in WO 99/55683 that the compound N-(3-ethynylphenyl)-6,7-bis(2-methoxyethoxy)quinazolin-4-amine, or a pharmaceutically-acceptable salt thereof (linked to the code numbers CP 358774 and OSI-774, identified hereinafter by the code number OSI-774) is an EGFR TKI.

It is further known from International Patent Application WO 97/38983 that certain other structurally-related quinazoline derivatives possessing an anilino substituent at the 4-position also possess EGFR tyrosine kinase inhibitory activity. It has been disclosed in J. Med. Chem., 1999, 42,1803-1815 and WO 00/31048 that the compound 6-acrylamido-N-(3-chloro-4-fluorophenyl)-7-(3 morpholinopropoxy)quinazolin-4-amine (linked to the code numbers PD 183805 and CI 1033, identified hereinafter by the code number CI 1033) is an EGFR TKI.

It is further known from International Patent Application WO 97/02266 that certain other structurally-related heterocyclic derivatives also possess EGFR tyrosine kinase inhibitory activity. For example, the compound 4-[(1R)-1-phenylethylamino]-6-(4-hydroxyphenyl)-7H-pyrrolo[2,3-d]pyrimidine (linked to the code numbers PKI-166, CGP 75166 and CGP 59326, identified hereinafter by the code number PKI-166) is an EGFR TKI.

It is further known from European Patent Application No. 0787722 and International Patent Applications WO 98/50038, WO 99/09016 and WO 99/24037 that certain other structurally-related quinazoline derivatives possessing an anilino substituent at the 4-position also possess EGFR tyrosine kinase inhibitory activity. For example, the compound N-[4-(3-bromoanilino)quinazolin-6-yl]but-2-ynamide (linked to the code numbers CL-387785 and EKB-785, identified hereinafter by the code number CL-387785) is an EGFR TKI.

It is further known from Nature Medicine, 2000, 6, 1024-1028 and U.S. Pat. No. 6,002,008 that certain other structurally-related quinoline derivatives possessing an anilino substituent at the 4-position also possess EGFR tyrosine kinase inhibitory activity. For example, the compound 4-(3-chloro-4-fluoroanilino)-3-cyano-6-(4-dimethylaminobut-2(E)-enamido)-7-ethoxyquinoline (identified hereinafter by the code number EKB-569) is an EGFR TKI.

It is also known from WO 99/35146 and WO 01/04111 that certain other quinazoline derivatives are inhibitors of one or more of the erbB receptor tyrosine kinase inhibitors. For example the compound N-{3-chloro-4-[(3-fluorobenzyl)oxy]phenyl}-6-[5-({[2-(methylsulfonyl)ethyl]amino}methyl)-2-furyl]quinazolin-4-amine (also identified as lapatinib or GW2016 identified hereinafter by the code GW2016) is thought to be an inhibitor of both EGF and erbB2 receptor tyrosine kinases. Novartis AE788 is another suitable inhibitor compound.

Inhibition of erbB receptor tyrosine kinase may also be achieved by inhibition of the extracellular ligand binding to a receptor using suitable antibodies against an erbB receptor. For example using the anti-erbB2 antibody trastuzumab [Herceptin™] and the anti-erbb1 antibody cetuximab [C225]). The use of such inhibitory antibodies have proven to be beneficial in the clinic for the treatment of selected solid tumours (reviewed in Mendelsohn et al, 2000, Oncogene, 19, 6550-6565).

As mentioned above, gefitinib is an oral active inhibitor of epidermal growth factor receptor-tyrosine kinase (EGFR-TK), which blocks signalling pathways responsible for driving proliferation, invasion, and survival of cancer cells [7]. Potent anti-tumour effects as well as rapid improvements in NSCLC-related symptoms and quality of life have been observed in clinical studies that enrolled patients with advanced NSCLC who did not respond to platinum-based chemotherapy. In the randomized double-blind phase II monotherapy trial (the IDEAL 1 trial), use of gefitinib as 2nd or 3rd line of chemotherapy to advanced NSCLC achieved tumour response rate of 18.4% (95% CI: 11.0-25.9%), and in the IDEAL 2 trial, use as 3rd or 4th line of chemotherapy achieved that of 11.8% (95% CI: 6.2-19.7%) [8],[27],[28].

Moreover in these trials, the treatment of this drug achieved high disease control rate (54.4% in IDEAL 1, 42.2% in IDEAL 2) and overall symptom improvement rate (40.3% in IDEAL 1, 43.1% in IDEAL 2).

Those results were promising when compared with responses to conventional cytotoxic agents, but the fact remained that about half of the patients enrolled in these studies received non-effective treatment with no improvement in symptoms. Moreover, the medication exposed non-responders to adverse effects, including life threatening ones such as interstitial pneumonia [11].

Patients responses to the various chemotherapy treatments differ, therefore there is a need to find methods of predicting which treatment regimes best suit a particular patient.

There is an increasing body of evidence that suggests that patients responses to numerous drugs may be related to a patients genetic profile and that determination of the genetic factors that influence, for example, response to a particular drug could be used to provide a patient with a personalised treatment regime. Such personalised treatment regimes offer the potential to maximise therapeutic benefit to the patient, whilst minimising, for example side effects that may be associated with alternative and less effective treatment regimes. There is therefore a need for methods that can predict a patients response to a drug.

### SUMMARY OF THE INVENTION

It has been found that the sensitivity of certain cancers to chemotherapeutic agents can be predicted by gene expression and hence that the suitability of cancer patients for treatment with such chemotherapeutic agents can be determined by measuring the relative levels of particular genes in patient tissue.

Accordingly, the present invention provides an isolated set of marker genes comprising at least one gene identified as having differential expression as between patients who are responders and non responders to an erbB receptor tyrosine kinase inhibitor, said gene set comprising one or more genes selected from at least the group consisting of the 51 genes listed in Table 4 herein including gene-specific oligonucleotides derived from said genes. In Table 4, accession numbers are given for the genes on the GenBank database.

Sequences of these genes are described in Table 4a and Table 4b. As will be appreciated by those skilled in the art, sequences available at the given accession numbers represent only examples of sequences of the genes referred to in the table; alternative sequences, including sequences which comprise sequencing error corrections, allelic or other variations, splice mutants and the like are also included in the definition of the gene represented by the name used. In a most preferred embodiment, the sequences referred to are the sequences set forth at the accession numbers and specific sequences given and set out in detail in Table 4a.

In a further aspect the present invention provides a set of isolated marker genes comprising at least one gene identified as having differential expression as between patients who are responders and non responders to an erbB receptor tyrosine kinase inhibitor; said gene set selected from the group consisting of the 51 genes listed in Table 4 herein including gene-specific oligonucleotides derived from said genes.

The present invention permits the improved prognosis and hence quality of life of cancer patients by matching the treatments to individual patients and so making more effective use of the types of drug available.

A preferred set is at least one or more of the first 40 genes listed in Table 4 herein.

A further preferred set is at least one or more of the first 20 genes listed in Table 4 herein.

A further preferred set is at least one or more of the first 12 genes listed in Table 4 herein.

A preferred set is at least one or more of the first 5 genes listed in Table 4 herein.

An especially preferred set is the first 12 genes listed in Table 4a herein, namely FLJ22622 (e.g. GenBank NM_{—}024829), AREG (e.g. GenBank BC009799), C0R01C (e.g. GenBank NM_{—}014325), AVEN (e.g. GenBank BC010488), DUSP3 (e.g. GenBank NM_{—}004090, DJ473B4 (e.g. GenBank AI026836), PHLDA2 (e.g. GenBank BU500509), RBM7 (e.g. GenBank NM_{—}0106090), EST (GenBank BX0952512), OSMR (e.g. GenBank AI436027), GCLC (e.g. GenBank AI971137), COL4A3BP (e.g. GenBank BQ024877).

Preferably the inhibitor is selected from gefitinib, OSI-774, PKI-166, EKB-569, GW2016, CI-1033 and an anti-erbB antibody such as trastuzumab and cetuximab.

Most preferably the inhibitor is gefitinib.

The present invention is particularly suitable for use in predicting the response to the aforementioned chemotherapeutic agents in those patients or patient population with a cancer mediated alone, or in part, by an erbB tyrosine kinase. Such cancers include, for example, non-solid tumours such as leukaemia, multiple myeloma or lymphoma, and also solid tumours, for example bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval tumours.

The present invention is particularly suitable for identifying those patients with NSCLC, more particularly advanced NSCLC including advanced adenocarcinoma that will respond to treatment with chemotherapeutic agents such as an erbB receptor tyrosine kinase inhibitor as hereinbefore defined.

The present invention offers considerable advantages in the treatment of cancers such as NSCLC, especially advanced NSCLC by identifying “individual cancer profiles” of NSCLC and so determining which tumours would respond to gefitinib. This includes 1^{st} line treatment and any other treatment regimen, such as, for example chemotherapy failed patients.

The present invention is particularly useful in the treatment of patients with advanced NSCLC who have failed previous chemotherapy, such as platinum-based chemotherapy.

The present invention is also particularly useful in the treatment of patients with locally advanced (stage lIIB) or metastasized (stage IV) NSCLC who have received previous chemotherapy, such as platinum-based chemotherapy.

The present invention also provides a method of predicting the responsiveness of a patient or patient population with cancer-, for example lung cancer, to treatment with chemotherapeutic agents, especially erbB receptor tyrosine kinase inhibitors, comprising comparing the differential expression of a set of marker genes said marker genes selected from the gene sets as defined above.

Preferably the assessment of expression is performed by gene expression profiling using oligonucleotide-based arrays or cDNA-based arrays of any type; RT-PCR (reverse transcription—Polymerase Chain Reaction), real-time PCR, in-situ hybridisation, Northern blotting, Serial analysis of gene expression (SAGE) for example as described by Velculescu et al Science 270 (5235): 484-487, or differential display. Details of these and other methods can be found for example in Sambrook et al, 1989, Molecular Cloning: A Laboratory Manual). Preferably the assessment uses a microarray assay.

Alternatively, or in addition, the assessment uses an immunohistochemical assay.

In a further aspect, the present invention provides a kit for use in a method of predicting the responsiveness of a patient or patient population with cancer, to treatment with chemotherapeutic agents, especially erbB receptor tyrosine kinase inhibitors, comprising a marker gene set as defined above on a suitable support medium. Preferably the marker gene is attached to a support material or membrane such as nitrocellulose, or nylon or a plastic film or slide.

Preferably the kit comprises a microarray.

### BRIEF DESCRIPTION OF THE FIGURES

FIG. 1: Images illustrating laser-microbeam microdissection of four representative lung adenocarcinomas. The upper row shows the samples before dissection; the lower row, dissected cancer cells (H.E. stain X100). TBB indicates transbronchial biopsy; LN, lymph-node.

FIG. 2: Establishing a scoring system to predict the efficacy of gefitinib treatment.

A. Different prediction scores appear when the number of discriminating genes is changed. The number of the discriminating gene sets (from 5 to 51) corresponds to the number of selected genes from the top of the rank-ordered list in table 4. A larger value of classification score (CS) indicates better separation of the two groups.

B. Hierarchical clustering of 17 “learning” cases using 51 candidate genes for gefitinib-sensitivity (left), and 12 prediction genes that were finally selected for the GRS (right). The dendrograms represent similarities in expression patterns among individual cases; longer branches indicate greater differences. The two groups were most clearly separated by the 12-gene set.

C. Schematic distinction of responder, non-responder and “test cases” verified on the basis of the GRS. Red diamonds denote prediction scores for learning PR cases and blue diamonds represent learning PD cases. A pink triangle indicates a test PR case that had not been used for establishing GRS, and blue triangles indicate test PD cases. Yellow triangles indicate test SD cases that kept the SD status throughout the 4-month observation period, and green triangles indicate test cases once judged as SD at a certain-time point of the study but showed progression of the disease within three or four months after the start of treatment.

FIG. 3: Validation of GRS with semi-quantitative RT-PCR and immunohistochemical analyses.

A. Representative image of semi-quantitative RT-PCR analysis of RNAs from the PR and PD groups. OSMR and GCLC genes were over-expressed in non-responders (PD). The integrity of each cDNA template was controlled through amplification of ACTB.

B. Immunohistochemical staining of representative samples from fiberscopic transbronchial biopsy (TBB) and lymph-node (LN) biopsy from the same PD-patient (No. LC21), using anti-AREG antibody (X 200).

C. Immunohistochemical staining of representative samples from PD patients, using antibodies for other 4 prediction markers (TGFA, ADAM9, CD9, and OSMR) (X200).

FIG. 4: Serologic concentration of TGFA determined by ELISA in 5 PR, 10 SD, and 20 PD adenocarcinoma cases. The averaged serum levels of TGFA were shown as black bars: 19·0±2·8 pg/ml (mean±SE) in PD patients, 13·9±1·9 pg/ml in SD patients, and 12·8±1·4 pg/ml in PR patients.

FIG. 5: Anti-apoptotic effect of secreted AREG on gefitinib-sensitive PC-9 cells.

A. Expression of AREG transcript examined by semi-quantitative RT-PCR in lung-adenocarcinoma cell lines PC-9, NCI-H358, and -H522.

B. PC-9 cells cultured in medium supplemented with 10% FCS, in serum-free medium, or in serum-free conditioned medium (CM) obtained from cultures of NCI-H358 or -H522 cells. Each medium was replaced once with the same medium at the 48-hour time point; 72 hours after adding gefitinib at concentrations of 0·5 or 1·0 μM, cell viability was measured by MTT assays. The experiments were done in triplicate. The Y-axis indicates the relative MTT value (MTT in the presence of 0·or 1·0 μM gefitinib/MTT in the absence of gefitinib) of the cells incubated in different media.

C. Effect of AREG, secreted in an autocrine manner, on the resistance of NSCLC cells to gefitinib. At the start of culture, PC-9 cells were inoculated into medium containing 1·0 μM gefitinib and recombinant AREG protein (final concentrations of 1-100 ng/ml); 72 hours later, cell viability was measured by triplicate MTT assays (blue bars). The Y-axis indicates the relative MTT values (MTT at individual concentrations of AREG/MTT without AREG) of the cells.

Effect of AREG on the viability of NSCLC cells in the absence of 1·0 μM gefitinib was also studied. Individual PC-9 cells were added to medium containing recombinant AREG protein but no gefitinib; 72 hours later, viability was measured by triplicate MTT assays (red bars).

FIG. 6: Immunohistochemical analysis of amphiregulin expression in sections derived from PD and PR patients.

### DETAILED DESCRIPTION

The invention will be described in more detail and illustrated by the following examples which are meant to serve to assist one of ordinary skill in the art in carrying out the invention and are not intended in any way to limit the scope of the invention. Certain elements of the invention are also described in more detail below.

“Set of Isolated Marker Genes”

These are, according to the context of the embodiments described herein, a group of genes which can be used in classification or categorisation of patent response according to the invention.

“Differential Expression”

Genes that are either expressed at a higher or lower level as between groups of responders or nonresponders.

“Responders/Non Responders”

Objective tumour responses according to Union International Contre le Cancer/World Health Organization (U ICC/WHO) Criteria are categorised as follows: complete response (CR): no residual tumour in all evaluable lesions; partial response (PR): residual tumour with evidence of chemotherapy-induced 50% or greater decrease under baseline in the sum of all measurable lesions and no new lesions; stable disease (SD) residual tumour not qualified for CR; and progressive disease (PD): residual tumour with evidence of 25% or greater increase under baseline in the sum of all measurable lesions or appearance of new lesions. As defined herein, non responders are PD.

The present invention is particularly effective for determining those patients which are CR or PR

“ErbB Receptor Inhibitors Including, Without Limitation, ErbB Receptor Tyrosine Kinase Inhibitors”

This family includes EGF, erbB2 (HER), erbB3 (note that erbB3 does not have a functional kinase domain) and erbB4 as described in the background to the invention above.

“Gene-Specific Oligonucleotides”

These are intended to be unique to the respective genes so that, for example, fragments of the gene that uniquely identify the gene. Advantageously, a gene-specific oligonucleotide is between 5 and 50 nucleotides in length, preferably about 15 to 30 nucleotides, and most preferably about 23 nucleotides.

“Arrays or Microarrays”

Array technology and the various techniques and applications associated with it are described generally in numerous textbooks and documents. Gene array technology is particularly suited to the practice of the present invention. Methods for preparing microarrays are well known in the art. These include Lemieux et al., (1998), Molecular Breeding 4, 277-289, Schena and Davis. Parallel Analysis with Biological Chips. in PCR Methods Manual (eds. M. Innis, D. Gelfand, J. Sninsky), Schena and Davis, (1999), Genes, Genomes and Chips. In DNA Microarrays: A Practical Approach (ed. M. Schena), Oxford University Press, Oxford, UK, 1999), The Chipping Forecast (Nature Genetics special issue; January 1999 Supplement), Mark Schena (Ed.), Microarray Biochip Technology, (Eaton Publishing Company), Cortes, 2000, The Scientist 14[17]:25, Gwynne and Page, Microarray analysis: the next revolution in molecular biology, Science, Aug. 6, 1999; and Eakins and Chu, 1999, Trends in Biotechnology, 17, 217-218.

The technology is described in PCT/US01/10063 and US 2002 090979 and references therein.

Commercial suppliers include Affymetrix (California) and Clontech Laboratories (California).

Major applications for array technology include the identification of sequence (nucleotide sequence/nucleotide sequence mutation) and the determination of expression level (abundance) of nucleotide sequences. Gene expression profiling may make use of array technology, optionally in combination with proteomics techniques (Celis et al, 2000, FEBS Lett, 480(1):2-16; Lockhart and Winzeler, 2000, Nature 405(6788):827-836; Khan et al., 1999, 20(2):223-9). Other applications of array technology are also known in the art; for example, nucleotide sequence discovery, cancer research (Marx, 2000, Science 289: 1670-1672; Scherf, et al, 2000, Nat Genet;24(3):236-44; Ross et al, 2000, Nat Genet. March 2000; 24(3):227-35), SNP analysis (Wang et al, 1998, Science, 280(5366):1077-82), drug discovery, pharmacogenomics, disease diagnosis (for example, utilising microfluidics devices: Chemical & Engineering News, Feb. 22, 1999, 77(8):27-36), toxicology (Rockett and Dix (2000), Xenobiotica, 30(2):155-77; Afshari et al., 1999, Cancer Res1;59(19):4759-60) and toxicogenomics (a hybrid of functional genomics and molecular toxicology). The goal of toxicogenomics is to find correlations between toxic responses to toxicants and changes in the nucleotide sequencetic profiles of the objects exposed to such toxicants (Nuwaysir, et al (1999), Molecular Carcinonucleotide sequencesis, 24:153-159).

In general, any library may be arranged in an orderly manner into an array, by spatially separating the members of the library. Examples of suitable libraries for arraying include nucleic acid libraries (including DNA, nucleotide sequence, oligonucleotide, etc libraries), peptide, polypeptide and protein libraries, as well as libraries comprising any molecules, such as ligand libraries, among others. Accordingly, where reference is made to a “library” such reference includes reference to a library in the form of an array.

The members of a library are generally fixed or immobilised onto a solid phase, preferably a solid substrate, to limit diffusion and admixing of the samples. In particular, the libraries may be immobilised to a substantially planar solid phase, including membranes and non-porous substrates such as plastic and glass. Furthermore, the samples are preferably arranged in such a way that indexing (i.e. reference or access to a particular sample) is facilitated. Typically the samples are applied as spots in a grid formation. Common assay systems may be adapted for this purpose. For example, an array may be immobilised on the surface of a microplate, either with multiple samples in a well, or with a single sample in each well. Furthermore, the solid substrate may be a membrane, such as a nitrocellulose or nylon membrane (for example, membranes used in blotting experiments). Alternative substrates include glass, or silica based substrates. Thus, the samples are immobilised by any suitable method known in the art, for example, by charge interactions, or by chemical coupling to the walls or bottom of the wells, or the surface of the membrane. Other means of arranging and fixing may be used, for example, pipetting, drop-touch, piezoelectric means, ink-jet and bubblejet technology, electrostatic application, etc. In the case of silicon-based chips, photolithography may be utilised to arrange and fix the samples on the chip. The samples may be arranged by being “spotted” onto the solid substrate; this may be done by hand or by making use of robotics to deposit the sample. In general, arrays may be described as macroarrays or microarrays, the difference being the size of the sample spots. Macroarrays typically contain sample spot sizes of about 300 microns or larger and may be easily imaged by existing gel and blot scanners. The sample spot sizes in microarrays are typically less than 200 microns in diameter and these arrays usually contain thousands of spots. Thus, microarrays may require specialised robotics and imaging equipment, which may need to be custom made. Instrumentation is described generally in a review by Cortese, 2000, The Scientist 14[11]:26.

Techniques for producing immobilised libraries of DNA molecules have been described in the art. Generally, most prior art methods describe how to prepare single-stranded nucleic acid molecule libraries, using for example masking techniques to build up various permutations of sequences at the various discrete positions on the solid substrate. U.S. Pat. No. 5,837,832 describes an improved method for producing DNA arrays immobilised to silicon substrates based on very large scale integration technology. In particular, U.S. Pat. No. 5,837,832 describes a strategy called “tiling” to prepare specific sets of probes at spatially-defined locations on a substrate which may be used to produced the immobilised DNA libraries of the present invention. U.S. Pat. No. 5,837,832 also provides references for earlier techniques that may also be used.

To aid detection, targets and probes may be labelled with any readily detectable reporter such as a fluorescent, bioluminescent, phosphorescent, radioactive reporter. Labelling of probes and targets is disclosed in Shalon et al., 1996, Genome Res 6(7):639-45.

The materials for use in the methods of the present invention are ideally suited for preparation of kits. A set of instructions will typically be included.

General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

In a specific embodiment of the invention, a cDNA microarray system representing 27, 648 genes was used to select a set of genes predicating the responsiveness to gefitinib for advanced NSCLC. Statistical analysis of the expression profiles identified dozens of genes differentially expressed between responders and non-responders to gefitinib. A drug response scoring (DRS) system based on the expression of these genes successfully predicted the response to gefitinib therapy.

Materials and Methods

Patients and Tissue Samples

A phase II clinical study was carried out comprising a multi-center trial to explore the dominant biological factors responsible for clinical anti-tumor effect, adverse drug reactions (ADR) and pharmacokinetics of ZD1839 dosed 250 mg daily in patients with advanced non-small-cell lung cancer who have failed previous chemotherapy. The primary endpoint was to clarify a gene-expression profile that could determine in advance a potential anti-tumor effect of gefitinib. At the start of the study, the sample size was estimated using studies conducted thus far as a rationale.^{12,13} Since the response rate for gefitinib has been less than 20% in patients with lung cancer,⁸⁻¹⁰ about 50 patients were estimated to be required to obtain learning cases estimated above. Patients whose locally advanced (stage IIIB) or metastasized (stage IV) NSCLCs were resistant to one or more regimens of conventional chemotherapy were enrolled in this trial. Inclusion criteria were (1) age greater than 20 years, (2) Performance Status (PS) 0-2, (3) adequate liver and kidney function tests. All patients were treated with 250 mg of gefitinib orally once a day at the Tokushima University or Kinki University hospitals in Japan. The treatment was continued until the patient was dropped from the study due to (1) progression of disease, (2) intolerable toxicity, or (3) withdrawal of consent.

Objective tumor responses were assessed every 4 weeks after the beginning of treatment, according to criteria outlined by the Union International Contre le Cancer/World Health Organization (UICC/WHO). Response categories were as follows: complete response (CR), no residual tumor in any evaluable lesion; partial response (PR), residual tumor with evidence of 50% or greater decrease under baseline in the sum of all measurable lesions, and no new lesions; progressive disease (PD), residual tumor with evidence of 25% or greater increase under baseline in the sum of all measurable lesions, or appearance of new lesions; and stable disease (SD), residual tumor not qualified for CR, PR, or PD. All evaluable lesions were measured bi-dimensionally (sum of products of longest diameter and its longest perpendicular of measurable lesions) using the same techniques as baseline, e.g. plain X-ray, CT, or MRI.

At the end of 4-month treatment (or withdrawal), the best overall response was evaluated for each patient based on definitions as follows: CR, patients who qualified for CR at two sequential examination points with an interval of at least 28 days between them; PR, patients judged as PR or better at two sequential examination points with an interval of at least 28 days between them; SD, patients who were SD or better at two sequential examination points at least 28 days apart but who did not qualify as CR or PR. The first judgment of an SD case must be done at or after the first tumor assessment point (28 days after randomization); PD, the patients determined as PD at or before the first tumor assessment point (28 days after randomization); Unknown, the patient does not qualify for a best response of increased disease, and all objective statuses after baseline (before randomization) and before progression are unknown.

Prior to the gefitinib treatment, tumor specimens were taken by trans-bronchial (TBB), skin, or lymph-node biopsy with written informed consent from each patient. Ethics approval was obtained from the ethics committee of the individual institutes. Biopsy samples were frozen immediately, embedded in TissueTek OCT medium (Sakura, Tokyo, Japan), and stored at −80° C. All samples were examined microscopically, and samples from 28 patients (17 learning and 11 test cases) that contained enough cancer cells for analysis of expression profiles were initially selected for further analysis. For validation of the prediction system, a blinded set of samples from 5 newly enrolled cases (4 PD and 1 SD) were also added to the 11 test cases. Clinical and histological information about these patients is summarized in Table 1-3.

Microdissection

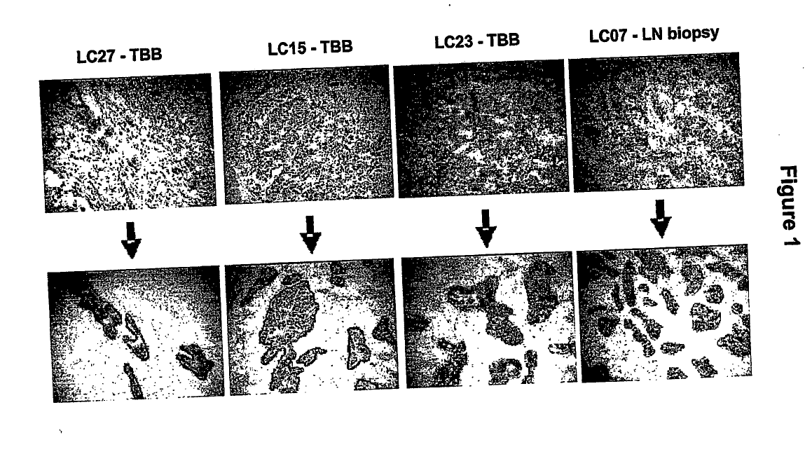

In view of significant differences in the proportions of cancer cells and various types of parenchymal cells that are present from one tumor to another, microdissection is a necessary means of obtaining precise gene-expression profiles on cDNA microarrays. Therefore we stained 8 μm-thick frozen sections with hematoxylin and eosin and collected cancer cells selectively, using the μCUT laser-microbeam microdissection system (Molecular Machines & Industries AG, Glattbrugg, Switzerland).¹⁴ In this system tissue sections are mounted on a thin supporting polyethylene membrane that will be cut together with the target tissue; a pulsed-ultraviolet (UV) narrow-beam-focus laser cuts out cancer cells along a pre-selected track that can be observed on a video screen. The material to be extracted is never directly exposed to the laser but only circumscribed by it; unlike other LMM systems, this one allows recovery of dissected cells to proceed without radiation. Moreover, the membrane protects the tissue on the slide against cross-contamination. Using this system we were able to isolate small areas of tissue rapidly, and to isolate single cells from histological sections (FIG. 1).

RNA Extraction and T7-Based RNA Amplification

Total RNA was extracted from individual microdissected populations of cancer cells using RNeasy mini kits and RNase-free DNase kits (QIAGEN, Hilden, Germany) according to the manufacturer's protocols. Total RNAs were subjected to T7-based RNA amplification, as described previously.¹⁵ Two rounds of amplification yielded 40-200 μg of aRNA (amplified RNA) (>100,000-fold) from each sample. As a control probe, normal human lung poly(A)⁺RNA (BD Biosciences Clontech, Palo Alto, Calif. and BIOCHAIN, Hayward, Calif., USA) was amplified in the same way. Aliquots (2·5 μg) of mRNA from individual samples and from the control were reversely transcribed in the presence of Cy5-dCTP and Cy3-dCTP respectively.

cDNA Microarray

Our “genome-wide” cDNA microarray system contains 27,648 cDNAs selected from the UniGene database of the National Center for Biotechnology Information.¹⁵ Fabrication of the microarray, hybridization, washing, and detection of signal intensities were described previously.¹⁵ To normalize the amount of mRNA between tumors and controls, the Cy5/Cy3 ratio for each gene's expression was adjusted so that the averaged Cy5/Cy3 ratio of 52 housekeeping genes was equal to one. We assigned a cutoff value to each microarray slide using analysis of variance, and the Cy5/Cy3 ratio of the gene was calculated as follows: (1) if Cy5 (cancer sample) was lower than the cut off level, then the Cy5/Cy3 ratio of the gene was substituted by 2-5 percentile among the Cy5/Cy3 ratios of other genes whose Cy5 and Cy3 were higher than the cut off level; (2) if Cy3 (control sample) was lower than the cut off level, then the Cy5/Cy3 ratio of the gene was substituted by 97·5 percentile among the Cy5/Cy3 ratios of other genes whose Cy5 and Cy3 were higher than the cut off level; (3) if both Cy5 and Cy3 were lower than the cut off level, then the Cy5/Cy3 ratio of the gene was left blank.

Extraction of Genes for Predicting Responsiveness to Gefitinib

To discover genes that might be associated with sensitivity to gefitinib, individual measurements of about 27,648 genes were compared between the two groups of patients, one classified as responders to gefitinib (PR) and the other as non-responders (PD). To reduce the dimensionality of the number of potent genes that could discriminate between the two classes, we extracted only genes that fulfilled two criteria: 1) signal intensities were higher than the cut-off level in at least 60% of either group, and 2) 1 MED_{PR}−MED_{PD}|≦1, where MED indicates the median calculated from log-transformed relative expression ratios in each group. Then random-permutation tests were applied to estimate the ability of individual genes to distinguish between the two classes (PR and PD); mean (μ) and standard deviations (σ) were calculated from the log-transformed relative expression ratios of each gene in both groups. A discrimination score (DS) for each gene was defined as follows:
DS=(μ_{PR}−μ_{PD})/( [symbol] _{PR}+σ_{PD}).

The samples were randomly permutated 10,000 times for each pair of groups. Since the DS dataset of each gene showed a normal distribution, we calculated a p-value for the user-defined grouping.

Calculation of Drug-Response Scores

We calculated the drug response scores for gefitinib (gefitinib response scores, or GRS) reflecting the expression levels of candidate prediction-genes according to procedures described previously.¹⁶⁻¹⁸ Each gene (gi) votes for either responder (PR) or non-responder (PD) depending on whether the expression level (xi) in the sample is closer to the mean expression level of one group or the other in reference samples. The magnitude of the vote (vi) reflects the deviation of the expression level in the sample from the average of the two classes:
Vi=|xi−(μ_{PR}+μ_{PD})/2|.

We summed the votes to obtain total votes for responders (V_{PR}) and non-responders (V_{PD}), and calculated GRS values as follows: GRS=((V_{PR}−V_{PD})/(V_{PR}+V_{PD}))×100, where the GRS value reflects the margin of victory in the direction of either responder or non-responder. GRS values range from −100 to 100; the higher an absolute value of GRS, the stronger the prediction.

Cross-Validation of Scores and Evaluation of the Prediction System

The prediction scores of all samples were obtained by a leave-one-out approach, in which one sample at a time was removed from the sample set; permutational p-values and mean values of the two classes were calculated for each gene using the remaining samples. The drug-response of the withheld sample was predicted by calculating the prediction score.

These procedures were repeated for each sample.¹⁶⁻¹⁷

To evaluate the reliability of the prediction system, we calculated a “classification score” (CS) using the GRS values of responders and non-responders in each gene set, as follows:
CS=(μ_{GRSpr}−μ_{GRSpd})/( [symbol] _{GRSpr}+ [symbol] _{GRSpd}).¹⁷

A larger value of CS indicates better separation of the two groups by the prediction system.

Hierarchical Clustering

We used web-available software (“Cluster” and “TreeView”) written by M. Eisen (http://genome-www5.stanford.edu/MicroArray/SMD/restech.html) to create a graphic representation of the microarray data and to create a dendrogram of hierarchical clustering. Before the clustering algorithm was applied, the fluorescence ratio for each spot was first log-transformed and then the data for each sample were median-centered to remove experimental biases.

Semi-Quantitative RT-PCR Analysis

Aliquots (5·0 μg) of the same aRNA hybridized to the microarray slides from individual samples and from the normal control lung were reversely transcribed using oligo(dT)₁₂₋₁₈ primer and SuperScript II reverse transcriptase (Invitrogen, Carlsbad, Calif., USA). Semi-quantitative RT-PCR experiments were carried out with the following sets of synthesized primers specific to the 12 top-ranked genes used for establishing a GRS or with beta-actin (ACTB)-specific primers as an internal control: FLJ22662, 5′-GCCATAAGTGGTCCCACAGT-3′ and 5′-GTCTTCTAGTCCGTCATCTCCCT-3′; Amphiregulin (AREG), 5′-CCATAGCTGCCTTTATGTCTGC-3′ and 5′-CTTTTTACCTTCGTGCACCTTT-3′, coronin, actin binding protein, IC (COROIC), 5′-TAATCTGCTGAGGACCTTTTGTC-3′ and 5′-TAATTCACTGTCCTCTTCTGGGA-3′; apoptosis, caspase activation inhibitor (AVEN), 5′-GCTCACAGCAGTAAATGCCTA-3′ and 5′-TGCTATGCTGTAAACACTGGCTA-3′; dual specificity phosphatase 3 (DUSP3), 5′-GGATCCTTTATTGGTGGTAGAGC-3′ and 5′-CCAGAGTGACCCTGAAGATAAAT-3′; DJ473B4, 5′-ACCTGATTCTCTAGGTGCAGTTT-3′ and 5′-GTCGTTTCAACCAGGTAGTTTTG-3′; pleckstrin homology-like domain, family A, member 2 (PHLDA2), 5′-GGGCGCCTTAAGTTATTGGA-3′ and 5′-GGATGGTAGAAAAGCAAACTGG-3′; RNA binding motifprotein 7 (RBM7), 5′-TGTAATGGAGATTGTACAGGTTG-3′ and 5′-AGGAACAGTACAAATGCTGTGGT-3′; BX092512 (EST), 5′-GCACTCCTTGAAGGTACACTAAC-3′ and 5′-ATTTGTATTCACTCAGCCATGC-3′; oncostatin M receptor (OSMR), 5′-ACCCAACTTCAAAACTAGGACTC-3′ and 5′-ACAGCTTGATGTCCTTTCTATGC-3′, glutamate-cysteine ligase, catalytic subunit (GCLC), 5′-TCATGAAAGGCACTGAGTTTTG-3′ and 5′-GTTAGCTGAAGCAGCTTTATTGC-3′; collagen, type IV, alpha 3 binding protein (COL4A3BP), 5′-ATATGCACAATCCTGGAAGTGA-3′ and 5′-TGCCTTACTAGCATTACCACCAT-3′; ACTB, 5′-GAGGTGATAGCATTGCTTTCG-3′ and 5′-CAAGTCAGTGTACAGGTAAGC-3′. PCR reactions were optimized for the number of cycles to ensure product intensity within the logarithmic phase of amplification. We did phosphor imager quantification analysis (Molecular Imager FX: Bio-Rad Laboratories, Hercules, Calif., USA), and RT-PCR band intensities were quantitatively compared with normalized Cy5/Cy3 ratio of gene expression from the microarray data.

RT-PCR was performed to screen the mutation at entire region of codon 709-870 (from p-loop to activation loop) of EGFR which was recently reported as a hot spot of mutation,¹⁸ using three primer sets: fragment-1,5′-TCTTACACCCAGTGGAGAAGC-3′ and 5′-GTCTTTGTGTTCCCGGACAT-3′; fragment-2,5′-ACTATGTCCGGGAACACAAA-3′ and 5′-TTCCGTCATATGGCTTGG-3′; fragment-3,5′-CGTCGCTATCAAGGAATTAAGAG-3′ and 5′-GTAGCTCCAGACATCACTCTGGT-3′. RT-PCR products from 19 NSCLC patients treated with gefitinib were analyzed by direct sequencing.

Immunohistochemical Analysis

To confirm the differential expression of AREG and transforming growth factor-alpha (TGFA) proteins, both of which encode the ligand for EGFR and other ERBB members, and other 3 candidate markers (a disintegrin and metalloproteinase domain 9 (ADAM9), D9 antigen (p24), and OSMR), which are also known to relate to the EGFR signalling, for predicting responders vs non-responders to gefitinib, we stained clinical tissue sections obtained by fiberscopic transbronchial biopsy (TBB) and lymph-node biopsy using ENVISION+ Kit/HRP (DakoCytomation, Glostrup Denmark). Briefly, after endogenous peroxidase and protein blocking reactions, anti-human AREG polyclonal antibody (Neo Markers, Fremont, Calif., USA), anti-human TGFA monoclonal antibody (Calbiochem, Darmstadt, Germany), anti-human ADAM9 monoclonal antibody (R&D Systems Inc. Minneapolis, Minn., USA), anti-human CD9 monoclonal antibody (Novocastra Laboratories Ltd, Newcastle upon Tyne, UK), or anti-human OSMR monoclonal antibody (Santa Cruz Biotechnology, Inc., Santa Cruz, Calif., USA), was added, and then HRP-labeled anti-rabbit or anti-mouse IgG as the secondary antibody. Substrate-chromogen was then added and the specimens were counterstained with hematoxylin.

Frozen tissue samples from 11 patients were selected for analysis of immunohistochemistry. Positivity of immunostaining was assessed semi-quantitatively by scoring intensity as absent or positive by three independent investigators without prior knowledge of the clinical follow-up data. Cases were accepted only as positive if reviewers independently defined them thus.

ELISA

Serum was obtained from an independent set of 35 lung-ADC patients who were treated with gefitinib based on the same protocol as this clinical study at Hiroshima University hospital in Japan (5 for PR, 10 for SD, and 20 for PD). The sera of all the patients were obtained with informed consent at the time of diagnosis and every 4 weeks after the beginning of treatment, and stored at −80° C. The serum TGFA levels were measured by an ELISA using a commercially available enzyme test kits (TGF-alpha ELISA kit: Oncogene Rsearch Products, San Diego, Calif., USA).

In Vitro Gefitinib Treatment and AREG-Autocrine Assay

Human NSCLC (adenocarcinoma) cell lines PC-9, NCI-H358, and NCI-H522 were purchased from the American Type Culture Collection (ATCC; Rockville, Md., USA). To detect expression of AREG in these NSCLC cells, total RNA from each line was reverse-transcribed for single-stranded cDNAs using oligo(dT)₁₂₋₁₈ primer and Superscript II (Invitrogen). Semi-quantitative reverse transcriptase-PCR (RT-PCR) was carried out as described previously.¹⁴ gefitinib (4-(3-chloro-4-fluoroanilino)-7-methoxy-6-(3-morpholinopropoxy)quinazoline: ZD 1839, Iressa), an inhibitor of epidermal growth factor receptor tyrosine kinase, was provided by AstraZeneca Pharmaceuticals (Macclesfield, UK). The drug was dissolved in DMSO at a concentration of 10 mM and kept at −20° C.

We performed flow-cytometry to determine the sensitivity of lung adenocarcinoma cell lines to gefitinib treatment. Cells were plated at densities of 5×10 cells/100-mm dish and treated with 1·0 μM of gefitinib in appropriate serum-free medium. The cells were trypsinized 72 hours after the treatment, collected in PBS, and fixed in 70% cold ethanol for 30 min. After treatment with 100 μg/ml RNase (Sigma-Aldrich Co., St. Louis, Mo., USA), the cells were stained with 50 μg/ml propidium iodide (Sigma-Aldrich Co.) in PBS. Flow cytometry was performed on a Becton Dickinson FACScan and analyzed by ModFit software (Verity Software House, Inc., Topsham, Me., USA). The percentages of nuclei in G0/G1, S, and G2/M phases of the cell cycle and sub-G1 population were determined from at least 20,000 ungated cells.

To investigate whether AREG functions as an autocrine anti-apoptotic factor in lung adenocarcinoma cells treated with gefitinib, we carried out the following assay. First, gefitinib-sensitive PC-9 cells, which do not express AREG, were cultured in serum-free medium for at least 8 hours prior to gefitinib treatment. These cells were then incubated with 0·5 or 1·0 μM of gefitinib for 72 hours in media that were either serum-free or supplemented with 10% FCS, or in serum-free conditioned medium collected from 72-hour cultures of AREG-expressing cells (NCI-H358 or NCI-H522). Each medium was replaced once with the same medium containing gefitinib at the 48-hour time point. To detect the response of each cell line to gefitinib, viability was evaluated by MTT assays using Cell Counting Kits (WAKO, Osaka, Japan).

To confirm the autocrine effect of AREG on the gefitinib-resistance of NSCLC cells, we cultured PC-9 cells for 72 hours in serum-free medium containing 1·0 μM of gefitinib and recombinant AREG protein (Genzyme-Techne, Minneapolis, Minn., USA) in final concentrations of 1-100 ng/ml. Cell viability was evaluated by MTT assays. A possible effect of AREG itself on the viability of NSCLC cells was evaluated also, by culturing the PC-9 cells in serum- and gefitinib-free medium containing only recombinant AREG protein. MTT assays were performed as above.

Results

Response to Gefitinib Treatment

Of the 53 patients enrolled in this trial, 46 had tumors diagnosed as adenocarcinomas (86·8%); five were squamous-cell carcinomas (9·4%); two were large cell carcinomas (3·8%). Fifteen patients achieved a PR and nobody revealed a CR; 17 patients were classified as SD, and 19 as PD. No clinical-response data were available for two of the patients. The tumor-response rate (CR+PR/CR+PR+SD+PD) for this treatment was 29·4%, and the disease control rate (CR+PR+SD/CR+PR+SD+PD) was 62·8% (table 1).

Tumor samples were collected from 43 patients. Samples from 32 of those 43 contained sufficient numbers of cancer cells for analysis of expression profiles on our cDNA microarray. The numbers of samples that were judged to be suitable for further microarray analysis, were 8 for PR, 7 for SD, and 13 for PD (table 2). 17 of the 28 samples were analyzed as learning cases (7 for PR and 10 for PD), and 11 were as test cases (1 for PR, 3 for PD, and 7 for SD) for establishing a predictive scoring system for the efficacy of gefitinib treatment. For further validation of the prediction system, another blinded set of samples from 5 newly enrolled test-cases (4 for PD and 1 for SD) were obtained and added finally to the initial 11 test cases above.

Identification of Genes Associated with Sensitivity to Gefitinib

We attempted to extract genes that were differentially expressed between tumors from seven patients in the PR group (defined as responders) and those from 10 patients in the PD group (defined as non-responders) by comparing expression levels of 27,648 genes. (tables 2, 3).

We carried out a random-permutation test to distinguish between the two subclasses defined by tumor response, and identified 51 genes whose permutational p-values were less than 0·001 (table 4). Expression levels of 40 genes were higher, and those of the other 11 were lower, in the non-responders.

Establishment of a predictive scoring system for the efficacy of gefitinib treatment Based on the expression profiles of the 51 genes selected above, we tried to establish a predictive scoring system for the efficacy of gefitinib treatment. Prediction scores, termed gefitinib response score (GRS), were calculated according to procedures described previously (see Methods). To determine the number of candidates that provided the best separation of the two groups, we ranked the 51 genes on the basis of the significance of their permutational p-values and calculated prediction scores by the leave-one-out test, in decrements of 1 starting from the bottom of the rank-ordered list (51, 50, 49, 48 etc.). We calculated a classification score (CS), a standard we had previously defined for evaluation of the ability to discriminate two classes, for each set of genes.¹⁷

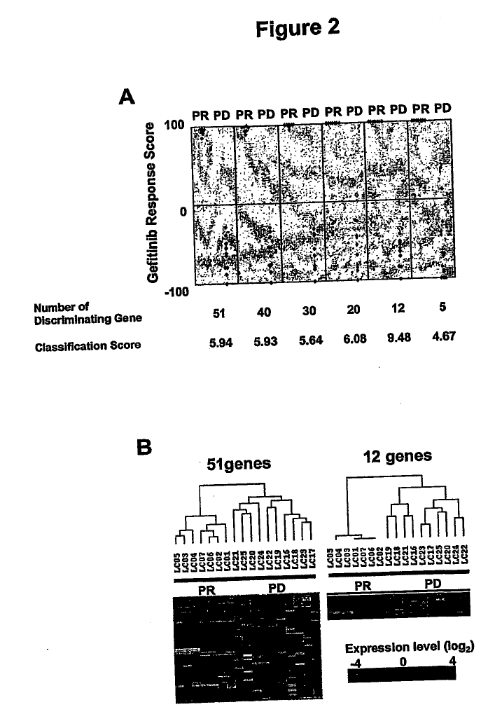

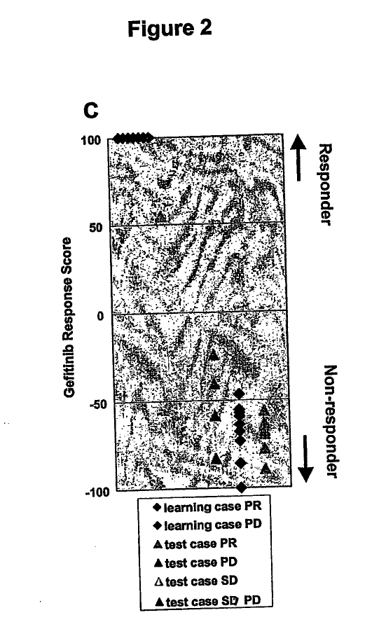

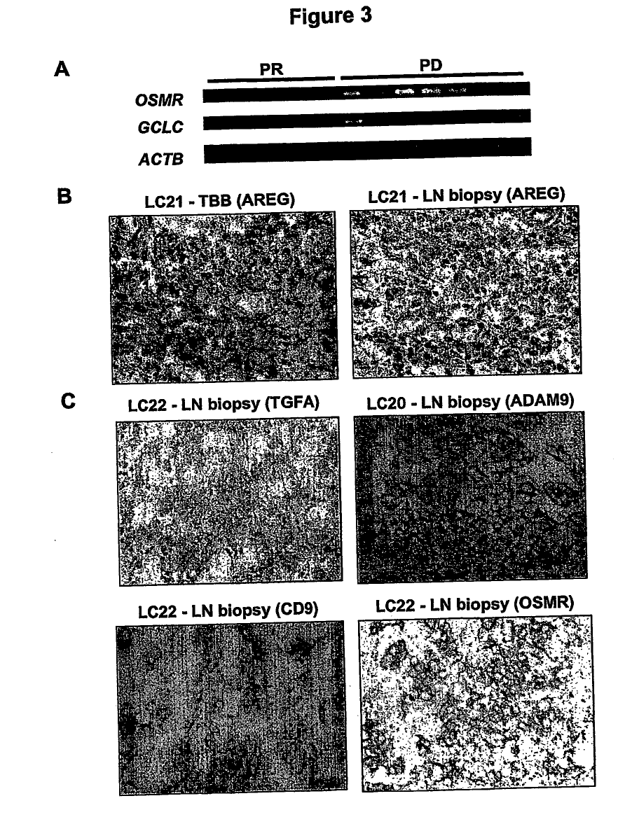

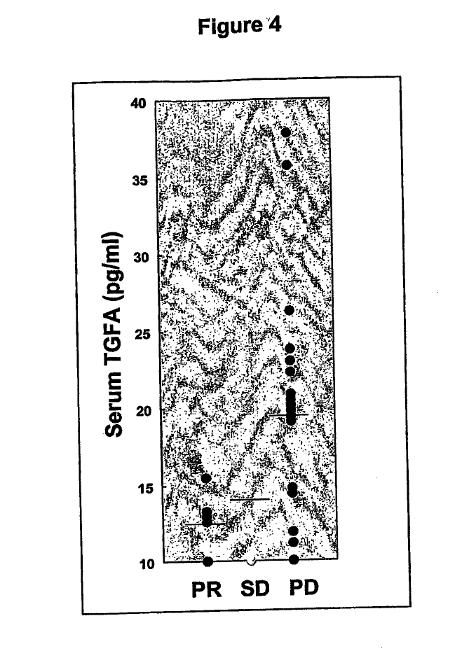

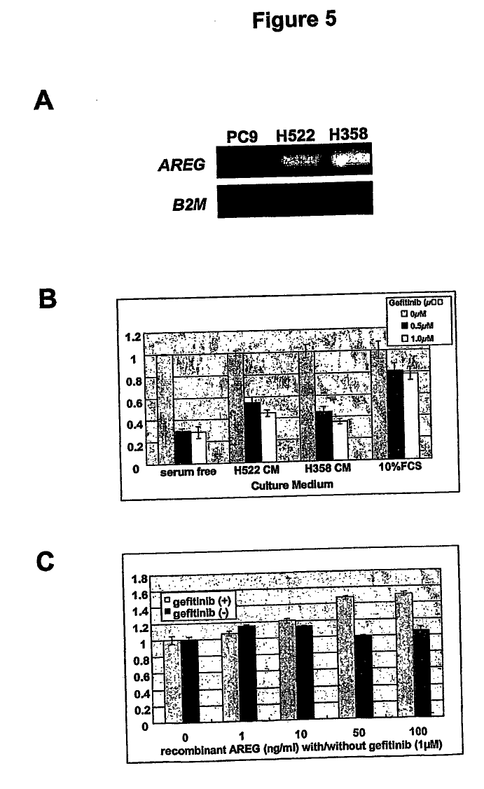

As shown in FIG. 2A, we obtained different prediction scores when the number of discriminating genes was changed. We obtained the best CS, meaning the best separation of responders from non-responders, when we calculated the scores using only the 12 top-ranked genes in our candidate list.

Hierarchical clustering analyses using all 51 genes, or only the top 12, classified all 17 cases into one of two groups according to the response to gefitinib (FIG. 2, B). The two groups were most clearly separated when we used the top 12 genes for cluster analysis. Finally, we established a numerical drug-response-scoring algorithm that might be clinically applicable for predicting sensitivity of an individual NSCLC to gefitinib, on the basis of expression levels of the 12 selected genes.

To validate this prediction system we investigated 8 additional (“test”) NSCLC cases (1 for PR and 7 for PD) that were completely independent of the 17 “learning” cases used for establishing the system. We examined gene-expression profiles in each of those samples and then calculated GRS on the basis of the expression levels of the 12 discriminating genes. As shown in FIG. 2C, scores obtained by the GRS system were concordant with the clinical responses to gefitinib in all eight “test” cases.

GRS Values for Patients with SD in Tumor Response

GRS values for the eight test-SD patients were calculated according to the predictive scoring system established above. Although the values were widely distributed from −83·0 (predicted as non-responder) to 61·6 (responder), the scores of patients who retained SD status throughout the observation period were likely to be higher than those of patients who had been judged as SD at a certain time-point of the study but showed progression of the disease within three or four months after the start of treatment (FIG. 2, C). Although the GRS system was established on the basis of gene-expression profiles that distinguished between patients with PR and patients with PD (without SD) in tumor response, these results suggest that the GRS serves in classifying SD patients into groups according to their response to gefitinib.

Validation of GRS with Semi-Quantitative RT-PCR Analysis

To confirm differential expression of the top 12 predictive genes between PR and PD cases, expression values derived from microarray data were correlated with values from semi-quantitative RT-PCR of RNAs from the same patients (5 PR and 7 PD) (FIG. 3, A, table 5, A). Spearman rank correlations were positive for all of the 12 genes and significantly positive for seven of 12 genes.

Immunohistochemical Validation of GRS

To validate differential expression of the predictive protein markers between PR and PD cases, we carried out immunohistochemical staining with five different antibodies for AREG, TGFA, ADAM9, CD9, and OSMR, all of which were known to be involved in the ligand-EGFRs signalling and whose permutational p-values were less than 0·01. We first stained paired tumor tissue sections obtained by TBB and lymph-node biopsy from the same patients using these 5 antibodies. No intra-patient differences on protein expression of these five markers were observed in three different patients (FIG. 3, B). We also validated the microarray data with the five markers in 11 NSCLC samples (5 for PR and 6 for PD). The results were consistent with the microarray data (FIG. 3, C, table 5, B).

Serum Levels of TGFA

To further evaluate the availability of the prediction system in routine clinical situations, we detected TGFA protein using ELISA in serum samples from 5 PR, 10 SD, and 20 PD patients that were independently collected for serological test and were not enrolled in microarray analysis. The serum levels of TGFA were 19·0±2·8 pg/ml (mean±SE) in PD patients, 13·9±1·9 pg/ml in SD patients, and 12·8±1·4 pg/ml in PR patients (FIG. 4). Twelve of 20 serum samples from PD patients were positive for TGFA and all samples from PR patients were negative, when 16·0 pg/ml was used as a cutoff.

In Vitro Gefitinib Treatment and AREG-Autocrine Assay

AREG, a ligand for EGFR and other ERBB members was significantly over-expressed in non-responders but not (or hardly) detectable in responders. To investigate whether AREG protein leads to resistance of NSCLCs to gefitinib therapy when it is secreted in an autocrine manner, we performed the following biological analyses. We initially identified expression of AREG mRNA in lung-adenocarcinoma cell lines NCI-H358 and -H522, but not in PC-9, by means of RT-PCR experiments (FIG. 5, A). Next, we performed flow-cytometric analysis 72 hours after treatment of PC-9 cells with 1·0 μM of gefitinib, and found that gefitinib increased the percentages of nuclei in sub-G1 (24%) compared with cells with no treatment (6%) (data not shown). This result suggested that gefitinib might induce apoptosis in PC-9 cells.

We then analyzed the viability of PC-9 cells, which are gefitinib-sensitive and do not express AREG, after culture in serum-free medium or in serum-free, conditioned medium obtained from NCI-H358 or -H522 cells grown in the presence or absence of 0·5 or 1·0 μM of gefitinib. As shown in FIG. 5B, the viability of PC-9 cells incubated in the serum-free, conditioned medium containing gefitinib was greater than that of PC-9 cells grown in serum-free medium with the same concentrations of gefitinib. As the supplier of gefinitib has reported previously, the anti-tumor effect of gefitinib decreases in the presence of 10% FCS, suggesting that this assay should be suitable for quantitative measurement of gefitinib dosage and activity.

To investigate whether AREG, secreted in an autocrine manner, inhibits apoptosis of NSCLC cells treated with gefitinib, we cultured PC-9 cells in serum-free medium containing recombinant AREG protein at final concentrations of 1-100 ng/ml, in the presence or absence of 1·0 μM gefitinib. The viability of PC-9 cells incubated with both AREG and 1·0 μM gefitinib was increased in comparison to cells incubated with 1·0 μM gefitinib only, in an AREG-dose-dependent manner (FIG. 5, C). On the other hand, recombinant AREG alone had no effect on the viability of PC-9 cells (FIG. 5, C). This observation appeared to indicate that AREG inhibits the apoptosis induced by gefitinib, but does not in itself affect cell viability. Immunostaining for AREG is shown in FIG. 6.

Discussion

A large body of evidence supports the view that molecules in the EGFR autocrine pathway are involved in a number of processes important to cancer formation and progression, including cell proliferation, angiogenesis, and metastatic spread.⁵ Therapeutic blockade of specific signalling, therefore, could be a promising strategy for cancer treatment. Gefitinib, a synthetic anilinoquinazoline, inhibits the tyrosine kinase activity of EGFR by competing with adenosine triphosphate for a binding site on the intracellular domain of the receptor.⁷ In phase II trials (IDEAL 1 and IDEAL 2), use of gefitinib as a 2nd-, 3rd-, or 4th-line monotherapy for advanced NSCLC achieved tumor-response rates of nearly 20%,⁸⁻¹⁰ which were superior to those achieved with conventional cytotoxic agents. Multivariate analysis of patients in the IDEAL 1 study suggested that the response rate in females might be higher than in males, and higher in patients with adenocarcinomas than in patients with squamous-cell carcinomas (odds ratios 2·7 and 3·5 respectively).⁹ Recent study suggested that individuals in whom gefitinib is efficacious are more likely to have adenocarcinomas of the bronchioloalveolar subtype and to be never smokers (odds ratios 13·5 and 4·2 respectively).¹⁹ The higher tumor-response rate (29·4%) documented in the clinical trial reported here might reflect a higher proportion of patients with adenocarcinoma (46 adenocarcinomas, five squamous-cell carcinomas and two large-cell carcinomas) than has been the case in other studies. The clinicopathological determinants of gefitinib sensitivity including bronchioloalveolar carcinoma (BAC) features are predictve to a certan extent,^{9,10,19,20} however, previous reports and our observations obviously suggest that no factors can perfectly predict the response of NSCLC to gefitinib treatment. Therefore novel methods to discriminate responders from non-responders in advance could allow a more focused use of gefitinib in clinical settings.

By statistical analysis of gene-expression profiles of advanced NSCLCs obtained on cDNA microarrays, we identified dozens of genes associated with sensitivity to gefitinib. We introduced a prediction-scoring system based on expression of the 12 genes that had shown the most significant differences in expression levels between responder (PR) and non-responder (PD) groups. This set of genes was selected from expression profiles of lung adenocarcinomas; however, the GRS system successfully classified all eight of our “test” PR and PD cases in accord with their clinical responses to gefitinib, and one of them was a squamous-cell carcinoma. Moreover, this system was likely to separate intermediate tumor responses (SD) into two groups, one representing patients who succeeded in maintaining the tumor-static effect for a long period and the other representing patients who failed to do so.

In practical terms, we need to predict the chemosensitivity of individual tumors using the minimally invasive techniques available at every hospital, because patients with advanced NSCLCs are rarely candidates for surgical resection of their tumors. Therefore we have tried to establish a prediction system that requires only the amount of cancerous tissue that can be obtained by, for example, flexible bronchofiberscopy. By verifying individual steps of the method, we were able to precisely profile gene expression in biopsy specimens as small as 1 mm. Relevant microarray results were confirmed by semi-quantitative RT-PCR for 12 genes that showed the most significant differences to establish a GRS system. Furthermore, we validated the effectiveness of antibodies for 5 different biomarkers (AREG, TGFA, ADAM9, CD9, and OSMR), all of which were reported to be involved in the ligand-EGFR signalling, for discriminating potential responders from non-responders, in both TBB and lymph-node biopsy samples. Moreover, we were able to detect serum TGFA proteins in lung-ADC patients by ELISA. Further evaluation of these markers for clinical use are necessary, however, the limited number of genes required for prediction should eventually enable laboratories to diagnose in advance the efficacy of gefitinib treatment for an NSCLC patient, using routine procedures such as serological examinations of blood, PCR experiments, or immunohistochemical analysis of biopsy specimens.

To our knowledge, this is the first report about gene-expression profiles of unresectable “advanced” lung cancers, although profiles of surgically resected specimens of “early” lung cancers have been reported.^{21,22} However, about 70% of tumors in patients diagnosed with NCSLC are already locally advanced or metastatic, which generally renders them resistant to conventional therapeutic modalities. Therefore the genes listed here should be useful for disclosing molecular mechanisms of lung-cancer progression and may be potential targets for drug development.

Gefitinib was developed as a “selective” inhibitor of EGFR-TK; however, no clear association between the level of EGFR activation and response to gefitinib has been found in vitro or in vivo.^{7,23} In clinical trials, gefitinib has been more effective against adenocarcinomas than against squamous-cell carcinomas,^{9,10} although over-expression of EGFR is less frequent in adenocarcinomas.²⁴ Therefore, it is important to identify which individual tumors are good targets for this treatment. In our analysis using clinical samples, the difference in EGFR protein expression between responders and non-responders were not statistically significant. On the other hand, amphiregulin (AREG) and transforming growth factor alpha (TGFA), both of which encode the ligand for EGFR and other ERBB members, were significantly over-expressed in non-responders but not (or hardly) detectable in responders p=0·0000000000093 and 0·0095 respectively; table 4).

The significance of the ligands and the EGFR autocrine loop in growth and survival of lung-cancer cells is indisputable,²⁴⁻²⁶ but the role of AREG in formation and progression of cancers is poorly understood. However, several lines of evidence suggest that over-expression of AREG is associated with shortened survival of patients with NSCLC.²⁴ Moreover, anti-apoptotic activity of AREG in human lung-adenocarcinoma cells was reported recently.²⁵ To investigate whether the anti-apoptotic activity of AREG leads to resistance of NSCLC cells to gefitinib therapy, we performed a biological assay using a gefitinib-sensitive but AREG-non-expressing NSCLC cell line, PC-9. We found that the anti-tumor activity of gefitinib on PC-9 cells was dramatically decreased by autocrine secretion of AREG. This evidence strongly suggests that although growth-factor signalling by the EGFR is markedly complicated at every step because of the multiplicity of ligands, dimerization partners, effectors, and downstream pathways,²⁶ AREG might be a principal activator of the ligands-receptor autocrine growth pathway that leads to cancer progression and resistance to gefitinib.

Several elements associated with the EGFR-TK pathway are present on our list of differentially-expressed genes. For example, genes encoding dual specificity phosphatase 3 (DUSP3), ADAM9, CD9, and OSMR were expressed predominantly in non-responders (p=0·00000000094, 0·01, 0·000022, and 0.0000011, respectively). DUSP3 gene modulates EGFR signalling by dephosphorylating mitogen activated protein kinase (MAPK), a key mediator of signal transduction,²⁷ and ADAM9 is involved in activation of EGFR signalling by shedding the ectodomain of proHB-EGF (pro Heparin-binding epidermal growth factor-like growth factor).²⁸ CD9 physically interacts with transmembrane TGFA. CD9 expression strongly decreases the growth factor- and PMA-induced proteolytic conversions of transmembrane to soluble TGFA and strongly enhances the TGFA-induced EGFR activation.²⁹ OSMR is reported to be constitutively associated with ERBB2 in breast cancer cells.³⁰ Although other target molecules for gefitinib have been suggested, our results suggest that EGFR signalling is at least one of the important processes involved in response to this drug.

Since gefitinib can induce apoptosis of some cancer cells in vivo, other molecules with anti-apoptotic activity, as well as AREG, may contribute to a tumor's resistance to the drug. AVEN (apoptosis, caspase-activation inhibitor), which was specifically expressed in our non-responders (p=0·00000000042), is known to enhance the anti-apoptotic activity of Bcl-xL and to suppress Apaf-1-mediated caspase activation.³¹ On the other hand, mechanisms regulating drug transport should also affect drug resistance. GCLC (glutamate-cysteine ligase, catalytic subunit), which plays an important role in cellular detoxification of anticancer drugs such as cisplatin, etoposide and doxorubicin,³² was over-expressed in our group of non-responders (p=0·00000012). As these genes correlated negatively with responses to chemotherapy in our panel of tumors (i.e. the higher the expression of these genes, the greater the resistance to gefitinib), they might be involved in the mechanism(s) leading to that resistance. It should be noted also that the functions of nearly half of our candidate prediction-genes are unknown. Therefore further investigations will be needed to reveal more clearly the biological events underlying responses of NSCLCs to gefitinib.

In summary, we identified 51 genes whose expression differed significantly between responders and non-responders to gefitinib among human lung carcinomas, and established a numerical scoring system, based on expression patterns of 12 of those genes, to predict the response of individual tumors to this drug. Although further validation using a larger set of clinical cases will be necessary, the data presented here may yield valuable insights into the molecular events underlying signal-suppressing strategies and provide important information about gefitinib treatment for individual NSCLC patients by testing a set of genes with high predictive values.

### REFERENCES

1. Fossella, F. V., et al., Randomized phase III trial of docetaxel versus vinorelbine or ifosfamide in patients with advanced non-small-cell lung cancer previously treated with platinum-containing chemotherapy regimens. The TAX 320 Non-Small Cell Lung Cancer Study Group. J Clin Oncol, 2000. 18(12): p. 2354-62.

2. Non-small Cell Lung Cancer Collaborative Group. Chemotherapy in non-small cell lung cancer: a meta-analysis using updated data on individual patients from 52 randomised clinical trials. Bmj, 1995. 311(7010): p. 899-909.

3. Schiller, J. H., et al., Comparison of four chemotherapy regimens for advanced non-small-cell lung cancer. N Engl J Med, 2002. 346(2): p. 92-8.

4. Kelly, K., et al., Randomized phase III trial of paclitaxel plus carboplatin versus vinorelbine plus cisplatin in the treatment of patients with advanced non-small-cell lung cancer: a Southwest Oncology Group trial. J Clin Oncol, 2001. 19(13): p. 3210-8.

5. Baselga, J., Why the epidermal growth factor receptor? The rationale for cancer therapy. Oncologist, 2002. 7 Suppl 4: p. 2-8.

6. Traxler, P., Tyrosine kinases as targets in cancer therapy—successes and failures. Expert Opin Ther Targets, 2003. 7(2): p. 215-34.

7. Wakeling, A. E., et al., ZD1839 (Iressa): an orally active inhibitor of epidermal growth factor signaling with potential for cancer therapy. Cancer Res, 2002. 62(20): p. 5749-54.

8. Herbst, R. S., Dose-comparative monotherapy trials of ZD1839 in previously treated non-small cell lung cancer patients. Semin Oncol, 2003. 30(1 Suppl 1): p. 30-8.

9. Fukuoka, M., et al., Final results from a phase □ trial of ZD1839 (‘Iressa’) for patients with advanced non-small cell lung cancer (IDEAL 1). Pro Am Soc Clin Oncol 2002. 21;298a(A1188).

10. Kris, M G., et al., A phase II trial of ZD1839 (‘Iressa’) in advanced non-small cell lung cancer (NSCLC) patients who had failed platinum-and docetaxel-based regimens (IDEAL 2). Pro Am Soc Clin Oncol 2002. 21;292a(A1166).

11. Inoue, A., et al., Severe acute interstitial pneumonia and gefitinib. Lancet, 2003. 361(9352): p. 137-9.

12. Bohm, M., et al., Microbeam MOMeNT: non-contact laser microdissection of membrane-mounted native tissue. Am J Pathol, 1997. 151(1): p. 63-7.

13. Okabe, H., et al., Genome-wide analysis of gene expression in human hepatocellular carcinomas using cDNA microarray: identification of genes involved in viral carcinogenesis and tumor progression. Cancer Res, 2001. 61(5): p. 2129-37.

14. Kitahara, O., et al., Alterations of gene expression during colorectal carcinogenesis revealed by cDNA microarrays after laser-capture microdissection of tumor tissues and normal epithelia. Cancer Res, 2001. 61(9): p. 3544-9.

15. Golub, T. R., et al., Molecular classification of cancer: class discovery and class prediction by gene expression monitoring. Science, 1999. 286(5439): p. 531-7.

16. MacDonald, T. J., et al., Expression profiling of medulloblastoma: PDGFRA and the RAS/MAPK pathway as therapeutic targets for metastatic disease. Nat Genet, 2001. 29(2): p. 143-52.

17. Kaneta. Y.,et al., Prediction of sensitivity to STI571 among chronic myeloid leukemia patients by genome-wide cDNA microarray analysis. Jpn J Cancer Res 2002. 93, p. 849-856.

18. Pavelic, K., et al., Evidence for a role of EGF receptor in the progression of human lung carcinoma. Anticancer Res, 1993. 13(4): p. 1133-7.

19. Kikuchi, T., et al., Expression profiles of non-small cell lung cancers on cDNA microarrays: Identification of genes for prediction of lymph-node metastasis and sensitivity to anti-cancer drugs. Oncogene, 2003. 22(14): p. 2192-205.

20. Heighway, J., et al., Expression profiling of primary non-small cell lung cancer for target identification. Oncogene, 2002. 21(50): p. 7749-63.

21. Beer, D. G., et al., Gene-expression profiles predict survival of patients with lung adenocarcinoma. Nat Med, 2002. 8(8): p. 816-24.

22. Miura, K., et al., Laser capture microdissection and microarray expression analysis of lung adenocarcinoma reveals tobacco smoking-and prognosis-related molecular profiles. Cancer Res, 2002. 62(11): p. 3244-50.

23. Moasser, M. M., et al., The tyrosine kinase inhibitor ZD1839 (“Iressa”) inhibits HER2-driven signaling and suppresses the growth of HER2-overexpressing tumor cells. Cancer Res, 2001. 61(19): p. 7184-8.

24. Rusch, V., et al., Overexpression of the epidermal growth factor receptor and its ligand transforming growth factor alpha is frequent in resectable non-small cell lung cancer but does not predict tumor progression. Clin Cancer Res, 1997. 3(4): p. 515-22.

25. Fontanini, G., et al., Evaluation of epidermal growth factor-related growth factors and receptors and of neoangiogenesis in completely resected stage I-IIIA non-small-cell lung cancer: amphiregulin and microvessel count are independent prognostic indicators of survival. Clin Cancer Res, 1998. 4(1): p. 241-9.

26. Brundage, M. D., D. Davies, and W. J. Mackillop, Prognostic factors in non-small cell lung cancer: a decade of progress. Chest, 2002. 122(3): p. 1037-57.

27. Hurbin, A., et al., Inhibition of apoptosis by amphiregulin via an insulin-like growth factor-1 receptor-dependent pathway in non-small cell lung cancer cell lines. J Biol Chem, 2002. 277(51): p. 49127-33.

28. Yarden, Y. and M. X. Sliwkowski, Untangling the ErbB signalling network. Nat Rev Mol Cell Biol, 2001. 2(2): p. 127-37.

29. Prenzel, N., et al., EGF receptor transactivation by G-protein-coupled receptors requires metalloproteinase cleavage of proHB-EGF. Nature 1999. 402(6764):884-8

30. Nelson, Chau, et al., Aven, a novel inhibitor of caspase activation, binds Bcl-xL and Apaf-1. Molec Cell 2000.6: p. 31-41.

31. Tipnis, S R., et al., Overexpression of the regulatory subunit of r-glutamylcysteine synthetase in Hela cells increases r-glutamylcysteine synthetase activity and confers drug resistance. Biochem J 1999. 337, p. 559-566.

TABLE 1
Summary of baseline patient characteristics and response

| Characteristics | Percentage (%) | Number of Patient |
|---|---|---|
| Sex | | |
| male | 58.5 | (31) |
| female | 41.5 | (22) |
| Age | | |
| median | 59 | |
| range | 35-80 | |
| Histology | | |
| adenocarcinoma | 86.8 | (46) |
| squamous cell carcinoma | 9.4 | (5) |
| large cell carcinoma | 3.8 | (2) |
| Stage | | |
| IIIA | 1.9 | (1) |
| IIIB | 7.5 | (4) |
| IV | 90.6 | (48) |
| Performance Status | | |
| 0 | 26.4 | (14) |
| 1 | 60.4 | (32) |
| 2 | 13.2 | (7) |
| Number of Prior Regimen | | |
| 1 | 24.5 | (13) |
| 2 | 35.9 | (19) |
| 3 | 28.3 | (15) |
| 4 | 0 | (0) |
| 5 | 7.5 | (4) |
| 6 | 3.8 | (2) |
| Response to Gefitinib Therapy | | |
| CR | 0 | (0) |
| PR | 28.3 | (15) |
| SD | 32.1 | (17) |
| PD | 35.8 | (19) |
| unknown | 3.8 | (2) |
| Tumor Response Rate ( [symbol] | 29.4 | (15) |
| (CR + PR/CR + PR + SD + PD) | | |
| Disease Control Rate (%) | 62.8 | (32) |
| (CR + PR + SD/CR + PR + | | |
| SD + PD) | | |

TABLE 2
Number of cases suitable for analysis and their best overall responses

| | Best Overall Response | | | | |
|---|---|---|---|---|---|
| Number of Cases | PR | SD | PD | Unknown | Total |
| All cases enrolled | 15 | 17 | 19 | 2 | 53 |
| Cases that consented to the | 15 | 14 | 13 | 1 | 43 |
| study | | | | | |
| Cases suitable for analysis | 8 | 10 | 13 | 1 | 32 |
| Learning cases (1) | 7 | 0 | 10 | 0 | 17 |
| Test cases (1,2) | 1 | 7 | 3 | 0 | 11 |

(1) Learning cases were used for developing the GRS, whereas test cases were used for validation of the
(2) Another blinded set of samples from 5 newly enrolled cases (4 PD and 1 SD) were also added to these 11 test cases later.

TABLE 3
Clinicopathological features of patients

| | | | | | | | | Number of | EGFR Stained | |
|---|---|---|---|---|---|---|---|---|---|---|
| Case | | | Histology | | | | Stage | Previous | Tumour Cell | EGFR |
| No. (*) | Sex | Age | Type (1) | T | N | M | Classification (2) | Chemotherapy | (%) | mutation (3) |
| LC01 | female | 36 | ADC | 1 | 0 | 1 | IV | 1 | | None detected |
| LC02 | male | 64 | ADC | 2 | 3 | 1 | IV | 3 | 80 | |
| LC03 | female | 54 | ADC | 2 | 0 | 1 | IV | 3 | 80 | |
| LC04 | female | 75 | ADC | 2 | 1 | 1 | IV | 1 | 20 | None detected |
| LC05 | female | 73 | ADC | 0 | 2 | 1 | IV | 5 | 30 | 46 A750del |
| | | | | | | | | | | (2481 2495 [symbol] |
| LC06 | female | 75 | ADC | 4 | 1 | 1 | IV | 3 | | None detected |
| LC07 | female | 70 | ADC | 2 | 1 | 1 | IV | 3 | 80 | 47_A750del |
| | | | | | | | | | | (2485_2496d |
| LC08 | female | 47 | ADC | 4 | 3 | 1 | IV | 2 | 95 | L858R |
| | | | | | | | | | | (2819T > G) |
| mean (range) | | 62 | | | | | | 2.6 (1-5) | 64 (20-95) | |
| | | (36-75) | | | | | | | | |
| LC09 | female | 63 | ADC | 4 | 0 | 1 | IV | 3 | 90 | |
| LC10 | male | 56 | ADC | 2 | 0 | 1 | IV | 6 | 70 | |
| LC11 | male | 67 | ADC | 4 | 0 | 1 | IV | 2 | 0 | |
| LC12 | male | 53 | ADC | 4 | 3 | 1 | IV | 2 | | None detected |
| LC13 | female | 56 | ADC | 4 | 2 | 0 | IIIB | 2 | 40 | |
| LC14 | female | 62 | ADC | 4 | 2 | 1 | IV | 3 | 60 | |
| LC15 | male | 61 | ADC | 0 | 0 | 1 | IV | 5 | 60 | |
| mean (range) | | 60 | | | | | | 3.3 (2-6) | 53 (0-90) | |
| | | (53-67) | | | | | | | | |
| LC16 | male | 42 | ADC | 4 | 3 | 1 | IV | 5 | 90 | None detected |
| LC17 | female | 54 | ADC | 2 | 3 | 1 | IV | 2 | 50 | None detected |
| LC18 | female | 61 | ADC | 1 | 3 | 0 | IIIB | 2 | | None detected |
| LC19 | male | 59 | ADC | 0 | 2 | 1 | IV | 2 | 30 | W817C |
| | | | | | | | | | | (2697G > T) |
| LC20 | male | 65 | ADC | 0 | 3 | 1 | IV | 3 | | None detected |
| LC21 | male | 55 | ADC | 4 | 3 | 1 | IV | 3 | 80 | None detected |
| LC22 | male | 80 | ADC | 4 | 3 | 1 | IV | 2 | 80 | Q787Q |
| | | | | | | | | | | (2607G > A) |
| LC23 | male | 35 | ADC | 4 | 0 | 1 | IV | 5 | | None detected |
| LC24 | male | 57 | ADC | 4 | 3 | 1 | IV | 1 | 0 | None detected |
| LC25 | female | 65 | ADC | 2 | 0 | 1 | IV | 1 | | None detected |
| LC26 | male | 64 | SCC | 3 | 3 | 1 | IV | 2 | | None detected |
| LC27 | female | 65 | ADC | 4 | 2 | 1 | IV | 1 | | L858R |
| | | | | | | | | | | (2819T > G) |
| LC28 | male | 74 | ADC | 2 | 1 | 1 | IV | 1 | 10 | |

| | | | |
|---|---|---|---|
| mean (range) | 60 | 2.3 (1-5) | 49 (0-90) |
| | (35-80) | | |

| | | | Plasma Gefitinib | Response to Gefitinib(4) | | | | | Use for | |
|---|---|---|---|---|---|---|---|---|---|---|
| Case | | | Concentration | 1st | 2nd | 3rd | 4th | Best Overall | Prediction | |
| No. (*) | Sex | Age | (ng/ml) | month | month | month | month | Response (5) | (6) | GRS (7) |
| LC01 | female | 36 | 258.9 | PR | PR | PR | PR | PR | learning | 100 |
| LC02 | male | 64 | 140.3 | PR | PR | PR | PR | PR | learning | 100 |
| LC03 | female | 54 | 167.0 | PR | PR | PR | PR | PR | learning | 100 |
| LC04 | female | 75 | 169.7 | PR | PR | PR | PR | PR | learning | 100 |
| LC05 | female | 73 | 300.6 | PR | PR | PR | PR | PR | learning | 100 |
| LC06 | female | 75 | 874.0 | SD | PR | PR | PR | PR | learning | 100 |
| LC07 | female | 70 | 460.8 | SD | PR | PR | PR | PR | learning | 100 |
| LC08 | female | 47 | 306.5 | PR | PR | PR | PR | PR | test | 54.8 |
| mean (range) | | 62 | 334.7 | | | | | | | |
| | | (36-75) | (140.3-874.0) | | | | | | | |
| LC09 | female | 63 | 743.4 | SD | SD | SD | SD | SD | test | 61.6 |
| LC10 | male | 56 | 511.8 | SD | SD | SD | SD | SD | test | −9.8 |
| LC11 | male | 67 | 631.3 | SD | SD | SD | SD | SD | test | −5.3 |
| LC12 | male | 53 | 306.1 | SD | SD | SD | PD | SD | test | −23.8 |
| LC13 | female | 56 | 364.8 | SD | SD | PD | | SD | test | −58.5 |
| LC14 | female | 62 | 322.4 | SD | SD | PD | | SD | test | −83 |
| LC15 | male | 61 | 278.9 | SD | SD | PD | | SD | test | −40.5 |
| mean (range) | | 60 | 451.2 | | | | | | | |
| | | (53-67) | (278.9-631.3) | | | | | | | |
| LC16 | male | 42 | 212.6 | SD | PD | | | PD | learning | −63.9 |
| LC17 | female | 54 | 320.6 | SD | PD | | | PD | learning | −86 |
| LC18 | female | 61 | 229.3 | SD | PD | | | PD | learning | −67.8 |
| LC19 | male | 59 | 150.7 | SD | PD | | | PD | learning | −57.1 |
| LC20 | male | 65 | 167.8 | SD | PD | | | PD | learning | −59.1 |
| LC21 | male | 55 | | PD | | | | PD | learning | −73.1 |
| LC22 | male | 80 | | PD | | | | PD | learning | −55.5 |
| LC23 | male | 35 | | PD | | | | PD | learning | −100 |
| LC24 | male | 57 | | PD | | | | PD | learning | −46.7 |
| LC25 | female | 65 | 356.3 | PD | | | | PD | learning | −86.1 |
| LC26 | male | 64 | 405.6 | SD | PD | | | PD | test | −67.7 |
| LC27 | female | 65 | | PD | | | | PD | test | −69.4 |
| LC28 | male | 74 | | PD | | | | PD | test | −64.8 |

| | | |
|---|---|---|
| mean (range) | 60 | 263.2 |
| | (35-80) | (150.7-405.6) |

(1) ADC, adenocarcinoma; SCC, squamous-cell carcinoma.
(2) TNM clinical classification and stage grouping were assessed based on the UICC/WHO classification.
(3) Mutation at codon position 709-870 (from p-loop to activation loop) of EGFR (GenBank Accession No. NM005228).
(4) Objective Tumor Response to Gefitinib was assessed every 4 weeks after the start of treatment using UICC/WHO Criteria. PR, partial response; SD, stable disease; PD, progressive disease
(5) Overall Best Response was evaluated based on the definitions as mentioned in materials and methods.
(6) learning, samples used for developing the GRS; test, samples used for validation of the GRS.
(7) GRS: gefitinib response score determined by prediction system
(*) For further validation of the GRS, another blinded set of samples from 5 newly enrolled cases (4 PD and 1 SD) were also added to these 28 cases later.

TABLE 4
List of 51 candidate genes for discriminating responder (PR) from non-responder (PD) to gefitinib (*)

| | | | | | | Median-fold |
|---|---|---|---|---|---|---|
| Rank | | | | Predominantly | Permutational | Difference |
| Or [symbol] | GenBank | Symbol | Gene Name | Expressed Clas | p-value | (log2) |
| 1 | NM_0248 [symbol] | FLJ2266 [symbol] | hypothetical protein FLJ22662 | PD | 8.1E−12 | 2.0 |
| 2 | BC009799 | AREG | amphiregulin (schwannoma-derived growth factor) | PD | 9.3E−12 | 8.0 |
| 3 | NM_0143 [symbol] | CORO1C | coronin, actin binding protein, 1C | PD | 2.3E−10 | 4.6 |
| 4 | BC010488 | AVEN | apoptosis, caspase activation inhibitor | PD | 4.2E−10 | 4.3 |
| 5 | NM_0040 [symbol] | DUSP3 | dual specificity phosphatase 3 (vaccinia virus phosphatase VH1-rel [symbol] | PD | 9.4E−10 | 4.4 |
| 6 | AI026836 | DJ473B4 | hypothetical protein dJ473B4 | PD | 1.7E−09 | 8.0 |
| 7 | BU500509 | PHLDA2 | pleckstrin homology-like domain, family A, member 2 | PD | 1.8E−09 | 8.0 |
| 8 | NM_0160 [symbol] | RBM7 | RNA binding motif protein 7 | PD | 1.8E−08 | 2.9 |
| 9 | BX092512 | | EST | PD | 7.7E−08 | 3.0 |
| 10 | AI436027 | OSMR | oncostatin M receptor | PD | 1.1E−07 | 3.7 |
| 11 | AI971137 | GCLC | glutamate-cysteine ligase, catalytic subunit | PD | 1.2E−07 | 3.9 |
| 12 | BQ02487 [symbol] | COL4A3 [symbol] | coltagen, type IV, alpha 3 (Goodpasture antigen) binding protein | PD | 2.0E−07 | 3.6 |
| 13 | U52522 | ARFIP2 | ADP-ribosylation factor interacting protein 2 (arfaptin 2) | PD | 2.6E−07 | 2.8 |
| 14 | BM99605 [symbol] | C10orf9 | chromosome 10 open reading frame 9 | PD | 4.2E−07 | 2.6 |
| 15 | AK025452 | NIP30 | NEFA-interacting nuclear protein NIP30 | PD | 5.1E−07 | 3.7 |
| 16 | N52048 | KIAA077 [symbol] | KIAA0776 protein | PD | 5.4E−07 | 7.2 |
| 17 | AA507009 | SLC35F2 | solute carrier family 35, member F2 | PD | 6.0E−07 | 5.8 |
| 18 | AA226243 | GAMLG | calcium modulating ligand | PD | 6.8E−07 | 5.0 |
| 19 | AF005888 | NOC4 | neighbor of COX4 | PD | 1.1E−06 | 4.0 |
| 20 | AF012281 | PDZK1 | PDZ domain containing 1 | PD | 1.3E−06 | 4.5 |
| 21 | AI188190 | DIS3 | mitotic control protein dis3 homolog | PD | 1.7E−06 | 3.8 |
| 22 | BC001535 | CGI-48 | CGI-48 protein | PD | 2.0E−06 | 3.5 |
| 23 | NM_0070 [symbol] | CPSF6 | cleavage and polyadenylation specific factor 6, 68 kDa | PD | 2.2E−06 | 3.4 |
| 24 | NM_0022 [symbol] | KIF3C | kinesin family member 3C | PD | 2.2E−06 | 3.5 |
| 25 | BQ135232 | CD9 | CD9 antigen (p24) | PD | 2.2E−06 | 1.7 |
| 26 | BC051322 | LRRC8 | leucine rich repeat containing 8 | PD | 2.5E−06 | 3.4 |
| 27 | BC03850 [symbol] | SNF1LK | SNF1-like kinase | PD | 2.6E−06 | 2.8 |
| 28 | U78556 | CRA | cisplatin resistance associated | PD | 2.7E−06 | 3.7 |
| 29 | BC035625 | EGR2 | early growth response 2 (Krox-20 homolog, Drosophila) | PD | 3.4E−06 | 3.0 |
| 30 | X52426 | KRT13 | keratin 13 | PD | 1.9E−05 | 3.4 |
| 31 | NM_0055 [symbol] | BCAT1 | branched chain aminotransferase 1, cytosolic | PD | 2.3E−05 | 1.7 |
| 32 | NM_0066 [symbol] | SDCCAG | serologically defined colon cancer antigen 3 | PR | 2.6E−05 | 3.7 |
| 33 | AA46409 [symbol] | PIGK | phosphatidylinositol glycan, class K | PD | 3.2E−05 | 1.1 |
| 34 | AA96118 [symbol] | MRPS9 | mitochondrial ribosomal protein S9 | PD | 9.8E−05 | 2.3 |
| 35 | NM_0181 [symbol] | ASPM | asp (abnormal spindle)-like, microcephaly associated (Drosophila) | PR | 2.3E−04 | 2.8 |
| 36 | NM_0227 [symbol] | ACBD3 | acyl-Coenzyme A binding domain containing 3 | PD | 2.4E−04 | 3.8 |
| 37 | AA160544 | ZNF325 | zinc finger protein 325 | PR | 2.7E−04 | 4.5 |
| 38 | AK05765 [symbol] | LOC2855 | hypothetical protein LOC285513 | PD | 2.7E−04 | 3.8 |
| 39 | NM_0033 [symbol] | TSSC1 | tumor suppressing subtransferable candidate 1 | PD | 2.9E−04 | 4.7 |
| 40 | BC007451 | XAB1 | XPA binding protein 1 | PD | 3.0E−04 | 1.3 |
| 41 | BC03546 [symbol] | HNLF | putative NFkB activating protein HNLF | PR | 3.5E−04 | 1.1 |
| 42 | CK00409 [symbol] | EIF4EBP | eukaryotic translation initiation factor 4E binding protein 2 | PR | 3.6E−04 | 1.4 |
| 43 | NM_1446 [symbol] | MGC232 [symbol] | hypothetical protein MGC23280 | PR | 4.2E−04 | 2.3 |
| 44 | NM_0046 [symbol] | SSA2 | Sjogren syndrome antigen A2 (60 kDa, ribonucleoprotein autoantige | PR | 4.2E−04 | 1.2 |
| 45 | NM_0027 [symbol] | PRKACA | protein kinase, cAMP-dependent, catalytic, alpha | PR | 5.0E−04 | 1.2 |
| 46 | NM_0051 [symbol] | FEZ2 | fasciculation and elongation [symbol] rotein zeta 2 (zygin II) | PD | 6.1E−04 | 3.3 |
| 47 | NM_0058 [symbol] | SRRM1 | serine/arginine repetitive matrix 1 | PR | 7.0E−04 | 1.4 |
| 48 | NM_0062 [symbol] | PDGFRL | platelet-derived growth factor receptor-like | PD | 7.0E−04 | 2.4 |
| 49 | AI096936 | SNX13 | sorting nexin 13 | PR | 8.4E−04 | 1.6 |
| 50 | NM_0147 [symbol] | KIAA025 [symbol] | KIAA0258 gene product | PD | 8.9E−04 | 2.5 |
| 51 | BF973104 | TOM7 | homolog of Tom7 (S. cerevisiae) | PR | 1.0E−03 | 1.5 |

(*) The 12 and 51 gene sets were listed as the rank-order of permutational p-values that were less than 0.001.

TABLE 4

A List of 132 Candidate Genes for Discriminating Responder
(PR) from Non-responder (PD) to Gefitinib

| | | | | |
|---|---|---|---|---|
| Rank | GenBank | Gene | | |
| Order | ID | Symbol | Gene Name | Nucleotides |

| | | | | |
|---|---|---|---|---|
| 1 | NM_024829 | FLJ22662 | hypothetical protein | ATACGGCATCCATGAAATATAT |
| | | | FLJ22662 | CATGCGATACAACAATTATAAG |
| | | | | AAGGATCCTTACAGTAGAGGTG |
| | | | | ACCCCTGTAATACCATCTGCTG |
| | | | | CCGTGAGGACCTGAACTCACCT |
| | | | | AACCCAAGTCCTGGAGGTTGTT |
| | | | | ATGACACAAAGGTGGCAGATAT |
| | | | | CTACCTAGCATCTCAGTACACA |
| | | | | TCCTATGCCATAAGTGGTCCCA |
| | | | | CAGTACAAGGTGGCCTCCCTGT |
| | | | | TTTTCGCTGGGACCGTTTCAAC |
| | | | | AAAACTCTACATCAGGGCATGC |
| | | | | CAGAGGTCTACAACTTTGATTT |
| | | | | TATTACCATGAAACCAATTTTG |
| | | | | AAACTTGATATAAAATGAAGGA |
| | | | | GGGAGATGACGGACTAGAAGAC |
| 2 | BC009799 | AREG | amphiregulin | CTCCACTCGCTCTTCCAACACC |
| | | | (schwannoma-derived | CGCTCGTTTTGCGGCAGCTCGT |
| | | | growth factor) | GTCCCAGAGACCGAGTTGCCCC |
| | | | | AGAGACCGAGACGCCGCCGCTG |
| | | | | CGAAGGACCAATGAGAGCCCCG |
| | | | | CTGCTACCGCCGGCGCCGGTGG |
| | | | | TGCTGTCGCTCTTGATACTCGG |
| | | | | CTCAGGCCATTATGCTGCTGGA |
| | | | | TTGGACCTCAATGACACCTACT |
| | | | | CTGGGAAGCGTGAACCATTTTC |
| | | | | TGGGGACCACAGTGCTGATGGA |
| | | | | TTTGAGGTTACCTCAAGAAGTG |
| | | | | AGATGTCTTCAGGGAGTGAGAT |
| | | | | TTCCCCTGTGAGTGAAATGCCT |
| | | | | TCTAGTAGTGAACCGTCCTCGG |
| | | | | GAGCCGACTATGACTACTCAGA |
| | | | | AGAGTATGATAACGAACCACAA |
| | | | | ATACCTGGCTATATTGTCGATG |
| | | | | ATTCAGTCAGAGTTGAACAGGT |
| | | | | AGTTAAGCCCCCCCAAAACAAG |
| | | | | ACGGAAAGTGAAAATACTTCAG |
| | | | | ATAAACCCAAAAGAAAGAAAAA |
| | | | | GGGAGGCAAAAATGGAAAAAAT |
| | | | | AGAAGAAACAGAAAGAAGAAAA |
| | | | | ATCCATGTAATGCAGAATTTCA |
| | | | | AAATTTCTGCATTCACGGAGAA |
| | | | | TGCAAATATATAGAGCACCTGG |
| | | | | AAGCAGTAACATGCAAATGTCA |
| | | | | GCAAGAATATTTCGGTGAACGG |
| | | | | TGTGGGGAAAAGTCCATGAAAA |
| | | | | CTCACAGCATGATTGACAGTAG |
| | | | | TTTATCAAAAATTGCATTAGCA |
| | | | | GCCATAGCTGCCTTTATGTCTG |
| | | | | CTGTGATCCTCACAGCTGTTGC |
| 3 | Nm_014325 | C0R01C | coronin, actin binding | GATAGGCCACATTCCAGTAAGA |
| | | | protein, 1C | ACTCAATTTGTCTCCCAAATTT |
| | | | | GCAGAAACAAAACGTGATTTAA |
| | | | | AAGCTGAGCTTTTTATCAGAAA |
| | | | | GCTTTTTTGATGTTTTAAGTGT |
| | | | | TATGTGACTTGTTGAACTTTTT |
| | | | | AAAAAGTGCTACTTTTAAAATC |
| | | | | CCAGATACTCTGAATTTTAGAA |
| | | | | AACAAACTAATTCTGATTGTGT |
| | | | | CGTGCCCAAGTACCCTTTTTTT |
| | | | | TTTAATGAGTAGGGACCAATGC |
| | | | | CACATTGCTTTTTATATTTCTT |
| | | | | TCTTTTTTAATGTTGCCAAAAC |
| | | | | CAAAAGTAGCTTTGTTTTCCTT |
| | | | | TGTATTTTGCTACTTTGCAGTA |
| | | | | TTTGTGTGTGTGGTTTTTTTTC |
| | | | | CTTAATTTGAAAGGGACAGCAC |
| | | | | TGTGTATGTTTA |
| 4 | BC010488 | AVEN | apoptosis, caspase acti- | AGGAGACCATTTGGAAGAAGAA |
| | | | vation inhibitor | CTAGATCTGTTGCTTAATTTAG |
| | | | | ATGCACCTATAAAAGAGGGAGA |
| | | | | TAACATCTTACCAGATCAGACG |
| | | | | TCTCAGGACCTGAAATCCAAGG |
| | | | | AAGATGGGGAGGTGGTCCAAGA |
| | | | | GGAAGAAGTTTGTGCAAAACCA |
| | | | | TCTGTGACTGAAGAAAAAAACA |
| | | | | TGGAACCTGAGCAACCAAGTAC |
| | | | | CTCCAAAAATGTTACCGAGGAA |
| | | | | GAGCTGGAAGACTGGTTGGACA |
| | | | | GCATGATTTCCTAAAAAGGGGA |
| | | | | AAAAAAGTGCCTGAAGCAAATC |
| | | | | TTGGTTGCCTTCTAACGGCAGG |
| | | | | TGGGCATAAGGCTGTCCTTCAG |
| | | | | GACCAGCCAGTTTACAAGCATG |
| | | | | TCTCAAGCTAGTGTGTTCCATT |
| | | | | ATGCTCACAGCAGTAAATGCCT |
| | | | | ACCTCTGTGTTTGACATCTGAA |
| | | | | AGAATACATTGAAGCAGCTTGT |
| | | | | TGCATTTGTTTTTCTGGCTTAG |
| | | | | TAATCTAATAGATTTCCTTAAG |
| | | | | GGCAGGAGATAGACTCTGGCCC |
| | | | | TTGTTTCTAGCCTCCTTCCTTG |
| | | | | CAGTGTTTACAACATAGCCAGT |
| | | | | GTTTACAGCATAGCA |

13: U52522 ARFIP2

| | |
|---|---|
| 1 | tggagcccga ggtccccgcg cggcccgggc ctggcgccct gaggggaaga gcggcccggc |
| 61 | ccgagccatg acggacggga tcctagggaa ggcagccaca atggagatcc ctatccacgg |
| 121 | gaacggcgaa gccaggcagc ttcctgaaga tgatgggctg gagcaggacc tccagcaggt |
| 181 | gatggtgtca ggacccaacc tcaatgaaac cagcattgtg tctggtggct atgggggctc |
| 241 | tggtgatgga ctcatcccca cagggtctgg ccgccatcca tctcacagca ccactccttc |
| 301 | tggccctgga gatgaggtgg ctcggggcat tgctggagaa aagtttgaca tcgtcaagaa |
| 361 | atggggcatc aacacctata agtgcacaaa gcaactgtta tcagaacgat ttggtcgagg |
| 421 | ctcacggact gtggacctgg agctagagct gcagattgag ttgctgcgtg agacgaagcg |
| 481 | caagtatgag agtgtcctgc agctgggccg ggcactgaca gcccacctct acagcctgct |
| 541 | gcagacccag catgcactgg gtgatgcctt tgctgacctc agccagaagt ccccagagct |
| 601 | tcaggaggaa tttggctaca atgcagagac acagaaacta ctatgcaaga atggggaaac |
| 661 | gctgctagga gccgtgaact tctttgtctc tagcatcaac acattggtca ccaagaccat |
| 721 | ggaagacacg ctcatgactg tgaaacagta tgaggctgcc aggctggaat atgatgccta |
| 781 | ccgaacagac ttagaggagc tgagtctagg cccccgggat gcagggacac gtggtcgact |
| 841 | tgagagtgcc caggccactt tccaggccca tcgggacaag tatgagaagc tgcggggaga |
| 901 | tgtggccatc aagctcaagt tcctggaaga aaacaagatc aaggtgatgc acaagcagct |
| 961 | gctgctcttc cacaatgctg tgtccgccta ctttgctggg aaccagaaac agctggagca |
| 1021 | gaccctgcag cagttcaaca tcaagctgcg gcctccagga gctgagaaac cctcctggct |
| 1081 | agaggagcag tgagctgctc ccagcccaac ttggctatca agaaagacat tgggaagggc |
| 1141 | agccccaggg tgtgggagat tggacatggt acatcctttg tcacttgccc tctggcttgg |
| 1201 | gctccttttt ctggctgggg cctgacacca gttttgccca cattgctatg gtgggaagag |
| 1261 | tgcctggagg cccagaagtt gctgccctgt ctatcttcct ggccacaggg cttcattccc |
| 1321 | agatcttttc cttccacttc acagccaacg gctatgacaa aaccactccc tggccaatgg |
| 1381 | catcactctt caggctgggg tgtgctccct gaccaatgac agagcctgaa aatgccctgt |
| 1441 | cagccaatgg cagctcttct cggactcccc tgggccaatg atgttgcgtc taataccctt |
| 1501 | tgtctctcct ctatgcgtgc ccattgcaga gaaggggact gggaccaaag gggtggggat |
| 1561 | aatggggagc cccattgctg gccttgcatc tgaataggcc taccctcacc cacccaccca |
| 1621 | gtttaattgt gcttagagcc caagaagatt ggga |

14: BM996053 C10orf9

| | |
|---|---|
| 1 | tttttttttt ttttttttaa agtagtttaa taaactccac aaaataatag cagatgcatt |
| 61 | gaaatattta cataattcga ttttcaaatc tctcattcaa ataaaaggga taaaaataaa |
| 121 | atttctgcct ttacggcagc agaacctctt tcctgaaatg gattggtaaa ataagatact |
| 181 | tcactggnag aggaactaat ttatgtttaa gaggtattca tattcagcta agaaaataca |
| 241 | accctttttc agctatatag attagggaat ataaaatgat attttctaca ttttttgacc |
| 301 | tgtattcaaa gttctaaatt caactttgac ttgaagagag aaggtgattt tggtacccat |
| 361 | acagagtaga tcatcacaat tacaatggaa agataattaa cgttttatat gctgtttatt |
| 421 | tgcttttgaa agtttgggtc agaaaggctg tgataataat tctggcccaa acaggtatgc |
| 481 | ttatacctga cacaaatttc actaaaacct aacacttttg gcttggagtt cttgggattt |
| 541 | cgactttctg agtcccttcc atttccaaag catgtttcat tgagagcagg caatgtttgg |
| 601 | ggatcaggtg tatgattcaa gactaattaa gatgccaaag ttttccaagc tc |

15: AK025452 NIP30

| | |
|---|---|
| 1 | attgtggcgg tgaggaacag gaagccctga agggtcaaaa gaaatacaaa agcaaaggct |
| 61 | attttctttt tttttttctt tctttcattc cttccttcct ctgtttcttt ctttcttcct |
| 121 | ttcatttttt tttctttttt aagagcgagc ggctctgcgg tggcggtttg gggtgggcgc |
| 181 | cgccgaggtg aggtcgtctc gcctcccgcg cgccggtaga ttggttgttt cattatggat |
| 241 | ggaggggatg atggtaacct tattatcaaa aagaggtttg tgtctgaggc agaactagat |
| 301 | gaacggcgca aaaggaggca agaagaatgg gagaaagttc gaaaacctga agatccagaa |
| 361 | gaatgtccag aggaggttta tgaccctcga tctctatatg aaaggctaca ggaacagaag |
| 421 | gacaggaagc agcaggagta cgaggaacag ttcaaattca aaaacatggt aagaggctta |
| 481 | gatgaagatg agaccaactt ccttgatgag gtttctcgac agcaggaact aatagaaaag |
| 541 | caacgaagag aagaagaact gaaagaactg aaggaataca gaaataacct caagaaggtt |
| 601 | ggaatttctc aagagaacaa gaaggaagtg gaaaagaaac tgactgtgaa gcctatagaa |
| 661 | accaagaaca agttctccca ggcgaagctg ttggcaggag ctgtgaagca taagagctca |
| 721 | gagagtggca acagtgtgaa aagactgaaa ccggaccctg agccagatga caagaatcaa |
| 781 | gagccctcat cctgcaagtc tctcggaaac acctccctga gtggcccctc catccactgc |
| 841 | ccctctgctg cagtatgtat cggcatcctc ccaggcctgg gtgcctactc tgggagcagc |
| 901 | gactccgagt ccagctcaga cagcgaaggc accatcaatg ccaccggaaa gattgtctcc |
| 961 | tccatcttcc gaaccaacac cttcctcgag gccccctagt ttctccgtcc ctacacaggg |
| 1021 | agctcctccc caagggtaga tcggaccgtt catgctgcct ataggcatta tgtccctcaa |
| 1081 | aaaaaaactc ctttgcctgc atcctgtgta caacatgaca tttttaacca atccaatcta |
| 1141 | aaaatgtgcc agaatccacc tgtggcccga atcgtgtttg gttcctcttt ctactccact |
| 1201 | gcagatgacc aaacctgtcc cgctgccact ttcctcactg atattgggag gagggcaagg |
| 1261 | cccagccgaa gttccactaa aaatgcccca ggagaatagg caccggctgg cttgccaaag |
| 1321 | ggtttgggtt ttattgcttt ctgttttttc ttttcccgac agcacaaaga agtaagggca |
| 1381 | gttattggac aggtgttatt taaacattct attgtaaatg aatgtgttgt ttggttctac |
| 1441 | tgcattgtgg agcatgcggg ggaagagaac tgacccaggt aatgaaatgg agcccttccc |
| 1501 | tggaactaac cagtccttga tgttgtgtga ctaagtaaag atgataaacc ccatctgctg |
| 1561 | ggggtgtcac ttcacactcg gcatgcattg tgaaagcttt ccataccctt ggccattccc |
| 1621 | tctctcctct ctctccaacc ccatttatgc aggaggggac tgctaacaag aacgcttcca |
| 1681 | tctcaaacct tttctctgcc tgggaaatta ttttatgttt gtttttgaaa taaaggattt |
| 1741 | agtttaagat tctaaatttt agagaaacaa acgtaggcct tgtttactaa tagccagaca |
| 1801 | tcagaactgc aggtaggtat gttaatgaga tgacttattt ctggcagctc ctggaatcct |
| 1861 | aatattgtaa atgagtggga cacacttgca tattgtgacc attctattga ggcccttctc |
| 1921 | tgtttaatgc atattatact tgtgctttta actgtggaat ctatttctaa cctaaaaaaa |
| 1981 | aaaaaaaaaa |

16: N52048 KIAA0776

| | |
|---|---|
| 1 | aaatttctgt attttagttt tgaagtgctt tctatttaaa aataaaaaac atgatttatt |
| 61 | ttcatttctg acacagaagt gtttctttta aaaaaaaaag accacatttt aaatttctgc |
| 121 | ttaaatgtat ataaagtata catttaagta tactggcact cgcaacaaat gaatccttcc |
| 181 | ccagggataa atggattgga aaatttgttt ttcattcaac atttggaaag agaacaaacc |
| 241 | tgaaatatgt aatttttaaa attatgtgaa aataatgtga aaaatttcat atagtatttg |
| 301 | tgtgaaaatc aggtggaaaa aaacttccat gaagaaccca atttaccaaa attcnccatc |
| 361 | nttttaagat ttacnttttn aataccatac tactggtttt aacnggaatt ggggtgtggt |
| 421 | atgagggggg ttttgcnggg gagangggga |

17: AA507009: SLC35F2

| | |
|---|---|
| 1 | tatttccatg aattcaaagc cttttaatga tgtgaacact tactccccat ttctttttta |
| 61 | cattgttaca aaaaatttac atacagtttt ctgaaagtgg cattttgttg gttgttatta |
| 121 | tactgatgac acatattaac actttgtatt gaagaagtat cataaaaatc acagggcatt |
| 181 | acagattttt gataagaagt agtaatagca ttgtctttta acagctggag gctcccaggc |
| 241 | atactctttg gtgagaaatg attaatttta tattttcatt ttgatgagaa tcttttcttg |
| 301 | tttttaccag ttataaaaac aaagcttttt ctttgttgtg atactgtgca ctaagactta |
| 361 | gtttcttgag ctgatgctaa ataaaatgag atcaatagga atattccggg aggtcgtgag |
| 421 | aagtttttag aaaggatggc atctacatat atatggagct ctgaaaactg ttggagagta |
| 481 | tgacctggga ctgaaactgt ggagcaca |

18: AA226243: GAMLG

| | |
|---|---|
| 1 | tcgacatcta atcctcattc ttatgaactt ggtattatgt gaatccattg ggaaatgaag |
| 61 | actcagagag gttaaaccat ttgcccaagg tcacacagct actaagcggt gctaggatta |
| 121 | aaccctggca gtttcagtcc ttaaccaaag ggttaagcgc tttacatata tatacctgta |
| 181 | tattatcaat tttcaaataa tttgaaatag taaaatgcag ttcctctggt cctgaatatg |
| 241 | aggtaatctt cctatggttc agaagacatt tcagcatttc ctaatatatc ataatgagtc |
| 301 | cattttggtt gtaccatgat gtagtcattt tgttttatag tatattaaag aatgcagaaa |
| 361 | agcatagctc atagttccag tgtcttgctc tgaggttttt ccattcagta gacattttaa |
| 421 | gtatatgtac caggcacata aatatccaga taattaaagt ttatatcatt aagcn |

19: AF005888 NOC4

| | |
|---|---|
| 1 | gcgccatcaa tcgccgccgc ctcgtcccgc ttctcggctg aggcgccgcg cggccaggca |
| 61 | gcgggtccag gcctcagccg cgcgcccagg ggcctccggg gccctcccgg gtcagcatgc |
| 121 | ccggggtgaa actgaccacc caggcctact gcaagatggt gctgcacggc gccaagtacc |
| 181 | cgcactgcgc cgtcaacggg ctcctggtgg ccgagaagca gaagccgcgt aaggagcacc |
| 241 | tccccctggg cggccccggc gcccaccaca ccctcttcgt ggactgcatc cccctcttcc |
| 301 | acggcaccct ggccctcgcc cccatgctgg aggtggctct caccctgatt gattcatggt |
| 361 | gcaaagatca tagctacgtg attgctggtt attatcaagc taatgagcga gtaaaggatg |
| 421 | ccagtccaaa ccaggttgca gagaaggtgg cctccagaat cgccgagggc ttcagcgaca |
| 481 | ctgcgctcat catggtagac aacaccaagt ttacgatgga ctgcgtagcg cctacgatcc |
| 541 | acgtgtacga gcaccatgag aacagatggc ggtgcagaga cccacaccat gactactgtg |
| 601 | aagactggcc agaggcacag aggatctcag cctcgctcct ggacagccgg tcctacgaga |
| 661 | cgctcgtgga tttcgataac cacctggatg acattcggaa tgactggaca aacccagaga |
| 721 | tcaataaagc tgtcctacac ttgtgctagg caggcaccgc tgtgactggg ctccgggcct |
| 781 | ttcccactac gttgaagaag aaaacctatt tttaaatgta aataaaatat ctggtagcct |
| 841 | gtgtggaaag ctgaccgttt taagaagtgg catgtgcctt gaaagggggc agaatgttca |
| 901 | gtcggtcgtg tttttaacac agagtctcta gaagaggtgc agacatcccg tctgactgtc |
| 961 | cctgtggact ctctcagttg tatgttgcta taatcctcca aatcaaagct ctttctgctt |
| 1021 | gtgcaagatt gttcctatta aacagtttta actaaccttt a |

20: AF012281 PDZK1

| | |
|---|---|
| 1 | gaattccggg cagctcctct tccatctcca gaaatgacct ccaccttcaa cccccgagaa |
| 61 | tgtaaactgt ccaagcaaga agggcaaaac tatggcttct tcctgcgaat tgagaaggac |
| 121 | accgagggcc acctggtccg ggtggttgag aagtgtagcc cagcagagaa ggctggcctt |
| 181 | caagatggag acagagttct taggatcaat ggtgtctttg tggacaaaga agaacatatg |
| 241 | caggttgtgg atctggtcag aaagagtggg aattcagtga ctttactagt tctggatggg |
| 301 | gattcctatg agaaagcagt gaaaacacgg gtggacttga aagagttggg tcaaagtcag |
| 361 | aaggagcaag gtttgagtga taatatactt tcccctgtga tgaatggagg tgtgcaaact |
| 421 | tggacccagc cccggctctg ctatctcgtg aaggaaggag gcagctatgg cttctctctg |
| 481 | aaaactgtcc aaggtaaaaa gggggtgtac atgactgata ttacacctca aggtgtggct |
| 541 | atgagagctg gagttctggc tgatgatcac ttgattgaag tgaatggaga gaatgtagag |
| 601 | gatgccagcc atgagaaagt ggttgaaaag gtgaagaagt caggaagccg tgtcatgttc |
| 661 | ctgctggtgg acaaagaaac tgacaagcgt catgttgagc agaagataca attcaaaaga |
| 721 | gaaacagcca gtttgaaact gttaccccac cagccccgaa ttgtggagat gaagaaagga |
| 781 | agcaatggct atggtttcta tctgagggca ggctcagaac agaaaggtca aatcatcaag |
| 841 | gacatagatt ctggaagtcc agcagaggag gctggcttga agaacaatga tctggtagtt |
| 901 | gctgtcaacg gcgagtctgt ggaaaccctg gatcatgaca gtgtggtaga aatgattaga |
| 961 | aagggtggag atcagacttc actgttggtg gtagacaaag agacggacaa catgtacaga |
| 1021 | ctggctcatt tttctccatt tctctactat caaagtcaag aactgcccaa tggctctgtc |
| 1081 | aaggaggctc cagctcctac tcccacttct ctggaagtct caagtccacc agatactaca |
| 1141 | gaggaagtag atcataagcc taaactctgc aggctggcta aaggtgaaaa tggctatggc |
| 1201 | tttcacttaa atgcgattcg gggtctgcca ggctcattca tcaaagaggt acagaagggc |
| 1261 | ggtcctgctg acttggctgg gctagaggat gaggatgtca tcattgaagt gaatggggtg |
| 1321 | aatgtgctag atgaacccta tgagaaggtg gtggatagaa tccagagcag tgggaagaat |
| 1381 | gtcacacttc tagtctgtgg aaagaaggcc tatgattatt tccaagctaa gaaaatccct |
| 1441 | attgtttcct ccctggctga tccacttgac acccctccag attctaaaga aggaatagtg |
| 1501 | gtggagtcaa accatgactc gcacatggca aaagaacggg cccacagtac agcctcacat |
| 1561 | tcttcttcca attctgaaga tacagagatg tgatgaaaac aagtaatagc tttggctgtt |
| 1621 | tatttgatag ctgtttctgg gtatttaata ggaatccttt ctcaaggaat gagttgtgac |
| 1681 | ctgtttactg tctctttaga agaaaaactc cactggaaac cattcaccat gtgtgactgt |
| 1741 | cttctgttat catttgtctt acaggcggct attgcagacg gctaatttat gcttaactta |
| 1801 | ggaagagata aggcaagagc tagatttttt tcatgtgatc ttttccaagc ttcaacttaa |
| 1861 | cttaactaca tttctctgta tgatgatgtc tcttacttct acaggttcct tgagcaccaa |
| 1921 | agatgattca taactctgta taggtgacag ctgcttataa aagcatctta gcagataagc |
| 1981 | ctattaaaat tgtgcttttg taacaatgtt gtggttgcta gaataaatac catgaacccg |

21: AI188190 DIS3

| | |
|---|---|
| 1 | tttaaaagcc actaattatc tgttttttat tttgtaagta acaagatata gacatttgaa |
| 61 | tgccaatgtc ttattctgga gagacactgg agctgaagtt caacaatgat cacacttatt |
| 121 | acctggcaat aaaaacacaa ccatctttcc agtcaggtca aaatatccta ctttttgcct |
| 181 | ttctaccaaa tcccaaacat tcacagtttt tcaaggacca ctaataaaat acaggaagct |
| 241 | tttaaagaca gtaagagaac acctagtgta agttaggtga attaaagatg gcaaaggaga |
| 301 | ttacatcctc aacactgaca gcttccaaga cttagaaaag agattgttcc ttgcttctaa |
| 361 | aattgttcta ttttcctctg taggaaaatg aaagtttttt cttacaaata ttaaataatc |
| 421 | aaagtactta cgcaaaatta atctgctcct caatgagatg agcactccat ttaaatgatc |
| 481 | tttacagatc cctgaagttg ctgtcctgtc actgtattta agtgatggat attcaattga |
| 541 | attattctgc ataaataatt ctggtcaacc cagacgtata gtagtatgat gggtcagata |
| 601 | cagtcaactg ttcaataaaa atgcagatgt ctg |

22: BC001535 CGI-48

| | |
|---|---|
| 1 | ctgcgtttct cctcaaacct aacgatgccg ccggagcgga ggagacgaat gaaactggac |
| 61 | cggagaaccg gagcgaagcc gaagcggaag cccggaatga ggccggactg gaaagccgga |
| 121 | gcggggccag gcgggcctcc ccaaaagcct gccccttcat cccagcggaa accgccggcc |
| 181 | cggccgagcg cggcggccgc tgcgattgca gtcgcggcgg cggaggaaga gagacggctc |
| 241 | cggcagcgga accgcctgag gctggaggag gacaaaccgg ccgtggagcg gtgcttggag |
| 301 | gagctggtct tcggcgacgt cgagaacgac gaggatgcgt tgctgcggcg tctgcgaggc |
| 361 | ccgagggttc aagaacatga agactcgggt gactcagaag tggagaatga agcaaaaggt |
| 421 | aattttccac ctcaaaagaa gccagtttgg gtggatgaag aagatgaaga tgaggaaatg |
| 481 | gttgacatga tgaacaatcg gtttcggaag gatatgatga aaaatgctag tgaaagtaaa |
| 541 | ctttcgaaag acaaccttaa aaagagactt aaagaagaat tccaacatgc catgggagga |
| 601 | gtacctgcct gggcagagac tactaagcgg aaaacatctt cagatgatga aagtgaagag |
| 661 | gatgaagatg atttgttgca aaggactggg aatttcatat ccacatcaac ttctcttcca |
| 721 | agaggaatct tgaagatgaa gaactgccag catgcgaatg ctgaacgtcc tactgttgct |
| 781 | cggatctcat ctgtgcagtt ccatcccggt gcacagattg tgatggttgc tggattagat |
| 841 | aatgctgtat cactatttca ggttgatggg aaaacaaatc ctaaaattca gagcatctat |
| 901 | ttggaaaggt ttccaatctt taaggcttgt tttagtgcta atggggaaga agttttagcc |
| 961 | acgagtaccc acagcaaggt tctttatgtc tatgacatgc tggctggaaa gttaattcct |
| 1021 | gtgcatcaag tgagaggttt gaaagagaag atagtgagga gctttgaagt ctccccagat |
| 1081 | gggtccttct tgctcataaa tggcattgct ggatatttgc atttgctagc aatgaagacc |
| 1141 | aaagaactga ttggaagcat gaaaattaat ggaagggttg cagcatccac attctcttca |
| 1201 | gatagtaaga aagtatacgc ctcttcgggg gatggagaag tttatgtttg ggatgtgaac |
| 1261 | tcaaggaagt gccttaacag atttgttgat gaaggcagtt tatatggatt aagcattgcc |
| 1321 | acatctagga atggacagta tgttgcttgt ggttctaatt gtggagtggt aaatatatac |
| 1381 | aatcaagatt cttgtctcca agaaacaaac ccaaagccaa taaaagctat aatgaacttg |
| 1441 | gttacaggtg ttacttctct gaccttcaat cctactacag aaatcttggc aattgcttca |
| 1501 | gaaaaaaatg aaagaagcag tcagattggt tcatcttcct tcctgtacag tattttcaaa |
| 1561 | cttcccagtc attaaaaata agaatatttc tcatgttcat accatggatt tttctccgag |
| 1621 | aagtggatac tttgccttgg ggaatgaaaa gggcaaggcc ctgatgtata ggttgcacca |
| 1681 | ttactcagac ttctaaagag actatttgaa gtccagttga gtcacaagag aagcctgtct |
| 1741 | tgatatatca tctcagaaac tttcctgaat atgtgataat atatggaaaa tgatttatag |
| 1801 | atccagctgt gcttaagagc cagtaatgtc ttaataaaca tgtggcagct tttgtttgaa |
| 1861 | aaaaaaaaaa aaaaaaaa |

23: NM_007007 CPSF6

| | |
|---|---|
| 1 | aattccgggc ggcggcggcc gaggctgaag gaagatggcg gacggcgtgg accacataaa |
| 61 | catttacgcg gatgtcggcg aagagttcaa ccaggaagct gaatatggtg ggcatgatca |
| 121 | gatagatttg tatgacgatg tcatatctcc atctgcaaat aatggagatg ccccagaaga |
| 181 | ccgagattac atggatactc tcccaccaac tgttggtgat gatgtgggta aaggagcagc |
| 241 | accaaatgtt gtctatacat atactggaaa gagaattgca ttatatattg gaaatctaac |
| 301 | atggtggaca acagatgaag acttaactga agcagttcat tctttgggag taaatgatat |
| 361 | tttggagata aaattttttg aaaatcgagc aaatggccag tcaaaggggt ttgcccttgt |
| 421 | tggtgttgga tctgaagcat cttcaaaaaa gttaatggat ctgttaccta aaagagaact |
| 481 | tcatggtcag aatcctgttg taactccatg caataaacag ttcctgagtc aatttgaaat |
| 541 | gcagtccagg aaaactacac aatcaggaca aatgtctggg gaaggtaaag ctggtcctcc |
| 601 | aggaggcagt tcccgtgcag catttccaca aggtggtaga ggacggggcc gttttccagg |
| 661 | ggctgttcct ggtggggaca gatttcctgg gccagcagga ccaggagggc cacccccacc |
| 721 | ttttccagct ggacagactc caccacgtcc acccttaggt cctccaggcc cacctggtcc |
| 781 | accaggtcct ccacctcctg gtcaggttct gcctcctcct ctagctgggc ctcctaatcg |
| 841 | aggagatcgc cctccaccac cagttctttt tcctggacaa ccttttgggc agcctccatt |
| 901 | gggtccactt cctcctggcc ctccacctcc agttccaggc tacggccccc ctcctggccc |
| 961 | accacctcca caacagggac cacctccacc tccaggcccc tttccacctc gtccacccgg |
| 1021 | tccacttggg ccacccctta cactagctcc tcctccgcat cttcctggac cacctccagg |
| 1081 | tgccccaccg ccagctccgc atgtgaaccc agctttcttt cctccaccaa ctaacagtgg |
| 1141 | catgcctaca tcagatagcc gaggtccacc accaacagat ccatatgggc gacctccacc |
| 1201 | atatgatagg ggtgactatg gcccccctgg aagggaaatg gatactgcaa gaacgccatt |
| 1261 | gagtgaagct gaatttgaag aaatcatgaa tagaaatagg gcaatctcaa gcagtgctat |
| 1321 | ttcgagagct gtgtctgatg ccagtgctgg tgattatggg agtgctattg agacactggt |
| 1381 | aactgcaatt tctttaatta aacaatccaa agtatctgct gatgatcgtt gcaaagttct |
| 1441 | tattagttct ttgcaagatt gccttcatgg aattgagtcc aagtcttatg gttctggatc |
| 1501 | aagacgtgaa cgatcaagag agagggacca tagtagatca cgagaaaaga gtcgacgtca |
| 1561 | taaatcccgt agtagagacc gtcatgacga ttattacaga gagagaagca gagaacgaga |
| 1621 | gaggcaccgg gatcgtgacc gagaccgtga ccgagagcgt gaccgagagc gcgaatatcg |
| 1681 | tcatcgttag aagctgaagg aagaggatca ccttccaaga caaaacagtc ttcatgggcc |
| 1741 | aaaaatgacg cttgtccagc agtttgcttc ttgtgattga actgaacctg taaggattca |
| 1801 | tggataaaat gaacaggaat agatctgaat aaagcaaatc tgcataaatg gtaaccagta |
| 1861 | gctctacttt tattttttat gttgcttaac tgttttattt gaaggaaacc tgtgtgattt |
| 1921 | aaaaagttat agcttttgca actttattac tggttatata catttggcca ttatgatgtg |
| 1981 | caagcaattg gaaaaaaagt caagtaaatg cttgtttttg tagtagtttg ttcttgttaa |
| 2041 | aaatgtttat atgataatgt ctgtaaacag catcactttg attacaatag atgtagtgtt |
| 2101 | gtaataaact gtttaatggg gctgatgtgt aaagctgttc aagttatttg atgtttacac |
| 2161 | ctcagggaaa gtcttgtgtt cagcaatatc taaagataat gttactatga caacattttt |
| 2221 | actgtccttt aaagcattgc aatagcgttt ttggatatgc ctcaatctaa tcttgcgttc |
| 2281 | agtgaattaa acatagtaat taagtgtctt ttgcccttga ttttgatatt agaataggtg |
| 2341 | attacatgga tatttaatat ttctatattc tgcttttcta gctgttttta cctagttagc |
| 2401 | ttgtgacttt gctgaatggt atgtaaactt gtaaaaatag agatttgaca gacatagcaa |
| 2461 | tctagtcaat gtgtaagggg tcaaaaaaaa cagaggtttt aacacataag taaaaacccg |
| 2521 | tacatatttg atgtgtaatg caggttaatt acaacacaga tgtaccgaaa cacttaattg |
| 2581 | tgaaccgcta acattgaaga aattttgaca attccgattt gatgctgcaa ttacttgctg |
| 2641 | tttttattga tcttatggtt tatttcttaa gccatagtca gtgtaaatac agccctgcag |
| 2701 | caggtaaatg tgagtaaaga gagccttata ttttccaatt ggtataaaat ttttgaagga |
| 2761 | tgtgatgttc attaacattc ggttgtattc cccagtattt gtaatgggaa attacagata |
| 2821 | aaccgtgtct gcacagttta aggaatacta tgtatattca tgcaccgtat tgattcatgc |
| 2881 | tatagttact taatcaaaga tttttttcaa acctgcctta catataggcc cactttaaaa |
| 2941 | gcacctgact agcatgtgtt cttgattgca aaattggcag aggcagggtg tcaacttgat |
| 3001 | taggtgtttt tatgggaatg taatttgaaa tcactacttc agaaatttga cttaaaattc |
| 3061 | ttgagcacgt taatatgttt ttaagatctg attatctttg agagatcttc tgttaataca |
| 3121 | cattggttgt taaagagtac ccaaattcta ggacaatgct taaagtgtta aaatacccta |
| 3181 | gatactgtgt tatgtgcaac tgtagaaacc ctccagaaat ttccactgct gttcttcact |
| 3241 | ttcatcttgt ctgctatcaa accacttctg acaaaattag ctgttttgaa ttacccatat |
| 3301 | cactgccagt tttattttaa aatattttgt gtttgaagta tctgtgcatg ggatcgttga |
| 3361 | tgtttatcag aactgttcac tttcagaaat gattttttaa agcattttgt tgaaatgcgg |
| 3421 | ttgctt |

24: NM_002254 KIF3C

| | |
|---|---|
| 1 | tcactctcgc tgccgctgct ccgccccatc cccttctgtt tttctctctc attctccagt |
| 61 | ggcggcggcg gggaaggcgg aggcagaggc agcagcagcc gcgctggctg caatgaatga |
| 121 | tcccccagct tggggggagg actccaggtg agcctctgcc ctcgggaggc ccgggacccc |
| 181 | cggccgccca cgaccggcag cccacgctat ggatccctag aggaaggagg agaagacagc |
| 241 | tcgccgccca cccccatccc attttcctct tcctttatct cattgttgcc gaagctgttt |
| 301 | acggcagcgc tccctctgct ccagcatggg gcgggctccg ggcacggctg ctcggcaggc |
| 361 | gctgctcccg cggcgactgg gggattctgc ctaattcacc tcccagccgg tgcagagagg |
| 421 | accggagagc ggtggaggcc cggactgcag cagcgttggg gccacctccc agcgtcccca |
| 481 | ccctaggagg ctgcatgcgg attgaagagc tgcgcctggg ggctgggccg gccccgctga |
| 541 | tcccgaccta gcgagcagga tagcaggacc gcccaggctg cggaggggct cgggggcagg |
| 601 | aaggtcagag cagcaagatg gccagtaaga ccaaggccag cgaggccctc aaggtggtgg |
| 661 | cccggtgccg ccccctcagc aggaaggagg aggctgctgg tcacgagcag atcctgacca |
| 721 | tggacgtgaa actgggccag gtgaccctgc ggaacccccg cgccgccccg ggggagctgc |
| 781 | ccaagacctt cacctttgac gccgtgtatg atgccagctc caagcaggcc gacctgtatg |
| 841 | acgaaaccgt gaggcccctg atagactccg tgctccaggg tttcaatggc acggtgtttg |
| 901 | cctatggcca gacgggcact ggcaagacct ataccatgca ggggacctgg gtggagcccg |
| 961 | agctgcgcgg ggtcatcccg aatgcctttg agcacatctt cacccacatc tcccgctccc |
| 1021 | agaaccaaca gtacctggtc cgggcctcct atttggagat ctaccaggaa gagattcgag |
| 1081 | acctgctctc caaggagccg ggcaagaggc tagagctgaa agagaacccc gagactggcg |
| 1141 | tctacatcaa ggacctctcc tccttcgtca ccaagaatgt caaggagatt gagcatgtga |
| 1201 | tgaacctggg gaaccagacc cgggctgtgg gcagcaccca catgaatgag gtcagctccc |
| 1261 | gctcccatgc catcttcatc atcactgtgg agtgcagcga acgtggctct gatggccagg |
| 1321 | accacatccg agtgggcaag ctcaacctcg tggacctggc tggcagcgag aggcagaaca |
| 1381 | aggcaggccc caacacagcg ggaggggcag ccacaccatc ctcgggtggc ggtggtggcg |
| 1441 | gtggaggcag tggtggtggt gctggtggag agaggcctaa ggaagcctcc aaaatcaacc |
| 1501 | tctcattatc tgccctgggc aacgtgattg ctgccctggc gggcaacagg agcacccaca |
| 1561 | ttccctaccg ggactccaag ctgacccggc tgctccagga ctccctgggg gggaatgcca |
| 1621 | agaccatcat ggtagccaca ctggggccag cttctcacag ctacgatgag agcctctcca |
| 1681 | ccttgcgctt tgccaaccga gccaagaaca tcaagaacaa gccccgggtg aacgaggacc |
| 1741 | ccaaggacac actgctgcgg gaattccaag aggagattgc ccgcctgaag gcccagctgg |
| 1801 | agaagagggg gatgctgggg aagcggcccc ggaggaagag cagccgcagg aagaaggccg |
| 1861 | tgtccgcccc gcctgggtac cctgagggcc cagtgattga ggcctgggtg gcagaagagg |
| 1921 | aggatgacaa caacaacaac caccgcccgc cccagcccat cctggagtca gccttggaga |
| 1981 | agaacatgga gaattacctg caggaacaga aggagcggct ggaggaggag aaggcagcca |
| 2041 | tccaggatga ccgcagcctg gtgagcgagg agaagcagaa gctgctggag gagaaggaga |
| 2101 | agatgctgga ggacctgcgg cgggaacagc aggccacaga gctgcttgcg gccaagtaca |
| 2161 | aggccatgga gagcaagctc ctcatcgggg gcaggaacat catggatcac accaacgaac |
| 2221 | agcagaagat gttggaactg aagaggcagg agattgccga gcagaaacgt cgtgagcggg |
| 2281 | agatgcagca ggagatgatg ctccgggacg aggagactat ggagctccgg ggcacctaca |
| 2341 | catccctgca gcaggaggtg gaggtcaaaa ccaagaaact caagaagctc tacgccaagc |
| 2401 | tgcaggcggt gaaggcggag atccaggacc agcatgatga gtatatccgc gtgcggcagg |
| 2461 | acctggagga ggcgcagaac gagcagaccc gcgaactcaa gctcaagtac ctaatcatcg |
| 2521 | agaacttcat cccgccggag gagaagaaca agatcatgaa ccggcttttc ctggactgtg |
| 2581 | aggaggagca gtggaagttc cagccactgg tgccagccgg cgtcagtagc agccagatga |
| 2641 | agaagcggcc aacatctgca gtgggctaca agaggcctat cagccagtat gctcgggttg |
| 2701 | ccatggcaat ggggtcccac cccaggtaca gggctgaaaa cataatgttt ctggagttgg |
| 2761 | atgtgtcccc tccagctgtc tttgagatgg aattctctca cgaccaagaa caagaccctc |
| 2821 | gtgcgctaca catggagagg ctcatgcgat tggacagctt tctggaaaga ccttccacgt |
| 2881 | ctaaagtccg aaagtccaga tcctggtgcc agagtcctca gcggcctcca ccttccacca |
| 2941 | cacatgcctc cctggcctct gcttctctgc gccctgcaac agtggcggac catgagtgac |
| 3001 | aaccatcacg tcaggctgcc catccaatag actcctggga tggggcagcc aaccctggct |
| 3061 | catctcatct gccgcttggt gcgtgtgcgt gtgcgtgcat gtgcgtgtgc gtgtgtgcag |
| 3121 | gggtgagaat ctggcagatg gtgcctctgc ctgctcttct tcgcctcctt tatttaattc |
| 3181 | atgttattta ttcgcggagc tctgttcgtg ttggggagat gccctcgcct gagccgtctg |
| 3241 | ggcctaccgt ggtcactgcg tagcctcttt ttcttctgac ttgagagctc ccccagtcag |
| 3301 | atctcaggct tgtccccctg tcagctgcct ccagaaggga aggtagccag tgcctgagaa |
| 3361 | gacagtccct tttctaccca ccgcactcca taacctccat cttctcccac actgatggcg |
| 3421 | agcagcccct gagcactttc tgggactggg agactgcttg gtgttccctg aggacaagag |
| 3481 | acatcctgac agtgttgggc atctgctccc cgtggacaca gccccactct ccactttctg |
| 3541 | agcctcagac aacctcattc agcctcttgg gctccttttc aaggacatta ataacctcac |
| 3601 | caacatagct catgcccttc agctttgaca agaactcacg gcttcccaaa ctctgctttc |
| 3661 | tgcccacctt ggatgggaac tgtggaccaa gcaattacca tcgccttgga acctgcagga |
| 3721 | aatggaacag caattgagac aacttgaaca gtcatcaacg gaagtccctc cactggattc |
| 3781 | ctttgtttct gtcccctccg aggagtcatt ttggtcgaca ggctctcaag gcaactcccc |
| 3841 | attttcaaga ggctgctcct gcctgcttcg atcatttctc cctgcagctg cctagacccc |
| 3901 | gttcacagtg ggaggagtca atgtcattct acccctcgct aaacgaagat attaacatct |
| 3961 | attgcttttt cccttcatct gtcacaggaa acagaagccc aggcacaatc ttttccagct |
| 4021 | ttgcctgtta cccctgtttc tgaattgcat ctttaaggta ttattttgtt gacaatagat |
| 4081 | cctttattca ctagttacgc aaattggttc ctagggggat actccttacc ttcctttgtg |
| 4141 | atggcccaaa atgtctctag gtatctcaag tgataagtaa atttctacaa aaaaaaatgg |
| 4201 | ttaatgttca ttgactggct ttttaagtgt atattttgga ggacgggtga agaggtcata |
| 4261 | acgaaagcaa gcgagtgaat taggatttca aagtgcccta atagtgtgag tctccagttc |
| 4321 | ctagaatatg aagagtgctg tcgttggggt gaaaccatga gactgacaga tctgcctgaa |
| 4381 | atggggggtg tgggaggtgg tggcgggggt tattctcttt ccttcaggaa atgaaccctt |
| 4441 | cttacatcat tcaagttctg ctctgaggat caagcttggg tctgatttaa ctcagcgaca |
| 4501 | ctgtcatttc tgcttcatta ctggactaga gggttgagcc acccacttgc catttgctcc |
| 4561 | tgtccttcca ggaaatcaca attttcatca gagcccaaga gattatttga gactcaggat |
| 4621 | tcagatcaga ggttcgactg tggctgggac aggagttgtg tgtagaaatt caccaggtgg |
| 4681 | cctgagcgca gggggacctc cagggctgcg ttgagcagcc tctcccactg acctctttct |
| 4741 | cgtttgtgga caaagcagca cgtatcacct cattcatcac ttggacacat cgcctttgca |
| 4801 | ttgtcttgtc acacctccct cacagtctta tagcacaata tacccaaatc agccccccca |
| 4861 | gtccgagggc tgggcccaag gtatggtcgg aggaggagct cctgcctgcg gttttgtgta |
| 4921 | tgtgtgtatg tgtgtgcgtg tttgtgtgcg tgtttacctc cacaggggac actctacact |
| 4981 | cagtgtaaga tctgctggga acagggccac caggagtggc tggatctcag tctctctgtc |
| 5041 | tctctttctc tccttttcct tttggtgtat caaatatttg attgacaaag taagggcctt |
| 5101 | gattaggacc aaattctcgt gtgttgctat ggtctttatt taggacaaca attaacaatg |
| 5161 | cagtggccca ttcttgtcac tctacacata tgactatacg ggacatatgt aatatataaa |
| 5221 | tatatatata aaacattccc ctctgtcccc ttggcttcgg atggaggcct ttctgttgag |
| 5281 | ctgaaatgca cctgcagctg ggtgctgcca gcagcttgca ggccccagcc ctgttccaat |
| 5341 | caatgcagtt gacaataaag gaatgagtat cgtcacggaa aaaaaaaaaa aaaaaaaaaa |
| 5401 | a |

25: BQ135232 CD9

| | |
|---|---|
| 1 | taaacaatgg tatcaacgca aagtaagcgg gcagccgcct gcatctgtat ccagcgccag |
| 61 | gtcccgccag tcccagtgcg cgcgcccccc agtcccgcac ccgttcggcc aggctaagtt |
| 121 | agccctcacc atgcggtcaa aggaggcagc aagtgcatca aatacctgct gttcggattt |
| 181 | aacttcatct tctggcttgc cgggattgct gtccttgcca ttggactatg ggtccgattc |
| 241 | gactctcaga ccaagagcat cttcgagcaa gaaatttcct aataataata attccagctt |
| 301 | ctacacagga gtctatattc tgatcggagc cggcgccctc atgatgctgg tgggcttcct |
| 361 | gggctgctgc ggggctgtgc aggagtccca gtgcatgctg ggactgttct tcggcttcct |
| 421 | cttggtgata ttcgccattg aaatagctgc ggccatctgg ggatattccc acaaggatga |
| 481 | ggtgattaag gaagtccagg agttttacaa ggacacctac aacaagctga aaaccaagga |
| 541 | tgagccccag cgggaaacgc tgaaagccat ccactatgcg ttgaactgct gtggtttggc |
| 601 | tgggggcgtg gaacagttta tctcagacat ctgccccaag aaggacgtac tcgaaacctt |
| 661 | caccgtgaag tcctgtcctg atgccatcaa agaggtcttc gaccaataaa ttccacatca |
| 721 | tcggcgcagt gggcatcggc attgccgtgg tcatgatatt tggcatgatc ttcagtatga |
| 781 | tcttgtgctg tgctatccgc aggaaccgcg agatggtcta gagtcagctt acatccctga |
| 841 | gcaggaaagt ttacccatga agattggtgg gattttttgt ttgtttgttt tgttttgttt |
| 901 | gttgtttgtt gtttgttttt ttgccactaa ttttagtatt cattctgcat tgctagataa |
| 961 | aagctgaagt tactttatgt ttgtctttta atgcttcatt caatattgac atttgtagtt |
| 1021 | gagcgggggg tttggtttgc tttggtttat attttttcag ttgtttgttt ttgcttgtta |
| 1081 | tattaagcag aaatcctgca atgaaaggta ctatatttgc tagactctag acaagatatt |
| 1141 | gtacataaaa gaattttttt gtctttaaat agatacaaat gtctatcaac tttaatcaag |
| 1201 | ttgtaactta tattgaagac aatttgatac ataataaaaa attatgacaa tggccaaaaa |
| 1261 | aaaaaaaaaa aaaaaaaaaa |

26: BC051322 LRRC8

| | |
|---|---|
| 1 | gcggccgggg cctggggctg cctgccgggc ggccgggcgc ggcgagccca gggaggcagc |
| 61 | gtccatggag caaaaggaat gccaggatcc tgcacaggca gacgcgggcc agcctcagca |
| 121 | ccgacagccg acgcgcagat agcagagcca tccttggggt tgaaccatga ttccggtgac |
| 181 | agagctccgc tactttgcgg acacgcagcc agcataccgg atcctgaagc cgtggtggga |
| 241 | tgtgttcaca gactacatct ctatcgtcat gctgatgatt gccgtcttcg gggggacgct |
| 301 | gcaggtcacc caagacaaga tgatctgcct gccttgtaag tgggtcacca aggactcctg |
| 361 | caatgattcg ttccggggct gggcagcccc tggcccggag cccacctacc ccaactccac |
| 421 | cattctgccg acccctgaca cgggccccac aggcatcaag tatgacctgg accggcacca |
| 481 | gtacaactac gtggacgctg tgtgctatga gaaccgactg cactggtttg ccaagtactt |
| 541 | cccctacctg gtgcttctgc acacgctcat cttcctggcc tgcagcaact tctggttcaa |
| 601 | attcccgcgc accagctcga agctggagca ctttgtgtct atcctgctga agtgcttcga |
| 661 | ctcgccctgg accacgaggg ccctgtcgga gacagtggtg gaggagagcg accccaagcc |
| 721 | ggccttcagc aagatgaatg ggtccatgga caaaaagtca tcgaccgtca gtgaggacgt |
| 781 | ggaggccacc gtgcccatgc tgcagcggac caagtcacgg atcgagcagg gtatcgtgga |
| 841 | ccgctcagag acgggcgtgc tggacaagaa ggagggggag caagccaagg cgctgtttga |
| 901 | gaaggtgaag aagttccgga cccatgtgga ggagggggac attgtgtacc gcctctacat |
| 961 | gcggcagacc atcatcaagg tgatcaagtt catcctcatc atctgctaca ccgtctacta |
| 1021 | cgtgcacaac atcaagttcg acgtggactg caccgtggac attgagagcc tgacgggcta |
| 1081 | ccgcacctac cgctgtgccc accccctggc cacactcttc aagatcctgg cgtccttcta |
| 1141 | catcagccta gtcatcttct acggcctcat ctgcatgtat acactgtggt ggatgctacg |
| 1201 | gcgctccctc aagaagtact cgtttgagtc gatccgtgag gagagcagct acagcgacat |
| 1261 | ccccgacgtc aagaacgact tcgccttcat gctgcacctc attgaccaat acgacccgct |
| 1321 | ctactccaag cgcttcgccg tcttcctgtc ggaggtgagt gagaacaagc tgcggcagct |
| 1381 | gaacctcaac aacgagtgga cgctggacaa gctccggcag cggctcacca agaacgcgca |
| 1441 | ggacaagctg gagctgcacc tgttcatgct cagtggcatc cctgacactg tgtttgacct |
| 1501 | ggtggagctg gaggtcctca agctggagct gatccccgac gtgaccatcc cgcccagcat |
| 1561 | tgcccagctc acgggcctca aggagctgtg gctctaccac acagcggcca agattgaagc |
| 1621 | gcccgcgctg gccttcctgc gcgagaacct gcgggcgctg cacatcaagt tcaccgacat |
| 1681 | caaggagatc ccgctgtgga tctatagcct gaagacactg gaggagctgc acctgacggg |
| 1741 | caacctgagc gcggagaaca accgctacat cgtcatcgac gggctgcggg agctcaaacg |
| 1801 | cctcaaggtg ctgcggctca agagcaacct aagcaagctg ccacaggtgg tcacagatgt |
| 1861 | gggcgtgcac ctgcagaagc tgtccatcaa caatgagggc accaagctca tcgtcctcaa |
| 1921 | cagcctcaag aagatggcga acctgactga gctggagctg atccgctgtg acctggagcg |
| 1981 | catcccccac tccatcttca gcctccacaa cctgcaggag attgacctca aggacaacaa |
| 2041 | cctcaagacc atcgaggaga tcatcagctt ccagcacctg caccgcctca cctgccttaa |
| 2101 | gctgtggtac aaccacatcg cctacatccc catccagatc ggcaacctca ccaacctgga |
| 2161 | gcgcctctac ctgaaccgca acaagatcga gaagatcccc acccagctct tctactgccg |
| 2221 | caagctgcgc tacctggacc tcagccacaa caacctgacc ttcctccctg ccgacatcgg |
| 2281 | cctcctgcag aacctccaga acctagccat cacggccaac cggatcgaga cgctccctcc |
| 2341 | ggagctcttc cagtgccgga agctgcgggc cctgcacctg ggcaacaacg tgctgcagtc |
| 2401 | actgccctcc agggtgggcg agctgaccaa cctgacgcag atcgagctgc ggggcaaccg |
| 2461 | gctggagtgc ctgcctgtgg agctgggcga gtgcccactg ctcaagcgca gcggcttggt |
| 2521 | ggtggaggag gacctgttca acacactgcc acccgaggtg aaggagcggc tgtggagggc |
| 2581 | tgacaaggag caggcctgag cgaggccggc ccagcacagc aagcagcagg accgctgccc |
| 2641 | agtcctcagg cccggagggg caggcctagc ttctcccaga actcccggac agccaggaca |
| 2701 | gcctcgtggc tgggcaggag cctggggccg cttgtgagtc aggccagagc gagaggacag |
| 2761 | tatctgtggg gctggcccct tttctccctc tgagactcac gtcccccagg gcaagtgctt |
| 2821 | gtggaggaga gcaagtctca agagcgcagt atttggataa tcagggtctc ctccctggag |
| 2881 | gccagctctg ccccaggggc tgagctgcca ccagaggtcc tgggaccctc actttagttc |
| 2941 | ttggtattta tttttctcca tctcccacct ccttcatcca gataacttat acattcccaa |
| 3001 | gaaagttcag cccagatgga aggtgttcag ggaaaggtgg gctgcctttt ccccttgtcc |
| 3061 | ttatttagcg atgccgccgg gcatttaaca cccacctgga cttcagcaga gtggtccggg |
| 3121 | gcgaaccagc catgggacgg tcacccagca gtgccgggct gggctctgcg gtgcggtcca |
| 3181 | cgggagagca ggcctccagc tggaaaggcc aggcctggag cttgcctctt cagtatttgt |
| 3241 | ggcagtttta gttttttgtt tttttttttt taatcaaaaa acaatttttt taaaaaaaaa |
| 3301 | gctttgaaaa tggatggttt gggtattaaa aaaaaaaaaa aaaaa |

27: BC038504 SNF1LK

| | |
|---|---|
| 1 | atgcggcgcg gccccggagg cagcagcagc ggcggcggca gccggagcag taggcacccg |
| 61 | agcagcgcca gcggccgagc gggcggcttc ctggcctggg cgctccggtg gcggcggagg |
| 121 | tgcgcgcgga gccatggtta tcatgtcgga gttcagcgcg gaccccgcgg gccagggtca |
| 181 | gggccagcag aagcccctcc gggtgggttt ttacgacatc gagcggaccc tgggcaaagg |
| 241 | caacttcgcg gtggtgaagc tggcgcggca tcgagtcacc aaaacgcagg ttgcaataaa |
| 301 | aataattgat aaaacacgat tagattcaag caatttggag aaaatctatc gtgaggttca |
| 361 | gctgatgaag cttctgaacc atccacacat cataaagctt taccaggtta tggaaacaaa |
| 421 | ggacatgctt tacatcgtca ctgaatttgc taaaaatgga gaaatgtttg attatttgac |
| 481 | ttccaacggg cacctgagtg agaacgaggc gcggaagaag ttctggcaaa tcctgtcggc |
| 541 | cgtggagtac tgtcacgacc atcacatcgt ccaccgggac ctcaagaccg agaacctcct |
| 601 | gctggatggc aacatggaca tcaagctggc agattttgga tttgggaatt tctacaagtc |
| 661 | aggagagcct ctgtccacgt ggtgtgggag ccccccgtat gccgccccgg aagtctttga |
| 721 | ggggaaggag tatgaaggcc cccagctgga catctggagc ctgggcgtgg tgctgtacgt |
| 781 | cctggtctgc ggttctctcc ccttcgatgg gcctaacctg ccgacgctga gacagcgggt |
| 841 | gctggagggc cgcttccgca tccccttctt catgtctcaa gactgtgaga gcctgatccg |
| 901 | ccgcatgctg gtggtggacc ccgccaggcg catcaccatc gcccagatcc ggcagcaccg |
| 961 | gtggatgcgg gctgagccct gcttgccggg acccgcctgc cccgccttct ccgcacacag |
| 1021 | ctacacctcc aacctgggcg actacgatga gcaggcgctg ggtatcatgc agaccctggg |
| 1081 | cgtggaccgg cagaggacgg tggagtcact gcaaaacagc agctataacc actttgctgc |
| 1141 | catttattac ctcctccttg agcggctcaa ggagtatcgg aatgcccagt gcgcccgccc |
| 1201 | cgggcctgcc aggcagccgc ggcctcggag ctcggacctc agtggtttgg aggtgcctca |
| 1261 | ggaaggtctt tccaccgacc ctttccgacc tgccttgctg tgcccgcagc cgcagacctt |
| 1321 | ggtgcagtcc gtcctccagg ccgagatgga ctgtgagctc cagagctcgc tgcagtggcc |
| 1381 | cttgttcttc ccggtggatg ccagctgcag cggagtgttc cggccccggc ccgtgtcccc |
| 1441 | aagcagcctg ctggacacag ccatcagtga ggaggccagg caggggccgg gcctagagga |
| 1501 | ggagcaggac acgcaggagt ccctgcccag cagcacgggc cggaggcaca ccctggccga |
| 1561 | ggtctccacc cgcctctccc cactcaccgc gccatgtata gtcgtctccc cctccaccac |
| 1621 | ggcaagtcct gcagagggaa ccagctctga cagttgtctg accttctctg cgagcaaaag |
| 1681 | ccccgcgggg ctcagtggca ccccggccac tcaggggctg ctgggcgcct gctccccggt |
| 1741 | caggctggcc tcgcccttcc tggggtcgca gtccgccacc ccagtgctgc aggctcaggg |
| 1801 | gggcttggga ggagctgttc tgctccctgt cagcttccag gagggacggc gggcgtcgga |
| 1861 | cacctcactg actcaagggc tgaaggcctt tcggcagcag ctgaggaaga ccacgcggac |
| 1921 | caaagggttt ctgggactga acaaaatcaa ggggctggct cgccaggtgt gccaggtccc |
| 1981 | tgccagccgg gccagcaggg gcggcctgag ccccttccac gcccctgcac agagcccagg |
| 2041 | cctgcacggc ggcgcagccg gcagccggga gggctggagc ctgctggagg aggtgctaga |
| 2101 | gcagcagagg ctgctccagt tacagcacca cccggccgct gcacccggct gctcccaggc |
| 2161 | cccccagccg gcccctgccc cgtttgtgat cgccccctgt gatggccctg gggctgcccc |
| 2221 | gctccccagc accctcctca cgtcggggct cccgctgctg ccgcccccac tcctgcagac |
| 2281 | cggcgcgtcc ccggtggcct cagcggcgca gctcctggac acacacctgc acattggcac |
| 2341 | cggccccacc gccctccccg ctgtgccccc accacgcctg gccaggctgg ccccaggttg |
| 2401 | tgagcccctg gggctgctgc agggggactg tgagatggag gacctgatgc cctgctccct |
| 2461 | aggcacgttt gtcctggtgc agtgagggca gccctgcatc ctggcacgga cactgactct |
| 2521 | tacagcaata acttcagagg aggtgaagac atctggcctc aaagccaaga actttctaga |
| 2581 | agcgaaataa gcaatacgtt aggtgttttg gctttttagt ttatttttgt tttatttttt |
| 2641 | tcttgcactg agtgacctca actttgagta gggactggaa actttaggaa gaaagataat |
| 2701 | tgaggggcgt gtctgggggc gggggcagga ggggagcggg gtggagggaa cacgtgcagt |
| 2761 | gccgtggtgt ggggatctcg gcccctctct ctgggttcgt cgtggttgag atgattacct |
| 2821 | cggacgtcta cggaaacgag cgggcgcatt gttgtccgct tgtgtgtgtg tgtgtgtgtg |
| 2881 | tgtgtgtgcg cgtgcattga ttactatcca tttctttagt caacgctctc cacttcctga |
| 2941 | tttctgcttt aaggaaaact gtgaactttc tgcttcatgt atcagtttta aagcagccca |
| 3001 | ggcaaagatc atctacagat tctaggaatt ctctcccctg aaatcaaaac ctggaagact |
| 3061 | tttttttctt attttagttg agaagtttca taaactgctc aaggattagt tttccaggac |
| 3121 | tctgcggagg aacggcagga agaacctcag agagggcaga ggtgacttca aagtgctggg |
| 3181 | gactccgtcc tgagggtcac ttggccctga gcccctgcgt gcccttgcgg aagcccagaa |
| 3241 | gcttcttcct gctgcacctc ccgtttccgc tgctgctgac gtttatgcat ttcatgatgg |
| 3301 | ggtccaacaa gaacacctga cttgggtgaa gttgtgcaat attggaggct gactgtaggg |
| 3361 | ctgggcagct gggagacagg ctcatggctc atggctcatg gctcagggcg gtgcctgcca |
| 3421 | tgggccggga cccccctccc caccccccac ctaggctttt tgggttttgt tcaaggaagg |
| 3481 | taaagtgaga ggtttaggtc agtgttttta agtttttgtt ttttttttaa agcaaatcct |
| 3541 | gtatatgtat ctacatggga gacaggtaga cactacttat ttgttacatt ttgtactaca |
| 3601 | cgtttgtgtt ccaggtttca gcttccctcg ctcctgttgt taagaagcgt ccctgtcagc |
| 3661 | acaggtgtgc attgaggaag gggccccagg gccttcgctc cctcagcact ggggtggagg |
| 3721 | cggcaggaag gggcggccct tacctggcag gtctgggcgc acctttagca ggtggactcc |
| 3781 | gtggggctcc accagccaga agcctttgga aggcaacgaa ggcaatgctg ctccctgagt |
| 3841 | ccagtccccg cccccaaacc cagcccaggt gccttcagct acttcggctt cttaaaccct |
| 3901 | gcagtgttaa acagaggcat tgagaaaggg gaaaggcggg tatttttaaa agccaaagat |
| 3961 | tgacccagtt acttgagggt agggaggcgg gcccagtgca ggaggctgca tccctggcct |
| 4021 | gctggtgccc accgggggct gtgcctgtgc cgggccgcag ggaagctggc tgcccccatt |
| 4081 | cctgctgctg ctgctgctgc tgctctgtgg ctgtttcaaa gactgggcga aaggctgtcc |
| 4141 | ggagggcaga ccaggtgcct tgccgcagag aaaacaccaa agtctcctgt tcgctcataa |
| 4201 | agaagttttt gggatgggag agaatccaga ccatcttggg gcagccaggc ccttgccttc |
| 4261 | atttttacag aggtagcaca attgattcca acacaaaact tccccttttt aaaatgattt |
| 4321 | ctgttctaat gccatagatc aaaggcctca gaaaccattg tgtgtttcct ctttgaagca |
| 4381 | atgacaagca ctttactttc acggtggttt ttgttttttc ttattgctgt ggaacctctt |
| 4441 | ttggaggacg ttaaaggcgt gttttacttg tttttttaag agtgtgtgat gtgtgttttg |
| 4501 | tagatttctt gacagtgctg taatacagac ggcaatgcaa tagcctattt aaagacacta |
| 4561 | cgtgatctga ttgagatgta catagttttt ttttttacca taactgaatt attttatctc |
| 4621 | ttatgttaac atgagaaatg tatgccaaat gattagttga tgtatgtttt ttaatttaat |
| 4681 | atttaaataa aatatttggg agtataaaaa aaaaaaaaaa aaaaaaaaaa aaaaaaaaaa |
| 4741 | aaaaaaaaaa aaaaaaaaaa aa |

28: U78556 CRA

| | |
|---|---|
| 1 | cacacctttc caaggacccc caaactctgc tccgtgcacg tcaaatgctc ctttcccttg |
| 61 | tgtccaaccc cctacccctc tccctaacac ccctcttctc aacaagactc agcctctccc |
| 121 | cgaggtgggt gagcatcctt gaggtttccc acccttaact gctgtgtccc cggatggagc |
| 181 | cagagaaatg tggtgggggg gccggggcag agtttcaaca ttgcccccca gaaggaggag |
| 241 | ccagagatgg ggtctgtcca ggaaaacagg atgccggagc ccaggagtcg tcagcctagc |
| 301 | agttgcctgg cctccagatg cctcccaggg gagcagatcc tagcatgggc cccaggggtg |
| 361 | aggaagggcc tggaaccaga attgtctgga accctgatct gtaccaactt tagggtcacc |
| 421 | ttccagccct gtggatggca gtggaatcag gacactccct tgaacagtga atacgatttt |
| 481 | gccctggtca acattggacg attagaggct gtgagcggct tgtcccgagt ccagctcctc |
| 541 | cgtccagggt ccctgcataa atttatccct gaggagattc tgattcatgg ccgagacttc |
| 601 | cggctgctca gagttggttt tgaggctgga ggcctagagc ctcaggcttt tcaggtgacc |
| 661 | atggccattg tccaagccag agctcagagc aatcaagccc aacagtattc ggggataacc |
| 721 | ctgagcaagg ctggccaggg ttctggctcc agaaaaccac caattcctct catggagaca |
| 781 | gcggaagact gggagactga gcggaagaag caggcagcca gaggctggag ggtcagcacg |
| 841 | gtcaacgaga ggttcgacgt agccaccagc ctcccccgtt acttctgggt ccctaaccga |
| 901 | attctggaca gtgaggtcag gagagcattt ggccactttc atcagggccg tggaccggtc |
| 961 | agtgtgatgg ttagggtaat ggctgtggat tagagggtca tgtgggccag ggacatcgtg |
| 1021 | gagggaggaa cctctgtgag gtcagtgtgg gggcaagggt agcgtggagc taggcatttc |
| 1081 | tcccacaatg accctcttct gccccatgtg aagcgcttgt cctggcatca ccctgggggc |
| 1141 | agtgatcttc tccgctgtgg aggcttctat acagccagtg accctaacaa ggaggatatc |
| 1201 | agagcagtgg agttgatgca ccaggctggg cattcagatg ttgtcctggt agacactatg |
| 1261 | gatgagctgc ccagccttgc agatgtccaa cttgcccacc tgaggctgag ggccctctgc |
| 1321 | ctgcctgatt catctgtagc tgaggataaa tgctttcagc cctggaagga acacgatggc |
| 1381 | tggactatgt cagggcttgt cttcgaaagg ccagtgacat ttcagtatta gtgacatcca |
| 1441 | gggttcgttc tgtaatactt caaggctccg gtgtttctcc tcttccttga ttgtgtctgg |
| 1501 | cagctcctcc agcagtttcc agctgatttt gaattctctg agtttttcct tcttgctctt |
| 1561 | catgacagtg tcagggttcc tgacaccctt accttcctga gaaatacccc ctgggagcgc |
| 1621 | ggaaagcaga gcggacaggt cagtgacttc tatttttgac tcgtgttttt ttttccattg |
| 1681 | agatgtactc tctgaagttt ggtcttgatt tgttttatga gaagtgaggt ctgtgagtgg |
| 1741 | ggagggggag atttattctc attttcagga cgagactttt gccctacatc tttcctagaa |
| 1801 | taagaggtga gaatctcatg atttgtctct agatgtggga ggattgtgtg taaccatcct |
| 1861 | ttttcttgct tcctctgtcc agttaaactc ctatacacaa gtctacaccc caggatactc |
| 1921 | cagcctccag ctgggaactc ttttaacctg cagctgtctg tctgggactg ggatttacgt |
| 1981 | tatagcaatg cacagatact acaattccag aatcctggct atgacccaga acactgtcca |
| 2041 | gattcctggc tccctagacc acagccaagc ttcatggttc ctggaccccc cagttttgtg |
| 2101 | tggctcttct ctagaggagc attgaccccc ctgaatcagc tctgtccttg gcgggacagt |
| 2161 | ccttccctgc tggcagtctc ttctcgttgg ctccctcgac ctgctatctc ctctgaaagc |
| 2221 | tggctgacca ggaatggggt ctcccctcac attggggagc ttgcccttta cctccagggc |
| 2281 | tgctgctgcc tgggtatctg ggaccccaga tcaggctctg gagacgctgc tacctgaggg |
| 2341 | gaaggcctga ggtccaggta agaagggaaa atagactggg agtgggacaa gggacttgac |
| 2401 | tctgctgaac cagatgaaca ggagctggaa aggcaaggag ctgaagcctc tgggagtctg |
| 2461 | ggaagtgaag ttctactcct cttggcatca aacaaggttt gggagtgtag gaggtgcggg |
| 2521 | aaagtgcttg tggcttagat taagtggaat ttagggcata gctgaaaggg gaaacagaat |
| 2581 | taaagacacc agaagtagca gagaagcagg gggccagagc tacaacagta ttcttctctg |
| 2641 | ttcctctttg cctcctcccc agatgggcct ctcatctccc acaatctctg gcctccagga |
| 2701 | tgagctatcc catcttcagg agttattacg gaaaggacac caagaatatc tcctgaggat |
| 2761 | cactccaaga aaagagatcc acataccatt ctcaatccca ctgaaattgc tggcattctc |
| 2821 | aaaggcaggg cagaggggga tctggggtag agggagggtt ctgtctaatc tttttttttt |
| 2881 | cttttgtatc tgcacttgca gcctcagctt tcatacttca gcccttaagt tcactaagaa |
| 2941 | ggtctgagtt tctgctgcag atagtggtgt taactgctcc aactcttgtc ttgcttagtt |
| 3001 | tctacaaata tttttgcttc ttgtcatttg aaggattaag aaacaaaaac aatccagaaa |
| 3061 | ttgatcggtt tttttaggcc aatcccatcc cttctggata accagatgtt aaatcatgag |
| 3121 | atcagagatg ctgttcatca gtcccaacaa gatggcctag aaatcgcatt ctcacctcgc |
| 3181 | cttgctgctg ctttaattcc aagttctatt tcttccctta tagttttcta tgggaatgag |
| 3241 | gcggatacag gaaacaccct atctcctctg tatttttgta gtggaatttc tatttaaggg |
| 3301 | gctcattaaa gcatagtatt tatacac |

29: BC035625 EGR2

| | |
|---|---|
| 1 | gagcaattga ttaatagctc ggcgagggga ctcactgact gttataataa cactacacca |
| 61 | gcaactcctg gcttcccagc agccggaaca cagacaggag agagtcagtg gcaaatagac |
| 121 | atttttctta tttcttaaaa aacagcaact tgtttgctac ttttatttct gttgattttt |
| 181 | ttttcttggt gtgtgtggtg gttgttttta agtgtggagg gcaaaaggag ataccatccc |
| 241 | aggctcagtc caacccctct ccaaaacggc ttttctgaca ctccaggtag cgagggagtt |
| 301 | gggtctccag gttgtgcgag gagcaaatga tgaccgccaa ggccgtagac aaaatcccag |
| 361 | taactctcag tggttttgtg caccagctgt ctgacaacat ctacccggtg gaggacctcg |
| 421 | ccgccacgtc ggtgaccatc tttcccaatg ccgaactggg aggccccttt gaccagatga |
| 481 | acggagtggc cggagatggc atgatcaaca ttgacatgac tggagagaag aggtcgttgg |
| 541 | atctcccata tcccagcagc tttgctcccg tctctgcacc tagaaaccag accttcactt |
| 601 | acatgggcaa gttctccatt gaccctcagt accctggtgc cagctgctac ccagaaggca |
| 661 | taatcaatat tgtgagtgca ggcatcttgc aaggggtcac ttccccagct tcaaccacag |
| 721 | cctcatccag cgtcacctct gcctccccca acccactggc cacaggaccc ctgggtgtgt |
| 781 | gcaccatgtc ccagacccag cctgacctgg accacctgta ctctccgcca ccgcctcctc |
| 841 | ctccttattc tggctgtgca ggagacctct accaggaccc ttctgcgttc ctgtcagcag |
| 901 | ccaccacctc cacctcttcc tctctggcct acccaccacc tccttcctat ccatccccca |
| 961 | agccagccac ggacccaggt ctcttcccaa tgatcccaga ctatcctgga ttctttccat |
| 1021 | ctcagtgcca gagagaccta catggtacag ctggcccaga ccgtaagccc tttccctgcc |
| 1081 | cactggacac cctgcgggtg ccccctccac tcactccact ctctacaatc cgtaagccct |
| 1141 | ttccctgccc actggacacc ctgcgggtgc cccctccact cactccactc tctacaatcc |
| 1201 | gtaactttac cctggggggc cccagtgctg gggtgaccgg accaggggcc agtggaggca |
| 1261 | gcgagggacc ccggctgcct ggtagcagct cagcagcagc agcagccgcc gccgccgccg |
| 1321 | cctataaccc acaccacctg ccactgcggc ccattctgag gcctcgcaag taccccaaca |
| 1381 | gacccagcaa gacgccggtg cacgagaggc cctacccgtg cccagcagaa ggctgcgacc |
| 1441 | ggcggttctc ccgctctgac gagctgacac ggcacatccg aatccacact gggcataagc |
| 1501 | ccttccagtg tcggatctgc atgcgcaact tcagccgcag tgaccacctc accacccata |
| 1561 | tccgcaccca caccggtgag aagcccttcg cctgtgacta ctgtggccga aagtttgccc |
| 1621 | ggagtgatga gaggaagcgc cacaccaaga tccacctgag acagaaagag cggaaaagca |
| 1681 | gtgccccctc tgcatcggtg ccagccccct ctacagcctc ctgctctggg ggcgtgcagc |
| 1741 | ctgggggtac cctgtgcagc agtaacagca gcagtcttgg cggagggccg ctcgcccctt |
| 1801 | gctcctctcg gacccggaca ccttgagatg agactcaggc tgatacacca gctcccaaag |
| 1861 | gtcccggagg ccctttgtcc actggagctg cacaacaaac actaccaccc tttcctgtcc |
| 1921 | ctctctccct ttgttgggca aagggctttg gtggagctag cactgccccc tttccaccta |
| 1981 | gaagcaggtt cttcctaaaa cttagcccat tctagtctct cttaggtgag ttgactatca |
| 2041 | acccaaggca aaggggaggc tcagaaggag gtggtgtggg gacccctggc caagagggct |
| 2101 | gaggtctgac cctgctttaa agggttgttt gactaggttt tgctacccca cttcccctta |
| 2161 | ttttgaccca tcacaggttt ttgaccctgg atgtcagagt tgatctaaga cgttttctac |
| 2221 | aataggttgg gagatgctga tcccttcaag tggggacagc aaaaagacaa gcaaaactga |
| 2281 | tgtgcacttt atggcttggg actgatttgg gggacattgt acagtgagtg aagtatagcc |
| 2341 | tttatgccac actctgtggc cctaaaatgg tgaatcagag catatctagt tgtctcaacc |
| 2401 | cttgaagcaa tatgtattat aaactcagag aacagaagtg caatgtgatg ggaggaacat |
| 2461 | agcaatatct gctccttttc gagttgtttg agaaatgtag gctatttttt cagtgtatat |
| 2521 | ccactcagat tttgtgtatt tttgatgtac actgttctct aaattctgaa tctttgggaa |
| 2581 | aaaatgtaaa gcatttatga tctcagaggt taacttattt aagggggatg tacatatatt |
| 2641 | ctctgaaact aggatgcatg caattgtgtt ggaagtgtcc ttggtgcctt gtgtgatgta |
| 2701 | gacaatgtta caaggtctgc atgtaaatgg gttgccttat tatggagaaa aaaatcactc |
| 2761 | cctgagttta gtatggctgt atatttctgc ctattaatat ttggaatttt ttttagaaag |
| 2821 | tatatttttg tatgctttgt tttgtgactt aaaagtgtta cctttgtagt caaatttcag |
| 2881 | ataagaatgt acataatgtt accggagctg atttgtttgg tcattagctc ttaatagttg |
| 2941 | tgaaaaaata aatctattct aacgcaaaac cactaactga agttcagata atggatggtt |
| 3001 | tgtgactata gtgtaaataa atacttttca acaataaaaa aaaaaaaaaa aaaaaaaaaa |
| 3061 | a |

30: X52426 KRT13

| | |
|---|---|
| 1 | ggccaagcaa gcttctatct gcacctgctc tcaatcctgc tctcaccatg agcctccgcc |
| 61 | tgcagagctc ctctgccagc tatggaggtg gtttcggggg tggctcttgc cagctgggag |
| 121 | gaggccgtgg tgtctctacc tgttcaactc ggtttgtgtc tgggggatca gctgggggct |
| 181 | atggaggcgg cgtgagctgt ggttttggtg gaggggctgg tagtggcttt ggaggtggct |
| 241 | atggaggtgg ccttggaggt ggctatggag gtggccttgg aggtggcttt ggtgggggtt |
| 301 | ttgctggtgg ctttgttgac tttggtgctt gtgatggcgg cctcctcact ggcaatgaga |
| 361 | agatcaccat gcagaacctc aacgaccgcc tggcttccta cctggagaag gtgcgcgccc |
| 421 | tggaggaggc caacgctgac ctggaggtga agatccgtga ctggcacctg aagcagagcc |
| 481 | cagctagccc tgagcgggac tacagcccct actacaagac cattgaagag ctccgggaca |
| 541 | agatcctgac cgccaccatt gaaaacaacc gggtcatcct ggagattgac aatgccaggc |
| 601 | tggctgtgga cgacttcagg ctcaagtatg agaatgagct ggccctgcgc cagagcgtgg |
| 661 | aggccgacat caacggcctg cgccgggtgc tggatgagct cactctgtct aagactgacc |
| 721 | tggagatgca gatcgagagc ctgaatgaag agctagccta catgaagaag aaccatgaag |
| 781 | aggagatgaa ggaatttagc aaccaggtgg tcggccaggt caacgtggag atggatgcca |
| 841 | ccccaggcat tgacctgacc cgcgtgctgg cagagatgag ggagcagtac gaggccatgg |
| 901 | cagagaggaa ccgccgggat gctgaggaat ggttccacgc caagagtgca gagctgaaca |
| 961 | aggaggtgtc taccaacact gccatgattc agaccagcaa gacagagatc acggagctca |
| 1021 | ggcgcacgct ccaaggcctg gagattgagc tgcagtccca gctgagcatg aaagcggggc |
| 1081 | tggagaacac ggtggcagag acggagtgcc gctatgccct gcagctgcag cagatccagg |
| 1141 | gactcatcag cagcatcgag gcccagctga gcgagctccg cagtgagatg gagtgccaga |
| 1201 | accaagagta caagatgctg ctggacatca agacacgtct ggagcaggag atcgccacct |
| 1261 | accgcagcct gctcgagggc caggacgcca agaagcgtca gcccccgtag cacctctgtt |
| 1321 | accacgactt ctagtgcctc tgttaccacc acctctaatg cctctggtcg ccgcacttct |
| 1381 | gatgtccgta ggccttaaat ctgcctggcg tcccctccct ctgtcttcag cacccagagg |
| 1441 | aggagagagc cggcagttcc ctgcaggaga gaggaggggc tgctggaccc aaggctcagt |
| 1501 | ccctctgctc tcaggacccc ctgtcctgac tctctcctga tggtgggccc tctgtgctct |
| 1561 | tctcttccgg tcggatctct ctcctctctg acctggatac gctttggttt ctcaacttct |
| 1621 | ctaccccaaa gaaaagatta ttcaataaag tttcctgcct ttctgcaaac ataaaaa |

31: NM_005504 BCAT1

| | |
|---|---|
| 1 | tttgcttgca acactggcac ctctgccctg caccccggga gtgagcagtg agtgaggctc |
| 61 | gggtctgggc gctggctccg aatcttcggg ctgggagaga ctccaccatc tgggggcggc |
| 121 | ctgggggagc agccttagtg tcttcctgct gatgcaatcc gctaggtcgc gagtctccgc |
| 181 | cgcgagaggg ccggtctgca atccagcccg ccacgtgtac tcgccgccgc ctcgggcact |
| 241 | gccccaggtc ttgctgcagc cgggaccgcg ctctgcagcc gcagacccgg tccacacggc |
| 301 | caggggctac gacccttggg atctgccctc cgctcagctc gagcttccct cgtggccgac |
| 361 | ggaacaatga aggattgcag taacggatgc tccgcagagt gtaccggaga aggaggatca |
| 421 | aaagaggtgg tggggacttt taaggctaaa gacctaatag tcacaccagc taccatttta |
| 481 | aaggaaaaac cagaccccaa taatctggtt tttggaactg tgttcacgga tcatatgctg |
| 541 | acggtggagt ggtcctcaga gtttggatgg gagaaacctc atatcaagcc tcttcagaac |
| 601 | ctgtcattgc accctggctc atcagctttg cactatgcag tggaattatt tgaaggattg |
| 661 | aaggcatttc gaggagtaga taataaaatt cgactgtttc agccaaacct caacatggat |
| 721 | agaatgtatc gctctgctgt gagggcaact ctgccggtat ttgacaaaga agagctctta |
| 781 | gagtgtattc aacagcttgt gaaattggat caagaatggg tcccatattc aacatctgct |
| 841 | agtctgtata ttcgtcctac attcattgga actgagcctt ctcttggagt caagaagcct |
| 901 | accaaagccc tgctctttgt actcttgagc ccagtgggac cttatttttc aagtggaacc |
| 961 | tttaatccag tgtccctgtg ggccaatccc aagtatgtaa gagcctggaa aggtggaact |
| 1021 | ggggactgca agatgggagg gaattacggc tcatctcttt ttgcccaatg tgaagcagta |
| 1081 | gataatgggt gtcagcaggt cctgtggctc tatggagagg accatcagat cactgaagtg |
| 1141 | ggaactatga atctttttct ttactggata aatgaagatg gagaagaaga actggcaact |
| 1201 | cctccactag atggcatcat tcttccagga gtgacaaggc ggtgcattct ggacctggca |
| 1261 | catcagtggg gtgaatttaa ggtgtcagag agatacctca ccatggatga cttgacaaca |
| 1321 | gccctggagg ggaacagagt gagagagatg tttggctctg gtacagcctg tgttgtttgc |
| 1381 | ccagtttctg atatactgta caaaggcgag acaatacaca ttccaactat ggagaatggt |
| 1441 | cctaagctgg caagccgcat cttgagcaaa ttaactgata tccagtatgg aagagaagag |
| 1501 | agcgactgga caattgtgct atcctgaatg gaaaatagag gatacaatgg aaaatagagg |
| 1561 | ataccaactg tatgctactg ggacagactg ttgcatttga attgtgatag atttctttgg |
| 1621 | ctacctgtgc ataatgtagt ttgtagtatc aatgtgttac aagagtgatt gtttcttcat |
| 1681 | gccagagaaa atgaattgca atcatcaaat ggtgtttcat aacttggtag tagtaactta |
| 1741 | ccttacctta cctagaaaaa cattaatgta agccatataa catgggattt tcctcaatga |
| 1801 | ttttagtgcc tccttttgta cttcactcag atactaaata gtagtttatt ctttaatata |
| 1861 | agttacattc tgctcctcaa acaaatgcaa ttttttgtgt gtgtttgaaa gctaatttga |
| 1921 | gaaaatttca taggttacat ttcctgcagc ctatctttat ccacagaaag tgttttcttt |
| 1981 | tttttaaatc aagactttta aaactggatt tcctcccatc actgtttttt gaaggtcctc |
| 2041 | caagtccgtg ttaaggtaaa tatctgtttt cttcctgatg tcacagcctg agcatactct |
| 2101 | gtgcattagg aagacctgag tgcatttccc accattgtcc tttccacatt atgttgtagc |
| 2161 | tggctggctg tcaggcgact acaagactga gggtcttgtg ccttatagat ctttgtatcc |
| 2221 | cccatggctg acatatagta ggtactcagt aaatggtttt ataatgaatc agtgaacatt |
| 2281 | ttgcttctat agaagtgtac cttctttgtt tctatattat gaaacctctt tattagaatt |
| 2341 | tgtgattgat tctgacagtg tatagattta ccttatattg tctttatttt ccatgagcta |
| 2401 | ctaagtcatt agagatactc tgaagcatag ttagtttagg aaatcacttc atattgattg |
| 2461 | tattagaatt atcttggaat tgaagatata tccctagagc aggggacccc aacccccagg |
| 2521 | ccatgggcca cacagcagga agaggtgagt ggtgggccat tgaggagctt catctgtatt |
| 2581 | tatggctact tcccatcact cgaattacca cctgaactcc acctcttgtc agctcagtgg |
| 2641 | cagcattaga ttctcatagg agcacaaatc ctattgtgaa ctctgcatgc aagggatcta |
| 2701 | ggctatgcgc tccttatgag aatctaatgc ttgatgacct gaggtgtaac agtttcatcc |
| 2761 | tgaaaccacc cttcaccctg cagtctgtgg aaaaattgtc ttccacaaaa ctggtccctg |
| 2821 | gtgccaaaaa tgttggggac cactgctcta gagagaggtc atgatatcat accaaccaaa |
| 2881 | tggaaatgac aaatgtttta tgtcaagtgt taattgcaga aataaatctt tttttttttt |
| 2941 | ttttggtaga aaacaaagag gcatactctg atttttatac tctgtttttg caggtgctct |
| 3001 | tttctttgaa tggagatttg atgagcaagt ggttaggatg cagggagagc tactatgggt |
| 3061 | gatattttcc ttgtttagga gctgtgagtt aaaattgtat cctttgtggt ttatctaagg |
| 3121 | aaagtcaaat cttgacagaa aacatttttc cttggaaggt caactctcag acattgtatt |
| 3181 | ttggtttccc tcagtcctca taacttcctt cttgctgaac atattttatt ctcttttcag |
| 3241 | agaaggaaaa taaaaaggat tctaaaagtt tgatgcattg gaaaaatttc cttgaggcat |
| 3301 | ttagcaacac atagaaaatg ggctttgatt cttttccaaa acttttagcc atagggtctt |
| 3361 | ttatagacag ggatagtaaa atgaaaattg agaaatataa gatgaaaagg aatgataaaa |
| 3421 | atatctttta gggggctttt aattggtgat ctgaaatctt gggagaagct gttcttttca |
| 3481 | ggcctgaggt gctcttgact gtcgcctgcg cactgtgtac cccgagcaac attctaaggg |
| 3541 | tgtgctttcg ccttggctaa ctcctttgac ctcattcttc atatagtagt ctaggaaaaa |
| 3601 | gttgcaggta atttaaactg tctagtggta catagtaact aaatttctat tcctatgaga |
| 3661 | aatgagaatt atttatttgc catcaacaca ttttatactt tgcatctcca aatttattgt |
| 3721 | ggcgagactt gtccattgtg aaagttagag aacattatgt ttgtatcatt tctttcataa |
| 3781 | aacctcaaga gcatttttaa gcccttttca tcagacccag tgaaaactaa ggatagatgt |
| 3841 | ttaaaaactg gaggtctcct gataaggaga acacaatcca ccattgtcat ttaagtaata |
| 3901 | agacaggaaa ttgaccttga cgctttcttg ttaaatagat ttaacaggaa catctgcaca |
| 3961 | tcttttttcc ttgtgcacta tttgtttaat tgcagtggat taatacagca agagtgccac |
| 4021 | attataacta ggcaattatc cattcttcaa gacttagtta ttgtcacact aattgatcgt |
| 4081 | ttaaggcata agatggtcta gcattaggaa catgtgaagc taatctgctc aaaaagatca |
| 4141 | acaaattaat attgttgctg atatttgcat aattggctgc aattatttaa tgtttaattg |
| 4201 | ggttgatcaa atgagattca gcaattcaca agtgcattaa tataaacaga actggtggca |
| 4261 | cttaaaatga taatgattaa cttatattgc atgttctctt cctttcactt ttttcagttt |
| 4321 | ctacatttca gaccgagctt gtcagctttt ttgaaaacac atcagtagaa accaagattt |
| 4381 | taaaatgaag tgtcaagaca aaggcaaaac ctgagcagtt cctaaaaaga tttgctgtta |
| 4441 | gaaattttct ttgtggcagt catttattaa ggattcaact cgtgatacac caaaagaaga |
| 4501 | gttgacttca gagatgtgtt ccatgctctc tagcacagga atgaataaat ttataacacc |
| 4561 | tgctttagcc tttgttttca aaagcacaaa ggaaaagtga aagggaaaga gaaacaagtg |
| 4621 | actgagaagt cttgttaagg aatcaggttt tttctacctg gtaaacattc tctattcttt |
| 4681 | tctcaaaaga ttgctgtaag aaaaaatgta agacaaaaaa aaaaaaaaaa aacaaacaga |
| 4741 | ggcagaggca ggcagtagca agaaagcaga gcgtaacatc agctagatgg taacatgcaa |
| 4801 | tgtcagctct cttgaagaca tgggaaacct aagttacacc ttgggttaaa attcttcacc |
| 4861 | atattagttt tgttgcttca taaaatttac ctaagcaagt ggtcttgctt gcctcaaatc |
| 4921 | caagcagtct tgaacacttg gaggcaatta atgagtatat cttagtcaaa agaattgttg |
| 4981 | gagcttttta ttaaagctac agtttcagtt ctgcttttgg ggaattgtgc tatgaaagca |
| 5041 | gctgccaaaa taagctcatt tattttcttc aatcccactc agtgctcagt cactatattc |
| 5101 | tgtttccttt ttttttttca agttgcatat ttggtttccc cttatgattg ggaaagatga |
| 5161 | attttcagca gaaaacattg tttgttcact ttcaaagagt gatagtttct aaaacattta |
| 5221 | gagcaataaa tattcatcag aggtaccaag taagccggca gaagagttaa gggttagaga |
| 5281 | aatcccttat ttcatgtctt gactctaaaa ttatcaaagt acttttcctt gtaatgtgga |
| 5341 | tttcttctta tgcggatatg caaaaacttc agttatacgt agtaatgcta gcaggtaatt |
| 5401 | ttagtagaca ttttataaca actgtcactt tgtttcgcca catgtagagt ttgttcagct |
| 5461 | attttccaga tatctcccca caaaaggagg caaagggtac cagcttttca atgagcatta |
| 5521 | cctattactt ggcaaagatg atgaagactc tattaatagt tcatttgata aatgttgaca |
| 5581 | taaccaacaa tagagattag gaagttagtt ttaagaaatc aatggcatat agacattacc |
| 5641 | ctcatggagt ttgtattcta ctacttgaac tgattgtagc tataaaagca tagttagata |
| 5701 | gctgaatagt tagatcataa gcaaagaagg ccagaacaca tctcttatca agaaatcaat |
| 5761 | gaatagttta tctcattttt aaagcaactt tatccttctt taattccttc ctttcttcta |
| 5821 | gtgcaaaact acttaataag gttggtgttt aggttagtgt tcacaccatt cctcatctgg |
| 5881 | tgtgaattac cttctctttc tttactattt actaccaacc tagtacatgt gttgactgaa |
| 5941 | ttcttttcaa acaatgttga gttatcatgg tgcacctaat aaattaacac cacagattac |
| 6001 | agcatccttg ctgattttct cagcaaagcc agattagatg gaaataaaca aagaaaatga |
| 6061 | tcctagagtg aatttttcta gaaaatatct attatgaacc atgctgttta aagtattagc |
| 6121 | ttgaaggtga tggatccagc tattcagaaa ataactttca tataaccatg attttgcaca |
| 6181 | gtatgaggtc ttaaatgtgt ggaaagagat aaatttttta tcattaccac aaaccccttt |
| 6241 | taaagattca aaggtggaag aaagtgattt attttttctc ttcagcatac atatataaaa |
| 6301 | gacttgtcag atgtttaatt tggggaggtt gataatgaaa catatcaaca gagtatagta |
| 6361 | gttatagtag tgtttgtggg taaataattt cctggggtca gacatatata aacatatttg |
| 6421 | cttcaaaatg ataaaggcat gaaatcagtc ttaaaaattg aaatgggggt gatgggggag |
| 6481 | aaaaagaaga acaaatttga agtgcccttt caaatctgct ggatacaagt attgaagttt |
| 6541 | taagtcatct tattctgtct gaaagtgtat ttttcattct acaatagacc caatcaacaa |
| 6601 | gacgtataac ttgagttgca tgatgttcag tttatgtaat ctactgttgg gatggtaaga |
| 6661 | attgatgtag gctgtggtgt aagaatgaat taaaatatag tttcactggc ttttctctac |
| 6721 | atatccacta tcacaatggc taggtttcct gttgctcact attggattct ggagaaaaat |
| 6781 | ttaatgaaag atgatatcag aggaagaata agtggaggta gagaagaaag gaatgataga |
| 6841 | ggaggggaaa aaaacaaaac atatttttgt gttatccaaa ggagcttttt ccttattctg |
| 6901 | tcaagcattg agatcttctt cagctttcaa tgtagttgct aaatacaaat aatgctacta |
| 6961 | ggtagtgact aaatatagca aacacttcat cagatattag aattaggtca cactattgag |
| 7021 | gttataatct gaaggttgtg ttacatagaa accactttag attattatca acttggacta |
| 7081 | ggctttattt tataatagca tagtaagtaa tatctattgt gtcatttctt caaccatttt |
| 7141 | attctaagat ccatgaagct tcttgaggcc aaataaaata ataagtttag acaagaagta |
| 7201 | gattgtgact tttttccctt agagatacta tttactatct cctatcctga taggtggaag |
| 7261 | gtttactgaa ttggaaattg gttgactatt agtttttaac taaaatgtgc aataacacat |
| 7321 | tgcagtttcc tcaaactagt ttcctatgat cattaaactc attctcaggg ttaagaaagg |
| 7381 | aatgtaaatt tctgcctcaa tttgtacttc atcaataagt ttttgaagag tgcagatttt |
| 7441 | tagtcaggtc ttaaaaataa actcacaaat ctggatgcat ttctaaattc tgcaaatgtt |
| 7501 | tcctggggtg acttaacaag gaataatccc acaatatacc tagctaccta atacatggag |
| 7561 | ctggggctca acccactgtt tttaaggatt tgcgcttact tgtggctgag gaaaaataag |
| 7621 | tagttcgagg aagtagtttt taaatgtgag cttatagata gaaacagaat atcaacttaa |
| 7681 | ttatgaaatt gttagaacct gttctcttgt atctgaatct gattgcaatt actattgtac |
| 7741 | tgatagactc cagccattgc aagtctcaga tatcttagct gtgtagtgat tcttgaaatt |
| 7801 | ctttttaaga aaaattgagt agaaagaaat aaaccctttg taaatgaggc ttggcttttg |
| 7861 | tgaaagatca tccgcaggct atgttaaaag gattttagct cactaaaagt gtaataatgg |
| 7921 | aaatgtggaa aatatcgtag gtaaaggaaa ctacctcatg ctctgaaggt tttgtagaag |
| 7981 | cacaattaaa catctaaaat ggctttgtta caccagagcc atctggtgtg aagaactcta |
| 8041 | tatttgtatg ttgagagggc atggaataat tgtattttgc tggcaataga cacattcttt |
| 8101 | attatttgca gattcctcat caaatctgta attatgcaca gtttctgtta tcaataaaac |
| 8161 | aaaagaatcc tgtttgtgtg gtttcatgaa a |

32: NM_006643 SDCCAG3

| | |
|---|---|
| 1 | cacgggcgga gccggggcca tggagccgcc gctgccgggc taggcaggtc gtgccccgcc |
| 61 | gggccggcgg cgatgtcggg ctaccagcgc cacccgggcg ccaccccgct gtcccgagcc |
| 121 | cggagcctcg ccattcccga cgctccagcg ttctatgagc gccggtcttg tctcccccag |
| 181 | ctaaattgtg agcgccccca tggcagggac ctggactccc ccttcttcgg cattcggccg |
| 241 | gcctttatgt gctatgtgcc cagcccggtg ctggcttccg tgggagacac agatgacaga |
| 301 | tttgaagatc tggaagaggc aaatccattc tcttttagag agtttctgaa gaccaagaac |
| 361 | ctcggcctct cgaaagagga tccggccagc agaatttatg caaaggaagc ctcgaggcat |
| 421 | tccctgggac ttgaccacaa ctccccaccc tcccaaaccg gcgggtatgg cctggagtat |
| 481 | cagcagccat ttttcgagga tccgacaggg gctggtgacc tcctggatgg ggaggaggat |
| 541 | gaggacaccg gatggagtgg ggcctacctg ccgtccgcca tcgagcagac tcaccccgag |
| 601 | agggtccctg ccggcacgtc gccctgcagc acataccttt cctttttctc caccccgtcg |
| 661 | gagctggcag ggcctgagtc tctgccctcg tgggcgttga gtgacactga ttctcgcgtg |
| 721 | tctccggcct ctccggcagg gagtcctagc gcagactttg cggttcatgg agagtctctg |
| 781 | ggagacaggc acctgcggac gctgcagata agttacgacg cactgaaaga tgaaaattct |
| 841 | aagctgagaa gaaagctgaa tgaggttcag agcttctctg aagctcaaac agaaatggtg |
| 901 | aggacgcttg agcggaagtt agaagcaaaa atgatcaagg aggaaagcga ctaccacgac |
| 961 | ctggagtcgg tggttcagca ggtggagcag aacctggagc tgatgaccaa acgggctgta |
| 1021 | aaggcagaaa accacgtcgt gaaactaaaa caggaaatca gtttgctcca ggcgcaggtc |
| 1081 | tccaacttcc agcgagagaa tgaagccctg cggtgcggcc agggcgccag cctgaccgtg |
| 1141 | gtgaagcaga acgccgacgt ggccctgcag aacctccggg tggtcatgaa cagtgcacag |
| 1201 | gcttccatca agcaactggt ttccggagct gagacactga atcttgttgc cgaaatcctt |
| 1261 | aaatctatag acagaatttc tgaaattaaa gacgaggagg aagactcttg aggacccctg |
| 1321 | ggtgttctca gcatgaagct ccgtgtatac cctgaggtca ccaccgctcg atctaaatgt |
| 1381 | gcagttgtgt ccttaaatat gcagtcttca cccagagtaa agtgttgatc gcaagagtcc |
| 1441 | agtgtcgtgc cctcagccag ttcttggcca ccacaatggg agcagccctg gccgagttgt |
| 1501 | ctctgtggtt tctatgcagc ccttcttggc gaaattcctg cgatcttata gattctaatg |
| 1561 | agctcttgga agacattgtc ataaaagcca gtgattttaa gaaaaagagt ggttctggaa |
| 1621 | tcagtgtttt ccagtcccat cccagaacat cagttgtaag ataagtacaa ttggttgtcc |
| 1681 | ttgatttcat aagtagaaca aacactaaat gtgcctctga gatggccacc ccgggcaggg |
| 1741 | acctgtgcct tccaccgatg ctcagggctc cctctggctc ccgggtcact cttgtggccc |
| 1801 | cagtgggtgg tccctgcagt catggcctga gtgcgcaggg gccaccgcgt ggctgccgct |
| 1861 | gtcctcctcc gggacccacg gggaccaagg tcacacgttc cgtgctgtga agctgtccag |
| 1921 | atgtgcctct ttggctgggg gttctggtgg acgtttcaag tggcattttg tacaatgcag |
| 1981 | gttagaattc aggaatttca agtatgtgcc cgggtctgtc aggtcccagt tgcctttctg |
| 2041 | acggcccccc tcagagggac ggcgatgagc actaaatgct tttttgacta ttttcctata |
| 2101 | gatttttttt aaaacttttt tttcctcctg ttccaattga tagctttctt atttaataaa |
| 2161 | ttctgtagtt caccaaaaaa aaaaaaaaaa a |

33: AA464095 PIGK

| | |
|---|---|
| 1 | atatattccc agctagttga aaatgatgat tcccacaaga agcataactc agcttgtttc |
| 61 | tgcttactga gtattttcta ctatggtata tattgataac atttcttcca ttatgtatgt |
| 121 | tgtataccag agttacagtt actgtgggaa tcataatttg aaattttgac tcctgtgttt |
| 181 | ctggaatctt tacaacaaat gttgcattaa catataactt ttttcagttg actttaccaa |
| 241 | aattaagccc atctttagta gatactgttt taacatgtga aagaaatacg ttataaacat |
| 301 | accacaagat atggctataa aacaatgaga tcagtatcca tttttgcttt aaagaattgg |
| 361 | ccttattgct tcagtgtcac atctcatact caagggcatt tactacaaay aaagagttct |
| 421 | ccaatattgc tgttctgttg ctgcctgccc tatttacaca tgt |

34: AA961188 MRPS9

| | |
|---|---|
| 1 | tttacactta tagtagactt tatttagtga atccaaatga catgtgataa ttgtttggaa |
| 61 | aggcctattg attttatatc tgatcattca atccagagac attaaattca gttgattaat |
| 121 | ggagttcccc aactgtaaga cttctttacg agattatttt caagctttga aaagatcttc |
| 181 | tgagataaag gggatcagca aacagtaaga gtgtgttgct atacccaagc aaaagaaata |
| 241 | aatcttaatc tctcagcaaa tcattcaaaa tgtcagaaat gttagtgttt ctatatcttg |
| 301 | gtaaaatgga ttgattgaga agtatgaaaa gtataacagt ggcatgcaga atattgtttt |
| 361 | tatgaatatt cagaatttca gttgtttaca taa |

35: NM_0181836 ASPM

| | |
|---|---|
| 1 | atggcgaacc ggcgagtggg gcgaggctgc tgggaagtga gcccgaccga gcggaggccg |
| 61 | cccgcggggc tgcggggccc cgcggccgag gaggaggcgt cttccccgcc ggtcctgtct |
| 121 | ctcagccact tctgcaggtc tcctttcctt tgcttcgggg acgttctcct gggagcctca |
| 181 | cggacgctgt ctctggccct agacaaccct aacgaggagg tggcagaagt gaagatctcc |
| 241 | cacttcccgg ccgcggacct gggcttcagt gtgtcgcagc gctgtttcgt gttgcagcct |
| 301 | aaagagaaaa ttgttatttc tgttaactgg acaccactca aagaaggccg agtaagagag |
| 361 | attatgacat ttcttgtaaa tgatgttctg aaacaccaag ctatattact aggaaatgca |
| 421 | gaagagcaga aaaagaaaaa gaggagtctt tgggatacca ttaaaaagaa gaaaatttca |
| 481 | gcctctacaa gtcacaacag aagggtttca aatattcaga atgttaataa aacatttagt |
| 541 | gtttcccaaa aagttgacag agttaggagc ccactacaag cttgtgaaaa cttggctatg |
| 601 | aatgaaggcg gtcccccaac agaaaacaat tctttaatac ttgaagaaaa taaaataccc |
| 661 | atatcaccta ttagccctgc tttcaatgaa tgccatggtg caacttgctt gccactctct |
| 721 | gtacgtcgat ctactaccta ctcatctctt catgcatcag aaaataggga actattaaat |
| 781 | gtacacagtg ccaacgtttc aaaagtttct tttaatgaga aagctgtaac tgaaacttcc |
| 841 | tttaattctg taaatgttaa tggccaaaga ggagagaata gtaaacttag tcttaccccc |
| 901 | aactgttctt caactttgaa cattacacaa agccaaatac attttctaag tccagattct |
| 961 | tttgtaaata atagtcatgg agctaataat gaactagaat tagtaacatg tctttcatca |
| 1021 | gatatgttta tgaaagataa ttcacagcct gtgcatttgg aatcaacaat tgcacatgaa |
| 1081 | atttatcaga aaattttaag tccagattct ttcataaaag ataattatgg actaaatcag |
| 1141 | gatctagaat cagagtcagt taatcctatt ttatccccta atcaattttt aaaagataac |
| 1201 | atggcatata tgtgtacatc tcagcaaaca tgtaaagtac cattatcaaa tgaaaattct |
| 1261 | caagtcccac agtctcctga agattggaga aaaagtgaag tttcgccacg tattcctgaa |
| 1321 | tgtcagggtt caaaatctcc caaagctatt tttgaagaac tagtagaaat gaagtcaaat |
| 1381 | tactacagtt ttataaaaca aaataatcct aaattttctg cagttcagga tatttctagt |
| 1441 | catagccaca ataaacaacc taagagacgt ccaatacttt ctgccactgt tactaaaagg |
| 1501 | aaggccacct gtaccagaga aaaccaaact gagattaata aaccaaaagc aaaaagatgt |
| 1561 | ctcaacagtg cagtgggtga acatgaaaaa gtaataaata atcaaaagga aaaagaagat |
| 1621 | tttcattctt atcttccaat tatagatcca atattaagta aatctaagag ttataaaaac |
| 1681 | gaggtaacac cctcttcgac aacagcttca gttgctcgga aaagaaagag cgatggaagc |
| 1741 | atggaagatg caaatgtgag agttgcaatt acagaacata cagaagtgcg agaaatcaaa |
| 1801 | agaatccatt tttctccctc agagcctaaa acatcagctg ttaagaaaac aaaaaatgtg |
| 1861 | acaacaccca tctcaaaacg tattagcaac agagagaaat taaacctgaa gaagaaaact |
| 1921 | gatttatcaa tattcagaac tccaatttct aaaacaaaca aaaggacaaa acccattatc |
| 1981 | gctgtggcac agtccagttt gaccttcata aaaccattaa aaacagatat tcccagacac |
| 2041 | ccgatgccat ttgctgcaaa aaacatgttt tatgatgaac gctggaagga aaagcaggaa |
| 2101 | cagggcttca cttggtggtt aaattttata ttaacccctg atgacttcac tgtaaaaaca |
| 2161 | aatatttctg aagtaaatgc tgctactctt cttttgggaa tagagaatca acataaaata |
| 2221 | agtgttccta gagcacctac aaaagaggaa atgtctctca gagcttatac tgctcggtgt |
| 2281 | aggttaaaca gactacgtcg tgcagcatgc cgtttgttta cttctgaaaa aatggttaaa |
| 2341 | gctattaaaa agcttgaaat tgaaattgaa gctaggcggt taattgttcg aaaagataga |
| 2401 | cacctatgga aagatgtggg agaacgtcag aaagtcctga attggctgtt gtcctacaat |
| 2461 | cctttgtggc ttcgaattgg tctagagaca acttatggag aactcatatc tttggaagat |
| 2521 | aacagtgatg tcacagggtt ggctatgttt attctgaatc gcctactttg gaatcctgat |
| 2581 | atagcagctg agtatagaca ccccactgtt cctcacctgt atagagatgg tcatgaagaa |
| 2641 | gctttgtcca agtttacatt gaaaaagtta ttgttgttgg tctgttttct tgattatgct |
| 2701 | aaaatttcca gactcattga tcatgatcct tgtctcttct gtaaagatgc cgaattcaag |
| 2761 | gctagtaaag aaatcctttt ggctttttca cgagatttcc taagtggtga aggtgacctt |
| 2821 | tcccgtcacc ttggcttatt gggattacct gttaaccatg ttcagacacc atttgatgaa |
| 2881 | tttgattttg ccgttacaaa tcttgccgta gacttgcaat gtggagtgcg ccttgtgcga |
| 2941 | accatggaac ttctcacaca gaactgggac ctctcaaaga aactcaggat tccggcaata |
| 3001 | agtcgtcttc aaaagatgca caatgttgac attgttcttc aagttcttaa atcacgagga |
| 3061 | attgaattaa gtgatgagca tggaaataca attctatcta aggatattgt ggataggcac |
| 3121 | agagaaaaaa ctctcaggtt gctttggaaa atagcgtttg cttttcaggt ggatatttcc |
| 3181 | cttaacttag atcaattaaa ggaagaaatt gcctttctaa aacacacaaa gagtataaag |
| 3241 | aaaacaatat ctctactatc atgccattct gatgatctta ttaataagaa aaaaggcaaa |
| 3301 | agggatagtg gttcctttga acaatatagt gaaaacataa agttattgat ggattgggta |
| 3361 | aatgctgttt gtgccttcta taataaaaag gtggagaatt ttacagtgtc tttctcagac |
| 3421 | ggccgtgtgt tatgttacct gatccaccat taccatcctt gctatgtgcc atttgacgct |
| 3481 | atatgtcagc gtactactca aactgtggaa tgtacgcaaa ctggttcagt ggtattaaat |
| 3541 | tcatcatctg aatctgatga cagttctctg gatatgtcac ttaaagcatt tgatcatgaa |
| 3601 | aatacttcag agctatacaa agagctccta gaaaatgaaa agaaaaattt tcacttggtt |
| 3661 | aggtctgcag ttagagacct tggtggaata cctgctatga ttaatcattc agatatgtca |
| 3721 | aatacaattc cagatgaaaa ggtggttatt acctatttgt catttctttg tgcaaggctt |
| 3781 | ttggatcttc gtaaagaaat aagagctgct cgactcatac aaacaacatg gagaaaatat |
| 3841 | aaactaaaaa cagatctcaa acgccatcag gagagagaga aagctgcaag aattattcaa |
| 3901 | ttggctgtaa tcaattttct agcaaaacaa agattgagaa aaagagttaa tgcagcactc |
| 3961 | gtcattcaga aatattggcg aagagtctta gcacagagaa aattattaat gttaaaaaag |
| 4021 | gaaaagctgg aaaaagttca aaataaagca gcatcactta ttcagggata ttggagaaga |
| 4081 | tattccacta gacaaagatt tctgaaattg aaatattatt caatcatcct gcaatctagg |
| 4141 | ataagaatga taattgctgt tacatcttat aaacgatatc tttgggctac agttacaatt |
| 4201 | cagaggcatt ggcgtgctta tttaagaaga aaacaagatc aacaaagata tgaaatgcta |
| 4261 | aaatcatcaa ctcttataat ccaatctatg ttcagaaaat ggaagcaacg taaaatgcaa |
| 4321 | tcacaagtaa aagctacagt aatattgcaa agagctttta gagaatggca tttaagaaaa |
| 4381 | caagctaaag aagaaaattc tgctattatc atacaatcat ggtatagaat gcataaagaa |
| 4441 | ttacggaagt atatttatat tagatcttgt gttgttatca ttcagaaaag atttcggtgc |
| 4501 | tttcaagccc aaaagttata taaaagaaga aaagagtcca tactaaccat ccagaagtac |
| 4561 | tacaaagcat atctgaaagg aaagattgag cgcaccaact atttgcagaa acgagctgca |
| 4621 | gccattcaat tacaagctgc ttttaggaga ctgaaagctc ataatttatg tagacaaatt |
| 4681 | agagctgctt gtgttattca gtcatactgg agaatgagac aagacagagt tcgattttta |
| 4741 | aaccttaaga agactattat caaatttcag gcacatgtaa gaaaacatca acaacgacag |
| 4801 | aaatataaga agatgaagaa agcagctgtt ataattcaga ctcatttccg agcttatatt |
| 4861 | tttgccatga aagttctagc atcttaccag aaaacacgct ctgctgtcat tgtgctgcag |
| 4921 | tctgcatata gagggatgca agccaggaaa atgtatattc acatcctcac atctgttata |
| 4981 | aagattcaat catattatcg tgcttatgtt tctaaaaagg aatttttgag cctaaaaaat |
| 5041 | gctacaataa aattgcagtc aactgttaag atgaaacaaa cacgtaaaca atatttgcat |
| 5101 | ttaagagcag ctgcactatt tatccagcaa tgttaccgtt ccaaaaaaat agctgcacaa |
| 5161 | aagagagaag agtatatgca gatgcgggaa tcttgtatca aactgcaagc atttgttaga |
| 5221 | ggataccttg tccgaaagca gatgaggtta caaagaaaag ctgttatttc actacagtct |
| 5281 | tatttcagaa tgagaaaggc tcggcagtat tatctgaaaa tgtataaagc aattattgtc |
| 5341 | attcagaatt actatcatgc atacaaagca caggtcaatc agaggaagaa cttcttgcaa |
| 5401 | gtcaaaaaag cagctacttg cttgcaagca gcttacagag gttataaagt acgccagcta |
| 5461 | atcaaacaac aatctatagc tgctcttaaa attcagtctg cttttagagg ctataataaa |
| 5521 | agggtaaaat atcaatctgt gcttcaatct ataataaaga ttcagagatg gtacagggcg |
| 5581 | tacaagactc ttcatgatac aagaacacat tttttgaaga caaaggcagc tgtgatttcc |
| 5641 | ctccagtctg cttatcgtgg ctggaaggtt cggaaacaga ttagaaggga acatcaagct |
| 5701 | gccttgaaga ttcagtctgc ttttagaatg gccaaggccc agaaacagtt tagattgttt |
| 5761 | aaaacagcag cattagtcat ccagcaaaat ttcagagcat ggactgcagg aaggaagcaa |
| 5821 | tgtatggagt atattgaact ccgtcatgcg gtactggtgc ttcaatctat gtggaaggga |
| 5881 | aaaacactga gaagacagct tcaaaggcaa cataaatgtg ctatcatcat acagtcatac |
| 5941 | tatagaatgc atgtgcaaca aaagaagtgg aaaatcatga aaaaagctgc tcttctgatt |
| 6001 | caaaagtatt atagggctta cagtattgga agagaacaga atcatttata tttgaaaaca |
| 6061 | aaagcagctg tagtaacttt acagtcagct tatcgtggta tgaaagtgag aaaaagaata |
| 6121 | aaggattgca acaaagcagc agtcactata cagtctaaat acagagctta caaaaccaaa |
| 6181 | aagaaatatg caacctatag agcttcagct attataattc agagatggta tcgaggtatt |
| 6241 | aaaattacaa accatcagca taaggagtat cttaatttga agaagacagc aattaaaatc |
| 6301 | caatctgttt atagaggtat tagagttaga agacatattc aacacatgca cagggcagcc |
| 6361 | acttttatta aagccatgtt taaaatgcat cagtcaagaa taagttacca tacaatgaga |
| 6421 | aaagcagcta ttgttattca agtaagatgt agagcatatt atcaaggtaa aatgcagcgt |
| 6481 | gaaaagtacc tgacaatttt gaaagctgtt aaagtccttc aggcaagttt tagaggagta |
| 6541 | agagttagac ggactcttag aaagatgcag actgcagcaa cactcattca gtcaaactac |
| 6601 | agaagataca gacagcaaac atactttaat aagttaaaga aaataacaaa aacagtacag |
| 6661 | caaagatact gggcaatgaa agaaagaaac atacaatttc aaaggtataa caaactgagg |
| 6721 | cattctgtaa tatacattca ggctattttt aggggaaaga aagctagaag acatttaaaa |
| 6781 | atgatgcata tagccgcaac tctcattcag aggagattta gaactctaat gatgagaaga |
| 6841 | agattcctct ctctcaagaa aactgctatt ttgattcaga gaaaatatcg ggcacatctt |
| 6901 | tgtacaaagc atcacttaca gttccttcag gtacaaaatg cagttattaa aatccagtca |
| 6961 | tcatacagaa gatggatgat aaggaaaagg atgcgagaga tgcacagggc tgctactttc |
| 7021 | atccagtcta ctttcagaat gcacagatta catatgagat atcgagcttt gaaacaggcc |
| 7081 | tccgttgtga tccaacagca ataccaagca aatagagctg caaaactgca gaggcagcat |
| 7141 | tatctcagac aaagacactc tgctgtgatc cttcaggctg cattcagggg tatgaaaact |
| 7201 | agaagacatt tgaagagtat gcattcctct gcaaccctta ttcagagtag gtttagatca |
| 7261 | ttactggtga ggagaagatt catttccctc aaaaaagcta ctatttttgt tcagaggaaa |
| 7321 | tatcgagcca ccatttgtgc caaacataaa ttgtaccaat tcttgcactt aagaaaggca |
| 7381 | gccattacaa tacagtcatc ttacagaaga ctgatggtaa agaagaagtt acaagaaatg |
| 7441 | caaagggctg cagttctcat tcaggctact ttcaggatgc acagaacata tattacattt |
| 7501 | cagacttgga aacatgcttc aattctaatt cagcaacatt atcgaacata tagagctgca |
| 7561 | aaattgcaaa gagaaaatta tatcagacaa tggcattctg ctgtggttat tcaggctgca |
| 7621 | tataaaggaa tgaaagcaag acaactttta agggaaaaac acaaagcttc tatcgtaata |
| 7681 | caaagcacct acagaatgta taggcagtat tgtttctacc aaaagcttca gtgggctaca |
| 7741 | aaaatcatac aagaaaaata tagagcaaat aaaaagaaac agaaagtatt tcaacacaat |
| 7801 | gaacttaaga aagagacttg tgttcaggca ggttttcagg acatgaacat aaaaaaacag |
| 7861 | attcaggaac agcaccaggc tgccattatt attcagaagc attgtaaagc ctttaaaata |
| 7921 | aggaagcatt atctccacct tagagcaaca gtagtttcta ttcaaagaag atacagaaaa |
| 7981 | ctaactgcag tgcgtaccca agcagttatt tgtatacagt cttattacag aggctttaaa |
| 8041 | gtacgaaagg atattcaaaa tatgcaccgg gctgccacac taattcagtc attctatcga |
| 8101 | atgcacaggg ccaaagttga ttatgaaaca aagaaaactg caattgtggt tatacagaat |
| 8161 | tattataggt tgtatgttag agtaaaaaca gaaagaaaaa actttttagc agttcagaaa |
| 8221 | tctgtacgaa ctattcaggc tgcttttaga ggcatgaaag ttagacaaaa attgaaaaat |
| 8281 | gtatcagagg aaaagatggc agccattgtt aaccaatctg cactctgctg ttacagaagt |
| 8341 | aaaactcagt atgaagctgt tcaaagtgaa ggtgttatga ttcaagagtg gtataaagct |
| 8401 | tctggccttg cttgttcaca ggaagcagag tatcattctc aaagtagggc tgcagtaaca |
| 8461 | attcaaaaag ctttttgtag aatggtcaca agaaaactgg aaacacagaa atgtgctgcc |
| 8521 | ctacggattc agttcttcct tcagatggct gtgtatcgga gaagatttgt tcagcagaaa |
| 8581 | agagctgcta tcactttaca gcattatttt aggacgtggc aaaccagaaa acagttttta |
| 8641 | ctatatagaa aagcagcagt ggttttacaa aatcactaca gagcatttct gtctgcaaaa |
| 8701 | catcaaagac aagtctattt acagatcaga agcagtgtta tcattattca agctagaagt |
| 8761 | aaaggattta tacagaaacg gaagtttcag gaaattaaaa atagcaccat aaaaattcag |
| 8821 | gctatgtgga ggagatatag agccaagaaa tatttatgta aagtgaaagc tgcctgcaag |
| 8881 | attcaagcct ggtatagatg ttggagagca cacaaagaat atctagctat attaaaagct |
| 8941 | gttaaaatta ttcaaggttg cttctatacc aaactagaga gaacacggtt tttgaatgtg |
| 9001 | agagcatcag caattatcat tcagagaaaa tggagagcta tacttcctgc aaagatagct |
| 9061 | catgaacact tcttaatgat aaaaagacat cgagctgctt gtttgatcca agcacattat |
| 9121 | agaggatata aaggaaggca ggtctttctt cggcagaaat ctgctgcttt gatcatacaa |
| 9181 | aaatatatac gagccaggga ggctggaaag catgaaagga taaaatatat tgaatttaaa |
| 9241 | aaatctacag ttatcctaca agcactggtg cgtggttggc tagtacgaaa aagattttta |
| 9301 | gaacagagag ccaaaattcg acttcttcac ttcactgcag ctgcatatta tcacctgaat |
| 9361 | gctgttagaa ttcaaagagc ctataaactt tacctggctg tgaagaatgc taacaagcag |
| 9421 | gttaattcag tcatctgtat tcagagatgg tttcgagcaa gattacaaga aaagagattt |
| 9481 | attcagaaat atcatagcat caaaaagatt gagcatgaag gtcaagaatg tctgagccag |
| 9541 | cgaaataggg ctgcatcagt aatacagaaa gcagtgcgcc attttctcct ccgtaaaaag |
| 9601 | caggaaaaat tcactagtgg aatcattaaa attcaggcat tatggagagg ctattcttgg |
| 9661 | aggaagaaaa atgattgtac aaaaattaaa gctatacgac taagtcttca agttgttaat |
| 9721 | agggagattc gagaagaaaa caaactctac aaaagaactg cacttgcact tcattacctt |
| 9781 | ttgacatata agcacctttc tgccattctt gaggccttaa aacacctaga ggtagttact |
| 9841 | agattgtctc cactttgttg tgagaacatg gcccagagtg gagcaatttc taaaatattt |
| 9901 | gttttgatcc gaagttgtaa tcgcagtatt ccttgtatgg aagtcatcag atatgctgtg |
| 9961 | caagtcttgc ttaatgtatc taagtatgag aaaactactt cagcagttta tgatgtagaa |
| 10021 | aattgtatag atatactatt ggagcttttg cagatatacc gagaaaagcc tggtaataaa |
| 10081 | gttgcagaca aaggcggaag catttttaca aaaacttgtt gtttgttggc tattttactg |
| 10141 | aagacaacaa atagagcctc tgatgtacga agtaggtcca aagttgttga ccgtatttac |
| 10201 | agtctctaca aacttacagc tcataaacat aaaatgaata ctgaaagaat actttacaag |
| 10261 | caaaagaaga attcttctat aagcattcct tttatcccag aaacacctgt aaggaccaga |
| 10321 | atagtttcaa gacttaagcc agattgggtt ttgagaagag ataacatgga agaaatcaca |
| 10381 | aatcccctgc aagctattca aatggtgatg gatacgcttg gcattcctta ttag |

36: NM_002735 ACBD3

| | |
|---|---|
| 1 | atacgtggct gccgtctgtc cccgctgagg aggtgcagca gccggagatg gcggcggtgc |
| 61 | tgaacgcaga gcgactcgag gtgtccgtcg acggcctcac gctcagcccg gacccggagg |
| 121 | agcggcctgg ggcggagggc gccccgctgc tgccgccacc gctgccaccg ccctcgccac |
| 181 | ctggatccgg tcgcggcccg ggcgcctcag gggagcagcc cgagcccggg gaggcggcgg |
| 241 | ctgggggcgc ggcggaggag gcgcggcggc tggagcagcg ctggggtttc ggcctggagg |
| 301 | agttgtacgg cctggcactg cgcttcttca aagaaaaaga tggcaaagca tttcatccaa |
| 361 | cttatgaaga aaaattgaag cttgtggcac tgcataagca agttcttatg ggcccatata |
| 421 | atccagacac ttgtcctgag gttggattct ttgatgtgtt ggggaatgac aggaggagag |
| 481 | aatgggcagc cctgggaaac atgtctaaag aggatgccat ggtggagttt gtcaagctct |
| 541 | taaataggtg ttgccatctc ttttcaacat atgttgcgtc ccacaaaata gagaaggaag |
| 601 | agcaagaaaa aaaaaggaag gaggaagagg agcgaaggcg gcgtgaagag gaagaaagag |
| 661 | aacgtctgca aaaggaggaa gagaaacgta ggagagaaga agaggaaagg cttcgacggg |
| 721 | aggaagagga aaggagacgg atagaagaag aaaggcttcg gttggagcag caaaagcagc |
| 781 | agataatggc agctttaaac tcccagactg ccgtgcagtt ccagcagtat gcagcccaac |
| 841 | agtatccagg gaactacgaa cagcagcaaa ttctcatccg ccagttgcag gagcaacact |
| 901 | atcagcagta catgcagcag ttgtatcaag tccagcttgc acagcaacag gcagcattac |
| 961 | agaaacaaca ggaagtagta gtggctgggt cttccttgcc tacatcatca aaagtgaatg |
| 1021 | caactgtacc aagtaatatg atgtcagtta atggacaggc caaaacacac actgacagct |
| 1081 | ccgaaaaaga actggaacca gaagctgcag aagaagccct ggagaatgga ccaaaagaat |
| 1141 | ctcttccagt aatagcagct ccatccatgt ggacacgacc tcagatcaaa gacttcaaag |
| 1201 | agaagattca gcaggatgca gattccgtga ttacagtggg ccgaggagaa gtggtcactg |
| 1261 | ttcgagtacc cacccatgaa gaaggatcat atctcttttg ggaatttgcc acagacaatt |
| 1321 | atgacattgg gtttggggtg tattttgaat ggacagactc tccaaacact gctgtcagcg |
| 1381 | tgcatgtcag tgagtccagc gatgacgacg aggaggaaga agaaaacatc ggttgtgaag |
| 1441 | agaaagccaa aaagaatgcc aacaagcctt tgctggatga gattgtgcct gtgtaccgac |
| 1501 | gggactgtca tgaggaggtg tatgctggca gccatcaata tccagggaga ggagtctatc |
| 1561 | tcctcaagtt tgacaactcc tactctttgt ggcggtcaaa atcagtctac tacagagtct |
| 1621 | attatactag ataaaaatgt tgttacaaag tctggagtct agggttgggc agaagatgac |
| 1681 | atttaatttg gaaatttctt tttacttttg tggagcatta gagtcacagt ttaccttatt |
| 1741 | gatattggtc tgatggtttg tgaactcttg ctgggaatca aaatttcctt gagactcttt |
| 1801 | agcattcata ctttggggtt aaaggagatt cctcagactc atccagccct tgggtgctga |
| 1861 | ccagcagagt cactagtgga tgctgaagtt acatgagcta catgttaaat atttaaagtc |
| 1921 | tccaaaataa aacaccccaa cgttgacctt acccggctga tggttagccc cttgctgcct |
| 1981 | gctccatgtg tcttatgaga gcccgtagtt acagtgtcct ctaatttgaa atccataagt |
| 2041 | taacaagtct atatcaggtg cagctggctt tgattaaagg ccatttttaa aacttaaaaa |
| 2101 | ctcaacacct cacagattat aatagaaaaa gaaatggcct cagtttgatc tcgttcagaa |
| 2161 | tgacccagat tgtttctgct ttgggtgcag ctgtttagtt cagagttata ttacagagaa |
| 2221 | ttattttctg agataatctt aaactagaat gttcaaaact aattgataat tgaagtatca |
| 2281 | agatacgtag aacacctcag agatttttct tcaggaactt ccacaaactt tgaatccttg |
| 2341 | tatctttatt tggtattcat actactagta gcaaaataca ggttttttgt tttgttttgt |
| 2401 | tttgttttgg cttcatagag tatctcaaat tgaaactttt ctgcacaaag aataaaatta |
| 2461 | aggattttat aaactcaaat tggcacctac tgaattaaaa tacataaaat catttaaata |
| 2521 | taattcagca tatgggaagt aacattgcac taatatggaa atcactgcca gagacagtct |
| 2581 | attttctttt aatttgttac tacttagtca caaaccccac attattccag tttggaatta |
| 2641 | cttattaagg agaattggaa atacatatgc ccatgcttaa attttatagc tttaatttgt |
| 2701 | gttatttctt tattgacggg aagaggtaca tctttttttc cttactgaaa acaaatatgg |
| 2761 | attaattgcc tcaaatttgt ataagtgatt ggctagtgat tcttgttttc agaagggaga |
| 2821 | gtggtataga tagaaaatga caaagatggc aatatacact taatgttgtt attgtatgtt |
| 2881 | gttactgaag tacttagatt tttaaaattt caaatcctaa atcacttctt gtaggagggt |
| 2941 | tttcattaac tgcagtatat acagttcact acatatgggt tgtttgagtt ttttgtgtgc |
| 3001 | tgtatttctt tctgtttttt aatacctggt tttgtacata tctaactctg ttctcttttg |
| 3061 | gttgttcaga aactggattt tttttttctt aagcagtgct taatttgtgt tttttaattt |
| 3121 | tgattcagaa gtagtcccag ctcataggtg ttcatactgt tacatccaga acatttgtca |
| 3181 | ggctctctgt cagctttcat gtacatatgg tatagaaacc atggagttag gcacttcctg |
| 3241 | gatttttttt ttatgagaaa aatactgtat ttaaaatgta aaataaactt ttaaaaagca |
| 3301 | ggcactaata tatatttctt ccagcctttg attacaaatt tgtccttgca catgttaaga |
| 3361 | tgaattatct cctaaaaata tcattgttct tgggagcagt gtatgttact ttacatagca |
| 3421 | gcggttcctg tcatgtgttc atgtcagaat atttttggtt ttaaactttc ttattgcctt |
| 3481 | tggctgttga ttagtacagt acaagtgcga tttcaaaaag atcttgaaag taatatattt |
| 3541 | aatcaattaa aatgtttatc tgtaaaaaaa aaaaaaaaaa a |

37: AA160544 ZNF325

| | |
|---|---|
| 1 | tttttttaca gttttcaaat attttactga aaatgcatat tgtacaatta atgtataatg |
| 61 | acacaccagt gtgagaaacc tccataggta tcatttccac aaatatgcta tgaatataga |
| 121 | gttcctacac aaaactatac aacttaccag atgtaattcc tgttacgtac catactcaca |
| 181 | atcgtcttga agaatatgga gaaaaagtgc tgagtgacaa aaacaggagc catgtgtgat |
| 241 | tttaataaat ggaaaacacg gcatttcagc tcagtggtaa agcagtaaac caatcagatg |
| 301 | cttagctatc aagtaatcat gtgagaggaa acagaattag atcctacctc atactatatg |
| 361 | ttgtcagcta acactgtagc agtggtatat gaatcactaa attacctcca acaaaatgta |
| 421 | ttcctgtatt gaaaaaagga ggtatggcca acattgtgtc acgttccaag gtgaattttg |
| 481 | cggtcacgat atgacgttca ggaagctact tttattgttc agttgatttc tatgctcaac |
| 541 | tattaggtca attccgaaat aatcncatat cacagctaaa ataatgncta ccaagtcnct |
| 601 | ctgactgct |

38: AK057653 LOC285513

| | |
|---|---|
| 1 | ctgttagcaa tgcttcctga tgttgtgcgt ggcccttttt ggttgattct ctccaaattc |
| 61 | gggtcagctg ctgccacctg gcaaataaca gaggatatgc tgaatctcct gtccatcctt |
| 121 | gtaacgatat ccttcttaat gaaattcttc aactggctga gcaattacaa atgtcatctg |
| 181 | tccagacaca tgggcttaag gatgtctaca aaattttaga catttttgca aatgggaaaa |
| 241 | aaaatagtct tgtaaatact gaaacagatt tccatgaact ttatcctact cttggaaaga |
| 301 | aaacaattct ccttggctgc agaaatcaaa taagctgggt ttgcaatgac caaggacata |
| 361 | aatgaagatg gattgaagtg gaaaaattct gtctcccaag tgatcagtga catctgccag |
| 421 | aggtcattac agctactttt aactgtgaac agtcaccagc taaactactc acttgccaca |
| 481 | acaaaataac ctctctcaaa gtaaatccag tgcatctgta tatatgtgta gatagcagca |
| 541 | acaaacaatc ctgaaacatt atttttggct gttaggtaag taaacgtgat gataattata |
| 601 | aacaacattc aaataacctt ggaccttggt gaaatgactt gtggtggcca gaatggtgca |
| 661 | acaagatgtt atttgcaagt ttttttaaga cacaaatatc tcagatacta ataatgagaa |
| 721 | taaagactgt tgaatatgaa attaaagcca agcaataatg tgccaaaaag aggcagttat |
| 781 | accagcaaat gcatctatta tgggcacacc attatataat gatggtttgc tttatgaaga |
| 841 | ctgactgtaa cccacaggat aaaataagca aaggcatagt ttctgctttc ttcctggaaa |
| 901 | aacttgttta gaagcttcat aaagaggtac agcactaatg agcattagtc aggatacagt |
| 961 | tggcatctat gtttttatgt gagcccagag ggaagaggag ccactcaaag tcttgctggt |
| 1021 | ttaaaactca agacagctgc aaccagaagt tttgttgaaa tggagacttt aaacttatgg |
| 1081 | taattactct ttctggacac tagcatgtag aaagcaattc agttaactct gcccagagga |
| 1141 | ttaccagctt tagctgtgaa aaaatgggct cccggatgta aaatcactaa aacatgagat |
| 1201 | cttgtatcca aagaggcttc aaatgatgcc ttacagaaaa cgatgctcca gatgggcact |
| 1261 | tctaaatgct aactcttcat caagtatctt tctggattca agctcaaaat taattggctg |
| 1321 | caaaatagta ggaataaaaa tcacatattt tacactttag aaaaggatat tgatgatcaa |
| 1381 | cctgcatggt gataattatg atgagatacc ccagtgattt aatgatgtta gaaagaatta |
| 1441 | aatgggagag aattgctaac agctttcttg atctcttaac tatggagatg tcattcattt |
| 1501 | atttctgggg tgaaaattat agcttgcttt ttgacattgc tgctagtatt gttctttgtt |
| 1561 | gctttaaaaa ttgtctctct ttagaaaaac tcttgagcag ttaaacagtt ctttttctga |
| 1621 | ttcatatcat tgcttttaat aacatgtaaa ggctgtgtgt agagcaaact atataaaatg |
| 1681 | agtagaaagg gcttgctcat gttaattggc atccttgatg attttagttg agattcctta |
| 1741 | acatttattt tagatcacat ctttacgtaa cttatttttc ctaatgtttt ccatcgtgtc |
| 1801 | ttaaaatgat gctggtatat caggagattg cagtattata gtcatactcc ccaatcccta |
| 1861 | gaggagagga aagactaatt cttgttttaa gggcccctgg agataccttt tattaaggtt |
| 1921 | gaaaaaggtc aacacagcct gaaaataaga aaaatatata ctagcaatta ctaattttct |
| 1981 | aaatgtgtgt atctctgctg tactaatgtg tgaacaatat gtcgtgcata atactgtagc |
| 2041 | tggtcgtggt atgtcaatac attctgtgag tgtgtacagt ctgagtgatc agttttctat |
| 2101 | ttttatgtgt aaaaaaaata acttgtcgta tcccatttaa aggccaattt ctgtattcag |
| 2161 | gcaggcatat gtacatacat gaataaagcc aacaaaagtg tgcacatgta ttcagt |

39: NM_003310 TSSC1

| | |
|---|---|
| 1 | aattcggcac gagaagactt ccagtttgga gtcgtttgct gcggggaggg aatgaatggg |
| 61 | cgctgggaac acgcccgcga ggtggggacg cgccggccgt agcgaggtcc ttagcgtgtg |
| 121 | agtggccggg gtcgggtcgc ttccccgcag catggaggac gatgcaccag tgatctacgg |
| 181 | gctggagttc caggcacgtg ccttaacacc tcaaactgca gaaacagatg ccattcggtt |
| 241 | tttggttggg acgcagtctc ttaaatatga taatcagatc catatcatag attttgacga |
| 301 | tgaaaacaac attataaata aaaatgtcct cctccatcaa gcgggtgaaa tctggcatat |
| 361 | tagcgctagc cctgcagaca gaggtgtgct gacgacctgc tacaacagaa cttcagacag |
| 421 | caaagtcctg acatgtgcag ccgtgtggag gatgccgaag gaattggaat caggcagcca |
| 481 | cgagtcccct gatgattcat ccagcactgc acagaccctg gagctgctct gtcaccttga |
| 541 | caacacagcc catggcaaca tggcctgtgt cgtgtgggag ccaatgggag atgggaagaa |
| 601 | aatcatttcc ttggctgata accatatcct gctgtgggat ttacaggaaa gctcgagcca |
| 661 | ggctgtgctg gccagctcag cgtccctgga agggaaggga caactgaagt tcacctcagg |
| 721 | acggtggagc ccacatcata actgcaccca ggtggccaca gcgaacgaca ccaccctccg |
| 781 | tggctgggac acccggagca tgagccagat ctactgcata gagaatgccc acggacagct |
| 841 | ggtgcgggac cttgacttta atcccaataa gcagtactac ttggccagct gcggagacga |
| 901 | ctgtaaggtg aagttctggg acacccgaaa tgtcaccgaa cccgtgaaga ccctggagga |
| 961 | gcactcccac tgggtgtgga acgtccgcta caaccactct catgaccagc tggtcctcac |
| 1021 | gggcagcagt gacagcagag tcatcctttc caacatggtg tccatctcgt cggagccctt |
| 1081 | cggccacttg gtagacgacg atgacatcag tgaccaggag gaccaccgtt ctgaagagaa |
| 1141 | gagcaaggag cccctgcagg acaacgtgat cgccacctac gaggagcacg aggacagcgt |
| 1201 | ctatgccgtg gactggtcct cggctgaccc gtggctgttt gcctccctga gctatgacgg |
| 1261 | gaggctcgtg atcaacaggg tgcccagggc cctgaagtac cacatcctgc tatgactccc |
| 1321 | gggcctgggt tatccaggtc ccattgagtg gttttcctct tggcagattc tcaaacagtc |
| 1381 | gcagctcttt ggaggtgact cgtgttccag gtggatccct ctctgggaga gccgctgttc |
| 1441 | ccttcctgta gcagcagcat ttatgaatgg ggtgaatggg gctattgtcg acggcacagc |
| 1501 | taatgcccga acccagcccc tgtcggcaga gacagagccc cacattatta tgtgaataac |
| 1561 | aatgttttct gttttaaggg tgtcaggagt ttcgcttttt aaaaaaatgt ctgttcctgc |
| 1621 | agtagtaact cttctttctc ttgagagtaa aaaatgaaat aaaataaatc cacgctgaca |
| 1681 | aaaaaaaaaa aaaaaaaaaa aaaaa |

40: BC007451 XAB1

| | |
|---|---|
| 1 | gaggaagatg gcggcgtccg cagctgccgc tgagctccag gcttctgggg gtccgcggca |
| 61 | cccagtgtgt ctgttggtgt tgggaatggc gggatccggg aaaaccactt ttgtacagag |
| 121 | gctcacagga cacctgcatg cccaaggcac tccaccgtat gtgatcaacc tggatccagc |
| 181 | agtacatgaa gttccctttc ctgccaatat tgatattcgt gatactgtaa agtataaaga |
| 241 | agtaatgaaa caatatggac ttggacccaa tggcggcata gtgacctcac tcaatctctt |
| 301 | tgctaccaga tttgatcagg tgatgaaatt tattgagaag gcccagaaca tgtccaaata |
| 361 | tgtgttgatt gacacacctg gacagattga ggtattcacc tggtcagctt ctgggacaat |
| 421 | tatcactgaa gcccttgcat cctcatttcc aacagttgtc atctatgtaa tggacacatc |
| 481 | gagaagtacc aacccagtga ccttcatgtc caacatgctc tatgcctgca gcatcttata |
| 541 | caaaaccaag ctgcctttca ttgtggtcat gaataaaact gacatcattg accacagctt |
| 601 | tgcagtggaa tggatgcagg attttgaggc tttccaagat gccttgaatc aagagactac |
| 661 | atacgtcagt aacctgactc gttcaatgag cctggtgtta gatgagtttt acagctcact |
| 721 | cagggtggtg ggtgtctctg ctgttctggg tactggatta gatgaactct ttgtgcaagt |
| 781 | taccagtgct gccgaagaat atgaaaggga gtatcgtcct gaatatgaac gtctgaaaaa |
| 841 | atcactggcc aacgcagaga gccaacagca gagagaacaa ctggaacgcc ttcgaaaaga |
| 901 | tatgggttct gtagccttgg atgcagggac tgccaaagac agcttatctc ctgtgctgca |
| 961 | cccttctgat ttgatcctga ctcgaggaac cttggatgaa gaggatgagg aagcagacag |
| 1021 | cgatactgat gacattgacc acagagttac agaggaaagc catgaagagc cagcattcca |
| 1081 | gaattttatg caagaatcga tggcacaata ctggaagaga aacaataaat aggagacttt |
| 1141 | agcacacttc acttgtttct agaagtccag aattttggac ctccacgtga aagaactgtt |
| 1201 | cttacctctg aactgggggc tcccataagg gataattttc ctcagagtag caaagtttct |
| 1261 | cttattagag aaatcttgtg actcagatga agtcagggat agaagaccct tggacctggc |
| 1321 | aggttaatgc tgattattcc ttggcctttc ccttgtattt atgcaaggaa ggatatactg |
| 1381 | agctgatact gttccaagcc tacaacttca agttttatca tttgaactca agtacttttg |
| 1441 | ctgctgagga atggaatcaa aagaacgtag tctcctggtg accacctcag atctctatta |
| 1501 | ttaggctaga tgtatagcct ctactccccc agcttcttgc tcttgaccct gcactgtaag |
| 1561 | ttgcccttct attagcagcc aaggaaaagg gaaacatgag cttatccaga acggtggcag |
| 1621 | agtctccttg gcaatcaacc aacgttgcta tgaaatatgc ctcacactgt atagctcatt |
| 1681 | ataggacgtc aggtttgttg aaaaaagtgg gcaagacatg attaatgaat cagaatcctg |
| 1741 | tttcattggt gacttggata aagacttttt aattttaaaa aaaaaaaaaa aaaaaaaaaa |

41: BC035467 HNLF

| | |
|---|---|
| 1 | ggctgaggcg cgatggcagg tgtcggggct gggcctctgc gggcgatggg gcggcaggcc |
| 61 | ctgctgcttc tcgcgctgtg cgccacaggc gcccaggggc tctacttcca catcggcgag |
| 121 | accgagaagc gctgtttcat cgaggaaatc cccgacgaga ccatggtcat cggcaactat |
| 181 | cgtacccaga tgtgggataa gcagaaggag gtcttcctgc cctcgacccc tggcctgggc |
| 241 | atgcacgtgg aagtgaagga ccccgacggc aaggtggtgc tgtcccggca gtacggctcg |
| 301 | gagggccgct tcacgttcac ctcccacacg cccggtgacc atcaaatctg tctgcactcc |
| 361 | aattctacca ggatggctct cttcgctggt ggcaaactgc gtgtgcatct cgacatccag |
| 421 | gttggggagc atgccaacaa ctaccctgag attgctgcaa aagataagct gacggagcta |
| 481 | cagctccgcg cccgccagtt gcttgatcag gtggaacaga ttcagaagga gcaggattac |
| 541 | caaaggtatc gtgaagagcg cttccgactg acgagcgaga gcaccaacca gagggtccta |
| 601 | tggtggtcca ttgctcagac tgtcatcctc atcctcactg gcatctggca gatgcgtcac |
| 661 | ctcaagagct tctttgaggc caagaagctg gtgtagtgcc ctctttgtat gacccttcct |
| 721 | ttttacctca tttatttggt actttcccca cacagtcctt tatccacctg gatttttagg |
| 781 | gaaaaaaatg aaaaagaata agtcacattg gttccatggc cacaaaccat tcagatcagc |
| 841 | cacttgctga ccctggttct taaggacaca tgacattagt ccaatctttc aaaatcttgt |
| 901 | cttagggctt gtgaggaatc agaactaacc caggactcag tcctgcttct tttgcctcga |
| 961 | gtgattttcc tctgtttttc actaaataag caaatgaaaa ctctctccat taccttctgc |
| 1021 | tttctctttg tccacttacg cagtaggtga ctggcatgtg ccacagagca ggccctgcct |
| 1081 | cactgtctgc tggtcagttc tgggttcact taatggcttt gtgaatgtaa ataaggggca |
| 1141 | ggtcttggcc ctagaggatt gagatgtttt tctatatctt agaactattt ttggataaat |
| 1201 | tatatatttt ccttcctagt agaagtgtta ctgcctgtaa ctagctcaaa ataccaatgc |
| 1261 | agtttctgca ttctgggttt tgtttttcct tttttttttt tttttttttt ttttgagttt |
| 1321 | tgctctcgtc gcccaggctg gagtgcaatg gcgtgatctc agctcactgg caacatctgc |
| 1381 | ctcccgggtt caaatgattc tcctgcctca gtctcctgag tagctgggat tacaggtgcc |
| 1441 | cgccaccacg ctcagctaat ttttgtattt ttagtagaga tggggtttta ccatgttggc |
| 1501 | caggctggtc ttagactcct gacctcagtt gatccacctg cctcagcctc tgcattcagt |
| 1561 | ttattcacat atttttggta actcccatgg cagctcctag gatttcagcg gtctgtgggc |
| 1621 | cagaaagcag gcaccagggc tgacctcaag gccgtatcag agggccaagc agagttcttt |
| 1681 | tggatacctg cttttcatcc cacagggcct tagagtcaga ggtaaggtag caacagagct |
| 1741 | agaatggggc aatgcactct taccctcctt ctcaactttt atttaagctg tgctaaatgt |
| 1801 | tttcttcaag ggaaccagat ttagttcttt acagaatttt ccagtgaaat aaaacatgtt |
| 1861 | gtaatagctg tgtttgagat gaaataagag gttgtgggta gaggggaggc acctaaagga |
| 1921 | aaagaggaaa ggtgcctggg ctacctatgc agataacctg gagtggactt cactgtggac |
| 1981 | tcgtggtact aaggcttggc ctggacaggc agtctagggg gtatgggaat acacggtgtg |
| 2041 | gttgttcaac tatttgcaaa ggtcaaccaa atagaccaca tgttcgcaaa gtatcatctg |
| 2101 | aggaaattaa gtaccttctt agccctctca gtcataaatt tgaacaaatt ttaatacact |
| 2161 | tccctcatgc ccttctatat aaaacttaat accattagtt ccccattctt gacattttat |
| 2221 | ttcagttttt attatatatt tatttgaaat atttattaaa ttatctgacc tacagaacta |
| 2281 | aaaaaaaaaa aaaaaaa |

42: CK004097 EIF4EBP

| | |
|---|---|
| 1 | gggacatttc caagggtatt taaactctca ctctgccacc tttctaaggg tgggaggctg |
| 61 | gcagagatgc tgcaatgctt gataatcatt tggccacact gaaatttcca aagggagctc |
| 121 | ttgccggtgc ttaaaaccaa aactcctgga cacttagaaa attccatgaa tctagcacaa |
| 181 | aatatccatt cttgcccaag tgtatcccct ttctctccag cttaatcttt tttttttttt |
| 241 | ttttttaaag cccaggccaa gggtactttt aactggaaac tggggaggag ggaagaacac |
| 301 | tagcagggag ctaagaggca ggttgctggg taagccatcc tgctcctacc tggtgcctgt |
| 361 | atctacattg ctgagtgctg tgcgccagtg cctttccttc atctgcagat ggagcccatc |
| 421 | tctttccacc tgggtgagga gaccctctgc tactccaggg gtaaacctta aagaaggtgt |
| 481 | cttgaagagc ccaaaggaca ctcacgtgct aaggtgtcca ttttatgcat ctttaaaata |
| 541 | ttttatttaa aaaaaaaaat agccctgccc tgtcttagtg ccactaacgg cccagattca |
| 601 | ttcattctga atggaaaaac ngagactgcc agcactttcc tttggtcctt ccn |

43: NM_144683 MGC32380

| | |
|---|---|
| 1 | catggaggcg ctgctgctgg gcgcggggtt gctgctgggc gcttacgtgc ttgtctacta |
| 61 | caacctggtg aaggccccgc cgtgcggcgg catgggcaac ctgcggggcc gcacggccgt |
| 121 | ggtcacgggt gagtgcggag gcgggtgagt gcgagctggc ggggcgcgcg gagaggaggc |
| 181 | cgggccggcg gtagcagcgg cccgccgggc tcagctcagc tcggctcccg cccgcggtcc |
| 241 | gcaggcgcca acagcggcat cggaaagatg acggcgctgg agctggcgcg ccggggagcg |
| 301 | cgcgtggtgc tggcctgccg cagccaggag cgcggggagg cggctgcctt cgacctccgc |
| 361 | caggagagtg ggaacaatga ggtcatcttc atggccttgg acttggccag tctggcctcg |
| 421 | gtgcgggcct ttgccactgc ctttctgagc tctgagccac ggttggacat cctcatccac |
| 481 | aatgccggta tcagttcctg tggccggacc cgtgaggcgt ttaacctgct gcttcgggtg |
| 541 | aaccatatcg gtccctttct gctgacacat ctgctgctgc cttgcctgaa ggcatgtgcc |
| 601 | cctagccgcg tggtggtggt agcctcagct gcccactgtc ggggacgtct tgacttcaaa |
| 661 | cgcctggacc gcccagtggt gggctggcgg caggagctgc gggcatatgc tgacactaag |
| 721 | ctggctaatg tactgtttgc ccgggagctc gccaaccagc ttgaggccac tggcgtcacc |
| 781 | tgctatgcag cccacccagg gcctgtgaac tcggagctgt tcctgcgcca tgttcctgga |
| 841 | tggctgcgcc cacttttgcg cccattggct tggctggtgc tccgggcacc aagagggggt |
| 901 | gcccagacac ccctgtattg tgctctacaa gagggcatcg agcccctcag tgggagatat |
| 961 | tttgccaact gccatgtgga agaggtgcct ccagctgccc gagacgaccg ggcagcccat |
| 1021 | cggctatggg aggccagcaa gaggctggca gggcttgggc ctggggagga tgctgaaccc |
| 1081 | gatgaagacc cccagtctga ggactcagag gccccatctt ctctaagcac cccccaccct |
| 1141 | gaggagccca cagtttctca accttacccc agccctcaga gctcaccaga tttgtctaag |
| 1201 | atgacgcacc gaattcaggc taaagttgag cctgagatcc agctctccta accctcaggc |
| 1261 | caggatgctt gccatggcac ttcatggtcc ttgaaaacct cggatgtgtg cgaggccatg |
| 1321 | ccctggacac tgacgggttt gtgatcttga cctccgtggt tactttctgg ggccccaagc |
| 1381 | tgtgccctgg acatctcttt tcctggttga aggaataatg ggtgattatt tcttcctgag |
| 1441 | agtgacagta accccagatg gagagatagg ggtatgctag acactgtgct tctcggaaat |
| 1501 | ttggatgtag tattttcagg ccccaccctt attgattctg atcagctctg gagcagaggc |
| 1561 | agggagtttg caatgtgatg cactgccaac attgagaatt agtgaactga tccctttgca |
| 1621 | accgtctagc taggtagtta aattaccccc atgttaatga agcggaatta ggctcccgag |
| 1681 | ctaagggact cgcctagggt ctcacagtga gtaggaggag ggcctgggat ctgaacccaa |
| 1741 | gggtctgagg ccagggccga ctgccgtaag atgggtgctg agaagtgagt cagggcaggg |
| 1801 | cagctggtat cgaggtgccc catgggagta aggggacgcc ttccgggcgg atgcagggct |
| 1861 | ggggtcatct gtatctgaag cccctcggaa taaagcgcgt tgaccgccaa aaaaaaaaaa |
| 1921 | aaaaaaaaaa aaaaaaaaaa aaaaaaaaaa aaaaa |

44: NM_004600 SSA2

| | |
|---|---|
| 1 | tcctgcttgt cggcatcgct ccccacaggc cgacgtcgag agggcctgct ttactcctcc |
| 61 | tctttctcct ccttctcccg cggcttctgc gcggagaggc gtcgcccggg atctgggttt |
| 121 | tggaagaagg atctttgtgg gaagacaggg tgaatttatc acagaggaat aacgagggag |
| 181 | aggagaaagg tttcctaaag acaaaaaaaa aaatggagga atctgtaaac caaatgcagc |
| 241 | cactgaatga gaagcagata gccaattctc aggatggata tgtatggcaa gtcactgaca |
| 301 | tgaatcgact acaccggttc ttatgtttcg gttctgaagg tgggacttat tatatcaaag |
| 361 | aacagaagtt gggccttgaa aatgctgaag ctttaattag attgattgaa gatggcagag |
| 421 | gatgtgaagt gatacaagaa ataaagtcat ttagtcaaga aggcagaacc acaaagcaag |
| 481 | agcctatgct ctttgcactt gccatttgtt cccagtgctc cgacataagc acaaaacaag |
| 541 | cagcatttaa agctgtttct gaagtttgtc gcattcctac ccatctcttt acttttatcc |
| 601 | agtttaagaa agatctgaag gaaagcatga aatgtggcat gtggggtcgt gccctccgga |
| 661 | aggctatagc ggactggtac aatgagaaag gtggcatggc ccttgctctg gcagttacaa |
| 721 | aatataaaca gagaaatggc tggtctcaca aagatctatt aagattgtca catcttaaac |
| 781 | cttccagtga aggacttgca attgtgacca aatatattac aaagggctgg aaagaagttc |
| 841 | atgaattgta taaagaaaaa gcactctctg tggagactga aaaattatta aagtatctgg |
| 901 | aggctgtaga gaaagtgaag cgcacaagag atgagctaga agtcattcat ctaatagaag |
| 961 | aacatagatt agttagagaa catcttttaa caaatcactt aaagtctaaa gaggtatgga |
| 1021 | aggctttgtt acaagaaatg ccgcttactg cattactaag gaatctagga aagatgactg |
| 1081 | ctaattcagt acttgaacca ggaaattcag aagtatcttt agtatgtgaa aaactgtgta |
| 1141 | atgaaaaact attaaaaaag gctcgtatac atccatttca tattttgatc gcattagaaa |
| 1201 | cttacaagac aggtcatggt ctcagaggga aactgaagtg gcgccctgat gaagaaattt |
| 1261 | tgaaagcatt ggatgctgct ttttataaaa catttaagac agttgaacca actggaaaac |
| 1321 | gtttcttact agctgttgat gtcagtgctt ctatgaacca aagagttttg ggtagtatac |
| 1381 | tcaacgctag tacagttgct gcagcaatgt gcatggttgt cacacgaaca gaaaaagatt |
| 1441 | cttatgtagt tgctttttcc gatgaaatgg taccatgtcc agtgactaca gatatgacct |
| 1501 | tacaacaggt tttaatggct atgagtcaga tcccagcagg tggaactgat tgctctcttc |
| 1561 | caatgatctg ggctcagaag acaaacacac ctgctgatgt cttcattgta ttcactgata |
| 1621 | atgagacctt tgctggaggt gtccatcctg ctattgctct gagggagtat cgaaagaaaa |
| 1681 | tggatattcc agctaaattg attgtttgtg gaatgacatc aaatggtttc accattgcag |
| 1741 | acccagatga tagaggcatg ttggatatgt gcggctttga tactggagct ctggatgtaa |
| 1801 | ttcgaaattt cacattagat atgatttaac cataagcagc agcacgatcc agagatccat |
| 1861 | tgccatcagt gatctcacta aaaatataca gctacttccc agctaatctc cacccaatga |
| 1921 | atgatgatgg tatagtatgt gcataatgga aagttacctt actgaaaaaa aaaaaagaag |
| 1981 | gaaaaataag atgggcccaa aggtctatct actaaactag ctcttgggga aatagcttca |
| 2041 | ggatactgta gtttcctcta tctaatagag aactttttgt taacagacac tgtaaaatag |
| 2101 | ttttgctttg ttgaataata catgtgtacc taaaagaggt aagagcaaaa agtgtaattc |
| 2161 | cacatcatgt tacttgagaa gtgcttaacg ttttcttaaa tgttttcatt gggaaaggac |
| 2221 | agctttgata atgtccaaat actctgaaat gcactagacc atataactgt gatgaaatat |
| 2281 | gaaactcatc tgtaaacttt tataccaagg gggtaaaaaa aaaaactaag gcatttgatt |
| 2341 | aaattatgaa tgagttttac aaattccttt cagagtttta ctaagatcac acaaataaca |
| 2401 | gctttcttat tcagtgaaaa agatatttta tttctgatgt tttatttgca ctcgtggaat |
| 2461 | atgttaccat taatcagaaa catcatggca acccctaaga atagactaag tttgtgttgg |
| 2521 | ctgagggatt ctatttggtt tgcttttttt tttttgcttt gttatatttt attgctacaa |
| 2581 | ggggtgtgac ttgataatga tttcctctga attataataa catagccaga tgtagtctca |
| 2641 | cactgttttt catactctta agtgtaaata atataaaatg tttcaagcgc ttaactcccc |
| 2701 | ctcattcaca aagtataaca attaaaatct caactataac cagtttagct ttttccttac |
| 2761 | ttttaaaata aaatttttta cttttaacta tttttttagt taatattttt aaaagtatac |
| 2821 | atgtcaatgg cctctttgtc cattattcat tttgtggcaa aatattcttc tttgatagtg |
| 2881 | taaacaaata ataaagcaat ctaggtcctt taggtttgaa aggcaatttt tgagtagcat |
| 2941 | attaccagct agccagtcac taggaatttt tttcagtatt atttgtatgt attaaacttt |
| 3001 | tcattacact aaagtgcatt attttattga gcaagtatcc ttcattgtga ggtttgacat |
| 3061 | taaagcaatc tgttgaaatg ccaaaaaaaa aaaaaaaa |

45: NM_002730 PRKACA

| | |
|---|---|
| 1 | gatcttgggc tgaggttccc gggcgggcgg gcgcggagag acgcgggaag caggggctgg |
| 61 | gcgggggtcg cggcgccgca gctagcgcag ccagcccgag ggccgccgcc gccgccgccc |
| 121 | agcgcgctcc ggggccgccg gccgcagcca gcacccgccg cgccgcagct ccgggaccgg |
| 181 | ccccggccgc cgccgccgcg atgggcaacg ccgccgccgc caagaagggc agcgagcagg |
| 241 | agagcgtgaa agaattctta gccaaagcca aagaagattt tcttaaaaaa tgggaaagtc |
| 301 | ccgctcagaa cacagcccac ttggatcagt ttgaacgaat caagaccctc ggcacgggct |
| 361 | ccttcgggcg ggtgatgctg gtgaaacaca aggagaccgg gaaccactat gccatgaaga |
| 421 | tcctcgacaa acagaaggtg gtgaaactga aacagatcga acacaccctg aatgaaaagc |
| 481 | gcatcctgca agctgtcaac tttccgttcc tcgtcaaact cgagttctcc ttcaaggaca |
| 541 | actcaaactt atacatggtc atggagtacg tgcccggcgg ggagatgttc tcacacctac |
| 601 | ggcggatcgg aaggttcagt gagccccatg cccgtttcta cgcggcccag atcgtcctga |
| 661 | cctttgagta tctgcactcg ctggatctca tctacaggga cctgaagccg gagaatctgc |
| 721 | tcattgacca gcagggctac attcaggtga cagacttcgg tttcgccaag cgcgtgaagg |
| 781 | gccgcacttg gaccttgtgc ggcacccctg agtacctggc ccctgagatt atcctgagca |
| 841 | aaggctacaa caaggccgtg gactggtggg ccctgggggt tcttatctat gaaatggccg |
| 901 | ctggctaccc gcccttcttc gcagaccagc ccatccagat ctatgagaag atcgtctctg |
| 961 | ggaaggtgcg cttcccttcc cacttcagct ctgacttgaa ggacctgctg cggaacctcc |
| 1021 | tgcaggtaga tctcaccaag cgctttggga acctcaagaa tggggtcaac gatatcaaga |
| 1081 | accacaagtg gtttgccaca actgactgga ttgccatcta ccagaggaag gtggaagctc |
| 1141 | ccttcatacc aaagtttaaa ggccctgggg atacgagtaa ctttgacgac tatgaggaag |
| 1201 | aagaaatccg ggtctccatc aatgagaagt gtggcaagga gttttctgag ttttaggggc |
| 1261 | atgcctgtgc ccccatgggt tttctttttt cttttttctt ttttttggtc gggggggtgg |
| 1321 | gagggttgga ttgaacagcc agagggcccc agagttcctt gcatctaatt tcacccccac |
| 1381 | cccaccctcc agggttaggg ggagcaggaa gcccagataa tcagagggac agaaacacca |
| 1441 | gctgctcccc ctcatcccct tcaccctcct gccccctctc ccacttttcc cttcctcttt |
| 1501 | ccccacagcc ccccagcccc tcagccctcc cagcccactt ctgcctgttt taaacgagtt |
| 1561 | tctcaactcc agtcagacca ggtcttgctg gtgtatccag ggacagggta tggaaagagg |
| 1621 | ggctcacgct taactccagc ccccacccac acccccatcc cacccaacca caggccccac |
| 1681 | ttgctaaggg caaatgaacg aagcgccaac cttcctttcg gagtaatcct gcctgggaag |
| 1741 | gagagatttt tagtgacatg ttcagtgggt tgcttgctag aattttttta aaaaaacaac |
| 1801 | aatttaaaat cttatttaag ttccaccagt gcctccctcc ctccttcctc tactcccacc |
| 1861 | cctcccatgt ccccccattc ctcaaatcca ttttaaagag aagcagactg actttggaaa |
| 1921 | gggaggcgct ggggtttgaa cctccccgct gctaatctcc cctgggcccc tccccgggga |
| 1981 | atcctctctg ccaatcctgc gagggtctag gcccctttag gaagcctccg ctctcttttt |
| 2041 | ccccaacaga cctgtcttca cccttgggct ttgaaagcca gacaaagcag ctgcccctct |
| 2101 | ccctgccaaa gaggagtcat cccccaaaaa gacagagggg gagccccaag cccaagtctt |
| 2161 | tcctcccagc agcgtttccc cccaactcct taattttatt ctccgctaga ttttaacgtc |
| 2221 | cagccttccc tcagctgagt ggggagggca tccctgcaaa agggaacaga agaggccaag |
| 2281 | tccccccaag ccacggcccg gggttcaagg ctagagctgc tggggagggg ctgcctgttt |
| 2341 | tactcaccca ccagcttccg cctcccccat cctgggcgcc cctcctccag cttagctgtc |
| 2401 | agctgtccat cacctctccc ccactttctc atttgtgctt ttttctctcg taatagaaaa |
| 2461 | gtggggagcc gctggggagc caccccattc atccccgtat ttccccctct cataacttct |
| 2521 | ccccatccca ggaggagttc tcaggcctgg ggtggggccc cgggtgggtg cgggggcgat |
| 2581 | tcaacctgtg tgctgcgaag gacgagactt cctcttgaac agtgtgctgt tgtaaacata |
| 2641 | tttgaaaact attaccaata aagttttgtt taaaaaaaaa aaaaaaaaa |

46: NM_005102 FEZ2

| | |
|---|---|
| 1 | ccggagcctc ctggaccagg agaactgtaa cgcgagcccc gagccatggg cgaaaggcgg |
| 61 | ggccgagacg ggttgggggc gccgacggtt tcccggccct ggctgcagct tggaggagaa |
| 121 | gctgagcctg tgcttccgcc cctcggatcc gggcgccgag cccgaggacg gccgtgcggc |
| 181 | catcacggag ctcaactcct gcagggggac gagatttgga atgccctgac agataattat |
| 241 | gggaatgtga tgcctgtaga ctggaagtca tcgcatacta ggaccttgca cttgcttact |
| 301 | ctgaacctct cagaaaaagg ggtaagtgac agtttgctct ttgatacatc agatgatgaa |
| 361 | gagctgagag aacagctgga tatgcactca atcatcgtct cctgtgttaa tgatgaaccc |
| 421 | ctcttcacgg cagaccaggt tattgaagaa attgaagaaa tgatgcagga atcaccggac |
| 481 | ccagaagatg atgaaacccc tacacagtca gatcggcttt caatgctttc ccaggaaatt |
| 541 | caaactctca agaggtctag taccggcagt tatgaagaga gagtgaaaag gctctcagtg |
| 601 | tctgagttaa atgaaatcct ggaagaaatt gagactgcca ttaaggagta ctctgaggag |
| 661 | ctggtgcagc agttggcttt acgagatgaa ctggagtttg aaaaggaagt gaaaaacagc |
| 721 | tttatttctg ttcttattga agtgcaaaac aaacagaaag agcacaaaga aacagcaaaa |
| 781 | aagaaaaaga aactaaaaaa tggcagctct cagaatggga agaatgagag aagtcatatg |
| 841 | cccggcacat atttgactac agtcattcct tatgagaaaa aaaacggacc accgtctgtt |
| 901 | gaagatcttc aaatattaac aaaaattctt cgtgccatga aggaggacag tgaaaaagtt |
| 961 | ccgagcttgt taactgatta tattctgaaa gttctgtgtc ctacatagag cagcaacttt |
| 1021 | atctgcggtg ggctccaagc tagatttccg acagcattat tctgagagct ggctaccatt |
| 1081 | acccttcttg ctattggaaa ctcagcacat ttgaacttgg gtttgattca gtattaacag |
| 1141 | atcttgacta cactaattct ttatattata gaaccaacgg aaatatgggc actattttga |
| 1201 | attctagaga tggtttttgt taaatctact aataaactgt tctcttagta gattaagaga |
| 1261 | gagtaatatt aattgtgcat gtgcagttgt atttctcatt aactgacagt atgcccattt |
| 1321 | gtttttatgg ctttcttatc taaactgcac tgatgaacta gattaaagcc ttgggagatt |
| 1381 | tatactataa attcagtgat ggcaagaacc aacactgttt ttttgtgaga attgtcagtg |
| 1441 | taactattac ctaccagtat tgttcagaga gattgaaaca gaataaacgg gctgttcttg |
| 1501 | aagaagcaaa accagaatat gcattacttt ggtttaatac ttagtgctaa cattgaaact |
| 1561 | gttggtggtg atggattttg tagcttgctg cttgtttcac cactggtcaa attttaacca |
| 1621 | ttaaattgcc attcactttt agaatcttgt atttaagtaa gttttgattt tcaaatgttc |
| 1681 | tgcttcatgt gtctgtgaag aattgtactt ttttaaaagt gtgtgtcctc tgaggtgctt |
| 1741 | gagaaagtgt acactgcaga actgcccatt ctcattactg tgtcctattt tattcatgcc |
| 1801 | tgtgtgtttt tcttaagtat gaattctaga tacagctact tatggattca tcaatatcat |
| 1861 | gagcactttt gctggttcca gtcaaatcaa tggcatttaa taaatttttt aagaagtaaa |
| 1921 | aaaaaaaaaa aaaaaa |

47: NM_005839 SRRM1

| | |
|---|---|
| 1 | ggagtttagg gcctgacaga agcccgcccc cgctggcgct cgtgcgcacg cgtggcgggc |
| 61 | tctcggcgca ctgagcaggc gcggcctcgt gtcggccgga gggggcgggc gcaacgacgc |
| 121 | gcgctgcgtc ccggcgctcg gctttccctc cgccggtccc gccctccgtc gcggcggcgc |
| 181 | ggtgtaccct gggataggga gcgatctccg agcgaggcgg caagatggac gcgggatttt |
| 241 | tccgcggaac aagtgcagaa caggataatc ggttcagcaa caaacagaag aaactactga |
| 301 | agcagctgaa atttgcagaa tgcctagaaa aaaaggtgga catgagcaaa gtaaatttgg |
| 361 | aggttataaa gccttggata acaaaaagag taacggaaat ccttgggttt gaagatgatg |
| 421 | ttgtgattga gtttatattc aaccagctgg aagtgaagaa tccagactcc aaaatgatgc |
| 481 | aaatcaacct gactggattt ttgaatggaa aaaatgctcg agaatttatg ggagaactgt |
| 541 | ggcccctgct gctaagtgca caagaaaaca tcgcgggaat cccttctgct ttcctagaac |
| 601 | tgaagaaaga agaaataaaa caaagacaga ttgaacaaga aaaactggca tctatgaaaa |
| 661 | agcaagatga agacaaagat aaaagagata aggaagaaaa agaaagcagc agagaaaaaa |
| 721 | gggagcggtc tcgtagccca agaagacgca aatccagatc tccttcccct agaagacgat |
| 781 | cttcccctgt caggagagag agaaagcgca gtcattctcg atctccccgt cacagaacca |
| 841 | agagccggag tccttcccct gctccagaaa agaaggaaaa aactccagag ctcccagaac |
| 901 | cttcagtgaa agtaaaagaa ccttcagtac aagaggctac ttctactagt gacattctga |
| 961 | aagttcccaa acctgaacct ataccagagc ctaaagaacc ttctccggaa aaaaattcca |
| 1021 | aaaaagaaaa ggagaaggag aagacccgac cacgatctcg gtcacgctcc aaatcaagat |
| 1081 | cccggacgcg gtcccgctct ccttctcaca ctcgacctag acggcgccat agatcccgat |
| 1141 | caagatcgta ttcacctaga aggcggccaa gcccaagaag gcggccatct cctcgaagaa |
| 1201 | gaactccgcc aagaagaatg cctcctccac caaggcatag aaggagtaga tctccagtaa |
| 1261 | gacgaagaag acgttcgtca gcatccttgt ctgggagtag ctcatcatcc tcttcatctc |
| 1321 | gttcacggtc accaccaaag aagcctccca agaggacatc cagcccccct cggaaaactc |
| 1381 | gtaggttatc tccttcagca agtcctccaa ggcgaaggca caggccatca cctcctgcaa |
| 1441 | ctccaccacc caaaactcgg cattccccta caccccagca gtcaaaccgt acaagaaaaa |
| 1501 | gtcgtgtttc tgtgtctcca gggagaactt caggtaaagt gacaaaacat aaaggtactg |
| 1561 | agaaaagaga atccccttca ccagcaccga agcctagaaa agtagagtta tctgaatcgg |
| 1621 | aagaagataa aggtggcaaa atggctgcag cagattctgt gcagcagaga cgccaataca |
| 1681 | gacgacaaaa ccagcagtct tcatctgact ctggctcctc ctcctcctca gaagatgaac |
| 1741 | gacccaagag atcccatgtg aagaatggtg aggttggcag gcggcggaga cattcccctt |
| 1801 | cccggagtgc ttctccatca ccacgaaagc gccaaaaaga gacttcccct cgtggtagac |
| 1861 | ggaggagaag tccatcccca ccacccacca gaaggcgacg gtctccttct cccgcccctc |
| 1921 | ctcctcgacg gcgcaggact cccacaccac caccacgacg aaggactcct tctcctcccc |
| 1981 | cacgtcggcg ctcaccttct cctagaagat actctcctcc aatacagagg agatactctc |
| 2041 | cttctccacc tccaaagaga agaacggctt cacctcctcc ccctcctaaa cgaagagcat |
| 2101 | caccatctcc accaccaaag cggcgggtct cccattctcc acctcccaaa caaagaagct |
| 2161 | ccccagtcac caagagacgt tcaccttcat tatcatccaa gcataggaaa gggtcttccc |
| 2221 | caagccgctc tacccgggag gcccgatcac cacaaccaaa caaacggcat tcgccctcac |
| 2281 | cacggcctcg agctcctcag acctcctcaa gtcctccacc cgttcgaaga ggagcgtcgt |
| 2341 | catcacccca aagaaggcag tccccgtctc caagtactag gcccattagg agagtctcca |
| 2401 | ggactccgga acctaaaaag ataaaaaagg ctgcttcccc aagcccacag tctgtaagaa |
| 2461 | gggtctcatc ctcccgatct gtctccgggt ctcctgagcc agcagctaaa aagcccccag |
| 2521 | cacctccatc ccccgtccag tctcagtcac cgtctacaaa ctggtcacca gctgtaccgg |
| 2581 | tcaaaaaggc caaaagccca acaccgagcc catcaccgcc aagaaattca gatcaggaag |
| 2641 | gaggtggaaa gaaaaagaag aaaaagaagg acaagaaaca caaaaaggat aagaagcaca |
| 2701 | agaagcacaa aaaacacaag aaggaaaagg ctgtggctgc agctgctgca gctgctgtga |
| 2761 | cccctgcagc cattgcagct gccacaacca cattagcaca ggaagagcca gtggcagcgc |
| 2821 | cagagccgaa gaaggagact gaaagtgaag ctgaagataa ccttgatgat ttagaaaagc |
| 2881 | acctgcgtga aaaggccctg agatcaatga ggaaggccca agtgtcccca cagtcttagg |
| 2941 | gggaaatgtt tgttatgatg taaattttat ttggtttgta cgcagttcaa tttcaaaatt |
| 3001 | gctaaaatgt gtttgagctt tagactataa catttgttgt aataattgct aggttgaagt |
| 3061 | tcaacatgta aaaaaagggg gcatggattt acattgcaaa aggtgtccac agtgtattag |
| 3121 | tgacattctt tcattgacag ctgacataat tcattgagtg aaatatttta agccaaaaaa |
| 3181 | aaattccctt tttaaaaaag ggggtttaaa tactgttggc atttttatgg ttcctttaaa |
| 3241 | tgccctagct attcccagag gggttttttt gtttgttttt ttggttttga ttttcttttt |
| 3301 | gtttttcttt cttcttctta tttttttcat ttgagtctta gctcccattt aagttatgct |
| 3361 | tctgaccttg tatggtctgt aagcttgccc agaaataaga ccactgtttt gaactaccac |
| 3421 | aaaagtataa atgaatattt taatgccaca atctttcctg ttgcctgtgg agtctctgct |
| 3481 | gaaatgaatc aggattcgag ctctaggatg agacagaaaa tgaaagcatg ttgtttgcca |
| 3541 | ggacactgtg ggtttatatt gatgtgtaac aagttgattt ggaacactgg actctcattc |
| 3601 | tgttattctg gttttgtttt ttttgttttg ttttttttct tttgtaaagg caatgagcta |
| 3661 | gtcccagaaa ggatccttca gttacataca atttgtttaa tgaaatgtca tggctctgtt |
| 3721 | catatttttg tcttgttctt ccaattggta tatacaactt tcagagcctc ttgtatttgg |
| 3781 | aaggctggaa gggcccagac tttggaatag tgtcttggtt tcactgtttt tgttttgatt |
| 3841 | ttttttttgt tttgattttt tttaaactaa agctatataa agcttgtgga ttaaacagaa |
| 3901 | taaatttcta aatttaaaaa tttaaaaaaa aaaaaaaaaa aaaaaaaaaa aaaaaaaaaa |
| 3961 | aaaaaaaaaa aaggaaaaaa aaaaaaaaaa |

48: NM_006207 PDGFRL

| | |
|---|---|
| 1 | cctgcgtccc cgccccgcgc agccgccgcg ctcctgcgct ccgaggtccg aggttcccga |
| 61 | gatgaaggtc tggctgctgc ttggtcttct gctggtgcac gaagcgctgg aggatgttac |
| 121 | tggccaacac cttcccaaga acaagcgtcc aaaagaacca ggagagaata gaatcaaacc |
| 181 | taccaacaag aaggtgaagc ccaaaattcc taaaatgaag gacagggact cagccaattc |
| 241 | agcaccaaag acgcagtcta tcatgatgca agtgctggat aaaggtcgct tccagaaacc |
| 301 | cgccgctacc ctgagtctgc tggcggggca aactgtagag cttcgatgta aagggagtag |
| 361 | aattgggtgg agctaccctg cgtatctgga cacctttaag gattctcgcc tcagcgtcaa |
| 421 | gcagaatgag cgctacggcc agttgactct ggtcaactcc acctcggcag acacaggtga |
| 481 | attcagctgc tgggtgcagc tctgcagcgg ctacatctgc aggaaggacg aggccaaaac |
| 541 | gggctccacc tacatctttt ttacagagaa aggagaactc tttgtacctt ctcccagcta |
| 601 | cttcgatgtt gtctacttga acccggacag acaggctgtg gttccttgtc gggtgaccgt |
| 661 | gctgtcggcc aaagtcacgc tccacaggga attcccagcc aaggagatcc cagccaatgg |
| 721 | aacggacatt gtttatgaca tgaagcgggg ctttgtgtat ctgcaacctc attccgagca |
| 781 | ccagggtgtg gtttactgca gggcggaggc cgggggcaga tctcagatct ccgtcaagta |
| 841 | ccagctgctc tacgtggcgg ttcccagtgg ccctccctca acaaccatct tggcttcttc |
| 901 | aaacaaagtg aaaagtgggg acgacatcag tgtgctctgc actgtcctgg gggagcccga |
| 961 | tgtggaggtg gagttcacct ggatcttccc agggcagaag gatgaaaggc ctgtgacgat |
| 1021 | ccaagacact tggaggttga tccacagagg actgggacac accacgagaa tctcccagag |
| 1081 | tgtcattaca gtggaagact tcgagacgat tgatgcagga tattacattt gcactgctca |
| 1141 | gaatcttcaa ggacagacca cagtagctac cactgttgag ttttcctgac ttggaaaagg |
| 1201 | aaatgtaatg aacttatgga aagcccattt gtgtacacag tcagctttgg ggttcctttt |
| 1261 | attagtgctt tgccagaggc tgatgtcaag caccacaccc caaccccagc gtctcgtgag |
| 1321 | tccgacccag acatccaaac taaaaggaag tcatccagtc tattcacaga agtgttaact |
| 1381 | tttctaacag aaagcatgat tttgattgct tacctacata cgtgttccta gtttttatac |
| 1441 | atgtgtaaac aattttatat aatcaatcat ttctattaaa tgagcacgtt tttgtaaaaa |
| 1501 | at |

49: AI096936 SNX13

| | |
|---|---|
| 1 | aaaaaaatta aggctaacca agtgcatcca ttgttcaatg gcacaattga tttcagcaac |
| 61 | tatttggaat atcctaatta taggaaatgc ccatctaagt gatatattta aataatacaa |
| 121 | tcaatttttt aaggtgaata aactatgatg gtttctaaat agtgtacatg ttacctgaaa |
| 181 | aatcagaaaa cacaaagaat gattaatttc gaaagttctt gcctaaaggc accactgact |
| 241 | taaaaaacat tcaaaatcaa ataccacaag acataaagcc tcttcatgta tatattcata |
| 301 | tatgcaataa atgcattaaa tgtaacttta ttaaacatag tacactgtac ttgacttatg |
| 361 | gttaaatatt ttacacacag cttga |

50: NM_014785 KIAA0258

| | |
|---|---|
| 1 | gccaggtccc tgaggggcgg gcagatgagg cctaggggtg ccgatcccta gtgtcgacta |
| 61 | tgcgagatct gattccggag ctgccatgat tgaagtggta gcagagctca gccggggtcc |
| 121 | tgtatttttg gctggggagg cgctggagtg tgtagtgacc gtcaccaacc cccttccgcc |
| 181 | cacggccact tctgcatcca gtgaggccct ggcctgggcc agtgcccaaa tccactgcca |
| 241 | gttccatgcc agtgagagtc gagtagcact gcctcctcct gactctagtc agccagatgt |
| 301 | ccagcccgac agccagactg tctttctgcc acaccgaggt gagaggggcc agtgtatcct |
| 361 | ttctactcca ccgaaaattc tattctgtga cctgaggctt gatcctggag agtccaaatc |
| 421 | atactcctac agtgaagtgc tgcccataga gggaccaccc tcctttcggg gtcagtcagt |
| 481 | caagtacgtc tacaaactga ccattggctg ccagcgtgtc aactccccta tcactttact |
| 541 | cagagtccct ctgagggttc ttgtgctgac tggccttcag gatgtccggt ttccccagga |
| 601 | tgaggctgta gccccatcca gtccattctt ggaggaggat gaaggtggga agaaagattc |
| 661 | atggctagct gagctggctg gggaacgcct aatggctgcc acatcctgcc gcagcctcca |
| 721 | tctatacaat atcagtgatg gccgagggaa agttgggacg tttggcatct tcaaatctgt |
| 781 | gtacagactt ggcgaggacg tggtggggac cttaaactta ggggaaggaa ccgtagcttg |
| 841 | tttgcagttt tcagtcagct tacagaccga ggagcgtgta cagcctgagt accagcggcg |
| 901 | acgtggggca gggggtgtcc cctctgtgtc acatgtgact cacgcccggc accaggaatc |
| 961 | ctgcctacat acaactagaa ccagcttctc cctcccaatc cctctcagct ccaccccagg |
| 1021 | cttctgtaca gccattgtgt ccttgaagtg gagattgcat tttgaatttg taacgtcccg |
| 1081 | agaaccagga ttggtactcc taccccctgt ggaacagccc gaacctacca cctggacagg |
| 1141 | acctgagcaa gtacctgtag acaccttcag ctgggacctg cccatcaagg tgctgcctac |
| 1201 | tagccccacc ctggcctcat atgctgcccc aggccccagc accagcacca taaccatctg |
| 1261 | aaactggccc accctggtgc tagttccttc cggatactga gaactcagca cctggactct |
| 1321 | aatgggaccc actttttcca cctggggtcc aatgtcgtgg acagtgagag tcgggctttc |
| 1381 | agctatagca ttaatttatt tgttcagaat acattggcag ctgctagtgg tttccctgga |
| 1441 | agtggcagca gcagtgagca gtcagcagat ggatgatcag ttgagtttag ctggagtggg |
| 1501 | gagcaggagc cccaggaaca ggggtgttgg ctgagcccca ttctgggtca ggccctcccc |
| 1561 | ctttgcaggg cagccgaggg tcagattttt gcaccaagga gaactggcag gttcctgcct |
| 1621 | cctgacgtac ctcacaccca gccgggaagt cgatgggatg ctgggacctg gggaaccaag |
| 1681 | gataggggaa ggagtcagca cagtgaaagg ctgcctttat ccctgcccac atgttccctc |
| 1741 | tctcacagtt ttccccccac agagcccctt tcagtggccc cttggtcctc ctaactaagc |
| 1801 | tgtcacctac catatgtggg cctttttgtt ttataacagg agtattttct ctccaggtcc |
| 1861 | accccaacct cccctgattt atagcctgaa gccttatctt tcacactagt gttggtccct |
| 1921 | tcaggtttgg cccatcttgt attgctcttc tgttcattct tacatcacag caatctagtc |
| 1981 | actccctggt catccctcag tcactcatat cagagtcatt ctctctggcc atctttggtc |
| 2041 | actcacgtgt cacagcagcc cacgccaaca ggatgcagac aggtgcaatg gaaacagtcc |
| 2101 | ttgcggagcc aagactcacc cagggtaaaa tatttcccct catagtgaca gggggctagg |
| 2161 | gaagaacggg aaatgttagt aggtgtagga gtgctgatga gaggcagagg ctcttctggt |
| 2221 | ctggggtgga gacagtaagt acgcactatc cccgtattta gtttgtcttt cctgtttcac |
| 2281 | agctggagga agcctgggta ttttgacacg ggatcatctg taaggcccca tcctccctgt |
| 2341 | gccctctctg ctgctcctcc attcctaacg cttcacccca ctttaccttg agcttggaag |
| 2401 | tagcacttgc tgtagactcc tgggtgctgg aggagtagag acatcaccaa gcagatgatc |
| 2461 | ccccagcctc ctaggatccc cttggcctgt ccagcccaga gcatccttag ggccattgct |
| 2521 | gctgcacagc cctctcagac ccttcttggc ctctgctcag ctactctggt cttgactcct |
| 2581 | tgactttgct ttgcgttgct ccttgagtct tagtttctgt ctttctcccc tgggctcctg |
| 2641 | tctcacacta tctccctgcc ctctgctctc acaggctggg gatgtttata aagtgaggac |
| 2701 | cctggccccc tgctgagtag agctggaaaa gttgtaactc tgtttcctga ggtgagggca |
| 2761 | tgaaaacaag aggtctagct ttaacaagct gtgagagctg attcatgccc cggcacagct |
| 2821 | agagggaggg aggtggccat ggagggggca ctggactggg cacttcccca gcaaggaggc |
| 2881 | aggaggggcg agggccccca ggtggtcccc agatctcttc cctgacctgg agagaaggaa |
| 2941 | gcattccacc ttcccccttt ctcccccact gccaccacca ggggtgtgta tgctgggatc |
| 3001 | cctgcctgga ccggagggag gcatttcctg gggatggtta atcctgtgcc ccagccaaac |
| 3061 | ccaggagctg caatagggtg cgacggccag aagctccagg agagtgagca ggcacctgga |
| 3121 | gtggagactg tgtttccctc agatcctagg gcagggtttc cctaatgtat ccaagaaata |
| 3181 | gggctgcccc tcagagatgg tggggagggt ctcttttcct caggcattcc agaggtgaac |
| 3241 | tgtccattgc ttatcacctt caaacataca gcagatgtgg gatcacccca catctgggga |
| 3301 | tggttctttc ccctttcaaa gaggagcatc tctaagtgcc ctgatgggat gaatcactcc |
| 3361 | aggttcacag aggtgtcctc tctttcctcc catatataat ggagtgaggt ttttaggaat |
| 3421 | ttatcatttg gcatcctctg agtttcccac aggttctgga ggagcccagg atggattatt |
| 3481 | gagagcatgg gctgtagaga cagtcttctt ggattcagat cctgactcca cttagctatg |
| 3541 | taacctggtc agattacttc acctctctga gcctgtttcc tcatctataa attggggata |
| 3601 | gtaatgccaa ctcattgggc tgttatgagg attactgaga taatgcgtgc agtgctctta |
| 3661 | tcaccatctc tggtgcgtaa gcgtcaggaa atagcagttg ctgtgattgg ggctaaagct |
| 3721 | ctgaggcaaa atgggcgaca ttattttctt tgaatgacat taagcagttt gtgcatagct |
| 3781 | gagggcttct attggggatg gctgtctcct ggcatagacc tctgcacctt tcacactcat |
| 3841 | actccttgtc agcagtcccc aacctttttg gtaccaggga ccggttttgt ggaaaacaat |
| 3901 | ttttccacca gtggatggag ggggatagca gcggggagat gattttggga tgaaactgtt |
| 3961 | tcatctcaga tcatcaggca ttagattctc ataaggagtg tgcaatctag atcccttgca |
| 4021 | tgcggagttc acagtggggt ttgcactcct gtgagaatct aatgcctctg ctgatctgcc |
| 4081 | aggaggagga gctcaggcgg taatgctcac tcgcctgccg cccacctcct gctttgtgct |
| 4141 | cccgcttcct aacaggccac agactggtac tggcctgtgg cctgggggat ggagacccct |
| 4201 | aatccatgtc acctttccca cctctttcaa aaacaggtac ctccaggaac attttggttt |
| 4261 | tggcccttgt attgacttct gaatgtctag tttgagaaac tgttcccaaa taagccttct |
| 4321 | tcccccagat ctgcaccctc gcctctaccc taggacaaga tgtccttttc tcatcatcct |
| 4381 | gccaggctaa ctttaagtct cctgcttttt ctcacttgga tttggatcca tttcttccta |
| 4441 | tttccgctca tgtgaactct ccagttctcc tttctcacca ctctcctgct agccatctct |
| 4501 | ttggcactaa aggccctggt caaattggat ttctttcatt tttccacact tcaaagaccc |
| 4561 | atgttctagg tattctccat agggatagtc tctttggcat ttatttggtt tttctacgtt |
| 4621 | ttcagtccca tttactccaa gactcactcc ctgccaccta gtgcatcaga tacagctact |
| 4681 | tctggctgac ttttcaaggg ggaccaccct acctgtcatc tcttcactgt tcagaaatga |
| 4741 | ctgtgtcagt gcacctcaaa ctcccttgct gtccttttcc aaggagacag ctaaggtgga |
| 4801 | tggagatgca gaatggacct cacgttcgcc ctagtcagga ctgataccct ttccgtttca |
| 4861 | gaggattgcc aagaaaaaac tcacagttga ggcagggtgc tctgaggtcg gctgcggtgt |
| 4921 | gggaggcacg gcctgggcct gctctctggg ctggagcagg tggattcgaa ggcctgtcta |
| 4981 | gcacgagggc ccaaaggtct tgtcagtggc cagtagctct gccgcctttc ccagagaggg |
| 5041 | ggtccagggg acatcctgga aggctgggcc ctgggccacc ttctgctctt gcaagctaga |
| 5101 | gccagcccaa tagggggcgg atgtgagtgg ggagctgggg cgcatgaagg tgggggtgat |
| 5161 | gccgaagggg aagggatcgc cagtggggat tggtgcgtgt gcggaaacgg ggacagaagt |
| 5221 | gaaggttcat cgcctataac gaagatgagg taggcatata ggggcttctg gaaagctaga |
| 5281 | ggctgggctg agccaggagt cctctcccag aagttggggg gcggtgcaga ggtgtgggtc |
| 5341 | gagcccgcat gcgtgcctgc tggggagggg gtgagtggtg aggaccaggc ccgctgggtc |
| 5401 | ctgggggcgc ggtggctggc gcgcaggtcc cggagggggc ggctggcgcg cactacacgc |
| 5461 | ttgggaacaa ggaaaacatc cgccggaggc ccggccgggc ggcgctccag cctcggggca |
| 5521 | ggtgcgcgga gaggaagtga gagcattccg gcccccccac cccaaccccg gccgctggcc |
| 5581 | ctctggtgag tcacagccga cccccgccgc cggagggaga ggggagctgc gggccagagc |
| 5641 | cccggagggt ctggaggagc caggagggtt tctgggagca gagggtcact tagtgggctt |
| 5701 | ctgtcgtggt gtcgctacgg gcgcgaaacg gacactgaac acagtctgac tgtatggagg |
| 5761 | caggtgggga gggatcccct gggagaactt ggcgggccga gagcagaccc cagggcaagg |
| 5821 | aggggccccc gagggggaaa ccgggagtcg ggcaggtggc gtaacccaga aagggaagga |
| 5881 | gagccggatt gattggggtg agagaggaag gaagcacgcc aagttaggcc tgggagaact |
| 5941 | gagggacctg aggagggagg agggagacca acacagggtg ggaaggcgga aatggccaaa |
| 6001 | ccccaggcat caggtctgtc cagaggctga cgtagacagt gaagggtgaa gggtaggttt |
| 6061 | taggagtagg gggagttatg attatttggt tacattttgg gattatttgg tctcacaggt |
| 6121 | agaagggagc ctgctggtct ctgtgtaacg gatggcttaa aagcaaggtt gtctgcgtct |
| 6181 | tggattactg tctgccattc agcctttgcc aaaaaatttg gcactgatct gcacattttt |
| 6241 | atagtcattt aaaattgtat gactctgtca aatgatttaa gtaattttgg tggattttta |
| 6301 | aaaataaaaa aat |

51: BF973104 TOM7

| | |
|---|---|
| 1 | ggtaaggggt cctccctgcg ccacacggcc gtcgccatgg tgaagctgag caaagaggcc |
| 61 | aagcagagac tacagcagct cttcaagggg agccagtttg ccattcgctg gggctttatc |
| 121 | cctcttgtga tttacctggg atttaagagg ggtgcagatc ccggaatgcc tgaaccaact |
| 181 | gttttgagcc tactttgggg ataaaggatt atttggtctt ctggatttgg aggcaatcag |
| 241 | cggacagcat ggaagatgtg tgctctggct cggataagag atgggacatc attcagtcac |
| 301 | tagttggatg gcacaaggct cttcacagac gcatctgtag cagagtggat cttgtactaa |
| 361 | cttatgatag aatgtatcag aataaatgtt tttaacagtg taaacaccac aaacaaaaaa |
| 421 | cacaacacac acatcataca cacaaaaaac acaaaaaaaa caaacaaatc acacaaaagc |
| 481 | tacggtagac ctactattat gcggtgggcc gaaacaagac gggtattata gacaagggaa |
| 541 | acgagtcgtc aaatcgtcgt agcctgacac acatcatatt gttagaccca gcgtgtgcaa |
| 601 | tatctcgccg gggtagctcc ctcatatgag ggacacgtta tatatgtctc agatagggcg |
| 661 | ccggggtata acctgcagtt ttatagatat gctggcaaca gaaaaaagcg atgtaaaaaa |
| 721 | aaaaatgaag acaacataaa cacacacaca aagatactat cacatatata ctataaccaa |
| 781 | aaaatctcaa agcgtaaatc aaaaatacac taaaacaatt cacagccata ttcactacac |
| 841 | cctatccacc ccacacaaaa aaaataagac acaaaacatc acacatatac acactaccta |
| 901 | tcattttata ctttaatcta atataattaa gtaacaaatc aacacaaata tacacacgat |
| 961 | cgatagatac actgataaaa ttcaacaaac aaaataccaa ataaaatata ctaaacacac |
| 1021 | cactagacga gcatcttata ttgcactttt acgtagacct ctgatcaata acaacagacc |
| 1081 | tactccacaa atatactact aacacacaaa caatgcaaac agcacagaat aac |

| | | | | |
|---|---|---|---|---|
| Rank | GenBank | Gene | | |
| Order | ID | Symbol | Gene Name | Nucleotides |

| | | | | |
|---|---|---|---|---|
| 5 | NM_004090 | DUSP3 | dual specificity phos- | GGATCCTTTATTGGTGGTAGAG |
| | | | phatase 3 (vaccinia | CAAAAAAACCCAAACACGATAA |
| | | | virus phosphatase VH1- | ACCTTTCAAAAGACTTTCTAAG |
| | | | related) | GATGATATTGGAATGCACCAGC |
| | | | | CCTCACATGTGTATGCACATTT |
| | | | | GCCAGAATATAAGAGTTTTGTT |
| | | | | TTAAATACAGTCTTGTTAGGAT |
| | | | | TTTACGTTATTGTTATTATGGA |
| | | | | AAGTGATTGTGATGCTATTTAT |
| | | | | CTTCAGGGTCACTCTGG |
| 6 | AI026836 | DJ473B4 | hypothetical protein | GCAGTCGTTTCAACCAGGTAGT |
| | | | dJ473B4 | TTTGGGTTGTTTTTAAAGCCCT |
| | | | | TTTGAGGTCTTACACATTATTA |
| | | | | ACTTTAAAATAATCAGGCAGCT |
| | | | | AAGAATAATTACTAGAAAAATC |
| | | | | ATCTACCACTTCAAACATGGTC |
| | | | | AACTACTTCAAAACTGCACCTA |
| | | | | GAGAATCAGGTACCTGAAGTAG |
| | | | | AACAAGAAGCCTGGAGGTGGAC |
| | | | | TTTGAGAGGAGGGAATACCC |
| 7 | BU500509 | PHLDA2 | pleckstrin homology-like | TACGTGTACTTCACCATCGTCA |
| | | | domain, family A, member | CCACCGACCACAAGGAGATCGA |
| | | | 2 | CTTCCGCTGCGCGGGCGAGAGC |
| | | | TGCTGGAACGCGGCCATCGCGC | |
| | | | | TGGCGCTCATCGATTTCCAGAA |
| | | | | CCGCCGCGCCCTGCAGGACTTT |
| | | | | CGCAGCCGCCAGGAACGCACCG |
| | | | | CACCCGCCGCACCCGCCGAGGA |
| | | | | CGCCGTGGCTGCCGCGGCCGCC |
| | | | | GCACCCTCCGAGCCCTCGGAGC |
| | | | | CCTCCAGGCCATCCCCGCAGCC |
| | | | | CAAACCCCGCACGCCATGAGCC |
| | | | | CGCCGCGGGCCATACGCTGGAC |
| | | | | GAGTCGGACCGAGGCTAGGACG |
| | | | | TGGCCGGCGCTCTCCAGCCCTG |
| | | | | CAGCAGAAGAACTTCCCGTGCG |
| | | | | CGCGGATCCTCGCTCCGTTGCA |
| | | | | CGGGCGCCTTAAGTTATTGGAC |
| | | | | TATCTAATATCTATGTATTTAT |
| | | | | TTCGCTGGTTCTTTGTAGTCAC |
| | | | | ATATTTTATAGTCTTAATATCT |
| | | | | TGTTTTTGCATCACTGTGCCCA |
| | | | | TTGCAAATAAATCACTTGGCCA |
| | | | | GTTTGCTTTTCTACCATCC |
| 8 | NM_016090 | RBM7 | RNA binding motif | CTGTGACATGCTCTTGAGCTTT |
| | | | protein 7 | ACCCTAGTTGAACATACATGTG |
| | | | | TAGATTTACACATACTGTTTCA |
| | | | | TTNNNNAATTTAGAAATTGTTC |
| | | | | ATTAAATCCCATTTGAGGTATA |
| | | | | AGTCACTCAGGAAGTTAAAATA |
| | | | | TCTCTACACGTATATTTTTACA |
| | | | | TTAAAAATACAGTGTTAGCATA |
| | | | | ANNNNCCCTTTNNNNNGAAGAA |
| | | | | CAAAAATGTCAGTGCATAGTTA |
| | | | | GATAAAATGGTAAAATGTTTTA |
| | | | | CTGAAAGCATACTTTTTTGGAA |
| | | | | AATAGATTCATGAAGCCTTTAA |
| | | | | GTGCTGCTTCTGTCAGTCAAAC |
| | | | | GTTAAAAACTTTAACATTTTCA |
| | | | | AAGTGCCCAGAGTGTGTACAAA |
| | | | | GACACATGTAATGGAGATTGTA |
| | | | | CAGGTTGTTTTTTTGTTTGAAC |
| | | | | CTTTGAAAGAGTTTAATCTTAA |
| | | | | CGTTTTCTAATTTTAAAATTTT |
| | | | | AAAATCTTGTTTAACAAAAGCT |
| | | | | TGTATTAAGATACTGTTTTCAT |
| | | | | TTCATTACAGAATTGTTTATAA |
| | | | | AAGTTCATTTGTTGAAAANNNA |
| | | | | GGATCCTTTTTAATACCACAGC |
| | | | | ATTTGTACTGTTCCT |
| 9 | BX092512 | EST | | ATATGTGCACACACACACTCAC |
| | | | | ACCCACACCCATAAAGATTTTG |
| | | | | CACTCCTTGAAGGTACACTAAC |
| | | | | TCACCATTTTTATCATACTTAT |
| | | | | CCCAGTGTGCCACAGTTACTGG |
| | | | | CTTATATGCCTGTCTCTGCTAT |
| | | | | CTTATTTTATCTGTCTCCACAA |
| | | | | CACAGCAAACTACCTGGCCTTC |
| | | | | AATAAAGGGCTTATGAATTATT |
| | | | | CATGAATCCATTTTGCCAGGTG |
| | | | | CCTAGCCCTGTGTCTGGCTTGA |
| | | | | AGCAGGTGTTCCCAAGGTGTGG |
| | | | | CATGGCTGAGTGAATACAAAT |
| 10 | AI436027 | OSMR | oncostatin M receptor | CACCAATGAGCTTACTACCCAA |
| | | | | CTTCAAAACTAGGACTCTAACA |
| | | | | ATAACTTCTGTCATATCTCATC |
| | | | | CTGTAACGCCCCCACCTTCGCT |
| | | | | CCTTCCGCCAAGATAATTATCA |
| | | | | CTTTAAATTGTGTGCGTGTGTA |
| | | | | TTCTCATTTCTTATGTGATGGT |
| | | | | AAAAATGCCTTTATTTTGTTTG |
| | | | | GTTTTAATGCATAGAAAGGACA |
| | | | | TCAAGCTGT |
| 11 | AI971137 | GCLC | glutamate-cysteine li- | CTCTAAAAGCCATTCACTCCAG |
| | | | gase, catalytic subunit | ATTTTACCTGGGGAATATTCTA |
| | | | | CATACTGCTTACTTTCTCTATA |
| | | | | AAACTCATCAATAAATCATGAA |
| | | | | AGGCACTGAGTTTTGTAAATCA |
| | | | | GGACCCTAAATGTTTAATTGTA |
| | | | | AATAAGTTTCAGATAATTATTA |
| | | | | TAGCTTTGCGTTGAAGTTNNNN |
| | | | | NNNNNTTTCTCTCAACTAGTTA |
| | | | | AGTCAACTGCTTCTGAAATAAC |
| | | | | TCTGTATTGTAGATTATGCAGA |
| | | | | TCTTTACAGGCATAAATATTTA |
| | | | | AACTGTAATATGCTAACTTGAA |
| | | | | GAGATTGCAATAAAGCTGCTTC |
| | | | | AGCTAAC |
| 12 | BQ024877 | COL4A3BP | collagen, type IV, alpha | CTCACTGAAGTTGAAATGACTG |
| | | | 3 (Goodpasture antigen) | CCCACTTCAAAATCTTCATTGT |
| | | | binding protein | GTTTACACACCAGTGTATTTAT |
| | | | ACAAATCAGAGGCATTTTGTAG | |
| | | | | ATGCTTTGCTGACTTGTTCAGC |
| | | | | TCTGTAAAAACACAGAAATCAG |
| | | | | ACCCATTTTGTAAAGCGGAAAA |
| | | | | TCATGTTACATGGAACATGTCC |
| | | | | TGTATATATCACATACATGGTA |
| | | | | ATGGAGTCTTAATGATAAGTGC |
| | | | | AAGATAATAATTTAATGATGGG |
| | | | | ATTAGTCTGATCGCTTAATATG |
| | | | | CACAATCCTGGAAGTGAATTAC |
| | | | | TTGCATCAGATATAGTGATATT |
| | | | | TATTATTCTGTACAGAGAGAAA |
| | | | | AATACATATAAAACATATGCTT |
| | | | | ACATTACATGCACGCGGATTTC |
| | | | | ATGCTCCATAATCTTTTCTATT |
| | | | | TTTTAATTTACCTTTCTGTAAA |
| | | | | TGATGTGCATGGAATATGCCTT |
| | | | | ATAGAAAAATGCTGTTCATAAT |
| | | | | TTGACTACGTGGAAAAGTGCCT |
| | | | | ATATGGTGGTAATGCTAGTAAG |
| | | | | GCA |

TABLE 5A
Correlation of cDNA microarray data with semi-quantatative RT-

[symbol]

Spearman
rank correlation

| Rank Order | Gene Symbol | ρ | p-value |
|---|---|---|---|
| 1 | FLJ22662 | 0.69 | 0.02 |
| 2 | AREG | 0.53 | 0.08 |
| 3 | CORO1C | 0.35 | 0.24 |
| 4 | AVEN | 0.63 | 0.04 |
| 5 | DUSP3 | 0.63 | 0.04 |
| 6 | DJ473B4 | 0.45 | 0.14 |
| 7 | PHLDA2 | 0.84 | 0.01 |
| 8 | RBM7 | 0.83 | 0.01 |
| 9 | EST(BX092512) | 0.63 | 0.04 |
| 10 | OSMR | 0.67 | 0.03 |
| 11 | GCLC | 0.46 | 0.13 |
| 12 | COL4A3BP | 0.27 | 0.24 |

Correlations positive for all 12 genes and significantly positive for 7 of 12 ge

[symbol]

TABLE 5B
Result of immunohistochemical staining

| | PR | PD |
|---|---|---|
| AREG | 1/5 | 5/6 |
| TGFA | 2/5 | 6/6 |
| ADAM9 | 1/5 | 4/6 |
| CD9 | 2/5 | 5/6 |
| OSMR | 2/5 | 6/6 |

We claim the following:

1. A set of isolated marker genes comprising at least one gene identified as having differential expression as between patients who are responders and non responders to an erbB receptor tyrosine kinase inhibitor; said gene set comprising one or more genes selected from at least the group consisting of the 51 genes listed in Table 4 herein including gene-specific oligonucleotides derived from said genes.

2. The set according to claim 1 comprising at least one or more of the first 40 genes listed in Table 4 herein.

3. The set according to claim 1 comprising at least one or more of the first 20 genes listed in Table 4 herein.

4. The set according to claim 1 comprising at least one or more of the first 12 genes listed in Table 4 herein.

5. The set according to claim 1 comprising at least one or more of the first 5 genes listed in Table 4 herein.

6. The set according to claim 1 which is first 12 genes listed in Table 4 herein, namely the genes FLJ22622 (e.g. GenBank NM_{—}024829), AREG (e.g. GenBank BC009799), C0R01C (e.g. GenBank NM_{—}014325), AVEN (e.g. GenBank BC010488), DUSP3 (e.g. GenBank NM_{—}004090, DJ473B4 (e.g. GenBank AI026836), PHLDA2 (e.g. GenBank BU500509), RBM7 (e.g. GenBank NM_{—}0106090), EST (GenBank BX0952512), OSMR (e.g. GenBank AI436027), GCLC (e.g. GenBank AI971137), COL4A3BP (e.g. GenBank BQ024877).

7. The set according to claim 6, wherein the genes comprise the sequences set forth in Table 4a.

8. The set according to claim 6, wherein the set comprises gene-specific oligonucleotides, said oligonucleotides comprising 5 to 50 nucleotides of the sequences set forth in Table 4a.

9. The set according to claim 1 wherein the inhibitor is selected from the group consisting of gefitinib, OSI-774, PKI-166, EKB-569, GW2016 and CT-1033.

10. The set according to claim 9 wherein the agent is gefitinib.

11. The set according to claim 1 wherein the inhibitor is an anti-erbB antibody.

12. The set according to claim 11 wherein the antibody is trastuzumab or cetuximab.

13. A method of predicting the responsiveness of a patient or patient population with cancer to treatment with an erbb receptor kinase inhibitor, or for selecting patients or patient populations that will respond to an erbB receptor kinase inhibitor comprising comparing the differential expression of one or more marker genes, said marker genes selected from the gene sets as defined in claim 1.

14. The method according to claim 13 wherein the responsiveness of the patients is represented by the generation of a Drug Response Score.

15. The method according to claim 13 wherein the comparison is performed by microarray assay.

16. The method according to claim 13, wherein the comparison is performed by immunohistochemistry.

17. The method according to claim 16, said method comprising detecting the differential expression of amphiregulin.

18. The method according to claim 13 wherein the inhibitor is selected from the group consisting of gefitinib, OSI-774, PKI-166, EKB-569, GW2016 and CI-1033.

19. A diagnostic kit for use in the method of claim 13 comprising a marker gene set selected from the group as defined in claim 1 on a suitable support medium.

20. The kit according to claim 19 which comprises a microarray.

21. A method of treating a patient with cancer comprising administering an inhibitor selected from the group consisting of gefitinib, OSI-774, PKI-166, EKB-569, GW2016 and CI-1033, and testing the differential expression of a set of marker genes, said set selected from the group as defined in claim 1.

22. A method of measuring expression level of genes selected from the group consisting of the 51 genes listed in Table 4 in a tissue sample from a patient having Non-small Cell Lung Cancer (NSCLC), comprising contacting an isolated gene sequence selected from the group consisting of the 51 genes listed in Table 4, including gene-specific oligonucleotides derived from said genes, with said sample.

23. A diagnostics kit comprising means for determining the level of expression, of one or more genes selected from selected from the group consisting of the 51 genes listed in Table 4 herein including gene-specific oligonucleotides derived from said genes in a tissue sample from a NSCLC patient, comprising a support material comprising a set of isolated marker genes, as defined in claim 1, at least one gene thereof attached thereto.

24. A method of treating NSCLC patients identified according to the method of claim 13, comprising administering an inhibitor selected from the group consisting of gefitinib, OSI-774, PKI-166, EKB-569, GW2016 and CI-1033.

25. A method of treating patents or patient populations having NSCLC identified according to the method of claims 13 comprising administering to said patients an erbB receptor tyrosine kinase inhibitor.

26. A pharmaceutical composition for the treatment of a patient having NSCLC, comprising an erbB receptor tyrosine kinase inhibitor.

27. A method of testing, or testing for, an erbb tyrosine kinase receptor inhibitor comprising treating the patient and assessing if the compound modulates gene expression of at least one of the gene from the marker gene set according to claim 1 relative to a relevant control.

28. A method of carrying out a clinical trial to measure the effect or effectiveness of erbb receptor tyrosine kinase inhibition or inhibitors comprising measuring the relative levels of expression of a gene set as defined in claim 1 in a patient or patient population.


SEQUENCE LISTING (83 sequences)

SEQ ID NO: 1 - DNA, 352 bp, Homo sapiens
atacggcatccatgaaatatatcatgcgatacaacaattataagaaggatccttacagtagaggtgacccctgtaataccatctgctgccgtgaggacctgaactcacctaacccaagtcctggaggttgttatgacacaaaggtggcagatatctacctagcatctcagtacacatcctatgccataagtggtcccacagtacaaggtggcctccctgtttttcgctgggaccgtttcaacaaaactctacatcagggcatgccagaggtctacaactttgattttattaccatgaaaccaattttgaaacttgatataaaatgaaggagggagatgacggactagaagac

SEQ ID NO: 2 - DNA, 1039 bp, Homo sapiens
ctccactcgctcttccaacacccgctcgttttgcggcagctcgtgtcccagagaccgagttgccccagagaccgagacgccgccgctgcgaaggaccaatgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtatataaaggtgcacgaaggtaaaaag

SEQ ID NO: 3 - DNA, 386 bp, Homo sapiens
gataggccacattccagtaagaactcaatttgtctcccaaatttgcagaaacaaaacgtgatttaaaagctgagctttttatcagaaagcttttttgatgttttaagtgttatgtgacttgttgaactttttaaaaagtgctacttttaaaatcccagatactctgaattttagaaaacaaactaattctgattgtgtcgtgcccaagtacccttttttttttaatgagtagggaccaatgccacattgctttttatatttctttcttttttaatgttgccaaaaccaaaagtagctttgttttcctttgtattttgctactttgcagtatttgtgtgtgtggttttttttccttaatttgaaagggacagcactgtgtatgttta

SEQ ID NO: 4 - DNA, 565 bp, Homo sapiens
aggagaccatttggaagaagaactagatctgttgcttaatttagatgcacctataaaagagggagataacatcttaccagatcagacgtctcaggacctgaaatccaaggaagatggggaggtggtccaagaggaagaagtttgtgcaaaaccatctgtgactgaagaaaaaaacatggaacctgagcaaccaagtacctccaaaaatgttaccgaggaagagctggaagactggttggacagcatgatttcctaaaaaggggaaaaaaagtgcctgaagcaaatcttggttgccttctaacggcaggtgggcataaggctgtccttcaggaccagccagtttacaagcatgtctcaagctagtgtgttccattatgctcacagcagtaaatgcctacctctgtgtttgacatctgaaagaatacattgaagcagcttgttgcatttgtttttctggcttagtaatctaatagatttccttaagggcaggagatagactctggcccttgtttctagcctccttccttgcagtgtttacaacatagccagtgtttacagcatagca

SEQ ID NO: 5 - DNA, 215 bp, Homo sapiens
ggatcctttattggtggtagagcaaaaaaacccaaacacgataaacctttcaaaagactttctaaggatgatattggaatgcaccagccctcacatgtgtatgcacatttgccagaatataagagttttgttttaaatacagtcttgttaggattttacgttattgttattatggaaagtgattgtgatgctatttatcttcagggtcactctgg

SEQ ID NO: 6 - DNA, 218 bp, Homo sapiens
gcagtcgtttcaaccaggtagttttgggttgtttttaaagcccttttgaggtcttacacattattaactttaaaataatcaggcagctaagaataattactagaaaaatcatctaccacttcaaacatggtcaactacttcaaaactgcacctagagaatcaggtacctgaagtagaacaagaagcctggaggtggactttgagaggagggaataccc

SEQ ID NO: 7 - DNA, 525 bp, Homo sapiens
tacgtgtacttcaccatcgtcaccaccgaccacaaggagatcgacttccgctgcgcgggcgagagctgctggaacgcggccatcgcgctggcgctcatcgatttccagaaccgccgcgccctgcaggactttcgcagccgccaggaacgcaccgcacccgccgcacccgccgaggacgccgtggctgccgcggccgccgcaccctccgagccctcggagccctccaggccatccccgcagcccaaaccccgcacgccatgagcccgccgcgggccatacgctggacgagtcggaccgaggctaggacgtggccggcgctctccagccctgcagcagaagaacttcccgtgcgcgcggatcctcgctccgttgcacgggcgccttaagttattggactatctaatatctatgtatttatttcgctggttctttgtagtcacatattttatagtcttaatatcttgtttttgcatcactgtgcccattgcaaataaatcacttggccagtttgcttttctaccatcc

SEQ ID NO: 8 - DNA, 565 bp, Homo sapiens
  misc_feature (69)..(72): n is a, c, g, or t
ctgtgacatgctcttgagctttaccctagttgaacatacatgtgtagatttacacatactgtttcattnnnnaatttagaaattgttcattaaatcccatttgaggtataagtcactcaggaagttaaaatatctctacacgtatatttttacattaaaaatacagtgttagcataannnnccctttnnnnngaagaacaaaaatgtcagtgcatagttagataaaatggtaaaatgttttactgaaagcatacttttttggaaaatagattcatgaagcctttaagtgctgcttctgtcagtcaaacgttaaaaactttaacattttcaaagtgcccagactgtgtacaaagacacatgtaatggagattgtacaggttgtttttttgtttgaacctttgaaagagtttaatcttaacgttttctaattttaaaattttaaaatcttgtttaacaaaagcttgtattaagatactgttttcatttcattacagaattgtttataaaagttcatttgttgaaaannnaggatcctttttaataccacagcatttgtactgttcct

SEQ ID NO: 9 - DNA, 285 bp, Homo sapiens
atatgtgcacacacacactcacacccacacccataaagattttgcactccttgaaggtacactaactcaccatttttatcatacttatcccagtgtgccacagttactggcttatatgcctgtctctgctatcttattttatctgtctccacaacacagcaaactacctggccttcaataaagggcttatgaattattcatgaatccattttgccaggtgcctagccctgtgtctggcttgaagcaggtgttcccaaggtgtggcatggctgagtgaatacaaat

SEQ ID NO: 10 - DNA, 207 bp, Homo sapiens
caccaatgagcttactacccaacttcaaaactaggactctaacaataacttctgtcatatctcatcctgtaacgcccccaccttcgctccttccgccaagataattatcactttaaattgtgtgcgtgtgtattctcatttcttatgtgatggtaaaaatgcctttattttgtttggttttaatgcatagaaaggacatcaagctgt

SEQ ID NO: 11 - DNA, 315 bp, Homo sapiens
  misc_feature (173)..(181): n is a, c, g, or t
ctctaaaagccattcactccagattttacctggggaatattctacatactgcttactttctctataaaactcatcaataaatcatgaaaggcactgagttttgtaaatcaggaccctaaatgtttaattgtaaataagtttcagataattattatagctttgcgttgaagttnnnnnnnnntttttctcaactagttaagtcaactgcttctgaaataactctgtattgtagattatgcagatctttacaggcataaatatttaaactgtaatatgctaacttgaagagattgcaataaagctgcttcagctaac

SEQ ID NO: 12 - DNA, 509 bp, Homo sapiens
ctcactgaagttgaaatgactgcccacttcaaaatcttcattgtgtttacacaccagtgtatttatacaaatcagaggcattttgtagatgctttgctgacttgttcagctctgtaaaaacacagaaatcagacccattttgtaaagcggaaaatcatgttacatggaacatgtcctgtatatatcacatacatggtaatggagtcttaatgataagtgcaagataataatttaatgatgggattagtctgatcgcttaatatgcacaatcctggaagtgaattacttgcatcagatatagtgatatttattattctgtacagagagaaaaatacatataaaacatatgcttacattacatgcacgcggatttcatgctccataatcttttctattttttaatttacctttctgtaaatgatgtgcatggaatatgccttatagaaaaatgctgttcataatttgactacgtggaaaagtgcctatatggtggtaatgctagtaaggca

SEQ ID NO: 13 - DNA, 1654 bp, Homo sapiens
tggagcccgaggtccccgcgcggcccgggcctggcgccctgaggggaagagcggcccggcccgagccatgacggacgggatcctagggaaggcagccacaatggagatccctatccacgggaacggcgaagccaggcagcttcctgaagatgatgggctggagcaggacctccagcaggtgatggtgtcaggacccaacctcaatgaaaccagcattgtgtctggtggctatgggggctctggtgatggactcatccccacagggtctggccgccatccatctcacagcaccactccttctggccctggagatgaggtggctcggggcattgctggagaaaagtttgacatcgtcaagaaatggggcatcaacacctataagtgcacaaagcaactgttatcagaacgatttggtcgaggctcacggactgtggacctggagctagagctgcagattgagttgctgcgtgagacgaagcgcaagtatgagagtgtcctgcagctgggccgggcactgacagcccacctctacagcctgctgcagacccagcatgcactgggtgatgcctttgctgacctcagccagaagtccccagagcttcaggaggaatttggctacaatgcagagacacagaaactactatgcaagaatggggaaacgctgctaggagccgtgaacttctttgtctctagcatcaacacattggtcaccaagaccatggaagacacgctcatgactgtgaaacagtatgaggctgccaggctggaatatgatgcctaccgaacagacttagaggagctgagtctaggcccccgggatgcagggacacgtggtcgacttgagagtgcccaggccactttccaggcccatcgggacaagtatgagaagctgcggggagatgtggccatcaagctcaagttcctggaagaaaacaagatcaaggtgatgcacaagcagctgctgctcttccacaatgctgtgtccgcctactttgctgggaaccagaaacagctggagcagaccctgcagcagttcaacatcaagctgcggcctccaggagctgagaaaccctcctggctagaggagcagtgagctgctcccagcccaacttggctatcaagaaagacattgggaagggcagccccagggtgtgggagattggacatggtacatcctttgtcacttgccctctggcttgggctcctttttctggctggggcctgacaccagttttgcccacattgctatggtgggaagagtgcctggaggcccagaagttgctgccctgtctatcttcctggccacagggcttcattcccagatcttttccttccacttcacagccaacggctatgacaaaaccactccctggccaatggcatcactcttcaggctggggtgtgctccctgaccaatgacagagcctgaaaatgccctgtcagccaatggcagctcttctcggactcccctgggccaatgatgttgcgtctaataccctttgtctctcctctatgcgtgcccattgcagagaaggggactgggaccaaaggggtggggataatggggagccccattgctggccttgcatctgaataggcctaccctcacccacccacccagtttaattgtgcttagagcccaagaagattggga

SEQ ID NO: 14 - DNA, 652 bp, Homo sapiens
  misc_feature (188)..(188): n is a, c, g, or t
ttttttttttttttttttaaagtagtttaataaactccacaaaataatagcagatgcattgaaatatttacataattcgattttcaaatctctcattcaaataaaagggataaaaataaaatttctgcctttacggcagcagaacctctttcctgaaatggattggtaaaataagatacttcactggnagaggaactaatttatgtttaagaggtattcatattcagctaagaaaatacaaccctttttcagctatatagattagggaatataaaatgatattttctacattttttgacctgtattcaaagttctaaattcaactttgacttgaagagagaaggtgattttggtacccatacagagtagatcatcacaattacaatggaaagataattaacgttttatatgctgtttatttgcttttgaaagtttgggtcagaaaggctgtgataataattctggcccaaacaggtatgcttatacctgacacaaatttcactaaaacctaacacttttggcttggagttcttgggatttcgactttctgagtcccttccatttccaaagcatgtttcattgagagcaggcaatgtttggggatcaggtgtatgattcaagactaattaagatgccaaagttttccaagctc

SEQ ID NO: 15 - DNA, 1990 bp, Homo sapiens
attgtggcggtgaggaacaggaagccctgaagggtcaaaagaaatacaaaagcaaaggctattttctttttttttttctttctttcattccttccttcctctgtttctttctttcttcctttcatttttttttcttttttaagagcgagcggctctgcggtggcggtttggggtgggcgccgccgaggtgaggtcgtctcgcctcccgcgcgccggtagattggttgtttcattatggatggaggggatgatggtaaccttattatcaaaaagaggtttgtgtctgaggcagaactagatgaacggcgcaaaaggaggcaagaagaatgggagaaagttcgaaaacctgaagatccagaagaatgtccagaggaggtttatgaccctcgatctctatatgaaaggctacaggaacagaaggacaggaagcagcaggagtacgaggaacagttcaaattcaaaaacatggtaagaggcttagatgaagatgagaccaacttccttgatgaggtttctcgacagcaggaactaatagaaaagcaacgaagagaagaagaactgaaagaactgaaggaatacagaaataacctcaagaaggttggaatttctcaagagaacaagaaggaagtggaaaagaaactgactgtgaagcctatagaaaccaagaacaagttctcccaggcgaagctgttggcaggagctgtgaagcataagagctcagagagtggcaacagtgtgaaaagactgaaaccggaccctgagccagatgacaagaatcaagagccctcatcctgcaagtctctcggaaacacctccctgagtggcccctccatccactgcccctctgctgcagtatgtatcggcatcctcccaggcctgggtgcctactctgggagcagcgactccgagtccagctcagacagcgaaggcaccatcaatgccaccggaaagattgtctcctccatcttccgaaccaacaccttcctcgaggccccctagtttctccgtccctacacagggagctcctccccaagggtagatcggaccgttcatgctgcctataggcattatgtccctcaaaaaaaaactcctttgcctgcatcctgtgtacaacatgacatttttaaccaatccaatctaaaaatgtgccagaatccacctgtggcccgaatcgtgtttggttcctctttctactccactgcagatgaccaaacctgtcccgctgccactttcctcactgatattgggaggagggcaaggcccagccgaagttccactaaaaatgccccaggagaataggcaccggctggcttgccaaagggtttgggttttattgctttctgttttttcttttcccgacagcacaaagaagtaagggcagttattggacaggtgttatttaaacattctattgtaaatgaatgtgttgtttggttctactgcattgtggagcatgcgggggaagagaactgacccaggtaatgaaatggagcccttccctggaactaaccagtccttgatgttgtgtgactaagtaaagatgataaaccccatctgctgggggtgtcacttcacactcggcatgcattgtgaaagctttccatacccttggccattccctctctcctctctctccaaccccatttatgcaggaggggactgctaacaagaacgcttccatctcaaaccttttctctgcctgggaaattattttatgtttgtttttgaaataaaggatttagtttaagattctaaattttagagaaacaaacgtaggccttgtttactaatagccagacatcagaactgcaggtaggtatgttaatgagatgacttatttctggcagctcctggaatcctaatattgtaaatgagtgggacacacttgcatattgtgaccattctattgaggcccttctctgtttaatgcatattatacttgtgcttttaactgtggaatctatttctaacctaaaaaaaaaaaaaaaaa

SEQ ID NO: 16 - DNA, 450 bp, Homo sapiens
  misc_feature (355)..(355): n is a, c, g, or t
aaatttctgtattttagttttgaagtgctttctatttaaaaataaaaaacatgatttattttcatttctgacacagaagtgtttcttttaaaaaaaaaagaccacattttaaatttctgcttaaatgtatataaagtatacatttaagtatactggcactcgcaacaaatgaatccttccccagggataaatggattggaaaatttgtttttcattcaacatttggaaagagaacaaacctgaaatatgtaatttttaaaattatgtgaaaataatgtgaaaaatttcatatagtatttgtgtgaaaatcaggtggaaaaaaacttccatgaagaacccaatttaccaaaattcnccatcnttttaagatttacnttttnaataccatactactggttttaacnggaattggggtgtggtatgaggggggttttgcnggggagangggga

SEQ ID NO: 17 - DNA, 508 bp, Homo sapiens
tatttccatgaattcaaagccttttaatgatgtgaacacttactccccatttcttttttacattgttacaaaaaatttacatacagttttctgaaagtggcattttgttggttgttattatactgatgacacatattaacactttgtattgaagaagtatcataaaaatcacagggcattacagatttttgataagaagtagtaatagcattgtcttttaacagctggaggctcccaggcatactctttggtgagaaatgattaattttatattttcattttgatgagaatcttttcttgtttttaccagttataaaaacaaagctttttctttgttgtgatactgtgcactaagacttagtttcttgagctgatgctaaataaaatgagatcaataggaatattccgggaggtcgtgagaagtttttagaaaggatggcatctacatatatatggagctctgaaaactgttggagagtatgacctgggactgaaactgtggagcaca

SEQ ID NO: 18 - DNA, 475 bp, Homo sapiens
  misc_feature (475)..(475): n is a, c, g, or t
tcgacatctaatcctcattcttatgaacttggtattatgtgaatccattgggaaatgaagactcagagaggttaaaccatttgcccaaggtcacacagctactaagcggtgctaggattaaaccctggcagtttcagtccttaaccaaagggttaagcgctttacatatatatacctgtatattatcaattttcaaataatttgaaatagtaaaatgcagttcctctggtcctgaatatgaggtaatcttcctatggttcagaagacatttcagcatttcctaatatatcataatgagtccattttggttgtaccatgatgtagtcattttgttttatagtatattaaagaatgcagaaaagcatagctcatagttccagtgtcttgctctgaggtttttccattcagtagacattttaagtatatgtaccaggcacataaatatccagataattaaagtttatatcattaagcn

SEQ ID NO: 19 - DNA, 1061 bp, Homo sapiens
gcgccatcaatcgccgccgcctcgtcccgcttctcggctgaggcgccgcgcggccaggcagcgggtccaggcctcagccgcgcgcccaggggcctccggggccctcccgggtcagcatgcccggggtgaaactgaccacccaggcctactgcaagatggtgctgcacggcgccaagtacccgcactgcgccgtcaacgggctcctggtggccgagaagcagaagccgcgtaaggagcacctccccctgggcggccccggcgcccaccacaccctcttcgtggactgcatccccctcttccacggcaccctggccctcgcccccatgctggaggtggctctcaccctgattgattcatggtgcaaagatcatagctacgtgattgctggttattatcaagctaatgagcgagtaaaggatgccagtccaaaccaggttgcagagaaggtggcctccagaatcgccgagggcttcagcgacactgcgctcatcatggtagacaacaccaagtttacgatggactgcgtagcgcctacgatccacgtgtacgagcaccatgagaacagatggcggtgcagagacccacaccatgactactgtgaagactggccagaggcacagaggatctcagcctcgctcctggacagccggtcctacgagacgctcgtggatttcgataaccacctggatgacattcggaatgactggacaaacccagagatcaataaagctgtcctacacttgtgctaggcaggcaccgctgtgactgggctccgggcctttcccactacgttgaagaagaaaacctatttttaaatgtaaataaaatatctggtagcctgtgtggaaagctgaccgttttaagaagtggcatgtgccttgaaagggggcagaatgttcagtcggtcgtgtttttaacacagagtctctagaagaggtgcagacatcccgtctgactgtccctgtggactctctcagttgtatgttgctataatcctccaaatcaaagctctttctgcttgtgcaagattgttcctattaaacagttttaactaaccttta

SEQ ID NO: 20 - DNA, 2040 bp, Homo sapiens
gaattccgggcagctcctcttccatctccagaaatgacctccaccttcaacccccgagaatgtaaactgtccaagcaagaagggcaaaactatggcttcttcctgcgaattgagaaggacaccgagggccacctggtccgggtggttgagaagtgtagcccagcagagaaggctggccttcaagatggagacagagttcttaggatcaatggtgtctttgtggacaaagaagaacatatgcaggttgtggatctggtcagaaagagtgggaattcagtgactttactagttctggatggggattcctatgagaaagcagtgaaaacacgggtggacttgaaagagttgggtcaaagtcagaaggagcaaggtttgagtgataatatactttcccctgtgatgaatggaggtgtgcaaacttggacccagccccggctctgctatctcgtgaaggaaggaggcagctatggcttctctctgaaaactgtccaaggtaaaaagggggtgtacatgactgatattacacctcaaggtgtggctatgagagctggagttctggctgatgatcacttgattgaagtgaatggagagaatgtagaggatgccagccatgagaaagtggttgaaaaggtgaagaagtcaggaagccgtgtcatgttcctgctggtggacaaagaaactgacaagcgtcatgttgagcagaagatacaattcaaaagagaaacagccagtttgaaactgttaccccaccagccccgaattgtggagatgaagaaaggaagcaatggctatggtttctatctgagggcaggctcagaacagaaaggtcaaatcatcaaggacatagattctggaagtccagcagaggaggctggcttgaagaacaatgatctggtagttgctgtcaacggcgagtctgtggaaaccctggatcatgacagtgtggtagaaatgattagaaagggtggagatcagacttcactgttggtggtagacaaagagacggacaacatgtacagactggctcatttttctccatttctctactatcaaagtcaagaactgcccaatggctctgtcaaggaggctccagctcctactcccacttctctggaagtctcaagtccaccagatactacagaggaagtagatcataagcctaaactctgcaggctggctaaaggtgaaaatggctatggctttcacttaaatgcgattcggggtctgccaggctcattcatcaaagaggtacagaagggcggtcctgctgacttggctgggctagaggatgaggatgtcatcattgaagtgaatggggtgaatgtgctagatgaaccctatgagaaggtggtggatagaatccagagcagtgggaagaatgtcacacttctagtctgtggaaagaaggcctatgattatttccaagctaagaaaatccctattgtttcctccctggctgatccacttgacacccctccagattctaaagaaggaatagtggtggagtcaaaccatgactcgcacatggcaaaagaacgggcccacagtacagcctcacattcttcttccaattctgaagatacagagatgtgatgaaaacaagtaatagctttggctgtttatttgatagctgtttctgggtatttaataggaatcctttctcaaggaatgagttgtgacctgtttactgtctctttagaagaaaaactccactggaaaccattcaccatgtgtgactgtcttctgttatcatttgtcttacaggcggctattgcagacggctaatttatgcttaacttaggaagagataaggcaagagctagatttttttcatgtgatcttttccaagcttcaacttaacttaactacatttctctgtatgatgatgtctcttacttctacaggttccttgagcaccaaagatgattcataactctgtataggtgacagctgcttataaaagcatcttagcagataagcctattaaaattgtgcttttgtaacaatgttgtggttgctagaataaataccatgaacccg

SEQ ID NO: 21 - DNA, 633 bp, Homo sapiens
tttaaaagccactaattatctgttttttattttgtaagtaacaagatatagacatttgaatgccaatgtcttattctggagagacactggagctgaagttcaacaatgatcacacttattacctggcaataaaaacacaaccatctttccagtcaggtcaaaatatcctactttttgcctttctaccaaatcccaaacattcacagtttttcaaggaccactaataaaatacaggaagcttttaaagacagtaagagaacacctagtgtaagttaggtgaattaaagatggcaaaggagattacatcctcaacactgacagcttccaagacttagaaaagagattgttccttgcttctaaaattgttctattttcctctgtaggaaaatgaaagttttttcttacaaatattaaataatcaaagtacttacgcaaaattaatctgctcctcaatgagatgagcactccatttaaatgatctttacagatccctgaagttgctgtcctgtcactgtatttaagtgatggatattcaattgaattattctgcataaataattctggtcaacccagacgtatagtagtatgatgggtcagatacagtcaactgttcaataaaaatgcagatgtctg

SEQ ID NO: 22 - DNA, 1878 bp, Homo sapiens
ctgcgtttctcctcaaacctaacgatgccgccggagcggaggagacgaatgaaactggaccggagaaccggagcgaagccgaagcggaagcccggaatgaggccggactggaaagccggagcggggccaggcgggcctccccaaaagcctgccccttcatcccagcggaaaccgccggcccggccgagcgcggcggccgctgcgattgcagtcgcggcggcggaggaagagagacggctccggcagcggaaccgcctgaggctggaggaggacaaaccggccgtggagcggtgcttggaggagctggtcttcggcgacgtcgagaacgacgaggatgcgttgctgcggcgtctgcgaggcccgagggttcaagaacatgaagactcgggtgactcagaagtggagaatgaagcaaaaggtaattttccacctcaaaagaagccagtttgggtggatgaagaagatgaagatgaggaaatggttgacatgatgaacaatcggtttcggaaggatatgatgaaaaatgctagtgaaagtaaactttcgaaagacaaccttaaaaagagacttaaagaagaattccaacatgccatgggaggagtacctgcctgggcagagactactaagcggaaaacatcttcagatgatgaaagtgaagaggatgaagatgatttgttgcaaaggactgggaatttcatatccacatcaacttctcttccaagaggaatcttgaagatgaagaactgccagcatgcgaatgctgaacgtcctactgttgctcggatctcatctgtgcagttccatcccggtgcacagattgtgatggttgctggattagataatgctgtatcactatttcaggttgatgggaaaacaaatcctaaaattcagagcatctatttggaaaggtttccaatctttaaggcttgttttagtgctaatggggaagaagttttagccacgagtacccacagcaaggttctttatgtctatgacatgctggctggaaagttaattcctgtgcatcaagtgagaggtttgaaagagaagatagtgaggagctttgaagtctccccagatgggtccttcttgctcataaatggcattgctggatatttgcatttgctagcaatgaagaccaaagaactgattggaagcatgaaaattaatggaagggttgcagcatccacattctcttcagatagtaagaaagtatacgcctcttcgggggatggagaagtttatgtttgggatgtgaactcaaggaagtgccttaacagatttgttgatgaaggcagtttatatggattaagcattgccacatctaggaatggacagtatgttgcttgtggttctaattgtggagtggtaaatatatacaatcaagattcttgtctccaagaaacaaacccaaagccaataaaagctataatgaacttggttacaggtgttacttctctgaccttcaatcctactacagaaatcttggcaattgcttcagaaaaaaatgaaagaagcagtcagattggttcatcttccttcctgtacagtattttcaaacttcccagtcattaaaaataagaatatttctcatgttcataccatggatttttctccgagaagtggatactttgccttggggaatgaaaagggcaaggccctgatgtataggttgcaccattactcagacttctaaagagactatttgaagtccagttgagtcacaagagaagcctgtcttgatatatcatctcagaaactttcctgaatatgtgataatatatggaaaatgatttatagatccagctgtgcttaagagccagtaatgtcttaataaacatgtggcagcttttgtttgaaaaaaaaaaaaaaaaaaaa

SEQ ID NO: 23 - DNA, 3426 bp, Homo sapiens
aattccgggcggcggcggccgaggctgaaggaagatggcggacggcgtggaccacataaacatttacgcggatgtcggcgaagagttcaaccaggaagctgaatatggtgggcatgatcagatagatttgtatgacgatgtcatatctccatctgcaaataatggagatgccccagaagaccgagattacatggatactctcccaccaactgttggtgatgatgtgggtaaaggagcagcaccaaatgttgtctatacatatactggaaagagaattgcattatatattggaaatctaacatggtggacaacagatgaagacttaactgaagcagttcattctttgggagtaaatgatattttggagataaaattttttgaaaatcgagcaaatggccagtcaaaggggtttgcccttgttggtgttggatctgaagcatcttcaaaaaagttaatggatctgttacctaaaagagaacttcatggtcagaatcctgttgtaactccatgcaataaacagttcctgagtcaatttgaaatgcagtccaggaaaactacacaatcaggacaaatgtctggggaaggtaaagctggtcctccaggaggcagttcccgtgcagcatttccacaaggtggtagaggacggggccgttttccaggggctgttcctggtggggacagatttcctgggccagcaggaccaggagggccacccccaccttttccagctggacagactccaccacgtccacccttaggtcctccaggcccacctggtccaccaggtcctccacctcctggtcaggttctgcctcctcctctagctgggcctcctaatcgaggagatcgccctccaccaccagttctttttcctggacaaccttttgggcagcctccattgggtccacttcctcctggccctccacctccagttccaggctacggcccccctcctggcccaccacctccacaacagggaccacctccacctccaggcccctttccacctcgtccacccggtccacttgggccaccccttacactagctcctcctccgcatcttcctggaccacctccaggtgccccaccgccagctccgcatgtgaacccagctttctttcctccaccaactaacagtggcatgcctacatcagatagccgaggtccaccaccaacagatccatatgggcgacctccaccatatgataggggtgactatggcccccctggaagggaaatggatactgcaagaacgccattgagtgaagctgaatttgaagaaatcatgaatagaaatagggcaatctcaagcagtgctatttcgagagctgtgtctgatgccagtgctggtgattatgggagtgctattgagacactggtaactgcaatttctttaattaaacaatccaaagtatctgctgatgatcgttgcaaagttcttattagttctttgcaagattgccttcatggaattgagtccaagtcttatggttctggatcaagacgtgaacgatcaagagagagggaccatagtagatcacgagaaaagagtcgacgtcataaatcccgtagtagagaccgtcatgacgattattacagagagagaagcagagaacgagagaggcaccgggatcgtgaccgagaccgtgaccgagagcgtgaccgagagcgcgaatatcgtcatcgttagaagctgaaggaagaggatcaccttccaagacaaaacagtcttcatgggccaaaaatgacgcttgtccagcagtttgcttcttgtgattgaactgaacctgtaaggattcatggataaaatgaacaggaatagatctgaataaagcaaatctgcataaatggtaaccagtagctctacttttattttttatgttgcttaactgttttatttgaaggaaacctgtgtgatttaaaaagttatagcttttgcaactttattactggttatatacatttggccattatgatgtgcaagcaattggaaaaaaagtcaagtaaatgcttgtttttgtagtagtttgttcttgttaaaaatgtttatatgataatgtctgtaaacagcatcactttgattacaatagatgtagtgttgtaataaactgtttaatggggctgatgtgtaaagctgttcaagttatttgatgtttacacctcagggaaagtcttgtgttcagcaatatctaaagataatgttactatgacaacatttttactgtcctttaaagcattgcaatagcgtttttggatatgcctcaatctaatcttgcgttcagtgaattaaacatagtaattaagtgtcttttgcccttgattttgatattagaataggtgattacatggatatttaatatttctatattctgcttttctagctgtttttacctagttagcttgtgactttgctgaatggtatgtaaacttgtaaaaatagagatttgacagacatagcaatctagtcaatgtgtaaggggtcaaaaaaaacagaggttttaacacataagtaaaaacccgtacatatttgatgtgtaatgcaggttaattacaacacagatgtaccgaaacacttaattgtgaaccgctaacattgaagaaattttgacaattccgatttgatgctgcaattacttgctgtttttattgatcttatggtttatttcttaagccatagtcagtgtaaatacagccctgcagcaggtaaatgtgagtaaagagagccttatattttccaattggtataaaatttttgaaggatgtgatgttcattaacattcggttgtattccccagtatttgtaatgggaaattacagataaaccgtgtctgcacagtttaaggaatactatgtatattcatgcaccgtattgattcatgctatagttacttaatcaaagatttttttcaaacctgccttacatataggcccactttaaaagcacctgactagcatgtgttcttgattgcaaaattggcagaggcagggtgtcaacttgattaggtgtttttatgggaatgtaatttgaaatcactacttcagaaatttgacttaaaattcttgagcacgttaatatgtttttaagatctgattatctttgagagatcttctgttaatacacattggttgttaaagagtacccaaattctaggacaatgcttaaagtgttaaaataccctagatactgtgttatgtgcaactgtagaaaccctccagaaatttccactgctgttcttcactttcatcttgtctgctatcaaaccacttctgacaaaattagctgttttgaattacccatatcactgccagttttattttaaaatattttgtgtttgaagtatctgtgcatgggatcgttgatgtttatcagaactgttcactttcagaaatgattttttaaagcattttgttgaaatgcggttgctt

SEQ ID NO: 24 - DNA, 5401 bp, Homo sapiens
tcactctcgctgccgctgctccgccccatccccttctgtttttctctctcattctccagtggcggcggcggggaaggcggaggcagaggcagcagcagccgcgctggctgcaatgaatgatcccccagcttggggggaggactccaggtgagcctctgccctcgggaggcccgggacccccggccgcccacgaccggcagcccacgctatggatccctagaggaaggaggagaagacagctcgccgcccacccccatcccattttcctcttcctttatctcattgttgccgaagctgtttacggcagcgctccctctgctccagcatggggcgggctccgggcacggctgctcggcaggcgctgctcccgcggcgactgggggattctgcctaattcacctcccagccggtgcagagaggaccggagagcggtggaggcccggactgcagcagcgttggggccacctcccagcgtccccaccctaggaggctgcatgcggattgaagagctgcgcctgggggctgggccggccccgctgatcccgacctagcgagcaggatagcaggaccgcccaggctgcggaggggctcgggggcaggaaggtcagagcagcaagatggccagtaagaccaaggccagcgaggccctcaaggtggtggcccggtgccgccccctcagcaggaaggaggaggctgctggtcacgagcagatcctgaccatggacgtgaaactgggccaggtgaccctgcggaacccccgcgccgccccgggggagctgcccaagaccttcacctttgacgccgtgtatgatgccagctccaagcaggccgacctgtatgacgaaaccgtgaggcccctgatagactccgtgctccagggtttcaatggcacggtgtttgcctatggccagacgggcactggcaagacctataccatgcaggggacctgggtggagcccgagctgcgcggggtcatcccgaatgcctttgagcacatcttcacccacatctcccgctcccagaaccaacagtacctggtccgggcctcctatttggagatctaccaggaagagattcgagacctgctctccaaggagccgggcaagaggctagagctgaaagagaaccccgagactggcgtctacatcaaggacctctcctccttcgtcaccaagaatgtcaaggagattgagcatgtgatgaacctggggaaccagacccgggctgtgggcagcacccacatgaatgaggtcagctcccgctcccatgccatcttcatcatcactgtggagtgcagcgaacgtggctctgatggccaggaccacatccgagtgggcaagctcaacctcgtggacctggctggcagcgagaggcagaacaaggcaggccccaacacagcgggaggggcagccacaccatcctcgggtggcggtggtggcggtggaggcagtggtggtggtgctggtggagagaggcctaaggaagcctccaaaatcaacctctcattatctgccctgggcaacgtgattgctgccctggcgggcaacaggagcacccacattccctaccgggactccaagctgacccggctgctccaggactccctgggggggaatgccaagaccatcatggtagccacactggggccagcttctcacagctacgatgagagcctctccaccttgcgctttgccaaccgagccaagaacatcaagaacaagccccgggtgaacgaggaccccaaggacacactgctgcgggaattccaagaggagattgcccgcctgaaggcccagctggagaagagggggatgctggggaagcggccccggaggaagagcagccgcaggaagaaggccgtgtccgccccgcctgggtaccctgagggcccagtgattgaggcctgggtggcagaagaggaggatgacaacaacaacaaccaccgcccgccccagcccatcctggagtcagccttggagaagaacatggagaattacctgcaggaacagaaggagcggctggaggaggagaaggcagccatccaggatgaccgcagcctggtgagcgaggagaagcagaagctgctggaggagaaggagaagatgctggaggacctgcggcgggaacagcaggccacagagctgcttgcggccaagtacaaggccatggagagcaagctcctcatcgggggcaggaacatcatggatcacaccaacgaacagcagaagatgttggaactgaagaggcaggagattgccgagcagaaacgtcgtgagcgggagatgcagcaggagatgatgctccgggacgaggagactatggagctccggggcacctacacatccctgcagcaggaggtggaggtcaaaaccaagaaactcaagaagctctacgccaagctgcaggcggtgaaggcggagatccaggaccagcatgatgagtatatccgcgtgcggcaggacctggaggaggcgcagaacgagcagacccgcgaactcaagctcaagtacctaatcatcgagaacttcatcccgccggaggagaagaacaagatcatgaaccggcttttcctggactgtgaggaggagcagtggaagttccagccactggtgccagccggcgtcagtagcagccagatgaagaagcggccaacatctgcagtgggctacaagaggcctatcagccagtatgctcgggttgccatggcaatggggtcccaccccaggtacagggctgaaaacataatgtttctggagttggatgtgtcccctccagctgtctttgagatggaattctctcacgaccaagaacaagaccctcgtgcgctacacatggagaggctcatgcgattggacagctttctggaaagaccttccacgtctaaagtccgaaagtccagatcctggtgccagagtcctcagcggcctccaccttccaccacacatgcctccctggcctctgcttctctgcgccctgcaacagtggcggaccatgagtgacaaccatcacgtcaggctgcccatccaatagactcctgggatggggcagccaaccctggctcatctcatctgccgcttggtgcgtgtgcgtgtgcgtgcatgtgcgtgtgcgtgtgtgcaggggtgagaatctggcagatggtgcctctgcctgctcttcttcgcctcctttatttaattcatgttatttattcgcggagctctgttcgtgttggggagatgccctcgcctgagccgtctgggcctaccgtggtcactgcgtagcctctttttcttctgacttgagagctcccccagtcagatctcaggcttgtccccctgtcagctgcctccagaagggaaggtagccagtgcctgagaagacagtcccttttctacccaccgcactccataacctccatcttctcccacactgatggcgagcagcccctgagcactttctgggactgggagactgcttggtgttccctgaggacaagagacatcctgacagtgttgggcatctgctccccgtggacacagccccactctccactttctgagcctcagacaacctcattcagcctcttgggctccttttcaaggacattaataacctcaccaacatagctcatgcccttcagctttgacaagaactcacggcttcccaaactctgctttctgcccaccttggatgggaactgtggaccaagcaattaccatcgccttggaacctgcaggaaatggaacagcaattgagacaacttgaacagtcatcaacggaagtccctccactggattcctttgtttctgtcccctccgaggagtcattttggtcgacaggctctcaaggcaactccccattttcaagaggctgctcctgcctgcttcgatcatttctccctgcagctgcctagaccccgttcacagtgggaggagtcaatgtcattctacccctcgctaaacgaagatattaacatctattgctttttcccttcatctgtcacaggaaacagaagcccaggcacaatcttttccagctttgcctgttacccctgtttctgaattgcatctttaaggtattattttgttgacaatagatcctttattcactagttacgcaaattggttcctagggggatactccttaccttcctttgtgatggcccaaaatgtctctaggtatctcaagtgataagtaaatttctacaaaaaaaaatggttaatgttcattgactggctttttaagtgtatattttggaggacgggtgaagaggtcataacgaaagcaagcgagtgaattaggatttcaaagtgccctaatagtgtgagtctccagttcctagaatatgaagagtgctgtcgttggggtgaaaccatgagactgacagatctgcctgaaatggggggtgtgggaggtggtggcgggggttattctctttccttcaggaaatgaacccttcttacatcattcaagttctgctctgaggatcaagcttgggtctgatttaactcagcgacactgtcatttctgcttcattactggactagagggttgagccacccacttgccatttgctcctgtccttccaggaaatcacaattttcatcagagcccaagagattatttgagactcaggattcagatcagaggttcgactgtggctgggacaggagttgtgtgtagaaattcaccaggtggcctgagcgcagggggacctccagggctgcgttgagcagcctctcccactgacctctttctcgtttgtggacaaagcagcacgtatcacctcattcatcacttggacacatcgcctttgcattgtcttgtcacacctccctcacagtcttatagcacaatatacccaaatcagcccccccagtccgagggctgggcccaaggtatggtcggaggaggagctcctgcctgcggttttgtgtatgtgtgtatgtgtgtgcgtgtttgtgtgcgtgtttacctccacaggggacactctacactcagtgtaagatctgctgggaacagggccaccaggagtggctggatctcagtctctctgtctctctttctctccttttccttttggtgtatcaaatatttgattgacaaagtaagggccttgattaggaccaaattctcgtgtgttgctatggtctttatttaggacaacaattaacaatgcagtggcccattcttgtcactctacacatatgactatacgggacatatgtaatatataaatatatatataaaacattcccctctgtccccttggcttcggatggaggcctttctgttgagctgaaatgcacctgcagctgggtgctgccagcagcttgcaggccccagccctgttccaatcaatgcagttgacaataaaggaatgagtatcgtcacggaaaaaaaaaaaaaaaaaaaaaaa

SEQ ID NO: 25 - DNA, 1280 bp, Homo sapiens
taaacaatggtatcaacgcaaagtaagcgggcagccgcctgcatctgtatccagcgccaggtcccgccagtcccagtgcgcgcgccccccagtcccgcacccgttcggccaggctaagttagccctcaccatgcggtcaaaggaggcagcaagtgcatcaaatacctgctgttcggatttaacttcatcttctggcttgccgggattgctgtccttgccattggactatgggtccgattcgactctcagaccaagagcatcttcgagcaagaaatttcctaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgaccaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatggccaaaaaaaaaaaaaaaaaaaaaaaaa

SEQ ID NO: 26 - DNA, 3345 bp, Homo sapiens
gcggccggggcctggggctgcctgccgggcggccgggcgcggcgagcccagggaggcagcgtccatggagcaaaaggaatgccaggatcctgcacaggcagacgcgggccagcctcagcaccgacagccgacgcgcagatagcagagccatccttggggttgaaccatgattccggtgacagagctccgctactttgcggacacgcagccagcataccggatcctgaagccgtggtgggatgtgttcacagactacatctctatcgtcatgctgatgattgccgtcttcggggggacgctgcaggtcacccaagacaagatgatctgcctgccttgtaagtgggtcaccaaggactcctgcaatgattcgttccggggctgggcagcccctggcccggagcccacctaccccaactccaccattctgccgacccctgacacgggccccacaggcatcaagtatgacctggaccggcaccagtacaactacgtggacgctgtgtgctatgagaaccgactgcactggtttgccaagtacttcccctacctggtgcttctgcacacgctcatcttcctggcctgcagcaacttctggttcaaattcccgcgcaccagctcgaagctggagcactttgtgtctatcctgctgaagtgcttcgactcgccctggaccacgagggccctgtcggagacagtggtggaggagagcgaccccaagccggccttcagcaagatgaatgggtccatggacaaaaagtcatcgaccgtcagtgaggacgtggaggccaccgtgcccatgctgcagcggaccaagtcacggatcgagcagggtatcgtggaccgctcagagacgggcgtgctggacaagaaggagggggagcaagccaaggcgctgtttgagaaggtgaagaagttccggacccatgtggaggagggggacattgtgtaccgcctctacatgcggcagaccatcatcaaggtgatcaagttcatcctcatcatctgctacaccgtctactacgtgcacaacatcaagttcgacgtggactgcaccgtggacattgagagcctgacgggctaccgcacctaccgctgtgcccaccccctggccacactcttcaagatcctggcgtccttctacatcagcctagtcatcttctacggcctcatctgcatgtatacactgtggtggatgctacggcgctccctcaagaagtactcgtttgagtcgatccgtgaggagagcagctacagcgacatccccgacgtcaagaacgacttcgccttcatgctgcacctcattgaccaatacgacccgctctactccaagcgcttcgccgtcttcctgtcggaggtgagtgagaacaagctgcggcagctgaacctcaacaacgagtggacgctggacaagctccggcagcggctcaccaagaacgcgcaggacaagctggagctgcacctgttcatgctcagtggcatccctgacactgtgtttgacctggtggagctggaggtcctcaagctggagctgatccccgacgtgaccatcccgcccagcattgcccagctcacgggcctcaaggagctgtggctctaccacacagcggccaagattgaagcgcccgcgctggccttcctgcgcgagaacctgcgggcgctgcacatcaagttcaccgacatcaaggagatcccgctgtggatctatagcctgaagacactggaggagctgcacctgacgggcaacctgagcgcggagaacaaccgctacatcgtcatcgacgggctgcgggagctcaaacgcctcaaggtgctgcggctcaagagcaacctaagcaagctgccacaggtggtcacagatgtgggcgtgcacctgcagaagctgtccatcaacaatgagggcaccaagctcatcgtcctcaacagcctcaagaagatggcgaacctgactgagctggagctgatccgctgtgacctggagcgcatcccccactccatcttcagcctccacaacctgcaggagattgacctcaaggacaacaacctcaagaccatcgaggagatcatcagcttccagcacctgcaccgcctcacctgccttaagctgtggtacaaccacatcgcctacatccccatccagatcggcaacctcaccaacctggagcgcctctacctgaaccgcaacaagatcgagaagatccccacccagctcttctactgccgcaagctgcgctacctggacctcagccacaacaacctgaccttcctccctgccgacatcggcctcctgcagaacctccagaacctagccatcacggccaaccggatcgagacgctccctccggagctcttccagtgccggaagctgcgggccctgcacctgggcaacaacgtgctgcagtcactgccctccagggtgggcgagctgaccaacctgacgcagatcgagctgcggggcaaccggctggagtgcctgcctgtggagctgggcgagtgcccactgctcaagcgcagcggcttggtggtggaggaggacctgttcaacacactgccacccgaggtgaaggagcggctgtggagggctgacaaggagcaggcctgagcgaggccggcccagcacagcaagcagcaggaccgctgcccagtcctcaggcccggaggggcaggcctagcttctcccagaactcccggacagccaggacagcctcgtggctgggcaggagcctggggccgcttgtgagtcaggccagagcgagaggacagtatctgtggggctggccccttttctccctctgagactcacgtcccccagggcaagtgcttgtggaggagagcaagtctcaagagcgcagtatttggataatcagggtctcctccctggaggccagctctgccccaggggctgagctgccaccagaggtcctgggaccctcactttagttcttggtatttatttttctccatctcccacctccttcatccagataacttatacattcccaagaaagttcagcccagatggaaggtgttcagggaaaggtgggctgccttttccccttgtccttatttagcgatgccgccgggcatttaacacccacctggacttcagcagagtggtccggggcgaaccagccatgggacggtcacccagcagtgccgggctgggctctgcggtgcggtccacgggagagcaggcctccagctggaaaggccaggcctggagcttgcctcttcagtatttgtggcagttttagttttttgtttttttttttttaatcaaaaaacaatttttttaaaaaaaaagctttgaaaatggatggtttgggtattaaaaaaaaaaaaaaaaaa

SEQ ID NO: 27 - DNA, 4762 bp, Homo sapiens
atgcggcgcggccccggaggcagcagcagcggcggcggcagccggagcagtaggcacccgagcagcgccagcggccgagcgggcggcttcctggcctgggcgctccggtggcggcggaggtgcgcgcggagccatggttatcatgtcggagttcagcgcggaccccgcgggccagggtcagggccagcagaagcccctccgggtgggtttttacgacatcgagcggaccctgggcaaaggcaacttcgcggtggtgaagctggcgcggcatcgagtcaccaaaacgcaggttgcaataaaaataattgataaaacacgattagattcaagcaatttggagaaaatctatcgtgaggttcagctgatgaagcttctgaaccatccacacatcataaagctttaccaggttatggaaacaaaggacatgctttacatcgtcactgaatttgctaaaaatggagaaatgtttgattatttgacttccaacgggcacctgagtgagaacgaggcgcggaagaagttctggcaaatcctgtcggccgtggagtactgtcacgaccatcacatcgtccaccgggacctcaagaccgagaacctcctgctggatggcaacatggacatcaagctggcagattttggatttgggaatttctacaagtcaggagagcctctgtccacgtggtgtgggagccccccgtatgccgccccggaagtctttgaggggaaggagtatgaaggcccccagctggacatctggagcctgggcgtggtgctgtacgtcctggtctgcggttctctccccttcgatgggcctaacctgccgacgctgagacagcgggtgctggagggccgcttccgcatccccttcttcatgtctcaagactgtgagagcctgatccgccgcatgctggtggtggaccccgccaggcgcatcaccatcgcccagatccggcagcaccggtggatgcgggctgagccctgcttgccgggacccgcctgccccgccttctccgcacacagctacacctccaacctgggcgactacgatgagcaggcgctgggtatcatgcagaccctgggcgtggaccggcagaggacggtggagtcactgcaaaacagcagctataaccactttgctgccatttattacctcctccttgagcggctcaaggagtatcggaatgcccagtgcgcccgccccgggcctgccaggcagccgcggcctcggagctcggacctcagtggtttggaggtgcctcaggaaggtctttccaccgaccctttccgacctgccttgctgtgcccgcagccgcagaccttggtgcagtccgtcctccaggccgagatggactgtgagctccagagctcgctgcagtggcccttgttcttcccggtggatgccagctgcagcggagtgttccggccccggcccgtgtccccaagcagcctgctggacacagccatcagtgaggaggccaggcaggggccgggcctagaggaggagcaggacacgcaggagtccctgcccagcagcacgggccggaggcacaccctggccgaggtctccacccgcctctccccactcaccgcgccatgtatagtcgtctccccctccaccacggcaagtcctgcagagggaaccagctctgacagttgtctgaccttctctgcgagcaaaagccccgcggggctcagtggcaccccggccactcaggggctgctgggcgcctgctccccggtcaggctggcctcgcccttcctggggtcgcagtccgccaccccagtgctgcaggctcaggggggcttgggaggagctgttctgctccctgtcagcttccaggagggacggcgggcgtcggacacctcactgactcaagggctgaaggcctttcggcagcagctgaggaagaccacgcggaccaaagggtttctgggactgaacaaaatcaaggggctggctcgccaggtgtgccaggtccctgccagccgggccagcaggggcggcctgagccccttccacgcccctgcacagagcccaggcctgcacggcggcgcagccggcagccgggagggctggagcctgctggaggaggtgctagagcagcagaggctgctccagttacagcaccacccggccgctgcacccggctgctcccaggccccccagccggcccctgccccgtttgtgatcgccccctgtgatggccctggggctgccccgctccccagcaccctcctcacgtcggggctcccgctgctgccgcccccactcctgcagaccggcgcgtccccggtggcctcagcggcgcagctcctggacacacacctgcacattggcaccggccccaccgccctccccgctgtgcccccaccacgcctggccaggctggccccaggttgtgagcccctggggctgctgcagggggactgtgagatggaggacctgatgccctgctccctaggcacgtttgtcctggtgcagtgagggcagccctgcatcctggcacggacactgactcttacagcaataacttcagaggaggtgaagacatctggcctcaaagccaagaactttctagaagcgaaataagcaatacgttaggtgttttggctttttagtttatttttgttttatttttttcttgcactgagtgacctcaactttgagtagggactggaaactttaggaagaaagataattgaggggcgtgtctgggggcgggggcaggaggggagcggggtggagggaacacgtgcagtgccgtggtgtggggatctcggcccctctctctgggttcgtcgtggttgagatgattacctcggacgtctacggaaacgagcgggcgcattgttgtccgcttgtgtgtgtgtgtgtgtgtgtgtgtgtgcgcgtgcattgattactatccatttctttagtcaacgctctccacttcctgatttctgctttaaggaaaactgtgaactttctgcttcatgtatcagttttaaagcagcccaggcaaagatcatctacagattctaggaattctctcccctgaaatcaaaacctggaagacttttttttcttattttagttgagaagtttcataaactgctcaaggattagttttccaggactctgcggaggaacggcaggaagaacctcagagagggcagaggtgacttcaaagtgctggggactccgtcctgagggtcacttggccctgagcccctgcgtgcccttgcggaagcccagaagcttcttcctgctgcacctcccgtttccgctgctgctgacgtttatgcatttcatgatggggtccaacaagaacacctgacttgggtgaagttgtgcaatattggaggctgactgtagggctgggcagctgggagacaggctcatggctcatggctcatggctcagggcggtgcctgccatgggccgggacccccctccccaccccccacctaggctttttgggttttgttcaaggaaggtaaagtgagaggtttaggtcagtgtttttaagtttttgttttttttttaaagcaaatcctgtatatgtatctacatgggagacaggtagacactacttatttgttacattttgtactacacgtttgtgttccaggtttcagcttccctcgctcctgttgttaagaagcgtccctgtcagcacaggtgtgcattgaggaaggggccccagggccttcgctccctcagcactggggtggaggcggcaggaaggggcggcccttacctggcaggtctgggcgcacctttagcaggtggactccgtggggctccaccagccagaagcctttggaaggcaacgaaggcaatgctgctccctgagtccagtccccgcccccaaacccagcccaggtgccttcagctacttcggcttcttaaaccctgcagtgttaaacagaggcattgagaaaggggaaaggcgggtatttttaaaagccaaagattgacccagttacttgagggtagggaggcgggcccagtgcaggaggctgcatccctggcctgctggtgcccaccgggggctgtgcctgtgccgggccgcagggaagctggctgcccccattcctgctgctgctgctgctgctgctctgtggctgtttcaaagactgggcgaaaggctgtccggagggcagaccaggtgccttgccgcagagaaaacaccaaagtctcctgttcgctcataaagaagtttttgggatgggagagaatccagaccatcttggggcagccaggcccttgccttcatttttacagaggtagcacaattgattccaacacaaaacttcccctttttaaaatgatttctgttctaatgccatagatcaaaggcctcagaaaccattgtgtgtttcctctttgaagcaatgacaagcactttactttcacggtggtttttgttttttcttattgctgtggaacctcttttggaggacgttaaaggcgtgttttacttgtttttttaagagtgtgtgatgtgtgttttgtagatttcttgacagtgctgtaatacagacggcaatgcaatagcctatttaaagacactacgtgatctgattgagatgtacatagtttttttttttaccataactgaattattttatctcttatgttaacatgagaaatgtatgccaaatgattagttgatgtatgttttttaatttaatatttaaataaaatatttgggagtataaaaaaaaaaaaaaaaaaaaaaaaaaaaaaaaaaaaaaaaaaaaaaaaaaaaaaaaa

SEQ ID NO: 28 - DNA, 3327 bp, Homo sapiens
cacacctttccaaggacccccaaactctgctccgtgcacgtcaaatgctcctttcccttgtgtccaaccccctacccctctccctaacacccctcttctcaacaagactcagcctctccccgaggtgggtgagcatccttgaggtttcccacccttaactgctgtgtccccggatggagccagagaaatgtggtgggggggccggggcagagtttcaacattgccccccagaaggaggagccagagatggggtctgtccaggaaaacaggatgccggagcccaggagtcgtcagcctagcagttgcctggcctccagatgcctcccaggggagcagatcctagcatgggccccaggggtgaggaagggcctggaaccagaattgtctggaaccctgatctgtaccaactttagggtcaccttccagccctgtggatggcagtggaatcaggacactcccttgaacagtgaatacgattttgccctggtcaacattggacgattagaggctgtgagcggcttgtcccgagtccagctcctccgtccagggtccctgcataaatttatccctgaggagattctgattcatggccgagacttccggctgctcagagttggttttgaggctggaggcctagagcctcaggcttttcaggtgaccatggccattgtccaagccagagctcagagcaatcaagcccaacagtattcggggataaccctgagcaaggctggccagggttctggctccagaaaaccaccaattcctctcatggagacagcggaagactgggagactgagcggaagaagcaggcagccagaggctggagggtcagcacggtcaacgagaggttcgacgtagccaccagcctcccccgttacttctgggtccctaaccgaattctggacagtgaggtcaggagagcatttggccactttcatcagggccgtggaccggtcagtgtgatggttagggtaatggctgtggattagagggtcatgtgggccagggacatcgtggagggaggaacctctgtgaggtcagtgtgggggcaagggtagcgtggagctaggcatttctcccacaatgaccctcttctgccccatgtgaagcgcttgtcctggcatcaccctgggggcagtgatcttctccgctgtggaggcttctatacagccagtgaccctaacaaggaggatatcagagcagtggagttgatgcaccaggctgggcattcagatgttgtcctggtagacactatggatgagctgcccagccttgcagatgtccaacttgcccacctgaggctgagggccctctgcctgcctgattcatctgtagctgaggataaatgctttcagccctggaaggaacacgatggctggactatgtcagggcttgtcttcgaaaggccagtgacatttcagtattagtgacatccagggttcgttctgtaatacttcaaggctccggtgtttctcctcttccttgattgtgtctggcagctcctccagcagtttccagctgattttgaattctctgagtttttccttcttgctcttcatgacagtgtcagggttcctgacacccttaccttcctgagaaataccccctgggagcgcggaaagcagagcggacaggtcagtgacttctatttttgactcgtgtttttttttccattgagatgtactctctgaagtttggtcttgatttgttttatgagaagtgaggtctgtgagtggggagggggagatttattctcattttcaggacgagacttttgccctacatctttcctagaataagaggtgagaatctcatgatttgtctctagatgtgggaggattgtgtgtaaccatcctttttcttgcttcctctgtccagttaaactcctatacacaagtctacaccccaggatactccagcctccagctgggaactcttttaacctgcagctgtctgtctgggactgggatttacgttatagcaatgcacagatactacaattccagaatcctggctatgacccagaacactgtccagattcctggctccctagaccacagccaagcttcatggttcctggaccccccagttttgtgtggctcttctctagaggagcattgacccccctgaatcagctctgtccttggcgggacagtccttccctgctggcagtctcttctcgttggctccctcgacctgctatctcctctgaaagctggctgaccaggaatggggtctcccctcacattggggagcttgccctttacctccagggctgctgctgcctgggtatctgggaccccagatcaggctctggagacgctgctacctgaggggaaggcctgaggtccaggtaagaagggaaaatagactgggagtgggacaagggacttgactctgctgaaccagatgaacaggagctggaaaggcaaggagctgaagcctctgggagtctgggaagtgaagttctactcctcttggcatcaaacaaggtttgggagtgtaggaggtgcgggaaagtgcttgtggcttagattaagtggaatttagggcatagctgaaaggggaaacagaattaaagacaccagaagtagcagagaagcagggggccagagctacaacagtattcttctctgttcctctttgcctcctccccagatgggcctctcatctcccacaatctctggcctccaggatgagctatcccatcttcaggagttattacggaaaggacaccaagaatatctcctgaggatcactccaagaaaagagatccacataccattctcaatcccactgaaattgctggcattctcaaaggcagggcagagggggatctggggtagagggagggttctgtctaatcttttttttttcttttgtatctgcacttgcagcctcagctttcatacttcagcccttaagttcactaagaaggtctgagtttctgctgcagatagtggtgttaactgctccaactcttgtcttgcttagtttctacaaatatttttgcttcttgtcatttgaaggattaagaaacaaaaacaatccagaaattgatcggtttttttaggccaatcccatcccttctggataaccagatgttaaatcatgagatcagagatgctgttcatcagtcccaacaagatggcctagaaatcgcattctcacctcgccttgctgctgctttaattccaagttctatttcttcccttatagttttctatgggaatgaggcggatacaggaaacaccctatctcctctgtatttttgtagtggaatttctatttaaggggctcattaaagcatagtatttatacac

SEQ ID NO: 29 - DNA, 3061 bp, Homo sapiens
gagcaattgattaatagctcggcgaggggactcactgactgttataataacactacaccagcaactcctggcttcccagcagccggaacacagacaggagagagtcagtggcaaatagacatttttcttatttcttaaaaaacagcaacttgtttgctacttttatttctgttgatttttttttcttggtgtgtgtggtggttgtttttaagtgtggagggcaaaaggagataccatcccaggctcagtccaacccctctccaaaacggcttttctgacactccaggtagcgagggagttgggtctccaggttgtgcgaggagcaaatgatgaccgccaaggccgtagacaaaatcccagtaactctcagtggttttgtgcaccagctgtctgacaacatctacccggtggaggacctcgccgccacgtcggtgaccatctttcccaatgccgaactgggaggcccctttgaccagatgaacggagtggccggagatggcatgatcaacattgacatgactggagagaagaggtcgttggatctcccatatcccagcagctttgctcccgtctctgcacctagaaaccagaccttcacttacatgggcaagttctccattgaccctcagtaccctggtgccagctgctacccagaaggcataatcaatattgtgagtgcaggcatcttgcaaggggtcacttccccagcttcaaccacagcctcatccagcgtcacctctgcctcccccaacccactggccacaggacccctgggtgtgtgcaccatgtcccagacccagcctgacctggaccacctgtactctccgccaccgcctcctcctccttattctggctgtgcaggagacctctaccaggacccttctgcgttcctgtcagcagccaccacctccacctcttcctctctggcctacccaccacctccttcctatccatcccccaagccagccacggacccaggtctcttcccaatgatcccagactatcctggattctttccatctcagtgccagagagacctacatggtacagctggcccagaccgtaagccctttccctgcccactggacaccctgcgggtgccccctccactcactccactctctacaatccgtaagccctttccctgcccactggacaccctgcgggtgccccctccactcactccactctctacaatccgtaactttaccctggggggccccagtgctggggtgaccggaccaggggccagtggaggcagcgagggaccccggctgcctggtagcagctcagcagcagcagcagccgccgccgccgccgcctataacccacaccacctgccactgcggcccattctgaggcctcgcaagtaccccaacagacccagcaagacgccggtgcacgagaggccctacccgtgcccagcagaaggctgcgaccggcggttctcccgctctgacgagctgacacggcacatccgaatccacactgggcataagcccttccagtgtcggatctgcatgcgcaacttcagccgcagtgaccacctcaccacccatatccgcacccacaccggtgagaagcccttcgcctgtgactactgtggccgaaagtttgcccggagtgatgagaggaagcgccacaccaagatccacctgagacagaaagagcggaaaagcagtgccccctctgcatcggtgccagccccctctacagcctcctgctctgggggcgtgcagcctgggggtaccctgtgcagcagtaacagcagcagtcttggcggagggccgctcgccccttgctcctctcggacccggacaccttgagatgagactcaggctgatacaccagctcccaaaggtcccggaggccctttgtccactggagctgcacaacaaacactaccaccctttcctgtccctctctccctttgttgggcaaagggctttggtggagctagcactgccccctttccacctagaagcaggttcttcctaaaacttagcccattctagtctctcttaggtgagttgactatcaacccaaggcaaaggggaggctcagaaggaggtggtgtggggacccctggccaagagggctgaggtctgaccctgctttaaagggttgtttgactaggttttgctaccccacttccccttattttgacccatcacaggtttttgaccctggatgtcagagttgatctaagacgttttctacaataggttgggagatgctgatcccttcaagtggggacagcaaaaagacaagcaaaactgatgtgcactttatggcttgggactgatttgggggacattgtacagtgagtgaagtatagcctttatgccacactctgtggccctaaaatggtgaatcagagcatatctagttgtctcaacccttgaagcaatatgtattataaactcagagaacagaagtgcaatgtgatgggaggaacatagcaatatctgctccttttcgagttgtttgagaaatgtaggctattttttcagtgtatatccactcagattttgtgtatttttgatgtacactgttctctaaattctgaatctttgggaaaaaatgtaaagcatttatgatctcagaggttaacttatttaagggggatgtacatatattctctgaaactaggatgcatgcaattgtgttggaagtgtccttggtgccttgtgtgatgtagacaatgttacaaggtctgcatgtaaatgggttgccttattatggagaaaaaaatcactccctgagtttagtatggctgtatatttctgcctattaatatttggaattttttttagaaagtatatttttgtatgctttgttttgtgacttaaaagtgttacctttgtagtcaaatttcagataagaatgtacataatgttaccggagctgatttgtttggtcattagctcttaatagttgtgaaaaaataaatctattctaacgcaaaaccactaactgaagttcagataatggatggtttgtgactatagtgtaaataaatacttttcaacaataaaaaaaaaaaaaaaaaaaaaaaaaa

SEQ ID NO: 30 - DNA, 1677 bp, Homo sapiens
ggccaagcaagcttctatctgcacctgctctcaatcctgctctcaccatgagcctccgcctgcagagctcctctgccagctatggaggtggtttcgggggtggctcttgccagctgggaggaggccgtggtgtctctacctgttcaactcggtttgtgtctgggggatcagctgggggctatggaggcggcgtgagctgtggttttggtggaggggctggtagtggctttggaggtggctatggaggtggccttggaggtggctatggaggtggccttggaggtggctttggtgggggttttgctggtggctttgttgactttggtgcttgtgatggcggcctcctcactggcaatgagaagatcaccatgcagaacctcaacgaccgcctggcttcctacctggagaaggtgcgcgccctggaggaggccaacgctgacctggaggtgaagatccgtgactggcacctgaagcagagcccagctagccctgagcgggactacagcccctactacaagaccattgaagagctccgggacaagatcctgaccgccaccattgaaaacaaccgggtcatcctggagattgacaatgccaggctggctgtggacgacttcaggctcaagtatgagaatgagctggccctgcgccagagcgtggaggccgacatcaacggcctgcgccgggtgctggatgagctcactctgtctaagactgacctggagatgcagatcgagagcctgaatgaagagctagcctacatgaagaagaaccatgaagaggagatgaaggaatttagcaaccaggtggtcggccaggtcaacgtggagatggatgccaccccaggcattgacctgacccgcgtgctggcagagatgagggagcagtacgaggccatggcagagaggaaccgccgggatgctgaggaatggttccacgccaagagtgcagagctgaacaaggaggtgtctaccaacactgccatgattcagaccagcaagacagagatcacggagctcaggcgcacgctccaaggcctggagattgagctgcagtcccagctgagcatgaaagcggggctggagaacacggtggcagagacggagtgccgctatgccctgcagctgcagcagatccagggactcatcagcagcatcgaggcccagctgagcgagctccgcagtgagatggagtgccagaaccaagagtacaagatgctgctggacatcaagacacgtctggagcaggagatcgccacctaccgcagcctgctcgagggccaggacgccaagaagcgtcagcccccgtagcacctctgttaccacgacttctagtgcctctgttaccaccacctctaatgcctctggtcgccgcacttctgatgtccgtaggccttaaatctgcctggcgtcccctccctctgtcttcagcacccagaggaggagagagccggcagttccctgcaggagagaggaggggctgctggacccaaggctcagtccctctgctctcaggaccccctgtcctgactctctcctgatggtgggccctctgtgctcttctcttccggtcggatctctctcctctctgacctggatacgctttggtttctcaacttctctaccccaaagaaaagattattcaataaagtttcctgcctttctgcaaacataaaaa

SEQ ID NO: 31 - DNA, 8191 bp, Homo sapiens
tttgcttgcaacactggcacctctgccctgcaccccgggagtgagcagtgagtgaggctcgggtctgggcgctggctccgaatcttcgggctgggagagactccaccatctgggggcggcctgggggagcagccttagtgtcttcctgctgatgcaatccgctaggtcgcgagtctccgccgcgagagggccggtctgcaatccagcccgccacgtgtactcgccgccgcctcgggcactgccccaggtcttgctgcagccgggaccgcgctctgcagccgcagacccggtccacacggccaggggctacgacccttgggatctgccctccgctcagctcgagcttccctcgtggccgacggaacaatgaaggattgcagtaacggatgctccgcagagtgtaccggagaaggaggatcaaaagaggtggtggggacttttaaggctaaagacctaatagtcacaccagctaccattttaaaggaaaaaccagaccccaataatctggtttttggaactgtgttcacggatcatatgctgacggtggagtggtcctcagagtttggatgggagaaacctcatatcaagcctcttcagaacctgtcattgcaccctggctcatcagctttgcactatgcagtggaattatttgaaggattgaaggcatttcgaggagtagataataaaattcgactgtttcagccaaacctcaacatggatagaatgtatcgctctgctgtgagggcaactctgccggtatttgacaaagaagagctcttagagtgtattcaacagcttgtgaaattggatcaagaatgggtcccatattcaacatctgctagtctgtatattcgtcctacattcattggaactgagccttctcttggagtcaagaagcctaccaaagccctgctctttgtactcttgagcccagtgggaccttatttttcaagtggaacctttaatccagtgtccctgtgggccaatcccaagtatgtaagagcctggaaaggtggaactggggactgcaagatgggagggaattacggctcatctctttttgcccaatgtgaagcagtagataatgggtgtcagcaggtcctgtggctctatggagaggaccatcagatcactgaagtgggaactatgaatctttttctttactggataaatgaagatggagaagaagaactggcaactcctccactagatggcatcattcttccaggagtgacaaggcggtgcattctggacctggcacatcagtggggtgaatttaaggtgtcagagagatacctcaccatggatgacttgacaacagccctggaggggaacagagtgagagagatgtttggctctggtacagcctgtgttgtttgcccagtttctgatatactgtacaaaggcgagacaatacacattccaactatggagaatggtcctaagctggcaagccgcatcttgagcaaattaactgatatccagtatggaagagaagagagcgactggacaattgtgctatcctgaatggaaaatagaggatacaatggaaaatagaggataccaactgtatgctactgggacagactgttgcatttgaattgtgatagatttctttggctacctgtgcataatgtagtttgtagtatcaatgtgttacaagagtgattgtttcttcatgccagagaaaatgaattgcaatcatcaaatggtgtttcataacttggtagtagtaacttaccttaccttacctagaaaaacattaatgtaagccatataacatgggattttcctcaatgattttagtgcctccttttgtacttcactcagatactaaatagtagtttattctttaatataagttacattctgctcctcaaacaaatgcaattttttgtgtgtgtttgaaagctaatttgagaaaatttcataggttacatttcctgcagcctatctttatccacagaaagtgttttcttttttttaaatcaagacttttaaaactggatttcctcccatcactgttttttgaaggtcctccaagtccgtgttaaggtaaatatctgttttcttcctgatgtcacagcctgagcatactctgtgcattaggaagacctgagtgcatttcccaccattgtcctttccacattatgttgtagctggctggctgtcaggcgactacaagactgagggtcttgtgccttatagatctttgtatcccccatggctgacatatagtaggtactcagtaaatggttttataatgaatcagtgaacattttgcttctatagaagtgtaccttctttgtttctatattatgaaacctctttattagaatttgtgattgattctgacagtgtatagatttaccttatattgtctttattttccatgagctactaagtcattagagatactctgaagcatagttagtttaggaaatcacttcatattgattgtattagaattatcttggaattgaagatatatccctagagcaggggaccccaacccccaggccatgggccacacagcaggaagaggtgagtggtgggccattgaggagcttcatctgtatttatggctacttcccatcactcgaattaccacctgaactccacctcttgtcagctcagtggcagcattagattctcataggagcacaaatcctattgtgaactctgcatgcaagggatctaggctatgcgctccttatgagaatctaatgcttgatgacctgaggtgtaacagtttcatcctgaaaccacccttcaccctgcagtctgtggaaaaattgtcttccacaaaactggtccctggtgccaaaaatgttggggaccactgctctagagagaggtcatgatatcataccaaccaaatggaaatgacaaatgttttatgtcaagtgttaattgcagaaataaatcttttttttttttttttggtagaaaacaaagaggcatactctgatttttatactctgtttttgcaggtgctcttttctttgaatggagatttgatgagcaagtggttaggatgcagggagagctactatgggtgatattttccttgtttaggagctgtgagttaaaattgtatcctttgtggtttatctaaggaaagtcaaatcttgacagaaaacatttttccttggaaggtcaactctcagacattgtattttggtttccctcagtcctcataacttccttcttgctgaacatattttattctcttttcagagaaggaaaataaaaaggattctaaaagtttgatgcattggaaaaatttccttgaggcatttagcaacacatagaaaatgggctttgattcttttccaaaacttttagccatagggtcttttatagacagggatagtaaaatgaaaattgagaaatataagatgaaaaggaatgataaaaatatcttttagggggcttttaattggtgatctgaaatcttgggagaagctgttcttttcaggcctgaggtgctcttgactgtcgcctgcgcactgtgtaccccgagcaacattctaagggtgtgctttcgccttggctaactcctttgacctcattcttcatatagtagtctaggaaaaagttgcaggtaatttaaactgtctagtggtacatagtaactaaatttctattcctatgagaaatgagaattatttatttgccatcaacacattttatactttgcatctccaaatttattgtggcgagacttgtccattgtgaaagttagagaacattatgtttgtatcatttctttcataaaacctcaagagcatttttaagcccttttcatcagacccagtgaaaactaaggatagatgtttaaaaactggaggtctcctgataaggagaacacaatccaccattgtcatttaagtaataagacaggaaattgaccttgacgctttcttgttaaatagatttaacaggaacatctgcacatcttttttccttgtgcactatttgtttaattgcagtggattaatacagcaagagtgccacattataactaggcaattatccattcttcaagacttagttattgtcacactaattgatcgtttaaggcataagatggtctagcattaggaacatgtgaagctaatctgctcaaaaagatcaacaaattaatattgttgctgatatttgcataattggctgcaattatttaatgtttaattgggttgatcaaatgagattcagcaattcacaagtgcattaatataaacagaactggtggcacttaaaatgataatgattaacttatattgcatgttctcttcctttcacttttttcagtttctacatttcagaccgagcttgtcagcttttttgaaaacacatcagtagaaaccaagattttaaaatgaagtgtcaagacaaaggcaaaacctgagcagttcctaaaaagatttgctgttagaaattttctttgtggcagtcatttattaaggattcaactcgtgatacaccaaaagaagagttgacttcagagatgtgttccatgctctctagcacaggaatgaataaatttataacacctgctttagcctttgttttcaaaagcacaaaggaaaagtgaaagggaaagagaaacaagtgactgagaagtcttgttaaggaatcaggttttttctacctggtaaacattctctattcttttctcaaaagattgctgtaagaaaaaatgtaagacaaaaaaaaaaaaaaaaaacaaacagaggcagaggcaggcagtagcaagaaagcagagcgtaacatcagctagatggtaacatgcaatgtcagctctcttgaagacatgggaaacctaagttacaccttgggttaaaattcttcaccatattagttttgttgcttcataaaatttacctaagcaagtggtcttgcttgcctcaaatccaagcagtcttgaacacttggaggcaattaatgagtatatcttagtcaaaagaattgttggagctttttattaaagctacagtttcagttctgcttttggggaattgtgctatgaaagcagctgccaaaataagctcatttattttcttcaatcccactcagtgctcagtcactatattctgtttcctttttttttttcaagttgcatatttggtttccccttatgattgggaaagatgaattttcagcagaaaacattgtttgttcactttcaaagagtgatagtttctaaaacatttagagcaataaatattcatcagaggtaccaagtaagccggcagaagagttaagggttagagaaatcccttatttcatgtcttgactctaaaattatcaaagtacttttccttgtaatgtggatttcttcttatgcggatatgcaaaaacttcagttatacgtagtaatgctagcaggtaattttagtagacattttataacaactgtcactttgtttcgccacatgtagagtttgttcagctattttccagatatctccccacaaaaggaggcaaagggtaccagcttttcaatgagcattacctattacttggcaaagatgatgaagactctattaatagttcatttgataaatgttgacataaccaacaatagagattaggaagttagttttaagaaatcaatggcatatagacattaccctcatggagtttgtattctactacttgaactgattgtagctataaaagcatagttagatagctgaatagttagatcataagcaaagaaggccagaacacatctcttatcaagaaatcaatgaatagtttatctcatttttaaagcaactttatccttctttaattccttcctttcttctagtgcaaaactacttaataaggttggtgtttaggttagtgttcacaccattcctcatctggtgtgaattaccttctctttctttactatttactaccaacctagtacatgtgttgactgaattcttttcaaacaatgttgagttatcatggtgcacctaataaattaacaccacagattacagcatccttgctgattttctcagcaaagccagattagatggaaataaacaaagaaaatgatcctagagtgaatttttctagaaaatatctattatgaaccatgctgtttaaagtattagcttgaaggtgatggatccagctattcagaaaataactttcatataaccatgattttgcacagtatgaggtcttaaatgtgtggaaagagataaattttttatcattaccacaaaccccttttaaagattcaaaggtggaagaaagtgatttattttttctcttcagcatacatatataaaagacttgtcagatgtttaatttggggaggttgataatgaaacatatcaacagagtatagtagttatagtagtgtttgtgggtaaataatttcctggggtcagacatatataaacatatttgcttcaaaatgataaaggcatgaaatcagtcttaaaaattgaaatgggggtgatgggggagaaaaagaagaacaaatttgaagtgccctttcaaatctgctggatacaagtattgaagttttaagtcatcttattctgtctgaaagtgtatttttcattctacaatagacccaatcaacaagacgtataacttgagttgcatgatgttcagtttatgtaatctactgttgggatggtaagaattgatgtaggctgtggtgtaagaatgaattaaaatatagtttcactggcttttctctacatatccactatcacaatggctaggtttcctgttgctcactattggattctggagaaaaatttaatgaaagatgatatcagaggaagaataagtggaggtagagaagaaaggaatgatagaggaggggaaaaaaacaaaacatatttttgtgttatccaaaggagctttttccttattctgtcaagcattgagatcttcttcagctttcaatgtagttgctaaatacaaataatgctactaggtagtgactaaatatagcaaacacttcatcagatattagaattaggtcacactattgaggttataatctgaaggttgtgttacatagaaaccactttagattattatcaacttggactaggctttattttataatagcatagtaagtaatatctattgtgtcatttcttcaaccattttattctaagatccatgaagcttcttgaggccaaataaaataataagtttagacaagaagtagattgtgacttttttcccttagagatactatttactatctcctatcctgataggtggaaggtttactgaattggaaattggttgactattagtttttaactaaaatgtgcaataacacattgcagtttcctcaaactagtttcctatgatcattaaactcattctcagggttaagaaaggaatgtaaatttctgcctcaatttgtacttcatcaataagtttttgaagagtgcagatttttagtcaggtcttaaaaataaactcacaaatctggatgcatttctaaattctgcaaatgtttcctggggtgacttaacaaggaataatcccacaatatacctagctacctaatacatggagctggggctcaacccactgtttttaaggatttgcgcttacttgtggctgaggaaaaataagtagttcgaggaagtagtttttaaatgtgagcttatagatagaaacagaatatcaacttaattatgaaattgttagaacctgttctcttgtatctgaatctgattgcaattactattgtactgatagactccagccattgcaagtctcagatatcttagctgtgtagtgattcttgaaattctttttaagaaaaattgagtagaaagaaataaaccctttgtaaatgaggcttggcttttgtgaaagatcatccgcaggctatgttaaaaggattttagctcactaaaagtgtaataatggaaatgtggaaaatatcgtaggtaaaggaaactacctcatgctctgaaggttttgtagaagcacaattaaacatctaaaatggctttgttacaccagagccatctggtgtgaagaactctatatttgtatgttgagagggcatggaataattgtattttgctggcaatagacacattctttattatttgcagattcctcatcaaatctgtaattatgcacagtttctgttatcaataaaacaaaagaatcctgtttgtgtggtttcatgaaa

SEQ ID NO: 32 - DNA, 2191 bp, Homo sapiens
cacgggcggagccggggccatggagccgccgctgccgggctaggcaggtcgtgccccgccgggccggcggcgatgtcgggctaccagcgccacccgggcgccaccccgctgtcccgagcccggagcctcgccattcccgacgctccagcgttctatgagcgccggtcttgtctcccccagctaaattgtgagcgcccccatggcagggacctggactcccccttcttcggcattcggccggcctttatgtgctatgtgcccagcccggtgctggcttccgtgggagacacagatgacagatttgaagatctggaagaggcaaatccattctcttttagagagtttctgaagaccaagaacctcggcctctcgaaagaggatccggccagcagaatttatgcaaaggaagcctcgaggcattccctgggacttgaccacaactccccaccctcccaaaccggcgggtatggcctggagtatcagcagccatttttcgaggatccgacaggggctggtgacctcctggatggggaggaggatgaggacaccggatggagtggggcctacctgccgtccgccatcgagcagactcaccccgagagggtccctgccggcacgtcgccctgcagcacatacctttcctttttctccaccccgtcggagctggcagggcctgagtctctgccctcgtgggcgttgagtgacactgattctcgcgtgtctccggcctctccggcagggagtcctagcgcagactttgcggttcatggagagtctctgggagacaggcacctgcggacgctgcagataagttacgacgcactgaaagatgaaaattctaagctgagaagaaagctgaatgaggttcagagcttctctgaagctcaaacagaaatggtgaggacgcttgagcggaagttagaagcaaaaatgatcaaggaggaaagcgactaccacgacctggagtcggtggttcagcaggtggagcagaacctggagctgatgaccaaacgggctgtaaaggcagaaaaccacgtcgtgaaactaaaacaggaaatcagtttgctccaggcgcaggtctccaacttccagcgagagaatgaagccctgcggtgcggccagggcgccagcctgaccgtggtgaagcagaacgccgacgtggccctgcagaacctccgggtggtcatgaacagtgcacaggcttccatcaagcaactggtttccggagctgagacactgaatcttgttgccgaaatccttaaatctatagacagaatttctgaaattaaagacgaggaggaagactcttgaggacccctgggtgttctcagcatgaagctccgtgtataccctgaggtcaccaccgctcgatctaaatgtgcagttgtgtccttaaatatgcagtcttcacccagagtaaagtgttgatcgcaagagtccagtgtcgtgccctcagccagttcttggccaccacaatgggagcagccctggccgagttgtctctgtggtttctatgcagcccttcttggcgaaattcctgcgatcttatagattctaatgagctcttggaagacattgtcataaaagccagtgattttaagaaaaagagtggttctggaatcagtgttttccagtcccatcccagaacatcagttgtaagataagtacaattggttgtccttgatttcataagtagaacaaacactaaatgtgcctctgagatggccaccccgggcagggacctgtgccttccaccgatgctcagggctccctctggctcccgggtcactcttgtggccccagtgggtggtccctgcagtcatggcctgagtgcgcaggggccaccgcgtggctgccgctgtcctcctccgggacccacggggaccaaggtcacacgttccgtgctgtgaagctgtccagatgtgcctctttggctgggggttctggtggacgtttcaagtggcattttgtacaatgcaggttagaattcaggaatttcaagtatgtgcccgggtctgtcaggtcccagttgcctttctgacggcccccctcagagggacggcgatgagcactaaatgcttttttgactattttcctatagattttttttaaaacttttttttcctcctgttccaattgatagctttcttatttaataaattctgtagttcaccaaaaaaaaaaaaaaaaa

SEQ ID NO: 33 - DNA, 463 bp, Homo sapiens
atatattcccagctagttgaaaatgatgattcccacaagaagcataactcagcttgtttctgcttactgagtattttctactatggtatatattgataacatttcttccattatgtatgttgtataccagagttacagttactgtgggaatcataatttgaaattttgactcctgtgtttctggaatctttacaacaaatgttgcattaacatataacttttttcagttgactttaccaaaattaagcccatctttagtagatactgttttaacatgtgaaagaaatacgttataaacataccacaagatatggctataaaacaatgagatcagtatccatttttgctttaaagaattggccttattgcttcagtgtcacatctcatactcaagggcatttactacaaagaaagagttctccaatattgctgttctgttgctgcctgccctatttacacatgt

SEQ ID NO: 34 - DNA, 393 bp, Homo sapiens
tttacacttatagtagactttatttagtgaatccaaatgacatgtgataattgtttggaaaggcctattgattttatatctgatcattcaatccagagacattaaattcagttgattaatggagttccccaactgtaagacttctttacgagattattttcaagctttgaaaagatcttctgagataaaggggatcagcaaacagtaagagtgtgttgctatacccaagcaaaagaaataaatcttaatctctcagcaaatcattcaaaatgtcagaaatgttagtgtttctatatcttggtaaaatggattgattgagaagtatgaaaagtataacagtggcatgcagaatattgtttttatgaatattcagaatttcagttgtttacataa

SEQ ID NO: 35 - DNA, 10434 bp, Homo sapiens
atggcgaaccggcgagtggggcgaggctgctgggaagtgagcccgaccgagcggaggccgcccgcggggctgcggggccccgcggccgaggaggaggcgtcttccccgccggtcctgtctctcagccacttctgcaggtctcctttcctttgcttcggggacgttctcctgggagcctcacggacgctgtctctggccctagacaaccctaacgaggaggtggcagaagtgaagatctcccacttcccggccgcggacctgggcttcagtgtgtcgcagcgctgtttcgtgttgcagcctaaagagaaaattgttatttctgttaactggacaccactcaaagaaggccgagtaagagagattatgacatttcttgtaaatgatgttctgaaacaccaagctatattactaggaaatgcagaagagcagaaaaagaaaaagaggagtctttgggataccattaaaaagaagaaaatttcagcctctacaagtcacaacagaagggtttcaaatattcagaatgttaataaaacatttagtgtttcccaaaaagttgacagagttaggagcccactacaagcttgtgaaaacttggctatgaatgaaggcggtcccccaacagaaaacaattctttaatacttgaagaaaataaaatacccatatcacctattagccctgctttcaatgaatgccatggtgcaacttgcttgccactctctgtacgtcgatctactacctactcatctcttcatgcatcagaaaatagggaactattaaatgtacacagtgccaacgtttcaaaagtttcttttaatgagaaagctgtaactgaaacttcctttaattctgtaaatgttaatggccaaagaggagagaatagtaaacttagtcttacccccaactgttcttcaactttgaacattacacaaagccaaatacattttctaagtccagattcttttgtaaataatagtcatggagctaataatgaactagaattagtaacatgtctttcatcagatatgtttatgaaagataattcacagcctgtgcatttggaatcaacaattgcacatgaaatttatcagaaaattttaagtccagattctttcataaaagataattatggactaaatcaggatctagaatcagagtcagttaatcctattttatcccctaatcaatttttaaaagataacatggcatatatgtgtacatctcagcaaacatgtaaagtaccattatcaaatgaaaattctcaagtcccacagtctcctgaagattggagaaaaagtgaagtttcgccacgtattcctgaatgtcagggttcaaaatctcccaaagctatttttgaagaactagtagaaatgaagtcaaattactacagttttataaaacaaaataatcctaaattttctgcagttcaggatatttctagtcatagccacaataaacaacctaagagacgtccaatactttctgccactgttactaaaaggaaggccacctgtaccagagaaaaccaaactgagattaataaaccaaaagcaaaaagatgtctcaacagtgcagtgggtgaacatgaaaaagtaataaataatcaaaaggaaaaagaagattttcattcttatcttccaattatagatccaatattaagtaaatctaagagttataaaaacgaggtaacaccctcttcgacaacagcttcagttgctcggaaaagaaagagcgatggaagcatggaagatgcaaatgtgagagttgcaattacagaacatacagaagtgcgagaaatcaaaagaatccatttttctccctcagagcctaaaacatcagctgttaagaaaacaaaaaatgtgacaacacccatctcaaaacgtattagcaacagagagaaattaaacctgaagaagaaaactgatttatcaatattcagaactccaatttctaaaacaaacaaaaggacaaaacccattatcgctgtggcacagtccagtttgaccttcataaaaccattaaaaacagatattcccagacacccgatgccatttgctgcaaaaaacatgttttatgatgaacgctggaaggaaaagcaggaacagggcttcacttggtggttaaattttatattaacccctgatgacttcactgtaaaaacaaatatttctgaagtaaatgctgctactcttcttttgggaatagagaatcaacataaaataagtgttcctagagcacctacaaaagaggaaatgtctctcagagcttatactgctcggtgtaggttaaacagactacgtcgtgcagcatgccgtttgtttacttctgaaaaaatggttaaagctattaaaaagcttgaaattgaaattgaagctaggcggttaattgttcgaaaagatagacacctatggaaagatgtgggagaacgtcagaaagtcctgaattggctgttgtcctacaatcctttgtggcttcgaattggtctagagacaacttatggagaactcatatctttggaagataacagtgatgtcacagggttggctatgtttattctgaatcgcctactttggaatcctgatatagcagctgagtatagacaccccactgttcctcacctgtatagagatggtcatgaagaagctttgtccaagtttacattgaaaaagttattgttgttggtctgttttcttgattatgctaaaatttccagactcattgatcatgatccttgtctcttctgtaaagatgccgaattcaaggctagtaaagaaatccttttggctttttcacgagatttcctaagtggtgaaggtgacctttcccgtcaccttggcttattgggattacctgttaaccatgttcagacaccatttgatgaatttgattttgccgttacaaatcttgccgtagacttgcaatgtggagtgcgccttgtgcgaaccatggaacttctcacacagaactgggacctctcaaagaaactcaggattccggcaataagtcgtcttcaaaagatgcacaatgttgacattgttcttcaagttcttaaatcacgaggaattgaattaagtgatgagcatggaaatacaattctatctaaggatattgtggataggcacagagaaaaaactctcaggttgctttggaaaatagcgtttgcttttcaggtggatatttcccttaacttagatcaattaaaggaagaaattgcctttctaaaacacacaaagagtataaagaaaacaatatctctactatcatgccattctgatgatcttattaataagaaaaaaggcaaaagggatagtggttcctttgaacaatatagtgaaaacataaagttattgatggattgggtaaatgctgtttgtgccttctataataaaaaggtggagaattttacagtgtctttctcagacggccgtgtgttatgttacctgatccaccattaccatccttgctatgtgccatttgacgctatatgtcagcgtactactcaaactgtggaatgtacgcaaactggttcagtggtattaaattcatcatctgaatctgatgacagttctctggatatgtcacttaaagcatttgatcatgaaaatacttcagagctatacaaagagctcctagaaaatgaaaagaaaaattttcacttggttaggtctgcagttagagaccttggtggaatacctgctatgattaatcattcagatatgtcaaatacaattccagatgaaaaggtggttattacctatttgtcatttctttgtgcaaggcttttggatcttcgtaaagaaataagagctgctcgactcatacaaacaacatggagaaaatataaactaaaaacagatctcaaacgccatcaggagagagagaaagctgcaagaattattcaattggctgtaatcaattttctagcaaaacaaagattgagaaaaagagttaatgcagcactcgtcattcagaaatattggcgaagagtcttagcacagagaaaattattaatgttaaaaaaggaaaagctggaaaaagttcaaaataaagcagcatcacttattcagggatattggagaagatattccactagacaaagatttctgaaattgaaatattattcaatcatcctgcaatctaggataagaatgataattgctgttacatcttataaacgatatctttgggctacagttacaattcagaggcattggcgtgcttatttaagaagaaaacaagatcaacaaagatatgaaatgctaaaatcatcaactcttataatccaatctatgttcagaaaatggaagcaacgtaaaatgcaatcacaagtaaaagctacagtaatattgcaaagagcttttagagaatggcatttaagaaaacaagctaaagaagaaaattctgctattatcatacaatcatggtatagaatgcataaagaattacggaagtatatttatattagatcttgtgttgttatcattcagaaaagatttcggtgctttcaagcccaaaagttatataaaagaagaaaagagtccatactaaccatccagaagtactacaaagcatatctgaaaggaaagattgagcgcaccaactatttgcagaaacgagctgcagccattcaattacaagctgcttttaggagactgaaagctcataatttatgtagacaaattagagctgcttgtgttattcagtcatactggagaatgagacaagacagagttcgatttttaaaccttaagaagactattatcaaatttcaggcacatgtaagaaaacatcaacaacgacagaaatataagaagatgaagaaagcagctgttataattcagactcatttccgagcttatatttttgccatgaaagttctagcatcttaccagaaaacacgctctgctgtcattgtgctgcagtctgcatatagagggatgcaagccaggaaaatgtatattcacatcctcacatctgttataaagattcaatcatattatcgtgcttatgtttctaaaaaggaatttttgagcctaaaaaatgctacaataaaattgcagtcaactgttaagatgaaacaaacacgtaaacaatatttgcatttaagagcagctgcactatttatccagcaatgttaccgttccaaaaaaatagctgcacaaaagagagaagagtatatgcagatgcgggaatcttgtatcaaactgcaagcatttgttagaggataccttgtccgaaagcagatgaggttacaaagaaaagctgttatttcactacagtcttatttcagaatgagaaaggctcggcagtattatctgaaaatgtataaagcaattattgtcattcagaattactatcatgcatacaaagcacaggtcaatcagaggaagaacttcttgcaagtcaaaaaagcagctacttgcttgcaagcagcttacagaggttataaagtacgccagctaatcaaacaacaatctatagctgctcttaaaattcagtctgcttttagaggctataataaaagggtaaaatatcaatctgtgcttcaatctataataaagattcagagatggtacagggcgtacaagactcttcatgatacaagaacacattttttgaagacaaaggcagctgtgatttccctccagtctgcttatcgtggctggaaggttcggaaacagattagaagggaacatcaagctgccttgaagattcagtctgcttttagaatggccaaggcccagaaacagtttagattgtttaaaacagcagcattagtcatccagcaaaatttcagagcatggactgcaggaaggaagcaatgtatggagtatattgaactccgtcatgcggtactggtgcttcaatctatgtggaagggaaaaacactgagaagacagcttcaaaggcaacataaatgtgctatcatcatacagtcatactatagaatgcatgtgcaacaaaagaagtggaaaatcatgaaaaaagctgctcttctgattcaaaagtattatagggcttacagtattggaagagaacagaatcatttatatttgaaaacaaaagcagctgtagtaactttacagtcagcttatcgtggtatgaaagtgagaaaaagaataaaggattgcaacaaagcagcagtcactatacagtctaaatacagagcttacaaaaccaaaaagaaatatgcaacctatagagcttcagctattataattcagagatggtatcgaggtattaaaattacaaaccatcagcataaggagtatcttaatttgaagaagacagcaattaaaatccaatctgtttatagaggtattagagttagaagacatattcaacacatgcacagggcagccacttttattaaagccatgtttaaaatgcatcagtcaagaataagttaccatacaatgagaaaagcagctattgttattcaagtaagatgtagagcatattatcaaggtaaaatgcagcgtgaaaagtacctgacaattttgaaagctgttaaagtccttcaggcaagttttagaggagtaagagttagacggactcttagaaagatgcagactgcagcaacactcattcagtcaaactacagaagatacagacagcaaacatactttaataagttaaagaaaataacaaaaacagtacagcaaagatactgggcaatgaaagaaagaaacatacaatttcaaaggtataacaaactgaggcattctgtaatatacattcaggctatttttaggggaaagaaagctagaagacatttaaaaatgatgcatatagccgcaactctcattcagaggagatttagaactctaatgatgagaagaagattcctctctctcaagaaaactgctattttgattcagagaaaatatcgggcacatctttgtacaaagcatcacttacagttccttcaggtacaaaatgcagttattaaaatccagtcatcatacagaagatggatgataaggaaaaggatgcgagagatgcacagggctgctactttcatccagtctactttcagaatgcacagattacatatgagatatcgagctttgaaacaggcctccgttgtgatccaacagcaataccaagcaaatagagctgcaaaactgcagaggcagcattatctcagacaaagacactctgctgtgatccttcaggctgcattcaggggtatgaaaactagaagacatttgaagagtatgcattcctctgcaacccttattcagagtaggtttagatcattactggtgaggagaagattcatttccctcaaaaaagctactatttttgttcagaggaaatatcgagccaccatttgtgccaaacataaattgtaccaattcttgcacttaagaaaggcagccattacaatacagtcatcttacagaagactgatggtaaagaagaagttacaagaaatgcaaagggctgcagttctcattcaggctactttcaggatgcacagaacatatattacatttcagacttggaaacatgcttcaattctaattcagcaacattatcgaacatatagagctgcaaaattgcaaagagaaaattatatcagacaatggcattctgctgtggttattcaggctgcatataaaggaatgaaagcaagacaacttttaagggaaaaacacaaagcttctatcgtaatacaaagcacctacagaatgtataggcagtattgtttctaccaaaagcttcagtgggctacaaaaatcatacaagaaaaatatagagcaaataaaaagaaacagaaagtatttcaacacaatgaacttaagaaagagacttgtgttcaggcaggttttcaggacatgaacataaaaaaacagattcaggaacagcaccaggctgccattattattcagaagcattgtaaagcctttaaaataaggaagcattatctccaccttagagcaacagtagtttctattcaaagaagatacagaaaactaactgcagtgcgtacccaagcagttatttgtatacagtcttattacagaggctttaaagtacgaaaggatattcaaaatatgcaccgggctgccacactaattcagtcattctatcgaatgcacagggccaaagttgattatgaaacaaagaaaactgcaattgtggttatacagaattattataggttgtatgttagagtaaaaacagaaagaaaaaactttttagcagttcagaaatctgtacgaactattcaggctgcttttagaggcatgaaagttagacaaaaattgaaaaatgtatcagaggaaaagatggcagccattgttaaccaatctgcactctgctgttacagaagtaaaactcagtatgaagctgttcaaagtgaaggtgttatgattcaagagtggtataaagcttctggccttgcttgttcacaggaagcagagtatcattctcaaagtagggctgcagtaacaattcaaaaagctttttgtagaatggtcacaagaaaactggaaacacagaaatgtgctgccctacggattcagttcttccttcagatggctgtgtatcggagaagatttgttcagcagaaaagagctgctatcactttacagcattattttaggacgtggcaaaccagaaaacagtttttactatatagaaaagcagcagtggttttacaaaatcactacagagcatttctgtctgcaaaacatcaaagacaagtctatttacagatcagaagcagtgttatcattattcaagctagaagtaaaggatttatacagaaacggaagtttcaggaaattaaaaatagcaccataaaaattcaggctatgtggaggagatatagagccaagaaatatttatgtaaagtgaaagctgcctgcaagattcaagcctggtatagatgttggagagcacacaaagaatatctagctatattaaaagctgttaaaattattcaaggttgcttctataccaaactagagagaacacggtttttgaatgtgagagcatcagcaattatcattcagagaaaatggagagctatacttcctgcaaagatagctcatgaacacttcttaatgataaaaagacatcgagctgcttgtttgatccaagcacattatagaggatataaaggaaggcaggtctttcttcggcagaaatctgctgctttgatcatacaaaaatatatacgagccagggaggctggaaagcatgaaaggataaaatatattgaatttaaaaaatctacagttatcctacaagcactggtgcgtggttggctagtacgaaaaagatttttagaacagagagccaaaattcgacttcttcacttcactgcagctgcatattatcacctgaatgctgttagaattcaaagagcctataaactttacctggctgtgaagaatgctaacaagcaggttaattcagtcatctgtattcagagatggtttcgagcaagattacaagaaaagagatttattcagaaatatcatagcatcaaaaagattgagcatgaaggtcaagaatgtctgagccagcgaaatagggctgcatcagtaatacagaaagcagtgcgccattttctcctccgtaaaaagcaggaaaaattcactagtggaatcattaaaattcaggcattatggagaggctattcttggaggaagaaaaatgattgtacaaaaattaaagctatacgactaagtcttcaagttgttaatagggagattcgagaagaaaacaaactctacaaaagaactgcacttgcacttcattaccttttgacatataagcacctttctgccattcttgaggccttaaaacacctagaggtagttactagattgtctccactttgttgtgagaacatggcccagagtggagcaatttctaaaatatttgttttgatccgaagttgtaatcgcagtattccttgtatggaagtcatcagatatgctgtgcaagtcttgcttaatgtatctaagtatgagaaaactacttcagcagtttatgatgtagaaaattgtatagatatactattggagcttttgcagatataccgagaaaagcctggtaataaagttgcagacaaaggcggaagcatttttacaaaaacttgttgtttgttggctattttactgaagacaacaaatagagcctctgatgtacgaagtaggtccaaagttgttgaccgtatttacagtctctacaaacttacagctcataaacataaaatgaatactgaaagaatactttacaagcaaaagaagaattcttctataagcattccttttatcccagaaacacctgtaaggaccagaatagtttcaagacttaagccagattgggttttgagaagagataacatggaagaaatcacaaatcccctgcaagctattcaaatggtgatggatacgcttggcattccttattag

SEQ ID NO: 36 - DNA, 3581 bp, Homo sapiens
atacgtggctgccgtctgtccccgctgaggaggtgcagcagccggagatggcggcggtgctgaacgcagagcgactcgaggtgtccgtcgacggcctcacgctcagcccggacccggaggagcggcctggggcggagggcgccccgctgctgccgccaccgctgccaccgccctcgccacctggatccggtcgcggcccgggcgcctcaggggagcagcccgagcccggggaggcggcggctgggggcgcggcggaggaggcgcggcggctggagcagcgctggggtttcggcctggaggagttgtacggcctggcactgcgcttcttcaaagaaaaagatggcaaagcatttcatccaacttatgaagaaaaattgaagcttgtggcactgcataagcaagttcttatgggcccatataatccagacacttgtcctgaggttggattctttgatgtgttggggaatgacaggaggagagaatgggcagccctgggaaacatgtctaaagaggatgccatggtggagtttgtcaagctcttaaataggtgttgccatctcttttcaacatatgttgcgtcccacaaaatagagaaggaagagcaagaaaaaaaaaggaaggaggaagaggagcgaaggcggcgtgaagaggaagaaagagaacgtctgcaaaaggaggaagagaaacgtaggagagaagaagaggaaaggcttcgacgggaggaagaggaaaggagacggatagaagaagaaaggcttcggttggagcagcaaaagcagcagataatggcagctttaaactcccagactgccgtgcagttccagcagtatgcagcccaacagtatccagggaactacgaacagcagcaaattctcatccgccagttgcaggagcaacactatcagcagtacatgcagcagttgtatcaagtccagcttgcacagcaacaggcagcattacagaaacaacaggaagtagtagtggctgggtcttccttgcctacatcatcaaaagtgaatgcaactgtaccaagtaatatgatgtcagttaatggacaggccaaaacacacactgacagctccgaaaaagaactggaaccagaagctgcagaagaagccctggagaatggaccaaaagaatctcttccagtaatagcagctccatccatgtggacacgacctcagatcaaagacttcaaagagaagattcagcaggatgcagattccgtgattacagtgggccgaggagaagtggtcactgttcgagtacccacccatgaagaaggatcatatctcttttgggaatttgccacagacaattatgacattgggtttggggtgtattttgaatggacagactctccaaacactgctgtcagcgtgcatgtcagtgagtccagcgatgacgacgaggaggaagaagaaaacatcggttgtgaagagaaagccaaaaagaatgccaacaagcctttgctggatgagattgtgcctgtgtaccgacgggactgtcatgaggaggtgtatgctggcagccatcaatatccagggagaggagtctatctcctcaagtttgacaactcctactctttgtggcggtcaaaatcagtctactacagagtctattatactagataaaaatgttgttacaaagtctggagtctagggttgggcagaagatgacatttaatttggaaatttctttttacttttgtggagcattagagtcacagtttaccttattgatattggtctgatggtttgtgaactcttgctgggaatcaaaatttccttgagactctttagcattcatactttggggttaaaggagattcctcagactcatccagcccttgggtgctgaccagcagagtcactagtggatgctgaagttacatgagctacatgttaaatatttaaagtctccaaaataaaacaccccaacgttgaccttacccggctgatggttagccccttgctgcctgctccatgtgtcttatgagagcccgtagttacagtgtcctctaatttgaaatccataagttaacaagtctatatcaggtgcagctggctttgattaaaggccatttttaaaacttaaaaactcaacacctcacagattataatagaaaaagaaatggcctcagtttgatctcgttcagaatgacccagattgtttctgctttgggtgcagctgtttagttcagagttatattacagagaattattttctgagataatcttaaactagaatgttcaaaactaattgataattgaagtatcaagatacgtagaacacctcagagatttttcttcaggaacttccacaaactttgaatccttgtatctttatttggtattcatactactagtagcaaaatacaggttttttgttttgttttgttttgttttggcttcatagagtatctcaaattgaaacttttctgcacaaagaataaaattaaggattttataaactcaaattggcacctactgaattaaaatacataaaatcatttaaatataattcagcatatgggaagtaacattgcactaatatggaaatcactgccagagacagtctattttcttttaatttgttactacttagtcacaaaccccacattattccagtttggaattacttattaaggagaattggaaatacatatgcccatgcttaaattttatagctttaatttgtgttatttctttattgacgggaagaggtacatctttttttccttactgaaaacaaatatggattaattgcctcaaatttgtataagtgattggctagtgattcttgttttcagaagggagagtggtatagatagaaaatgacaaagatggcaatatacacttaatgttgttattgtatgttgttactgaagtacttagatttttaaaatttcaaatcctaaatcacttcttgtaggagggttttcattaactgcagtatatacagttcactacatatgggttgtttgagttttttgtgtgctgtatttctttctgttttttaatacctggttttgtacatatctaactctgttctcttttggttgttcagaaactggattttttttttcttaagcagtgcttaatttgtgttttttaattttgattcagaagtagtcccagctcataggtgttcatactgttacatccagaacatttgtcaggctctctgtcagctttcatgtacatatggtatagaaaccatggagttaggcacttcctggattttttttttatgagaaaaatactgtatttaaaatgtaaaataaacttttaaaaagcaggcactaatatatatttcttccagcctttgattacaaatttgtccttgcacatgttaagatgaattatctcctaaaaatatcattgttcttgggagcagtgtatgttactttacatagcagcggttcctgtcatgtgttcatgtcagaatatttttggttttaaactttcttattgcctttggctgttgattagtacagtacaagtgcgatttcaaaaagatcttgaaagtaatatatttaatcaattaaaatgtttatctgtaaaaaaaaaaaaaaaaaa

SEQ ID NO: 37 - DNA, 609 bp, Homo sapiens
  misc_feature (565)..(565): n is a, c, g, or t
tttttttacagttttcaaatattttactgaaaatgcatattgtacaattaatgtataatgacacaccagtgtgagaaacctccataggtatcatttccacaaatatgctatgaatatagagttcctacacaaaactatacaacttaccagatgtaattcctgttacgtaccatactcacaatcgtcttgaagaatatggagaaaaagtgctgagtgacaaaaacaggagccatgtgtgattttaataaatggaaaacacggcatttcagctcagtggtaaagcagtaaaccaatcagatgcttagctatcaagtaatcatgtgagaggaaacagaattagatcctacctcatactatatgttgtcagctaacactgtagcagtggtatatgaatcactaaattacctccaacaaaatgtattcctgtattgaaaaaaggaggtatggccaacattgtgtcacgttccaaggtgaattttgcggtcacgatatgacgttcaggaagctacttttattgttcagttgatttctatgctcaactattaggtcaattccgaaataatcncatatcacagctaaaataatgnctaccaagtcnctctgactgct

SEQ ID NO: 38 - DNA, 2216 bp, Homo sapiens
ctgttagcaatgcttcctgatgttgtgcgtggccctttttggttgattctctccaaattcgggtcagctgctgccacctggcaaataacagaggatatgctgaatctcctgtccatccttgtaacgatatccttcttaatgaaattcttcaactggctgagcaattacaaatgtcatctgtccagacacatgggcttaaggatgtctacaaaattttagacatttttgcaaatgggaaaaaaaatagtcttgtaaatactgaaacagatttccatgaactttatcctactcttggaaagaaaacaattctccttggctgcagaaatcaaataagctgggtttgcaatgaccaaggacataaatgaagatggattgaagtggaaaaattctgtctcccaagtgatcagtgacatctgccagaggtcattacagctacttttaactgtgaacagtcaccagctaaactactcacttgccacaacaaaataacctctctcaaagtaaatccagtgcatctgtatatatgtgtagatagcagcaacaaacaatcctgaaacattatttttggctgttaggtaagtaaacgtgatgataattataaacaacattcaaataaccttggaccttggtgaaatgacttgtggtggccagaatggtgcaacaagatgttatttgcaagtttttttaagacacaaatatctcagatactaataatgagaataaagactgttgaatatgaaattaaagccaagcaataatgtgccaaaaagaggcagttataccagcaaatgcatctattatgggcacaccattatataatgatggtttgctttatgaagactgactgtaacccacaggataaaataagcaaaggcatagtttctgctttcttcctggaaaaacttgtttagaagcttcataaagaggtacagcactaatgagcattagtcaggatacagttggcatctatgtttttatgtgagcccagagggaagaggagccactcaaagtcttgctggtttaaaactcaagacagctgcaaccagaagttttgttgaaatggagactttaaacttatggtaattactctttctggacactagcatgtagaaagcaattcagttaactctgcccagaggattaccagctttagctgtgaaaaaatgggctcccggatgtaaaatcactaaaacatgagatcttgtatccaaagaggcttcaaatgatgccttacagaaaacgatgctccagatgggcacttctaaatgctaactcttcatcaagtatctttctggattcaagctcaaaattaattggctgcaaaatagtaggaataaaaatcacatattttacactttagaaaaggatattgatgatcaacctgcatggtgataattatgatgagataccccagtgatttaatgatgttagaaagaattaaatgggagagaattgctaacagctttcttgatctcttaactatggagatgtcattcatttatttctggggtgaaaattatagcttgctttttgacattgctgctagtattgttctttgttgctttaaaaattgtctctctttagaaaaactcttgagcagttaaacagttctttttctgattcatatcattgcttttaataacatgtaaaggctgtgtgtagagcaaactatataaaatgagtagaaagggcttgctcatgttaattggcatccttgatgattttagttgagattccttaacatttattttagatcacatctttacgtaacttatttttcctaatgttttccatcgtgtcttaaaatgatgctggtatatcaggagattgcagtattatagtcatactccccaatccctagaggagaggaaagactaattcttgttttaagggcccctggagataccttttattaaggttgaaaaaggtcaacacagcctgaaaataagaaaaatatatactagcaattactaattttctaaatgtgtgtatctctgctgtactaatgtgtgaacaatatgtcgtgcataatactgtagctggtcgtggtatgtcaatacattctgtgagtgtgtacagtctgagtgatcagttttctatttttatgtgtaaaaaaaataacttgtcgtatcccatttaaaggccaatttctgtattcaggcaggcatatgtacatacatgaataaagccaacaaaagtgtgcacatgtattcagt

SEQ ID NO: 39 - DNA, 1705 bp, Homo sapiens
aattcggcacgagaagacttccagtttggagtcgtttgctgcggggagggaatgaatgggcgctgggaacacgcccgcgaggtggggacgcgccggccgtagcgaggtccttagcgtgtgagtggccggggtcgggtcgcttccccgcagcatggaggacgatgcaccagtgatctacgggctggagttccaggcacgtgccttaacacctcaaactgcagaaacagatgccattcggtttttggttgggacgcagtctcttaaatatgataatcagatccatatcatagattttgacgatgaaaacaacattataaataaaaatgtcctcctccatcaagcgggtgaaatctggcatattagcgctagccctgcagacagaggtgtgctgacgacctgctacaacagaacttcagacagcaaagtcctgacatgtgcagccgtgtggaggatgccgaaggaattggaatcaggcagccacgagtcccctgatgattcatccagcactgcacagaccctggagctgctctgtcaccttgacaacacagcccatggcaacatggcctgtgtcgtgtgggagccaatgggagatgggaagaaaatcatttccttggctgataaccatatcctgctgtgggatttacaggaaagctcgagccaggctgtgctggccagctcagcgtccctggaagggaagggacaactgaagttcacctcaggacggtggagcccacatcataactgcacccaggtggccacagcgaacgacaccaccctccgtggctgggacacccggagcatgagccagatctactgcatagagaatgcccacggacagctggtgcgggaccttgactttaatcccaataagcagtactacttggccagctgcggagacgactgtaaggtgaagttctgggacacccgaaatgtcaccgaacccgtgaagaccctggaggagcactcccactgggtgtggaacgtccgctacaaccactctcatgaccagctggtcctcacgggcagcagtgacagcagagtcatcctttccaacatggtgtccatctcgtcggagcccttcggccacttggtagacgacgatgacatcagtgaccaggaggaccaccgttctgaagagaagagcaaggagcccctgcaggacaacgtgatcgccacctacgaggagcacgaggacagcgtctatgccgtggactggtcctcggctgacccgtggctgtttgcctccctgagctatgacgggaggctcgtgatcaacagggtgcccagggccctgaagtaccacatcctgctatgactcccgggcctgggttatccaggtcccattgagtggttttcctcttggcagattctcaaacagtcgcagctctttggaggtgactcgtgttccaggtggatccctctctgggagagccgctgttcccttcctgtagcagcagcatttatgaatggggtgaatggggctattgtcgacggcacagctaatgcccgaacccagcccctgtcggcagagacagagccccacattattatgtgaataacaatgttttctgttttaagggtgtcaggagtttcgctttttaaaaaaatgtctgttcctgcagtagtaactcttctttctcttgagagtaaaaaatgaaataaaataaatccacgctgacaaaaaaaaaaaaaaaaaaaaaaaaaa

SEQ ID NO: 40 - DNA, 1800 bp, Homo sapiens
gaggaagatggcggcgtccgcagctgccgctgagctccaggcttctgggggtccgcggcacccagtgtgtctgttggtgttgggaatggcgggatccgggaaaaccacttttgtacagaggctcacaggacacctgcatgcccaaggcactccaccgtatgtgatcaacctggatccagcagtacatgaagttccctttcctgccaatattgatattcgtgatactgtaaagtataaagaagtaatgaaacaatatggacttggacccaatggcggcatagtgacctcactcaatctctttgctaccagatttgatcaggtgatgaaatttattgagaaggcccagaacatgtccaaatatgtgttgattgacacacctggacagattgaggtattcacctggtcagcttctgggacaattatcactgaagcccttgcatcctcatttccaacagttgtcatctatgtaatggacacatcgagaagtaccaacccagtgaccttcatgtccaacatgctctatgcctgcagcatcttatacaaaaccaagctgcctttcattgtggtcatgaataaaactgacatcattgaccacagctttgcagtggaatggatgcaggattttgaggctttccaagatgccttgaatcaagagactacatacgtcagtaacctgactcgttcaatgagcctggtgttagatgagttttacagctcactcagggtggtgggtgtctctgctgttctgggtactggattagatgaactctttgtgcaagttaccagtgctgccgaagaatatgaaagggagtatcgtcctgaatatgaacgtctgaaaaaatcactggccaacgcagagagccaacagcagagagaacaactggaacgccttcgaaaagatatgggttctgtagccttggatgcagggactgccaaagacagcttatctcctgtgctgcacccttctgatttgatcctgactcgaggaaccttggatgaagaggatgaggaagcagacagcgatactgatgacattgaccacagagttacagaggaaagccatgaagagccagcattccagaattttatgcaagaatcgatggcacaatactggaagagaaacaataaataggagactttagcacacttcacttgtttctagaagtccagaattttggacctccacgtgaaagaactgttcttacctctgaactgggggctcccataagggataattttcctcagagtagcaaagtttctcttattagagaaatcttgtgactcagatgaagtcagggatagaagacccttggacctggcaggttaatgctgattattccttggcctttcccttgtatttatgcaaggaaggatatactgagctgatactgttccaagcctacaacttcaagttttatcatttgaactcaagtacttttgctgctgaggaatggaatcaaaagaacgtagtctcctggtgaccacctcagatctctattattaggctagatgtatagcctctactcccccagcttcttgctcttgaccctgcactgtaagttgcccttctattagcagccaaggaaaagggaaacatgagcttatccagaacggtggcagagtctccttggcaatcaaccaacgttgctatgaaatatgcctcacactgtatagctcattataggacgtcaggtttgttgaaaaaagtgggcaagacatgattaatgaatcagaatcctgtttcattggtgacttggataaagactttttaattttaaaaaaaaaaaaaaaaaaaaaaaa

SEQ ID NO: 41 - DNA, 2297 bp, Homo sapiens
ggctgaggcgcgatggcaggtgtcggggctgggcctctgcgggcgatggggcggcaggccctgctgcttctcgcgctgtgcgccacaggcgcccaggggctctacttccacatcggcgagaccgagaagcgctgtttcatcgaggaaatccccgacgagaccatggtcatcggcaactatcgtacccagatgtgggataagcagaaggaggtcttcctgccctcgacccctggcctgggcatgcacgtggaagtgaaggaccccgacggcaaggtggtgctgtcccggcagtacggctcggagggccgcttcacgttcacctcccacacgcccggtgaccatcaaatctgtctgcactccaattctaccaggatggctctcttcgctggtggcaaactgcgtgtgcatctcgacatccaggttggggagcatgccaacaactaccctgagattgctgcaaaagataagctgacggagctacagctccgcgcccgccagttgcttgatcaggtggaacagattcagaaggagcaggattaccaaaggtatcgtgaagagcgcttccgactgacgagcgagagcaccaaccagagggtcctatggtggtccattgctcagactgtcatcctcatcctcactggcatctggcagatgcgtcacctcaagagcttctttgaggccaagaagctggtgtagtgccctctttgtatgacccttcctttttacctcatttatttggtactttccccacacagtcctttatccacctggatttttagggaaaaaaatgaaaaagaataagtcacattggttccatggccacaaaccattcagatcagccacttgctgaccctggttcttaaggacacatgacattagtccaatctttcaaaatcttgtcttagggcttgtgaggaatcagaactaacccaggactcagtcctgcttcttttgcctcgagtgattttcctctgtttttcactaaataagcaaatgaaaactctctccattaccttctgctttctctttgtccacttacgcagtaggtgactggcatgtgccacagagcaggccctgcctcactgtctgctggtcagttctgggttcacttaatggctttgtgaatgtaaataaggggcaggtcttggccctagaggattgagatgtttttctatatcttagaactatttttggataaattatatattttccttcctagtagaagtgttactgcctgtaactagctcaaaataccaatgcagtttctgcattctgggttttgtttttcctttttttttttttttttttttttttgagttttgctctcgtcgcccaggctggagtgcaatggcgtgatctcagctcactggcaacatctgcctcccgggttcaaatgattctcctgcctcagtctcctgagtagctgggattacaggtgcccgccaccacgctcagctaatttttgtatttttagtagagatggggttttaccatgttggccaggctggtcttagactcctgacctcagttgatccacctgcctcagcctctgcattcagtttattcacatatttttggtaactcccatggcagctcctaggatttcagcggtctgtgggccagaaagcaggcaccagggctgacctcaaggccgtatcagagggccaagcagagttcttttggatacctgcttttcatcccacagggccttagagtcagaggtaaggtagcaacagagctagaatggggcaatgcactcttaccctccttctcaacttttatttaagctgtgctaaatgttttcttcaagggaaccagatttagttctttacagaattttccagtgaaataaaacatgttgtaatagctgtgtttgagatgaaataagaggttgtgggtagaggggaggcacctaaaggaaaagaggaaaggtgcctgggctacctatgcagataacctggagtggacttcactgtggactcgtggtactaaggcttggcctggacaggcagtctagggggtatgggaatacacggtgtggttgttcaactatttgcaaaggtcaaccaaatagaccacatgttcgcaaagtatcatctgaggaaattaagtaccttcttagccctctcagtcataaatttgaacaaattttaatacacttccctcatgcccttctatataaaacttaataccattagttccccattcttgacattttatttcagtttttattatatatttatttgaaatatttattaaattatctgacctacagaactaaaaaaaaaaaaaaaaaa

SEQ ID NO: 42 - DNA, 653 bp, Homo sapiens
  misc_feature (621)..(621): n is a, c, g, or t
gggacatttccaagggtatttaaactctcactctgccacctttctaagggtgggaggctggcagagatgctgcaatgcttgataatcatttggccacactgaaatttccaaagggagctcttgccggtgcttaaaaccaaaactcctggacacttagaaaattccatgaatctagcacaaaatatccattcttgcccaagtgtatcccctttctctccagcttaatctttttttttttttttttttaaagcccaggccaagggtacttttaactggaaactggggaggagggaagaacactagcagggagctaagaggcaggttgctgggtaagccatcctgctcctacctggtgcctgtatctacattgctgagtgctgtgcgccagtgcctttccttcatctgcagatggagcccatctctttccacctgggtgaggagaccctctgctactccaggggtaaaccttaaagaaggtgtcttgaagagcccaaaggacactcacgtgctaaggtgtccattttatgcatctttaaaatattttatttaaaaaaaaaaatagccctgccctgtcttagtgccactaacggcccagattcattcattctgaatggaaaaacngagactgccagcactttcctttggtccttccn

SEQ ID NO: 43 - DNA, 1955 bp, Homo sapiens
catggaggcgctgctgctgggcgcggggttgctgctgggcgcttacgtgcttgtctactacaacctggtgaaggccccgccgtgcggcggcatgggcaacctgcggggccgcacggccgtggtcacgggtgagtgcggaggcgggtgagtgcgagctggcggggcgcgcggagaggaggccgggccggcggtagcagcggcccgccgggctcagctcagctcggctcccgcccgcggtccgcaggcgccaacagcggcatcggaaagatgacggcgctggagctggcgcgccggggagcgcgcgtggtgctggcctgccgcagccaggagcgcggggaggcggctgccttcgacctccgccaggagagtgggaacaatgaggtcatcttcatggccttggacttggccagtctggcctcggtgcgggcctttgccactgcctttctgagctctgagccacggttggacatcctcatccacaatgccggtatcagttcctgtggccggacccgtgaggcgtttaacctgctgcttcgggtgaaccatatcggtccctttctgctgacacatctgctgctgccttgcctgaaggcatgtgcccctagccgcgtggtggtggtagcctcagctgcccactgtcggggacgtcttgacttcaaacgcctggaccgcccagtggtgggctggcggcaggagctgcgggcatatgctgacactaagctggctaatgtactgtttgcccgggagctcgccaaccagcttgaggccactggcgtcacctgctatgcagcccacccagggcctgtgaactcggagctgttcctgcgccatgttcctggatggctgcgcccacttttgcgcccattggcttggctggtgctccgggcaccaagagggggtgcccagacacccctgtattgtgctctacaagagggcatcgagcccctcagtgggagatattttgccaactgccatgtggaagaggtgcctccagctgcccgagacgaccgggcagcccatcggctatgggaggccagcaagaggctggcagggcttgggcctggggaggatgctgaacccgatgaagacccccagtctgaggactcagaggccccatcttctctaagcaccccccaccctgaggagcccacagtttctcaaccttaccccagccctcagagctcaccagatttgtctaagatgacgcaccgaattcaggctaaagttgagcctgagatccagctctcctaaccctcaggccaggatgcttgccatggcacttcatggtccttgaaaacctcggatgtgtgcgaggccatgccctggacactgacgggtttgtgatcttgacctccgtggttactttctggggccccaagctgtgccctggacatctcttttcctggttgaaggaataatgggtgattatttcttcctgagagtgacagtaaccccagatggagagataggggtatgctagacactgtgcttctcggaaatttggatgtagtattttcaggccccacccttattgattctgatcagctctggagcagaggcagggagtttgcaatgtgatgcactgccaacattgagaattagtgaactgatccctttgcaaccgtctagctaggtagttaaattacccccatgttaatgaagcggaattaggctcccgagctaagggactcgcctagggtctcacagtgagtaggaggagggcctgggatctgaacccaagggtctgaggccagggccgactgccgtaagatgggtgctgagaagtgagtcagggcagggcagctggtatcgaggtgccccatgggagtaaggggacgccttccgggcggatgcagggctggggtcatctgtatctgaagcccctcggaataaagcgcgttgaccgccaaaaaaaaaaaaaaaaaaaaaaaaaaaaaaaaaaaaaaaaaaaaaaa

SEQ ID NO: 44 - DNA, 3098 bp, Homo sapiens
tcctgcttgtcggcatcgctccccacaggccgacgtcgagagggcctgctttactcctcctctttctcctccttctcccgcggcttctgcgcggagaggcgtcgcccgggatctgggttttggaagaaggatctttgtgggaagacagggtgaatttatcacagaggaataacgagggagaggagaaaggtttcctaaagacaaaaaaaaaaatggaggaatctgtaaaccaaatgcagccactgaatgagaagcagatagccaattctcaggatggatatgtatggcaagtcactgacatgaatcgactacaccggttcttatgtttcggttctgaaggtgggacttattatatcaaagaacagaagttgggccttgaaaatgctgaagctttaattagattgattgaagatggcagaggatgtgaagtgatacaagaaataaagtcatttagtcaagaaggcagaaccacaaagcaagagcctatgctctttgcacttgccatttgttcccagtgctccgacataagcacaaaacaagcagcatttaaagctgtttctgaagtttgtcgcattcctacccatctctttacttttatccagtttaagaaagatctgaaggaaagcatgaaatgtggcatgtggggtcgtgccctccggaaggctatagcggactggtacaatgagaaaggtggcatggcccttgctctggcagttacaaaatataaacagagaaatggctggtctcacaaagatctattaagattgtcacatcttaaaccttccagtgaaggacttgcaattgtgaccaaatatattacaaagggctggaaagaagttcatgaattgtataaagaaaaagcactctctgtggagactgaaaaattattaaagtatctggaggctgtagagaaagtgaagcgcacaagagatgagctagaagtcattcatctaatagaagaacatagattagttagagaacatcttttaacaaatcacttaaagtctaaagaggtatggaaggctttgttacaagaaatgccgcttactgcattactaaggaatctaggaaagatgactgctaattcagtacttgaaccaggaaattcagaagtatctttagtatgtgaaaaactgtgtaatgaaaaactattaaaaaaggctcgtatacatccatttcatattttgatcgcattagaaacttacaagacaggtcatggtctcagagggaaactgaagtggcgccctgatgaagaaattttgaaagcattggatgctgctttttataaaacatttaagacagttgaaccaactggaaaacgtttcttactagctgttgatgtcagtgcttctatgaaccaaagagttttgggtagtatactcaacgctagtacagttgctgcagcaatgtgcatggttgtcacacgaacagaaaaagattcttatgtagttgctttttccgatgaaatggtaccatgtccagtgactacagatatgaccttacaacaggttttaatggctatgagtcagatcccagcaggtggaactgattgctctcttccaatgatctgggctcagaagacaaacacacctgctgatgtcttcattgtattcactgataatgagacctttgctggaggtgtccatcctgctattgctctgagggagtatcgaaagaaaatggatattccagctaaattgattgtttgtggaatgacatcaaatggtttcaccattgcagacccagatgatagaggcatgttggatatgtgcggctttgatactggagctctggatgtaattcgaaatttcacattagatatgatttaaccataagcagcagcacgatccagagatccattgccatcagtgatctcactaaaaatatacagctacttcccagctaatctccacccaatgaatgatgatggtatagtatgtgcataatggaaagttaccttactgaaaaaaaaaaaagaaggaaaaataagatgggcccaaaggtctatctactaaactagctcttggggaaatagcttcaggatactgtagtttcctctatctaatagagaactttttgttaacagacactgtaaaatagttttgctttgttgaataatacatgtgtacctaaaagaggtaagagcaaaaagtgtaattccacatcatgttacttgagaagtgcttaacgttttcttaaatgttttcattgggaaaggacagctttgataatgtccaaatactctgaaatgcactagaccatataactgtgatgaaatatgaaactcatctgtaaacttttataccaagggggtaaaaaaaaaaactaaggcatttgattaaattatgaatgagttttacaaattcctttcagagttttactaagatcacacaaataacagctttcttattcagtgaaaaagatattttatttctgatgttttatttgcactcgtggaatatgttaccattaatcagaaacatcatggcaacccctaagaatagactaagtttgtgttggctgagggattctatttggtttgcttttttttttttgctttgttatattttattgctacaaggggtgtgacttgataatgatttcctctgaattataataacatagccagatgtagtctcacactgtttttcatactcttaagtgtaaataatataaaatgtttcaagcgcttaactccccctcattcacaaagtataacaattaaaatctcaactataaccagtttagctttttccttacttttaaaataaaattttttacttttaactatttttttagttaatatttttaaaagtatacatgtcaatggcctctttgtccattattcattttgtggcaaaatattcttctttgatagtgtaaacaaataataaagcaatctaggtcctttaggtttgaaaggcaatttttgagtagcatattaccagctagccagtcactaggaatttttttcagtattatttgtatgtattaaacttttcattacactaaagtgcattattttattgagcaagtatccttcattgtgaggtttgacattaaagcaatctgttgaaatgccaaaaaaaaaaaaaaaa

SEQ ID NO: 45 - DNA, 2689 bp, Homo sapiens
gatcttgggctgaggttcccgggcgggcgggcgcggagagacgcgggaagcaggggctgggcgggggtcgcggcgccgcagctagcgcagccagcccgagggccgccgccgccgccgcccagcgcgctccggggccgccggccgcagccagcacccgccgcgccgcagctccgggaccggccccggccgccgccgccgcgatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaggggcatgcctgtgcccccatgggttttcttttttcttttttcttttttttggtcgggggggtgggagggttggattgaacagccagagggccccagagttccttgcatctaatttcacccccaccccaccctccagggttagggggagcaggaagcccagataatcagagggacagaaacaccagctgctccccctcatccccttcaccctcctgccccctctcccacttttcccttcctctttccccacagccccccagcccctcagccctcccagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctccccggggaatcctctctgccaatcctgcgagggtctaggcccctttaggaagcctccgctctctttttccccaacagacctgtcttcacccttgggctttgaaagccagacaaagcagctgcccctctccctgccaaagaggagtcatcccccaaaaagacagagggggagccccaagcccaagtctttcctcccagcagcgtttccccccaactccttaattttattctccgctagattttaacgtccagccttccctcagctgagtggggagggcatccctgcaaaagggaacagaagaggccaagtccccccaagccacggcccggggttcaaggctagagctgctggggaggggctgcctgttttactcacccaccagcttccgcctcccccatcctgggcgcccctcctccagcttagctgtcagctgtccatcacctctcccccactttctcatttgtgcttttttctctcgtaatagaaaagtggggagccgctggggagccaccccattcatccccgtatttccccctctcataacttctccccatcccaggaggagttctcaggcctggggtggggccccgggtgggtgcgggggcgattcaacctgtgtgctgcgaaggacgagacttcctcttgaacagtgtgctgttgtaaacatatttgaaaactattaccaataaagttttgtttaaaaaaaaaaaaaaaaaa

SEQ ID NO: 46 - DNA, 1936 bp, Homo sapiens
ccggagcctcctggaccaggagaactgtaacgcgagccccgagccatgggcgaaaggcggggccgagacgggttgggggcgccgacggtttcccggccctggctgcagcttggaggagaagctgagcctgtgcttccgcccctcggatccgggcgccgagcccgaggacggccgtgcggccatcacggagctcaactcctgcagggggacgagatttggaatgccctgacagataattatgggaatgtgatgcctgtagactggaagtcatcgcatactaggaccttgcacttgcttactctgaacctctcagaaaaaggggtaagtgacagtttgctctttgatacatcagatgatgaagagctgagagaacagctggatatgcactcaatcatcgtctcctgtgttaatgatgaacccctcttcacggcagaccaggttattgaagaaattgaagaaatgatgcaggaatcaccggacccagaagatgatgaaacccctacacagtcagatcggctttcaatgctttcccaggaaattcaaactctcaagaggtctagtaccggcagttatgaagagagagtgaaaaggctctcagtgtctgagttaaatgaaatcctggaagaaattgagactgccattaaggagtactctgaggagctggtgcagcagttggctttacgagatgaactggagtttgaaaaggaagtgaaaaacagctttatttctgttcttattgaagtgcaaaacaaacagaaagagcacaaagaaacagcaaaaaagaaaaagaaactaaaaaatggcagctctcagaatgggaagaatgagagaagtcatatgcccggcacatatttgactacagtcattccttatgagaaaaaaaacggaccaccgtctgttgaagatcttcaaatattaacaaaaattcttcgtgccatgaaggaggacagtgaaaaagttccgagcttgttaactgattatattctgaaagttctgtgtcctacatagagcagcaactttatctgcggtgggctccaagctagatttccgacagcattattctgagagctggctaccattacccttcttgctattggaaactcagcacatttgaacttgggtttgattcagtattaacagatcttgactacactaattctttatattatagaaccaacggaaatatgggcactattttgaattctagagatggtttttgttaaatctactaataaactgttctcttagtagattaagagagagtaatattaattgtgcatgtgcagttgtatttctcattaactgacagtatgcccatttgtttttatggctttcttatctaaactgcactgatgaactagattaaagccttgggagatttatactataaattcagtgatggcaagaaccaacactgtttttttgtgagaattgtcagtgtaactattacctaccagtattgttcagagagattgaaacagaataaacgggctgttcttgaagaagcaaaaccagaatatgcattactttggtttaatacttagtgctaacattgaaactgttggtggtgatggattttgtagcttgctgcttgtttcaccactggtcaaattttaaccattaaattgccattcacttttagaatcttgtatttaagtaagttttgattttcaaatgttctgcttcatgtgtctgtgaagaattgtacttttttaaaagtgtgtgtcctctgaggtgcttgagaaagtgtacactgcagaactgcccattctcattactgtgtcctattttattcatgcctgtgtgtttttcttaagtatgaattctagatacagctacttatggattcatcaatatcatgagcacttttgctggttccagtcaaatcaatggcatttaataaattttttaagaagtaaaaaaaaaaaaaaaaaaa

SEQ ID NO: 47 - DNA, 3990 bp, Homo sapiens
ggagtttagggcctgacagaagcccgcccccgctggcgctcgtgcgcacgcgtggcgggctctcggcgcactgagcaggcgcggcctcgtgtcggccggagggggcgggcgcaacgacgcgcgctgcgtcccggcgctcggctttccctccgccggtcccgccctccgtcgcggcggcgcggtgtaccctgggatagggagcgatctccgagcgaggcggcaagatggacgcgggatttttccgcggaacaagtgcagaacaggataatcggttcagcaacaaacagaagaaactactgaagcagctgaaatttgcagaatgcctagaaaaaaaggtggacatgagcaaagtaaatttggaggttataaagccttggataacaaaaagagtaacggaaatccttgggtttgaagatgatgttgtgattgagtttatattcaaccagctggaagtgaagaatccagactccaaaatgatgcaaatcaacctgactggatttttgaatggaaaaaatgctcgagaatttatgggagaactgtggcccctgctgctaagtgcacaagaaaacatcgcgggaatcccttctgctttcctagaactgaagaaagaagaaataaaacaaagacagattgaacaagaaaaactggcatctatgaaaaagcaagatgaagacaaagataaaagagataaggaagaaaaagaaagcagcagagaaaaaagggagcggtctcgtagcccaagaagacgcaaatccagatctccttcccctagaagacgatcttcccctgtcaggagagagagaaagcgcagtcattctcgatctccccgtcacagaaccaagagccggagtccttcccctgctccagaaaagaaggaaaaaactccagagctcccagaaccttcagtgaaagtaaaagaaccttcagtacaagaggctacttctactagtgacattctgaaagttcccaaacctgaacctataccagagcctaaagaaccttctccggaaaaaaattccaaaaaagaaaaggagaaggagaagacccgaccacgatctcggtcacgctccaaatcaagatcccggacgcggtcccgctctccttctcacactcgacctagacggcgccatagatcccgatcaagatcgtattcacctagaaggcggccaagcccaagaaggcggccatctcctcgaagaagaactccgccaagaagaatgcctcctccaccaaggcatagaaggagtagatctccagtaagacgaagaagacgttcgtcagcatccttgtctgggagtagctcatcatcctcttcatctcgttcacggtcaccaccaaagaagcctcccaagaggacatccagcccccctcggaaaactcgtaggttatctccttcagcaagtcctccaaggcgaaggcacaggccatcacctcctgcaactccaccacccaaaactcggcattcccctacaccccagcagtcaaaccgtacaagaaaaagtcgtgtttctgtgtctccagggagaacttcaggtaaagtgacaaaacataaaggtactgagaaaagagaatccccttcaccagcaccgaagcctagaaaagtagagttatctgaatcggaagaagataaaggtggcaaaatggctgcagcagattctgtgcagcagagacgccaatacagacgacaaaaccagcagtcttcatctgactctggctcctcctcctcctcagaagatgaacgacccaagagatcccatgtgaagaatggtgaggttggcaggcggcggagacattccccttcccggagtgcttctccatcaccacgaaagcgccaaaaagagacttcccctcgtggtagacggaggagaagtccatccccaccacccaccagaaggcgacggtctccttctcccgcccctcctcctcgacggcgcaggactcccacaccaccaccacgacgaaggactccttctcctcccccacgtcggcgctcaccttctcctagaagatactctcctccaatacagaggagatactctccttctccacctccaaagagaagaacggcttcacctcctccccctcctaaacgaagagcatcaccatctccaccaccaaagcggcgggtctcccattctccacctcccaaacaaagaagctccccagtcaccaagagacgttcaccttcattatcatccaagcataggaaagggtcttccccaagccgctctacccgggaggcccgatcaccacaaccaaacaaacggcattcgccctcaccacggcctcgagctcctcagacctcctcaagtcctccacccgttcgaagaggagcgtcgtcatcaccccaaagaaggcagtccccgtctccaagtactaggcccattaggagagtctccaggactccggaacctaaaaagataaaaaaggctgcttccccaagcccacagtctgtaagaagggtctcatcctcccgatctgtctccgggtctcctgagccagcagctaaaaagcccccagcacctccatcccccgtccagtctcagtcaccgtctacaaactggtcaccagctgtaccggtcaaaaaggccaaaagcccaacaccgagcccatcaccgccaagaaattcagatcaggaaggaggtggaaagaaaaagaagaaaaagaaggacaagaaacacaaaaaggataagaagcacaagaagcacaaaaaacacaagaaggaaaaggctgtggctgcagctgctgcagctgctgtgacccctgcagccattgcagctgccacaaccacattagcacaggaagagccagtggcagcgccagagccgaagaaggagactgaaagtgaagctgaagataaccttgatgatttagaaaagcacctgcgtgaaaaggccctgagatcaatgaggaaggcccaagtgtccccacagtcttagggggaaatgtttgttatgatgtaaattttatttggtttgtacgcagttcaatttcaaaattgctaaaatgtgtttgagctttagactataacatttgttgtaataattgctaggttgaagttcaacatgtaaaaaaagggggcatggatttacattgcaaaaggtgtccacagtgtattagtgacattctttcattgacagctgacataattcattgagtgaaatattttaagccaaaaaaaaattccctttttaaaaaagggggtttaaatactgttggcatttttatggttcctttaaatgccctagctattcccagaggggtttttttgtttgtttttttggttttgattttctttttgtttttctttcttcttcttatttttttcatttgagtcttagctcccatttaagttatgcttctgaccttgtatggtctgtaagcttgcccagaaataagaccactgttttgaactaccacaaaagtataaatgaatattttaatgccacaatctttcctgttgcctgtggagtctctgctgaaatgaatcaggattcgagctctaggatgagacagaaaatgaaagcatgttgtttgccaggacactgtgggtttatattgatgtgtaacaagttgatttggaacactggactctcattctgttattctggttttgttttttttgttttgttttttttcttttgtaaaggcaatgagctagtcccagaaaggatccttcagttacatacaatttgtttaatgaaatgtcatggctctgttcatatttttgtcttgttcttccaattggtatatacaactttcagagcctcttgtatttggaaggctggaagggcccagactttggaatagtgtcttggtttcactgtttttgttttgattttttttttgttttgattttttttaaactaaagctatataaagcttgtggattaaacagaataaatttctaaatttaaaaatttaaaaaaaaaaaaaaaaaaaaaaaaaaaaaaaaaaaaaaaaaaaaaaaaaggaaaaaaaaaaaaaaaa

SEQ ID NO: 48 - DNA, 1502 bp, Homo sapiens
cctgcgtccccgccccgcgcagccgccgcgctcctgcgctccgaggtccgaggttcccgagatgaaggtctggctgctgcttggtcttctgctggtgcacgaagcgctggaggatgttactggccaacaccttcccaagaacaagcgtccaaaagaaccaggagagaatagaatcaaacctaccaacaagaaggtgaagcccaaaattcctaaaatgaaggacagggactcagccaattcagcaccaaagacgcagtctatcatgatgcaagtgctggataaaggtcgcttccagaaacccgccgctaccctgagtctgctggcggggcaaactgtagagcttcgatgtaaagggagtagaattgggtggagctaccctgcgtatctggacacctttaaggattctcgcctcagcgtcaagcagaatgagcgctacggccagttgactctggtcaactccacctcggcagacacaggtgaattcagctgctgggtgcagctctgcagcggctacatctgcaggaaggacgaggccaaaacgggctccacctacatcttttttacagagaaaggagaactctttgtaccttctcccagctacttcgatgttgtctacttgaacccggacagacaggctgtggttccttgtcgggtgaccgtgctgtcggccaaagtcacgctccacagggaattcccagccaaggagatcccagccaatggaacggacattgtttatgacatgaagcggggctttgtgtatctgcaacctcattccgagcaccagggtgtggtttactgcagggcggaggccgggggcagatctcagatctccgtcaagtaccagctgctctacgtggcggttcccagtggccctccctcaacaaccatcttggcttcttcaaacaaagtgaaaagtggggacgacatcagtgtgctctgcactgtcctgggggagcccgatgtggaggtggagttcacctggatcttcccagggcagaaggatgaaaggcctgtgacgatccaagacacttggaggttgatccacagaggactgggacacaccacgagaatctcccagagtgtcattacagtggaagacttcgagacgattgatgcaggatattacatttgcactgctcagaatcttcaaggacagaccacagtagctaccactgttgagttttcctgacttggaaaaggaaatgtaatgaacttatggaaagcccatttgtgtacacagtcagctttggggttccttttattagtgctttgccagaggctgatgtcaagcaccacaccccaaccccagcgtctcgtgagtccgacccagacatccaaactaaaaggaagtcatccagtctattcacagaagtgttaacttttctaacagaaagcatgattttgattgcttacctacatacgtgttcctagtttttatacatgtgtaaacaattttatataatcaatcatttctattaaatgagcacgtttttgtaaaaaat

SEQ ID NO: 49 - DNA, 385 bp, Homo sapiens
aaaaaaattaaggctaaccaagtgcatccattgttcaatggcacaattgatttcagcaactatttggaatatcctaattataggaaatgcccatctaagtgatatatttaaataatacaatcaattttttaaggtgaataaactatgatggtttctaaatagtgtacatgttacctgaaaaatcagaaaacacaaagaatgattaatttcgaaagttcttgcctaaaggcaccactgacttaaaaaacattcaaaatcaaataccacaagacataaagcctcttcatgtatatattcatatatgcaataaatgcattaaatgtaactttattaaacatagtacactgtacttgacttatggttaaatattttacacacagcttga

SEQ ID NO: 50 - DNA, 6313 bp, Homo sapiens
gccaggtccctgaggggcgggcagatgaggcctaggggtgccgatccctagtgtcgactatgcgagatctgattccggagctgccatgattgaagtggtagcagagctcagccggggtcctgtatttttggctggggaggcgctggagtgtgtagtgaccgtcaccaacccccttccgcccacggccacttctgcatccagtgaggccctggcctgggccagtgcccaaatccactgccagttccatgccagtgagagtcgagtagcactgcctcctcctgactctagtcagccagatgtccagcccgacagccagactgtctttctgccacaccgaggtgagaggggccagtgtatcctttctactccaccgaaaattctattctgtgacctgaggcttgatcctggagagtccaaatcatactcctacagtgaagtgctgcccatagagggaccaccctcctttcggggtcagtcagtcaagtacgtctacaaactgaccattggctgccagcgtgtcaactcccctatcactttactcagagtccctctgagggttcttgtgctgactggccttcaggatgtccggtttccccaggatgaggctgtagccccatccagtccattcttggaggaggatgaaggtgggaagaaagattcatggctagctgagctggctggggaacgcctaatggctgccacatcctgccgcagcctccatctatacaatatcagtgatggccgagggaaagttgggacgtttggcatcttcaaatctgtgtacagacttggcgaggacgtggtggggaccttaaacttaggggaaggaaccgtagcttgtttgcagttttcagtcagcttacagaccgaggagcgtgtacagcctgagtaccagcggcgacgtggggcagggggtgtcccctctgtgtcacatgtgactcacgcccggcaccaggaatcctgcctacatacaactagaaccagcttctccctcccaatccctctcagctccaccccaggcttctgtacagccattgtgtccttgaagtggagattgcattttgaatttgtaacgtcccgagaaccaggattggtactcctaccccctgtggaacagcccgaacctaccacctggacaggacctgagcaagtacctgtagacaccttcagctgggacctgcccatcaaggtgctgcctactagccccaccctggcctcatatgctgccccaggccccagcaccagcaccataaccatctgaaactggcccaccctggtgctagttccttccggatactgagaactcagcacctggactctaatgggacccactttttccacctggggtccaatgtcgtggacagtgagagtcgggctttcagctatagcattaatttatttgttcagaatacattggcagctgctagtggtttccctggaagtggcagcagcagtgagcagtcagcagatggatgatcagttgagtttagctggagtggggagcaggagccccaggaacaggggtgttggctgagccccattctgggtcaggccctccccctttgcagggcagccgagggtcagatttttgcaccaaggagaactggcaggttcctgcctcctgacgtacctcacacccagccgggaagtcgatgggatgctgggacctggggaaccaaggataggggaaggagtcagcacagtgaaaggctgcctttatccctgcccacatgttccctctctcacagttttccccccacagagcccctttcagtggccccttggtcctcctaactaagctgtcacctaccatatgtgggcctttttgttttataacaggagtattttctctccaggtccaccccaacctcccctgatttatagcctgaagccttatctttcacactagtgttggtcccttcaggtttggcccatcttgtattgctcttctgttcattcttacatcacagcaatctagtcactccctggtcatccctcagtcactcatatcagagtcattctctctggccatctttggtcactcacgtgtcacagcagcccacgccaacaggatgcagacaggtgcaatggaaacagtccttgcggagccaagactcacccagggtaaaatatttcccctcatagtgacagggggctagggaagaacgggaaatgttagtaggtgtaggagtgctgatgagaggcagaggctcttctggtctggggtggagacagtaagtacgcactatccccgtatttagtttgtctttcctgtttcacagctggaggaagcctgggtattttgacacgggatcatctgtaaggccccatcctccctgtgccctctctgctgctcctccattcctaacgcttcaccccactttaccttgagcttggaagtagcacttgctgtagactcctgggtgctggaggagtagagacatcaccaagcagatgatcccccagcctcctaggatccccttggcctgtccagcccagagcatccttagggccattgctgctgcacagccctctcagacccttcttggcctctgctcagctactctggtcttgactccttgactttgctttgcgttgctccttgagtcttagtttctgtctttctcccctgggctcctgtctcacactatctccctgccctctgctctcacaggctggggatgtttataaagtgaggaccctggccccctgctgagtagagctggaaaagttgtaactctgtttcctgaggtgagggcatgaaaacaagaggtctagctttaacaagctgtgagagctgattcatgccccggcacagctagagggagggaggtggccatggagggggcactggactgggcacttccccagcaaggaggcaggaggggcgagggcccccaggtggtccccagatctcttccctgacctggagagaaggaagcattccaccttccccctttctcccccactgccaccaccaggggtgtgtatgctgggatccctgcctggaccggagggaggcatttcctggggatggttaatcctgtgccccagccaaacccaggagctgcaatagggtgcgacggccagaagctccaggagagtgagcaggcacctggagtggagactgtgtttccctcagatcctagggcagggtttccctaatgtatccaagaaatagggctgcccctcagagatggtggggagggtctcttttcctcaggcattccagaggtgaactgtccattgcttatcaccttcaaacatacagcagatgtgggatcaccccacatctggggatggttctttcccctttcaaagaggagcatctctaagtgccctgatgggatgaatcactccaggttcacagaggtgtcctctctttcctcccatatataatggagtgaggtttttaggaatttatcatttggcatcctctgagtttcccacaggttctggaggagcccaggatggattattgagagcatgggctgtagagacagtcttcttggattcagatcctgactccacttagctatgtaacctggtcagattacttcacctctctgagcctgtttcctcatctataaattggggatagtaatgccaactcattgggctgttatgaggattactgagataatgcgtgcagtgctcttatcaccatctctggtgcgtaagcgtcaggaaatagcagttgctgtgattggggctaaagctctgaggcaaaatgggcgacattattttctttgaatgacattaagcagtttgtgcatagctgagggcttctattggggatggctgtctcctggcatagacctctgcacctttcacactcatactccttgtcagcagtccccaacctttttggtaccagggaccggttttgtggaaaacaatttttccaccagtggatggagggggatagcagcggggagatgattttgggatgaaactgtttcatctcagatcatcaggcattagattctcataaggagtgtgcaatctagatcccttgcatgcggagttcacagtggggtttgcactcctgtgagaatctaatgcctctgctgatctgccaggaggaggagctcaggcggtaatgctcactcgcctgccgcccacctcctgctttgtgctcccgcttcctaacaggccacagactggtactggcctgtggcctgggggatggagacccctaatccatgtcacctttcccacctctttcaaaaacaggtacctccaggaacattttggttttggcccttgtattgacttctgaatgtctagtttgagaaactgttcccaaataagccttcttcccccagatctgcaccctcgcctctaccctaggacaagatgtccttttctcatcatcctgccaggctaactttaagtctcctgctttttctcacttggatttggatccatttcttcctatttccgctcatgtgaactctccagttctcctttctcaccactctcctgctagccatctctttggcactaaaggccctggtcaaattggatttctttcatttttccacacttcaaagacccatgttctaggtattctccatagggatagtctctttggcatttatttggtttttctacgttttcagtcccatttactccaagactcactccctgccacctagtgcatcagatacagctacttctggctgacttttcaagggggaccaccctacctgtcatctcttcactgttcagaaatgactgtgtcagtgcacctcaaactcccttgctgtccttttccaaggagacagctaaggtggatggagatgcagaatggacctcacgttcgccctagtcaggactgataccctttccgtttcagaggattgccaagaaaaaactcacagttgaggcagggtgctctgaggtcggctgcggtgtgggaggcacggcctgggcctgctctctgggctggagcaggtggattcgaaggcctgtctagcacgagggcccaaaggtcttgtcagtggccagtagctctgccgcctttcccagagagggggtccaggggacatcctggaaggctgggccctgggccaccttctgctcttgcaagctagagccagcccaatagggggcggatgtgagtggggagctggggcgcatgaaggtgggggtgatgccgaaggggaagggatcgccagtggggattggtgcgtgtgcggaaacggggacagaagtgaaggttcatcgcctataacgaagatgaggtaggcatataggggcttctggaaagctagaggctgggctgagccaggagtcctctcccagaagttggggggcggtgcagaggtgtgggtcgagcccgcatgcgtgcctgctggggagggggtgagtggtgaggaccaggcccgctgggtcctgggggcgcggtggctggcgcgcaggtcccggagggggcggctggcgcgcactacacgcttgggaacaaggaaaacatccgccggaggcccggccgggcggcgctccagcctcggggcaggtgcgcggagaggaagtgagagcattccggcccccccaccccaaccccggccgctggccctctggtgagtcacagccgacccccgccgccggagggagaggggagctgcgggccagagccccggagggtctggaggagccaggagggtttctgggagcagagggtcacttagtgggcttctgtcgtggtgtcgctacgggcgcgaaacggacactgaacacagtctgactgtatggaggcaggtggggagggatcccctgggagaacttggcgggccgagagcagaccccagggcaaggaggggcccccgagggggaaaccgggagtcgggcaggtggcgtaacccagaaagggaaggagagccggattgattggggtgagagaggaaggaagcacgccaagttaggcctgggagaactgagggacctgaggagggaggagggagaccaacacagggtgggaaggcggaaatggccaaaccccaggcatcaggtctgtccagaggctgacgtagacagtgaagggtgaagggtaggttttaggagtagggggagttatgattatttggttacattttgggattatttggtctcacaggtagaagggagcctgctggtctctgtgtaacggatggcttaaaagcaaggttgtctgcgtcttggattactgtctgccattcagcctttgccaaaaaatttggcactgatctgcacatttttatagtcatttaaaattgtatgactctgtcaaatgatttaagtaattttggtggatttttaaaaataaaaaaat

SEQ ID NO: 51 - DNA, 1133 bp, Homo sapiens
ggtaaggggtcctccctgcgccacacggccgtcgccatggtgaagctgagcaaagaggccaagcagagactacagcagctcttcaaggggagccagtttgccattcgctggggctttatccctcttgtgatttacctgggatttaagaggggtgcagatcccggaatgcctgaaccaactgttttgagcctactttggggataaaggattatttggtcttctggatttggaggcaatcagcggacagcatggaagatgtgtgctctggctcggataagagatgggacatcattcagtcactagttggatggcacaaggctcttcacagacgcatctgtagcagagtggatcttgtactaacttatgatagaatgtatcagaataaatgtttttaacagtgtaaacaccacaaacaaaaaacacaacacacacatcatacacacaaaaaacacaaaaaaaacaaacaaatcacacaaaagctacggtagacctactattatgcggtgggccgaaacaagacgggtattatagacaagggaaacgagtcgtcaaatcgtcgtagcctgacacacatcatattgttagacccagcgtgtgcaatatctcgccggggtagctccctcatatgagggacacgttatatatgtctcagatagggcgccggggtataacctgcagttttatagatatgctggcaacagaaaaaagcgatgtaaaaaaaaaaatgaagacaacataaacacacacacaaagatactatcacatatatactataaccaaaaaatctcaaagcgtaaatcaaaaatacactaaaacaattcacagccatattcactacaccctatccaccccacacaaaaaaaataagacacaaaacatcacacatatacacactacctatcattttatactttaatctaatataattaagtaacaaatcaacacaaatatacacacgatcgatagatacactgataaaattcaacaaacaaaataccaaataaaatatactaaacacaccactagacgagcatcttatattgcacttttacgtagacctctgatcaataacaacagacctactccacaaatatactactaacacacaaacaatgcaaacagcacagaataac

SEQ ID NO: 52 - DNA, 20 bp, Artificial
  Oligonucleotide primer
gccataagtggtcccacagt

SEQ ID NO: 53 - DNA, 23 bp, Artificial
  Oligonucleotide primer
gtcttctagtccgtcatctccct

SEQ ID NO: 54 - DNA, 22 bp, Artificial
  Oligonucleotide primer
ccatagctgcctttatgtctgc

SEQ ID NO: 55 - DNA, 22 bp, Artificial
  Oligonucleotide primer
ctttttaccttcgtgcaccttt

SEQ ID NO: 56 - DNA, 23 bp, Artificial
  Oligonucleotide primer
taatctgctgaggaccttttgtc

SEQ ID NO: 57 - DNA, 23 bp, Artificial
  Oligonucleotide primer
taattcactgtcctcttctggga

SEQ ID NO: 58 - DNA, 21 bp, Artificial
  Oligonucleotide primer
gctcacagcagtaaatgccta

SEQ ID NO: 59 - DNA, 23 bp, Artificial
  Oligonucleotide primer
tgctatgctgtaaacactggcta

SEQ ID NO: 60 - DNA, 23 bp, Artificial
  Oligonucleotide primer
ggatcctttattggtggtagagc

SEQ ID NO: 61 - DNA, 23 bp, Artificial
  Oligonucleotide primer
ccagagtgaccctgaagataaat

SEQ ID NO: 62 - DNA, 23 bp, Artificial
  Oligonucleotide primer
acctgattctctaggtgcagttt

SEQ ID NO: 63 - DNA, 23 bp, Artificial
  Oligonucleotide primer
gtcgtttcaaccaggtagttttg

SEQ ID NO: 64 - DNA, 20 bp, Artificial
  Oligonucleotide primer
gggcgccttaagttattgga

SEQ ID NO: 65 - DNA, 22 bp, Artificial
  Oligonucleotide primer
ggatggtagaaaagcaaactgg

SEQ ID NO: 66 - DNA, 23 bp, Artificial
  Oligonucleotide primer
tgtaatggagattgtacaggttg

SEQ ID NO: 67 - DNA, 23 bp, Artificial
  Oligonucleotide primer
aggaacagtacaaatgctgtggt

SEQ ID NO: 68 - DNA, 23 bp, Artificial
  Oligonucleotide primer
gcactccttgaaggtacactaac

SEQ ID NO: 69 - DNA, 22 bp, Artificial
  Oligonucleotide primer
atttgtattcactcagccatgc

SEQ ID NO: 70 - DNA, 23 bp, Artificial
  Oligonucleotide primer
acccaacttcaaaactaggactc

SEQ ID NO: 71 - DNA, 23 bp, Artificial
  Oligonucleotide primer
acagcttgatgtcctttctatgc

SEQ ID NO: 72 - DNA, 22 bp, Artificial
  Oligonucleotide primer
tcatgaaaggcactgagttttg

SEQ ID NO: 73 - DNA, 23 bp, Artificial
  Oligonucleotide primer
gttagctgaagcagctttattgc

SEQ ID NO: 74 - DNA, 22 bp, Artificial
  Oligonucleotide primer
atatgcacaatcctggaagtga

SEQ ID NO: 75 - DNA, 23 bp, Artificial
  Oligonucleotide primer
tgccttactagcattaccaccat

SEQ ID NO: 76 - DNA, 21 bp, Artificial
  Oligonucleotide primer
gaggtgatagcattgctttcg

SEQ ID NO: 77 - DNA, 21 bp, Artificial
  Oligonucleotide primer
caagtcagtgtacaggtaagc

SEQ ID NO: 78 - DNA, 21 bp, Artificial
  Oligonucleotide primer
tcttacacccagtggagaagc

SEQ ID NO: 79 - DNA, 20 bp, Artificial
  Oligonucleotide primer
gtctttgtgttcccggacat

SEQ ID NO: 80 - DNA, 20 bp, Artificial
  Oligonucleotide primer
actatgtccgggaacacaaa

SEQ ID NO: 81 - DNA, 18 bp, Artificial
  Oligonucleotide primer
ttccgtcatatggcttgg

SEQ ID NO: 82 - DNA, 23 bp, Artificial
  Oligonucleotide primer
cgtcgctatcaaggaattaagag

SEQ ID NO: 83 - DNA, 23 bp, Artificial
  Oligonucleotide primer
gtagctccagacatcactctggt